(19) United States
(12) Patent Application Publication
(10) Pub. No.: US 2024/0366728 A1
(43) Pub. Date: Nov. 7, 2024

(54) Treatment of Diabetes Mellitus

(71) Applicant: Carmot Therapeutics Inc., Berkeley, CA (US)

(72) Inventors: Manu Chakravarthy, Newton, MA (US); Ruben Rodriguez, Richmond, CA (US); Gabriele Sonnenberg, West Hollywood, CA (US)

(73) Assignee: Carmot Therapeutics Inc., Berkeley, CA (US)

(21) Appl. No.: 18/644,041

(22) Filed: Apr. 23, 2024

Related U.S. Application Data
(60) Provisional application No. 63/497,737, filed on Apr. 23, 2023.
(60) Provisional application No. 63/509,743, filed on Jun. 22, 2023.
(60) Provisional application No. 63/590,388, filed on Oct. 13, 2023.

(51) Int. Cl. A61K 38/26; A61K 9/00; A61K 47/54; A61P 3/10

(52) U.S. Cl. CPC A61K 38/26; A61K 9/0019; A61K 47/542; A61K 47/543; A61K 47/545; A61P 3/10

(57) ABSTRACT

The present disclosure provides methods and compositions for treating patients with diabetes mellitus and/or obesity.

### CROSS-REFERENCE TO RELATED APPLICATIONS

The present application claims priority from U.S. Applications 63/497,737, filed Apr. 23, 2023; 63/509,743, filed Jun. 22, 2023; and 63/590,388, filed Oct. 13, 2023. The contents of the priority applications are incorporated by reference herein in their entirety.

### SEQUENCE LISTING

The instant application contains a Sequence Listing that has been submitted electronically in XML format and is hereby incorporated by reference in its entirety. Said XML copy, created on Apr. 23, 2024, is named 124921US004.xml and is 4,912 bytes in size.

### BACKGROUND OF THE INVENTION

Diabetes mellitus (DM), commonly known as diabetes, is a disease caused by inadequate control of blood glucose levels. It has two primary phenotypes-Type 1 (T1DM or TID) and Type 2 (T2DM or T2D). T1DM, once known as juvenile diabetes or insulin-dependent diabetes, is a chronic condition. T1DM is thought to be caused by autoimmunity against the beta cells in the pancreas such that the patients lose the ability to make any or enough insulin, a hormone that helps cellular intake of blood glucose. While genetic predisposition plays a role, a trigger in the environment, such as a virus infection, is often associated with the onset of T1DM. Although T1DM often appears during childhood or adolescence, it can develop in adulthood.

T2DM results from the inability of insulin to properly control blood glucose. This state of insulin resistance can cause the pancreas to secrete more insulin, resulting in hyperinsulinemia; yet blood glucose levels remain abnormally elevated. Over time, this condition results in beta cell failure and insufficient insulin production, cardiovascular disease, and a significant increase in the risk of heart attacks, strokes, neuropathy, limb amputation, blindness, and kidney failure. Obesity is a major risk factor for T2DM.

Treatment of diabetes typically focuses on glycemic control, e.g., by intake of exogenous insulin (e.g., by subcutaneous injection or powder inhalation). However, insulin therapy often results in chronic hyperinsulinemia. Indeed, the basal insulin levels in diabetes patients may be as many as several times higher than in healthy people (see, e.g., Gregory et al., Diabetes (2019) 68 (8): 1565-76). Hyperinsulinemia, especially in the setting of increased adiposity, induces insulin resistance, which can lead to even higher weight gain, peripheral adiposity, and obesity (see, e.g., Van der Schueren et al., Lancet Diabetes Endocrinol (2021) 9 (11): 776-85; Schauer et al., Diabetes (2011) 60 (1): 306-14). This combined state of insulin resistance with insulin deficiency is often termed “double diabetes.” Furthermore, even patients with ideal glycemic control (hemoglobin A1c [HbA1c]<7%) remain at high risk for cardiovascular disease and are prone to obesity, and these issues can be exacerbated by insulin therapy.

Another chronic disease that affects large swathes of the world population is obesity. Obesity is associated with numerous complications including cardiovascular disease and T2DM, posing a substantial health and economic burden on patients and society.

Thus, there remains an acute need for developing a safe, effective, and well-tolerated therapy for people with chronic conditions such as diabetes and obesity.

### SUMMARY OF THE INVENTION

The present disclosure provides methods of treating T1DM or T2DM or improving glycemic control in a patient in need thereof. In some embodiments, the patient is insulin-dependent, i.e., a patient that requires insulin intake or therapy (through, e.g., injections). In further embodiments, the patient has T1DM or T2DM. In some embodiments, the patient may be insulin resistant (e.g., a patient with T2DM). Also provided is a method of weight management in a patient in need thereof.

In one aspect, the present disclosure provides an adjunctive therapy for an insulin-dependent patient (e.g., a patient with T1DM or T2DM), comprising administering (e.g., by subcutaneous injection) to a patient in need thereof a dual agonist of glucagon-like peptide 1 (GLP-1) receptor (GLP-1R) and glucose-dependent insulinotropic polypeptide (GIP) receptor (GIPR), wherein the agonist comprises the following structure or a pharmaceutically acceptable salt or ester thereof:

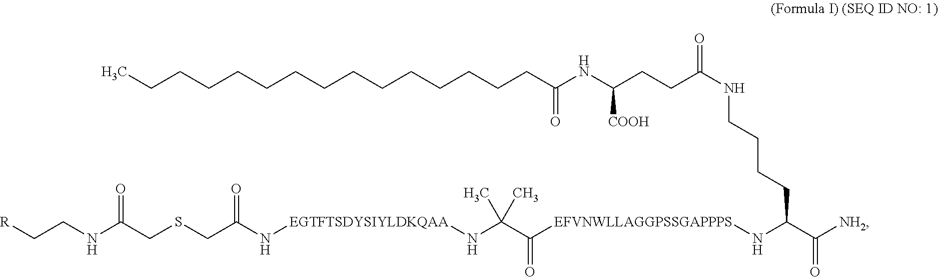

wherein R is a cycloalkyl or heterocyclic group that is 4-8 atoms in size, optionally substituted with a carbonyl, hydroxyl, methyl, phenyl, isopropyl, trifluoromethyl, or nitro group; and wherein the agonist is administered at a body weight-independent dose (i.e., a flat dose or fixed dose) of 1 mg to 20 mg.

In one aspect, the present disclosure provides a method of treating T1DM or T2DM in a patient, or improving glycemic control in a patient with T1DM or T2DM, comprising administering (e.g., by subcutaneous injection) to the patient in need thereof a dual agonist of glucagon-like peptide 1 (GLP-1) receptor and glucose-dependent insulinotropic polypeptide (GIP) receptor, wherein the agonist comprises the structure of Formula I or a pharmaceutically acceptable salt or ester thereof, wherein R is a cycloalkyl or heterocyclic group that is 4-8 atoms in size, optionally substituted with a carbonyl, hydroxyl, methyl, phenyl, isopropyl, trifluoromethyl, or nitro group; and wherein the agonist is administered at a body weight-independent dose (i.e., a flat dose or fixed dose) of 1 mg to 20 mg.

In one aspect, the present disclosure provides a method of increasing insulin sensitivity or reducing insulin resistance in a patient with T1DM or T2DM, comprising administering (e.g., by subcutaneous injection) to the patient in need thereof a dual agonist of glucagon-like peptide 1 (GLP-1) receptor and glucose-dependent insulinotropic polypeptide (GIP) receptor, wherein the agonist comprises the structure of Formula I or a pharmaceutically acceptable salt or ester thereof, wherein R is a cycloalkyl or heterocyclic group that is 4-8 atoms in size, optionally substituted with a carbonyl, hydroxyl, methyl, phenyl, isopropyl, trifluoromethyl, or nitro group; and wherein the agonist is administered at a body weight-independent dose (i.e., a flat dose or fixed dose) of 1 mg to 20 mg.

In one aspect, the present disclosure provides a method of increasing insulin-independent glucose disposal in a patient with T1DM or T2DM, comprising administering (e.g., by subcutaneous injection) to the patient in need thereof a dual agonist of glucagon-like peptide 1 (GLP-1) receptor and glucose-dependent insulinotropic polypeptide (GIP) receptor, wherein the agonist comprises the structure of Formula I or a pharmaceutically acceptable salt or ester thereof, wherein R is a cycloalkyl or heterocyclic group that is 4-8 atoms in size, optionally substituted with a carbonyl, hydroxyl, methyl, phenyl, isopropyl, trifluoromethyl, or nitro group; and wherein the agonist is administered at a body weight-independent dose (i.e., a flat dose or fixed dose) of 1 mg to 20 mg.

In one aspect, the present disclosure provides a method of reducing the need for therapeutic insulin in a patient with T1DM or T2DM, comprising administering (e.g., by subcutaneous injection) to the patient in need thereof a dual agonist of glucagon-like peptide 1 (GLP-1) receptor and glucose-dependent insulinotropic polypeptide (GIP) receptor, wherein the agonist comprises the structure of Formula I or a pharmaceutically acceptable salt or ester thereof, wherein R is a cycloalkyl or heterocyclic group that is 4-8 atoms in size, optionally substituted with a carbonyl, hydroxyl, methyl, phenyl, isopropyl, trifluoromethyl, or nitro group; and wherein the agonist is administered at a body weight-independent dose (i.e., a flat dose or fixed dose) of 1 mg to 20 mg.

In one aspect, the present disclosure provides a method of alleviating hypertension in a patient in need thereof, such as a patient with T1DM or T2DM, comprising administering (e.g., by subcutaneous injection) to the patient in need thereof a dual agonist of glucagon-like peptide 1 (GLP-1) receptor and glucose-dependent insulinotropic polypeptide (GIP) receptor, wherein the agonist comprises the structure of Formula I or a pharmaceutically acceptable salt or ester thereof, wherein R is a cycloalkyl or heterocyclic group that is 4-8 atoms in size, optionally substituted with a carbonyl, hydroxyl, methyl, phenyl, isopropyl, trifluoromethyl, or nitro group; and wherein the agonist is administered at a body weight-independent dose (i.e., a flat dose or fixed dose) of 1 mg to 20 mg.

In one aspect, the present disclosure provides a method of reducing atherogenic lipids (e.g., cholesterols, triglycerides, and/or low-density lipoprotein cholesterol) in a patient in need thereof, such as a patient with T1DM or T2DM, comprising administering (e.g., by subcutaneous injection) to the patient in need thereof a dual agonist of glucagon-like peptide 1 (GLP-1) receptor and glucose-dependent insulinotropic polypeptide (GIP) receptor, wherein the agonist comprises the structure of Formula I or a pharmaceutically acceptable salt or ester thereof, wherein R is a cycloalkyl or heterocyclic group that is 4-8 atoms in size, optionally substituted with a carbonyl, hydroxyl, methyl, phenyl, isopropyl, trifluoromethyl, or nitro group; and wherein the agonist is administered at a body weight-independent dose (i.e., a flat dose or fixed dose) of 1 mg to 20 mg.

In one aspect, the present disclosure provides a method of weight management (weight loss) in a patient in need thereof, such as a patient with T1DM or T2DM, comprising administering (e.g., by subcutaneous injection) to the patient in need thereof a dual agonist of glucagon-like peptide 1 (GLP-1) receptor and glucose-dependent insulinotropic polypeptide (GIP) receptor, wherein the agonist comprises the structure of Formula I or a pharmaceutically acceptable salt or ester thereof, wherein R is a cycloalkyl or heterocyclic group that is 4-8 atoms in size, optionally substituted with a carbonyl, hydroxyl, methyl, phenyl, isopropyl, trifluoromethyl, or nitro group; and wherein the agonist is administered at a body weight-independent dose (i.e., a flat dose or fixed dose) of 1 mg to 20 mg.

In some embodiments, the dual GLP-1R/GIPR agonist comprises the following structure or a pharmaceutically acceptable salt or ester thereof:

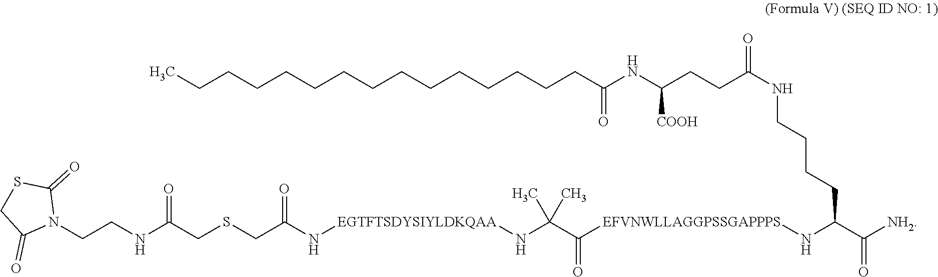

In some embodiments, the dual GLP-1R/GIPR agonist comprises the following structure or a pharmaceutically acceptable salt or ester thereof:

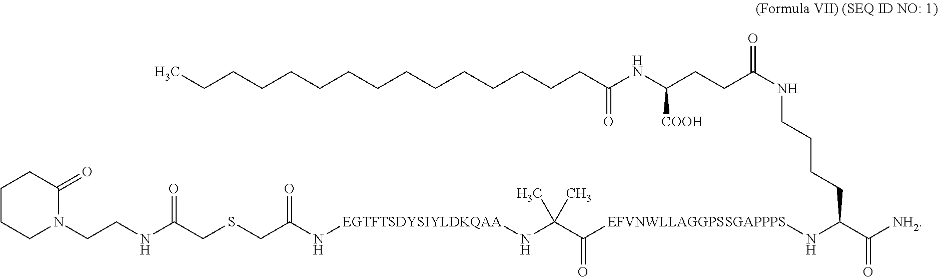

In some embodiments, the administering step in the method is repeated at an interval of one to seven days. In further embodiments, the administering step is repeated once daily.

In some embodiments, each administered dose is a dose that agonizes both GLP-1 receptor and GIP receptor. In some embodiments, the administered dose is about 1.5, 1.8, 2, 3, 4, 5, 6, 7, 8, 9, 10, 11, 12, 13, 14, 15, 16, 17, 18, 19, or 20 mg.

In some embodiments, the patient has a BMI of 25 kg/m² or higher.

In some embodiments, the patient is an adult patient, an adolescent patient, or a pediatric patient.

In some embodiments, the patient receives another therapy for T1DM or T2DM, such as insulin therapy, diet therapy, exercise therapy, hypertension therapy, and/or blood lipid-lowering therapy.

The present disclosure also provides the present dual GLP-1R/GIPR agonist for use in a therapeutic method herein; and the use of the present dual GLP-1R/GIPR agonist for the manufacture of a medicament for use in a therapeutic method herein.

In another aspect, the present disclosure provides an article of manufacture (e.g., a kit, a syringe such as a pre-filled syringe, or an injector such as a pre-filled injector) for use in the present therapeutic method. The article of manufacture comprises one or more units of the dose to be administered (e.g., about 1 to 20 mg, such as 1.8, 2, 3, 4, 5, 6, 7, 8, 9, 10, 11, 12, 13, 14, or 15 mg, per dose). In some embodiments, the syringe or injector comprises one single dose unit and may be single-use.

Also provided is a pharmaceutical composition comprising the present dual GLP-1R/GIPR agonist, or a pharmaceutically acceptable salt or ester thereof, at a concentration of 15 mg/mL; (ii) di-sodium hydrogen phosphate heptahydrate/sodium dihydrogen phosphate monobasic buffer solution, optionally at a concentration of 20 mM; (iii) propylene glycol; and (iv) phenol, further optionally wherein the pharmaceutical composition is at pH 7.0.

Other features, objectives, and advantages of the invention are apparent in the detailed description that follows. It should be understood, however, that the detailed description, while indicating embodiments and aspects of the invention, is given by way of illustration only, not limitation. Various changes and modification within the scope of the invention will become apparent to those skilled in the art from the detailed description.

### BRIEF DESCRIPTION OF THE FIGURES

FIG. 1A depicts a left plot comparing cAMP producing activities of GLP-1, liraglutide, and CT-859 (also termed herein “CT-9859”); and a right plot comparing cAMP producing activities of GIP, liraglutide, and CT-859. X-axis shows concentration in nmol, and Y-axis shows % activity.

FIG. 1B depicts a left plot comparing cAMP producing activities of GLP-1, liraglutide, exendin-4 (Ex4), exendin-Phe1 (Ex-Phe1) and CT-859; and a right plot comparing cAMP producing activities of GIP and CT-859. X-axis shows log concentration in nM, and Y-axis shows % activity.

FIG. 2A depicts a left plot comparing β-arrestin coupling activities of GLP-1, liraglutide, and CT-859; and a right plot comparing β-arrestin coupling activities of GIP and CT-859. X-axis shows concentration in nmol, and Y-axis shows % activity.

FIG. 2B depicts a left plot comparing β-arrestin coupling activities of GLP-1, liraglutide, exendin-4 (Ex4), exendin-Phe1 (Ex-Phe1) and CT-859; and a right plot comparing β-arrestin coupling activities of GIP and CT-859. X-axis shows log concentration in nM, and Y-axis shows % activity.

FIG. 3 depicts plots showing blood glucose levels (mg/dL) (left plot), AUC blood glucose (min*mg/dL) (middle plot), and plasma insulin levels (μIU/mL) (right plot) during an intra peritoneal glucose tolerance test in regular mice (GLP1R^{+/+}) and GLP-1R knockout mice (GLP1R^{-/-}) treated with vehicle, and 20 nmol/kg or 200 nmol/kg doses of CT-859.

FIG. 4A depicts plots showing blood glucose levels (mg/dL) in lean mice treated with vehicle, 20 nmol/kg liraglutide (“lira”) or 20 nmol/kg CT-859 after 4 hours (left plot), 24 hours (middle plot), and 48 hours (right plot).

FIG. 4B depicts a plot showing AUC blood glucose (min*mg/dL) in lean mice treated with vehicle, 20 nmol/kg liraglutide, or 20 nmol/kg CT-859 after 4 hours, 24 hours, and 48 hours.

FIG. 5 depicts a plot showing plasma concentration of liraglutide and CT-859 in lean mice 0.5, 1, 2, 4, 6, 8, 10, 12, 24, and 32 hours after treatment with 1 mg/kg CT-859 or 1 mg/kg liraglutide.

FIG. 6 depicts a plot showing fasting blood glucose levels (mg/dL) in diet-induced obese (DIO) mice dosed for 20 days with vehicle, 20 nmol/kg liraglutide, or 20 nmol/kg CT-859.

FIG. 7 depicts plots showing fasting plasma insulin levels (μIU/mL) (left plot) and log (HOMA-IR) a surrogate measurement of insulin resistance (right plot) in diet-induced obese (DIO) mice dosed for 20 days with vehicle, 20 nmol/kg liraglutide, or 20 nmol/kg CT-859.

FIG. 8 depicts a plot showing weight loss (WL) in diet-induced obese (DIO) mice dosed for 20 days with vehicle, 20 nmol/kg liraglutide, or 20 nmol/kg CT-859.

FIG. 9 depicts a plot showing cumulative food intake (FI) (g) in diet-induced obese (DIO) mice dosed for 20 days with vehicle, 20 nmol/kg liraglutide, or 20 nmol/kg CT-859.

FIG. 10 depicts plots showing plasma insulin levels (μIU/mL) (left plot) and log (HOMA-IR), a surrogate measurement of insulin resistance (right plot), in diet-induced obese (DIO) mice dosed for 20 days with vehicle, 200 nmol/kg liraglutide, or 200 nmol/kg CT-859.

FIG. 11 depicts a plot showing weight loss in diet-induced obese (DIO) mice dosed for 20 days with vehicle, 200 nmol/kg liraglutide, or 200 nmol/kg CT-859.

FIGS. 12A and 12B depict plots showing food intake (g/24 h) in ob/ob mice dosed for either 1, 7, or 14 days (FIG. 12A) or 8 days (FIG. 12B) with vehicle, 200 nmol/kg liraglutide, or 200 nmol/kg CT-868.

FIG. 13 depicts a plot showing weight change in wildtype mice measured at baseline, 4-hours, 24-hours, 48-hours, and 72-hours following administration of vehicle, 0.025 nmol/kg of exendin-4 (Ex-4), and 0.025 nmol/kg of exendin-Phe1 (Ex-Phe1). X-axis shows time in hours (hrs), and Y-axis shows % change in body weight from baseline.

FIG. 14 depicts a plot showing cumulative food consumption in wildtype mice measured at baseline, 4-hours, 24-hours, 48-hours, and 72-hours following administration of vehicle, 0.025 nmol/kg of exendin-4 (Ex-4), and 0.025 nmol/kg of exendin-Phe1 (Ex-Phe1). X-axis shows time in hours (hrs), and Y-axis shows cumulative food consumption in gram (g).

FIGS. 15A-15J present data showing that CT-859 is a biased dual GLP-1R/GIPR agonist. These figures show the mean (+SE) CAMP accumulation (FIG. 15A) and β-arrestin coupling (FIG. 15B) at the GLP-IR, as well as GLP-IR internalization (FIG. 15C), time course for GLP-IR internalization after treatment with 100 nM GLP-1, liraglutide, and CT-859 (FIG. 15D), and inhibition of GLP-1 mediated β-arrestin coupling by CT-859 at the GLP-IR (FIG. 15E). FIG. 15F shows cAMP accumulation, and FIG. 15G shows β-arrestin coupling at the GIPR, while FIG. 15H shows GIPR internalization and FIG. 15I shows a time course for GIPR internalization at 100 nM after GIP and CT-859. FIG. 15J shows inhibition of GIP mediated β-arrestin coupling by CT-859 at the GIPR.

FIG. 16 shows the in vitro properties for GLP-1, liraglutide, and CT-859.

FIGS. 17A-17D present data demonstrating that CT-859 is more potent than liraglutide in vivo. FIG. 17A shows the mean (±SE) glucose response to an IPGTT in lean C57BL/6J mice four hours after vehicle, CT-859 (0.01 nmol/kg), CT-859 (0.1 nmol/kg), CT-859 (1 nmol/kg), or CT-859 (10 nmol/kg) administration and FIG. 17B shows the corresponding area under the glucose curves. FIG. 17C shows the glucose response to an IPGTT in lean C57BL/6J mice 4 hours after vehicle, liraglutide (1 nmol/kg), liraglutide (3 nmol/kg), liraglutide (10 nmol/kg), or liraglutide (30 nmol/kg) administration and FIG. 17D shows the corresponding area under the glucose curves. Statistical differences were evaluated using a one-way ANOVA followed by Bonferroni post hot test. *p<0.05; **p<0.01, ***p<0.001; ****p<0.0001.

FIGS. 18A-18F present data demonstrating that CT-859 engages the GIPR at higher doses. FIGS. 18A and 18B show the mean (+SE) Glucose response to an IPGTT in GLP-IR^{+/+} and GLP-IR^{-/-} mice, respectively, four hours after vehicle, CT-859 (20 nmol/kg), and CT-859 (200 nmol/kg) administration, and FIG. 18C shows the area under the glucose curves. FIGS. 18D and 18E show the glucose response to an IPGTT in GIPR^{+/+} and (E) GIPR^{-/-} mice, respectively, four hours after vehicle, CT-859 (20 nmol/kg), and CT-859 (200 nmol/kg) administration and FIG. 18F shows the area under the glucose curves. Statistical differences were evaluated using a two-way ANOVA followed by Bonferroni post hot test. ****p<0.0001.

FIGS. 19A-19L present data showing that biased GLP-IR agonists lower glucose over a longer duration than unbiased GLP-IR agonists. FIGS. 19A-19C show mean (±SE) glucose response to an IPGTT in GIPR−/− mice 4 hours (FIG. 19A), 24 hours (FIG. 19B), and 48 hours (FIG. 19C) after vehicle, liraglutide (20 nmol/kg), and CT-859 (20 nmol/kg) administration, and FIG. 19D shows the area under the glucose curves. FIGS. 19E-19G show glucose response to an IPGTT in lean C57BL/6J mice 4 hours (FIG. 19E), 24 hours (FIG. 19F), and 48 hours (FIG. 19G) after vehicle, liraglutide (20 nmol/kg), and CT-859 (20 nmol/kg) administration, and FIG. 19H shows the area under the glucose curves. FIGS. 19I-19K show the glucose response to an IPPTT in C57BL/6J-DIO mice 4 hours (FIG. 19I), 24 hours (FIG. 19J) and 48 hours (FIG. 19K) after vehicle, liraglutide (20 nmol/kg), and CT-859 (20 nmol/kg) administration and FIG. 19L shows the area under the glucose curves. Statistical differences were evaluated using a two-way ANOVA followed by Bonferroni post hot test. *p<0.05, **p<0.01, ***p<0.001, ****p<0.0001.

FIG. 20 presents data demonstrating that CT-859 has less exposure than liraglutide and engages the GIPR only at high doses. The mean (±SE) plasma drug concentrations for liraglutide and CT-859 in CD-1 mice are shown.

FIGS. 21A-21H present data demonstrating that Exendin-Phe1 improves glucose tolerance and decreases body weight more than exendin-4. FIG. 21A shows the mean (±SE) CAMP accumulation, FIG. 21B shows the β-arrestin coupling at the GLP-IR, FIG. 21C shows GLP-IR internalization, and FIG. 21D shows a time course for GLP-1R internalization after treatment with 100 nM GLP-1, Exendin-4 (Ex-4), and Exendin-Phe1 (Ex-Phe1). FIG. 21E shows glucose levels during an IPGTT eight hours after administration of Ex-4 (2.4 nmol/kg) and Ex-Phe1 (2.4 nmol/kg) in C57BL/6J-DIO mice and FIG. 21F shows the corresponding area under the glucose curves. FIG. 21G shows body weight FIG. 21H shows cumulative food consumption in lean C57BL/6J mice after a single ICV injection of Ex-4 (0.025 nmol) and Ex-Phe1 (0.025 nmol). Statistical differences were evaluated using a two-way ANOVA followed by Bonferroni post hot test. *p<0.05; ****p<0.0001; for (G and H) *=p<0.05; vehicle vs. Ex-4 (0.025 nmol), #=p<0.05: vehicle vs. Ex-Phe1 (0.025 nmol), a=p<0.05; Ex-4 (0.025 nmol) vs. Ex-Phe1 (0.025 nmol).

FIGS. 22A-22D present data demonstrating that ICV administration of CT-859 decreased food consumption and body weight more than liraglutide. FIG. 22A shows mean (±SE) body weight and FIG. 22B shows cumulative food consumption after ICV administration of vehicle, liraglutide (0.025 nmol), and CT-859 (0.025 nmol) in lean C57BL/6J mice. FIG. 22C shows body weight and FIG. 22D shows cumulative food consumption after ICV administration of vehicle and CT-859 (0.025 nmol) in GLP-IR and GLP-IR^{-/-} mice. Statistical differences were evaluated using a two-way ANOVA followed by Bonferroni post hot test. *=p<0.05; vehicle vs. liraglutide (0.025 nmol), #=p<0.05: vehicle vs. CT-859 (0.025 nmol), a=p<0.05; liraglutide (0.025 nmol) vs. CT-859 (0.025 nmol).

FIGS. 23A-23J present data demonstrating that CT-859 decreases body weight more than liraglutide at non-GIPR and GIPR engaging doses in DIO mice. FIGS. 23A-23E shows the mean (±SE) body weight, food consumption, fasting blood glucose, fasting plasma insulin, and Log (HOMA-IR), respectively, in C57BL/6J DIO mice treated with vehicle, liraglutide (20 nmol/kg), and CT-859 (20 nmol/kg) for 19 days. FIGS. 23F-23J show body weight, food consumption, fed blood glucose, fed plasma insulin, and Log (HOMA-IR), respectively, in C57BL/6J DIO mice treated with vehicle, liraglutide (200 nmol/kg), and CT-859 (200 nmol/kg) for 19 days. Statistical differences were evaluated using a one-way ANOVA or two-way ANOVA followed by Bonferroni post hot test. *p<0.05; **p<0.01, ***p<0.001; ****p<0.0001; for (A) *=p<0.05; vehicle vs. liraglutide (20 nmol/kg), #=p<0.05: vehicle vs. CT-859 (20 nmol/kg), a=p<0.05; liraglutide (20 nmol/kg) vs. CT-859 (20 nmol/kg); for (F) *=p<0.05; vehicle vs. liraglutide (200 nmol/kg), #=p<0.05: vehicle vs. CT-859 (200 nmol/kg), a=p<0.05; liraglutide (200 nmol/kg) vs. CT-859 (200 nmol/kg).

FIGS. 24A-24F present data demonstrating that CT-859 decreases adipose tissue weight more than liraglutide in DIO mice. FIGS. 24A-24 C show, respectively, mean (±SE) inguinal white adipose (iWAT), epidydimal white adipose (eWAT), and liver weights in C57BL/6J DIO mice treated with vehicle, liraglutide (20 nmol/kg), and CT-859 (20 nmol/kg) for 19 days. FIGS. 24D-F show, respectively, iWAT, eWAT, and liver weights in C57BL/6J DIO mice treated with vehicle, liraglutide (200 nmol/kg), and CT-859 (200 nmol/kg) for 19 days. Statistical differences were evaluated using a one-way ANOVA followed by Bonferroni post hot test. *p<0.05; **p<0.01, ***p<0.001; ****p<0.0001.

FIG. 25 depicts a plot showing weight change in ob/ob mice following treatment with vehicle (open white circles), 200 nmol/kg liraglutide, or 30 nmol/kg CT-868.

FIG. 26 depicts plots showing blood glucose levels (mg/dL) 4 hours (extreme left plot), 24 hours (second plot from left), 48 hours (third plot from left), and the corresponding AUC blood glucose (min*mg/dL; extreme right plot) during an intra peritoneal glucose tolerance test in GIPR-knockout mice (GIPR″) dosed with vehicle, 20 nmol/kg liraglutide, or 20 nmol/kg CT-859.

FIG. 27 depicts a pair of plots showing change in body weight for 15 days (%; left plot) and blood glucose levels (mg/dL; right plot) at baseline, and after 8 days and 15 days of dosing in Akita mice with vehicle, 20 nmol/kg liraglutide, or 20 nmol/kg CT-868.

FIG. 28 depicts a pair of plots showing change in body weight for 14 days (%; left plot) and blood glucose levels (mg/dL; right plot) at baseline and after 7 days and 14 days of dosing in Akita mice with vehicle+0.5 pellets of LinBit insulin, 200 nmol/kg liraglutide+0.5 pellets of LinBit insulin, 200 nmol/kg CT-868+0.5 pellets of LinBit insulin.

FIG. 29 depicts a pair of plots showing change in body weight (%; left plot) and blood glucose levels (mg/dL; right plot) in DIO-STZ mice after 12 days of dosing with vehicle+1 pellet of LinBit insulin, 200 nmol/kg liraglutide+1 pellet of LinBit insulin, or 200 nmol/kg CT-868+1 pellet of LinBit insulin.

FIG. 30 depicts a pair of plots showing change in body weight (%; left plot) and blood glucose levels (mg/dL; left plot) in Akita mice after 14 days of dosing with (vehicle+0.5 pellets of LinBit insulin), (vehicle+1.5 pellets of LinBit insulin), 300 nmol/kg CT-868+1 blank pellets, or (300 nmol/kg CT-868+0.5 pellets of LinBit insulin).

FIG. 31 depicts a pair of plots showing 4 hours fasting blood glucose levels (mg/dL; left plot) and fasting plasma insulin levels (uIU/mL; right plot) in Akita mice after 14 days of dosing with (vehicle+0.5 pellets of LinBit insulin) (open white bars and circles), (vehicle+1.5 pellets of LinBit insulin), 300 nmol/kg CT-868+1 blank pellets, or (300 nmol/kg CT-868+0.5 pellets of LinBit insulin).

FIG. 32 depicts plots showing blood glucose levels (mg/dL; top left plot) and the corresponding glucose AUC (% of vehicle; top right plot) along with plots showing plasma insulin levels (μIU/mL); bottom left plot) and the corresponding insulin AUC (% of vehicle; bottom right plot) during an intra peritoneal glucose tolerance test in GLP-1R-KO mice 1 hour after being dosed with vehicle or 300 nmol/kg CT-868.

FIG. 33 depicts plots showing the results of a pyruvate tolerance test to assess endogenous glucose production; blood glucose levels (mg/dL; left plot) and AUC blood glucose (min*mg/dL; right plot) in Akita mice following dosing with vehicle, 10 nmol/kg CT-868, 30 nmol/kg CT-868, or 100 nmol/kg CT-868, each dose in the background of 3 U/kg insulin.

FIG. 34 depicts plots showing the results of a pyruvate tolerance test to assess endogenous glucose production; blood glucose levels (mg/dL) in wildtype (left plot) and GLP-1R-KO (middle plot) mice and the corresponding AUC blood glucose (mg/dL*min; right plot) in WT and GLP-1R-KO mice following dosing with vehicle, 30 nmol/kg CT-868, or 100 nmol/kg CT-868.

FIG. 35 depicts three plots showing % change in body weight from baseline (top), food consumption (g) (bottom left), and plasma glucose (mg/ml) (bottom right) in wildtype mice measured at baseline, 24-hours, 48-hours, and 72-hours following administration of vehicle, 0.025 nmol/kg of CT-859, or 1 nmol/kg of CT-859.

FIG. 36 depicts three plots showing % change in body weight from baseline (top), food consumption (g) (bottom left), and plasma glucose (mg/ml) (bottom right) in GLP-1R-KO (GLP-1R−/−) mice measured at baseline, 24-hours, 48-hours, and 72-hours following administration of vehicle, 0.025 nmol/kg of CT-859, or 1 nmol/kg of CT-859.

FIG. 37A depicts a plot showing weight change in wildtype mice measured at baseline, 4-hours, and 19-hours following administration of vehicle, 0.025 nmol/kg of CT-859, and 0.025 nmol/kg of liraglutide (Lira). X-axis shows time in hours (hrs), and Y-axis shows % change in body weight from baseline.

FIG. 37B depicts a plot showing food consumption in wildtype mice measured at baseline, 4-hours, and 19-hours following administration of vehicle, 0.025 nmol/kg of CT-859, and 0.025 nmol/kg of liraglutide (Lira). X-axis shows time in hours (hrs), and Y-axis shows food consumption in gram (g).

FIGS. 38A-B depict the crossover clinical study design described in Example 4. Pbo: placebo; 868: CT-868.

FIG. 39 depicts a plot showing insulin secretory responses in type 2 diabetes mellitus (T2DM) patients to liraglutide and CT-868 compared to placebo-treated participants. The insulin secretion rates (ISR; pmol/kg/min) are plotted on the Y-axis and glucose levels (mmol/L) are plotted on the X-axis.

FIG. 40 depicts a pair of plots showing change in glucose and insulin levels in overweight and obese adults with T2DM following administration of placebo, liraglutide, and CT-868.

FIG. 41 depicts a plot showing the plasma exposure of CT-868 in T2DM patients. CT-868 plasma concentrations (ng/mL) are plotted on the Y-axis and time (hour) is plotted on the X-axis.

FIGS. 42A-42H present data showing that CT-868 is a cAMP-biased dual agonist for GLP-1/GIP receptors with imbalanced selectivity favoring GLP-IR. FIGS. 42B and 42C show dose-response curves for cAMP assay in cells expressing human GLP-IR (FIG. 42B) and human GIPR (FIG. 42C). Curves depict responses to GLP-1, Liraglutide, GIP and/or CT-868. FIGS. 42D and 42E show dose-response curves for β-arrestin 2 recruitment in cells expressing human GLP-1R (D) and human GIPR (E). Curves show responses to GLP-1, Liraglutide, GIP, and/or CT-868. FIGS. 24F-24G show a time-course analysis of receptor internalization for human GLP-IR (F) and GIPR (G) in response to 100 nM of GLP-1, Liraglutide, GIP, and/or CT-868. Internalization was monitored over 120 minutes at two-minute intervals, with values normalized to vehicle control. FIGS. 24H-24I show receptor internalization analysis for human GLP-IR (H) and GIPR (I) in response to GLP-1, GIP, Liraglutide, and/or CT-868 at 120 minutes (H) and 60 minutes (I) post-treatment. Data for all panels represent mean±SEM of n=6-9 replicates per treatment.

FIGS. 43A-I present data showing that CT-868 enhances glucose homeostasis through GLP-IR and GIPR-mediated mechanisms. FIGS. 43A-43B show blood glucose and plasma insulin levels during the IPGTT in lean C57BL/6NJ mice treated with vehicle or CT-868 at 3 nmol/kg, 10 nmol/kg, and 30 nmol/kg. Data are presented as mean±SEM of n=8 animals per group. FIGS. 43C-43F show blood glucose and plasma insulin levels during the IPGTT in wild type and GLP-1 KO mice treated with vehicle, or CT-868 at 30 nmol/kg and 300 nmol/kg. Data are presented as mean±SEM of n=5-6 animals per group. FIG. 43G shows AUC analysis of blood glucose levels during ipPTT in wild type and GLP-1 KO mice treated with vehicle, or CT-868 at 30 nmol/kg and 300 nmol/kg. Data are presented as mean±SEM of n=6 animals per group. FIGS. 43H-43I show MMTT in DIO mice at 23 weeks of age, conducted 24 h after equimolar dosing of CT-868, liraglutide, or vehicle at 30 nmol/kg via subcutaneous injection (10 mL/kg). Prior to the MMTT, mice underwent a 16 h fasting period. Blood glucose and plasma insulin levels were monitored at 15-, 30-, 60-, and 120-minutes post-meal administration. Data are presented as mean±SEM of n=8 animals per group. Statistical significance is denoted as follows: **p<0.01, ***p<0.001, ****p<0.0001, a p<0.05 for vehicle vs CT-868 at 30 nmol/kg, and b p<0.05 for liraglutide at 30 nmol/kg vs CT-868 at 30 nmol/kg. One-way ANOVA with Tukey's multiple comparison test. Abbreviations: IPGTT=intraperitoneal glucose tolerance tests; IVGTT=intravenous glucose tolerance test; MMTT=mixed meal tolerance test; DIO=diet-induced obese; ipPTT=pyruvate tolerance test.

FIGS. 44A-44D present data showing that chronic CT-868 administration dose-dependently reduced body weight in DIO and ob/ob mice. FIG. 44A shows the dose-dependent effect of CT-868 (3, 10, or 30 nmol/kg/day) on body weight over 14 days in DIO mice. Changes in body weight are expressed as a percentage of the initial body weight recorded on Day 1. The data represent mean±SEM for n=10 animals per treatment group. Statistical significance is denoted as follows: *p<0.05 for vehicle vs CT-868 at 3 nmol/kg, #p<0.05 for vehicle vs CT-868 at 10 nmol/kg, \hat{ }p<0.05 for vehicle vs CT-868 at 30 nmol/kg, a p<0.05 for CT-868 at 3 nmol/kg vs 10 nmol/kg, b p<0.05 for CT-868 at 3 nmol/kg vs 30 nmol/kg, and c p<0.05 for CT-868 at 10 nmol/kg vs 30 nmol/kg. Analysis was conducted using two-way ANOVA with Tukey's multiple comparisons test. FIG. 44B shows end-of-study tissue weight analysis in DIO mice treated with CT-868. Panels represent the dose-dependent effect of CT-868 (3, 10, or 30 nmol/kg/day) on the weights of iWAT, eWAT, and liver, respectively. The data represent mean±SEM for n=10 animals per treatment group. *p<0.05, **p<0.01, ***p<0.001, ****p<0.0001. Analysis was conducted using two-way ANOVA with Tukey's multiple comparisons test. FIGS. 44C-44D show comparative analysis of body weight change (44C) and 24 h food consumption (44D) in ob/ob mice over 24 days dosed daily with vehicle, CT-868 (10 or 30 nmol/kg/day) or liraglutide (200 nmol/kg/day). Changes in body weight are expressed as a percentage of the initial body weight recorded on Day 1 (44C). The food intake data on Day 1, Day 8 and Day 15 are represented in FIG. 44D. The data represent mean±SEM for n=8 animals per treatment group. Statistical significance for FIG. 44C is denoted as follows: *p<0.05 for vehicle vs liraglutide at 200 nmol/kg, #p<0.05 for vehicle vs CT-868 at 10 nmol/kg, \hat{ }p<0.05 for vehicle vs CT-868 at 30 nmol/kg, a p<0.05 for liraglutide at 200 nmol/kg vs CT-868 at 10 nmol/kg, b p<0.05 for liraglutide at 200 nmol/kg vs CT-868 at 30 nmol/kg, and c p<0.05 for CT-868 at 10 nmol/kg vs 30 nmol/kg. Statistical significance for panel F is denoted as follows: *p<0.05, **p<0.01, ***p<0.001, ****p<0.0001. Analysis was conducted using two-way ANOVA with Tukey's multiple comparisons test. Abbreviations: DIO=diet-induced obese; iWAT=inguinal white adipose tissue; eWAT=epididymal white adipose tissue; ob/ob mice=Leptin-deficient mice.

FIGS. 45A-45B present data showing that CT-868 positively influenced energy metabolism. FIG. 45A shows the effect of CT-868 on RER (the ratio of VCO2/VO2), and FIG. 45B shows the ambulatory activity in DIO mice over 12 days dosed daily with either vehicle or CT-868 at 20 nmol/kg/day. The data represent back-transformed adjusted means+SEM for n=8 animals per treatment group, and dark cycles (x p.m. to x a.m.) are shown by a shaded gray box. Significant differences vs vehicle: *p<0.05, **p<0.01, ***p<0.001. Analysis was conducted by general linear model with treatment as a factor and Day 1 body weight and log (baseline) (mean from Day −2 to 0) as covariates. Abbreviations: VO2=oxygen consumption; VCO2=carbon dioxide production; RER=respiratory exchange ratio.

FIGS. 46A-46F present schematics and data related to the first-in-human single ascending dose and multiple ascending dose study of CT-868. FIG. 46A shows the study design for Part 1: Single ascending dose (SAD) and FIG. 46B shows the study design for Part 2: Multiple ascending dose (MAD). In Part 1 of the study (SAD phase), participants received a single dose of study drug (CT-868 or matching placebo) by subcutaneous injection. In Part 2 (MAD phase), participants were randomized to receive CT-868 or placebo by subcutaneous injection (once daily on Days 1 to 14). Except for the ‘Lean volunteers’ (cohort S7), all participants in Part 1 and Part 2 were individuals with overweight/obesity but otherwise healthy. FIGS. 46C and 46D show plasma concentration-time profile of CT-868 after single or multiple doses, respectively. Plots show mean CT-868 plasma concentrations by dose cohort after single or multiple doses of CT-868. Values are plotted using a semi-log scale. Concentrations below the lower limit of quantification (0.2 ng/mL) and zero mean values were not plotted. FIG. 46E shows relationship between CT-868 AUC₀₋ₗₐₛₜ and baseline-adjusted AUC for glucose, insulin and C-peptide following a meal tolerance test. Red data points indicate all doses up to 7.5 mg; blue data points represent 11 mg dose only. Data were analyzed with and without the 11 mg dose (Cohort S9) as this MTT was conducted 24 hours after dosing whereas the MTTs for placebo and Cohorts S1 to S8 were conducted 3 hours after dosing. Daily CT-868 administration for 14 days resulted in dose-dependent body weight reduction in participants with overweight/obesity (FIG. 46F). Abbreviations: AUC=area under the curve; BMI=body mass index; BW=body weight; D=Day; MAD=multiple ascending dose; MD=multiple dose; SAD=single ascending dose; T2DM=type 2 diabetes mellitus.

FIGS. 47A and 47B show schematics of the placebo- and comparator-controlled crossover study (CT-868-003). Part 1 included 12 participants with obesity. In Group 1, six participants received crossover treatment with CT-868 (Day 1:5.0 mg, Day 2:3.25 mg), matching placebo, and liraglutide (Day 1:0.6 mg, Day 2:1.2 mg) for two days each, with a 14-day washout period between treatments (refer to medication dispensing chart, STAR Methods). In Group 2, six participants received crossover treatment with CT-868 and matching placebo for two days each, with a 14-day washout in between. Part 2 included 20 participants with T2DM, 13 in Group 1 (crossovers between CT-868 [Day 1:5.0 mg, Day 2: 3.25 mg, Day 3:3.25 mg], matching placebo, and liraglutide [Day 1:0.6 mg, Day 2:1.2 mg, Day 3:1.2 mg]), and seven in Group 2 (crossovers between CT-868 and matching placebo). Abbreviations: GE, Gastric emptying test; GGI=Graded glucose infusion; T2DM=type 2 diabetes mellitus; w/o=washout.

FIGS. 48A and 48B present data demonstrating that CT-868 shows robust pancreatic effects in participants with overweight/obesity and participants with T2DM. FIG. 48A shows relationship between insulin secretion rate (ISR) and ambient glucose during graded glucose infusion in participants with overweight/obesity after 2 days of CT-868 dosing, and FIG. 48B shows relationship between insulin secretion rate (ISR) and ambient glucose during graded glucose infusion in participants with T2DM after 2 days of CT-868 dosing. Abbreviations: G=glucose; ISR=insulin secretion rate; LS=least-squares; T2DM=type 2 diabetes mellitus.

FIG. 49 presents data showing the effects of CT-868 on plasma glucose, insulin, and glucagon during a mixed-meal tolerance test in participants with T2DM. Plots show percentage change from baseline in plasma glucose, insulin and glucagon during a mixed meal tolerance test conducted after 3 days of CT-868 dosing.

FIG. 50 presents data showing that CT-868 and liraglutide demonstrated similar gastric emptying delay versus placebo. Acetaminophen plasma concentration-time profile for CT-868, placebo and liraglutide in participants with T2DM during an acetaminophen absorption test. Abbreviations: AUC=area under the curve; Cₘₐₓ=maximum plasma concentration; CV=coefficient of variation; SD=standard deviation, Tₘₐₓ=time at which the maximum plasma concentration was attained.

FIG. 51 depicts a titration scheme for the Phase 2 clinical study described in Example 7.

FIG. 52 depicts two plots showing % change in HbA1c from baseline (plot A; left) up to Week 26, and % of subjects achieving target HbA1c (plot B; right) at Week 26 following administration of placebo, 1.75 mg of CT-868, 3.25 mg of CT-868, or 4 mg of CT-868 in overweight or obese adults with type 2 diabetes mellitus (T2DM).

FIG. 53 depicts six plots showing change in total cholesterol (top left), low-density lipoprotein (LDL; top center), high-density lipoprotein (HDL; top right), triglycerides (bottom left), very low-density lipoprotein (VLDL; bottom center), and apolipoprotein B (ApoB; bottom right) measured at baseline, 4-weeks, 12-weeks, and 26-weeks following administration of placebo, 1.75 mg of CT-868, 3.25 mg of CT-868, or 4 mg of CT-868 in overweight or obese adults with type 2 diabetes mellitus (T2DM).

FIG. 54 depicts two plots showing mean change in blood pressure (plot A; left) and liver enzyme levels (plot B; right) measured at Week 26 following administration of placebo, 1.75 mg of CT-868, 3.25 mg of CT-868, or 4 mg of CT-868 in overweight or obese adults with type 2 diabetes mellitus (T2DM). SBP: systolic blood pressure; DBP: diastolic blood pressure, ALT: alanine transaminase; AST: aspartate transferase; GGT: gamma-glutamyl transferase.

FIG. 55 shows the overall trial design, including arms and doses, of a Phase 2 trial of CT-868 in Overweight and Obese adults with TID.

FIG. 56 shows the main study activities in pre- and post-randomization of a Phase 2 trial of CT-868 in Overweight and Obese adults with TID, including 2-week CGM and insulin dose data collection and diet and exercise patient education.

FIG. 57 shows the main study activities in pre- and post-randomization of a Phase 2 trial of CT-868 in Overweight and Obese adults with TID, including HbA1c, PROs, CRU visits, blood sample, study drug concentration (˜24 hours post dose), and extended PK sampling in subset.

### DETAILED DESCRIPTION OF THE INVENTION

The present disclosure provides a therapy (e.g., monotherapy or an adjunctive therapy) for insulin-dependent or insulin-resistant patients (e.g., patients with type 1 and type 2 diabetes mellitus) by using a unimolecular agonist for both glucagon-like peptide-1 (GLP-1) receptor (GLP-1R) and glucose-dependent insulinotropic polypeptide (GIP) receptor (GIPR). The therapy herein may improve outcomes of diabetes therapy such as insulin therapy in several ways.

In some embodiments, the therapy herein (e.g., monotherapy or an adjunctive therapy) improves glycemic control in T1DM or T2DM patients. Despite advances in diabetes therapy such as insulin therapy, drug delivery, and glucose monitoring, most T1DM and T2DM patients still do not meet current standard-of-care glycemic control goals. The therapy herein may improve the effectiveness of existing diabetes therapy including insulin therapy. Furthermore, the therapy herein may improve insulin-independent glucose disposal due in part to GIP-mediated effects. In particular embodiments, the therapy herein (e.g., monotherapy or an adjunctive therapy) improves glycemic control in T1DM patients.

In some embodiments, the therapy herein (e.g., monotherapy or an adjunctive therapy) reduces insulin doses needed to maintain glucose control, while improving hemoglobin A1c (HbA1c) levels without exacerbating the risk of hypoglycemia and diabetic ketoacidosis (DKA). In particular embodiments, the therapy herein (e.g., monotherapy or an adjunctive therapy) reduces insulin doses needed to maintain glucose control, while improving hemoglobin A1c (HbA1c) levels without exacerbating the risk of hypoglycemia and diabetic ketoacidosis (DKA), in T1DM patients.

In some embodiments, the therapy herein (e.g., monotherapy or an adjunctive therapy) improves insulin sensitivity and may concomitantly address pathophysiologic defects in T1DM or T2DM patients. T1DM and T2DM patients often have insulin resistance in adipose, hepatic, and skeletal muscle tissues. They also have decreased insulin secretion and increased glucagon secretion, both of which exacerbate hyperglycemia. T1DM and T2DM patients exhibit increased glucose reabsorption by the kidney and accelerated gastric emptying. Given the dual mechanism of action of the agonists herein, the present therapy has the potential to address these core defects. In particular embodiments, the therapy herein (e.g., monotherapy or an adjunctive therapy) improves insulin sensitivity and may concomitantly address pathophysiologic defects in T1DM patients.

In some embodiments, the therapy herein (e.g., monotherapy or an adjunctive therapy) reduces body weight in T1DM or T2DM patients in need thereof. Approximately two-thirds of T1DM and T2DM patients are overweight or obese. Insulin therapy exacerbates weight gain, which risks precipitating the negative health effects of obesity. Thus, interventions that can decrease adiposity and improve insulin sensitivity and consequently reduce the total daily insulin burden of T1DM and T2DM patients would be an important component of therapy. In particular embodiments, the therapy herein (e.g., monotherapy or an adjunctive therapy) reduces body weight in T1DM patients in need thereof.

In some embodiments, the therapy herein (e.g., monotherapy or an adjunctive therapy) reduces cardiovascular disease risk factors in T1DM and T2DM patients. T1DM and T2DM patients with ideal glucose control still have an increased risk of cardiovascular disease. This is thought to be induced by several precipitating factors, including hyperinsulinemia, insulin resistance, increased inflammatory tone, increased atherogenic lipids, and endothelial dysfunction. Therapies that reduce insulin dosage and enhance insulin sensitivity may reduce cardiovascular disease risk. In particular embodiments, the therapy herein (e.g., monotherapy or an adjunctive therapy) reduces cardiovascular disease risk factors in T1DM patients.

In some embodiments, the therapy herein (e.g., monotherapy or an adjunctive therapy) reduces blood pressure (e.g., systolic blood pressure) and/or atherogenic lipids in T1DM or T2DM patients. In particular embodiments, the therapy herein (e.g., monotherapy or an adjunctive therapy) reduces blood pressure (e.g., systolic blood pressure) and/or atherogenic lipids in T1DM patients.

### I. DUAL GLP-1R/GIPR AGONISTS

GLP-1 and GIP are primary incretins released from the gastrointestinal tract in response to food intake. Both hormones modulate glucose-dependent insulin secretion. GLP-1 also decreases secretion of glucagon, slows gastric emptying, promotes satiety, and reduces food intake. Several GLP-1 mimetics have been approved for the treatment of type 2 diabetes mellitus (T2DM), but their efficacy is limited by gastrointestinal side effects such as nausea, vomiting, and diarrhea.

The primary action of GIP is the stimulation of glucose-dependent insulin secretion. It also modulates the secretion of glucagon in an inverse glucose-dependent manner to protect against hypoglycemia and does not delay gastric emptying. GIP also stimulates glucose uptake in adipocytes and promotes bone strength.

The dual GLP-1R/GIPR agonist used in the present therapy potently activates production of cyclic adenosine monophosphate (cAMP) but has little or no activity on the β-arrestin signaling pathways on either GLP-IR or GIPR. That is, the agonist is fully biased towards cAMP activation, as opposed to being partially biased (i.e., with some β-arrestin activity) or unbiased (i.e., with full β-arrestin activity), on both GLP-IR and GIPR. β-arrestin activates kinase signaling pathways, but also causes the GLP-IR and GIPR to be turned off and internalized (Hsia et al., Curr Opin Endocrinol Diabetes Obes. (2017) 24 (1): 73-9). The present agonist does not cause internalization and consequently, desensitization of either GLP-1R or GIPR, and thus has enhanced signaling efficacy.

The present dual GLP-1R/GIPR agonist may have the following structural formula:

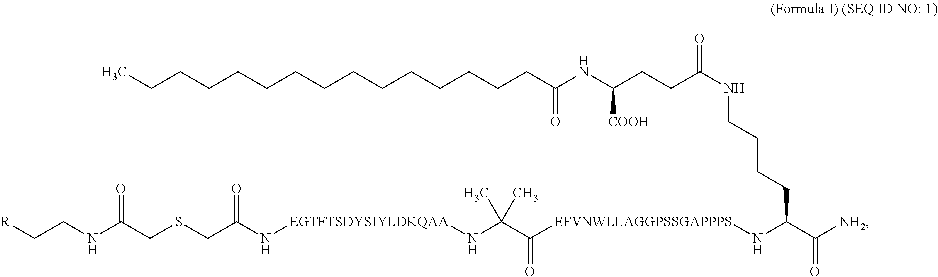

wherein R is a ring. In some embodiments, the ring is a cycloalkyl or heterocyclic group that is 4-8 atoms in size. The ring may be substituted with, without limitation, one or more carbonyl, hydroxyl, methyl, phenyl, isopropyl, trifluoromethyl, or nitro group(s). The ring may be aromatic or non-aromatic, and may be fused to one or more additional ring(s) to form a fused, bridged, or spiro bicyclic moiety. In some embodiments, R is:

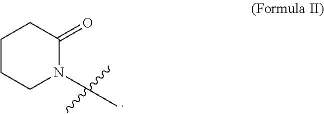 [SMILES: *N1CCCCC1=O]

In some embodiments, R is

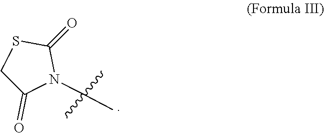 [SMILES: *N1C(=O)CSC1=O]

In some embodiments, the present agonist has the following structural formula:

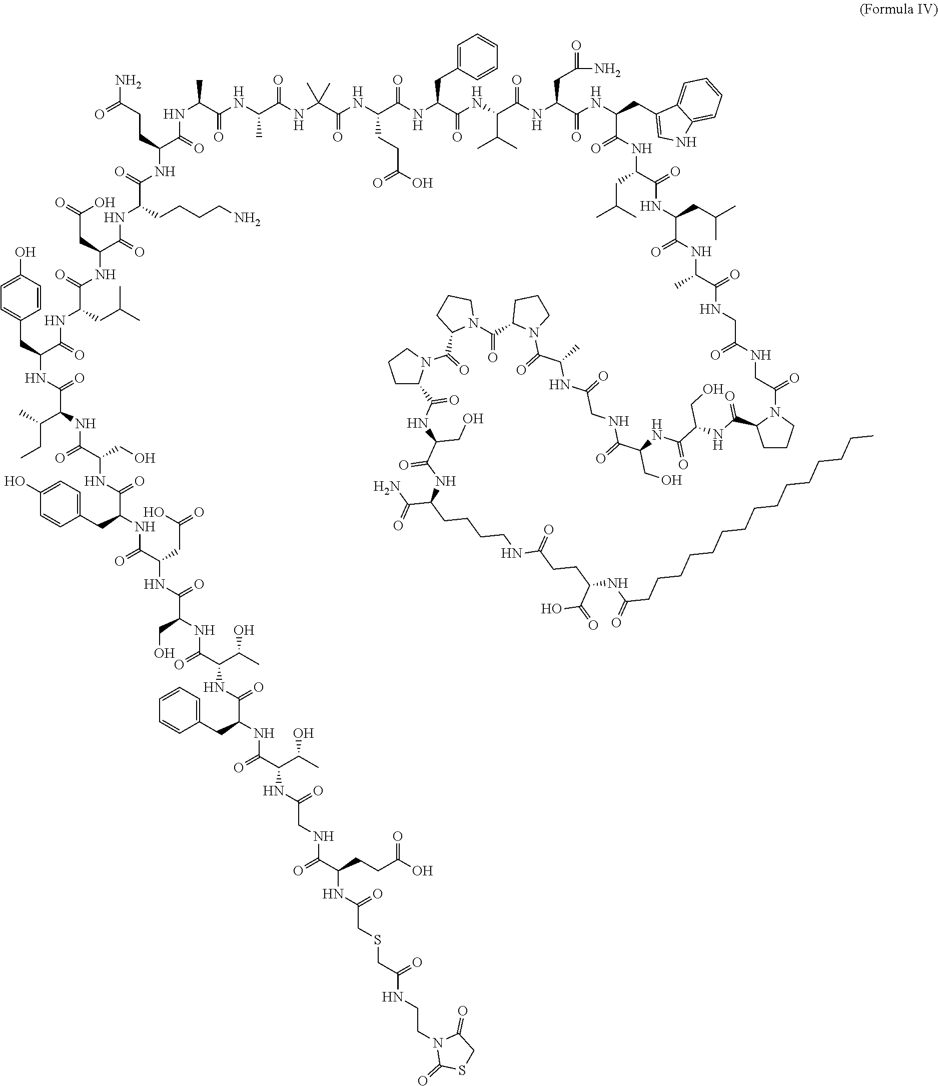

This compound is also referred to as CT-859 herein, and may be depicted as follows:

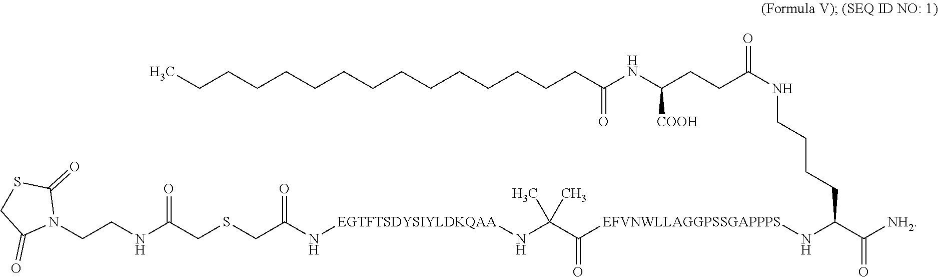

In some embodiments, the present agonist has the following structural formula:

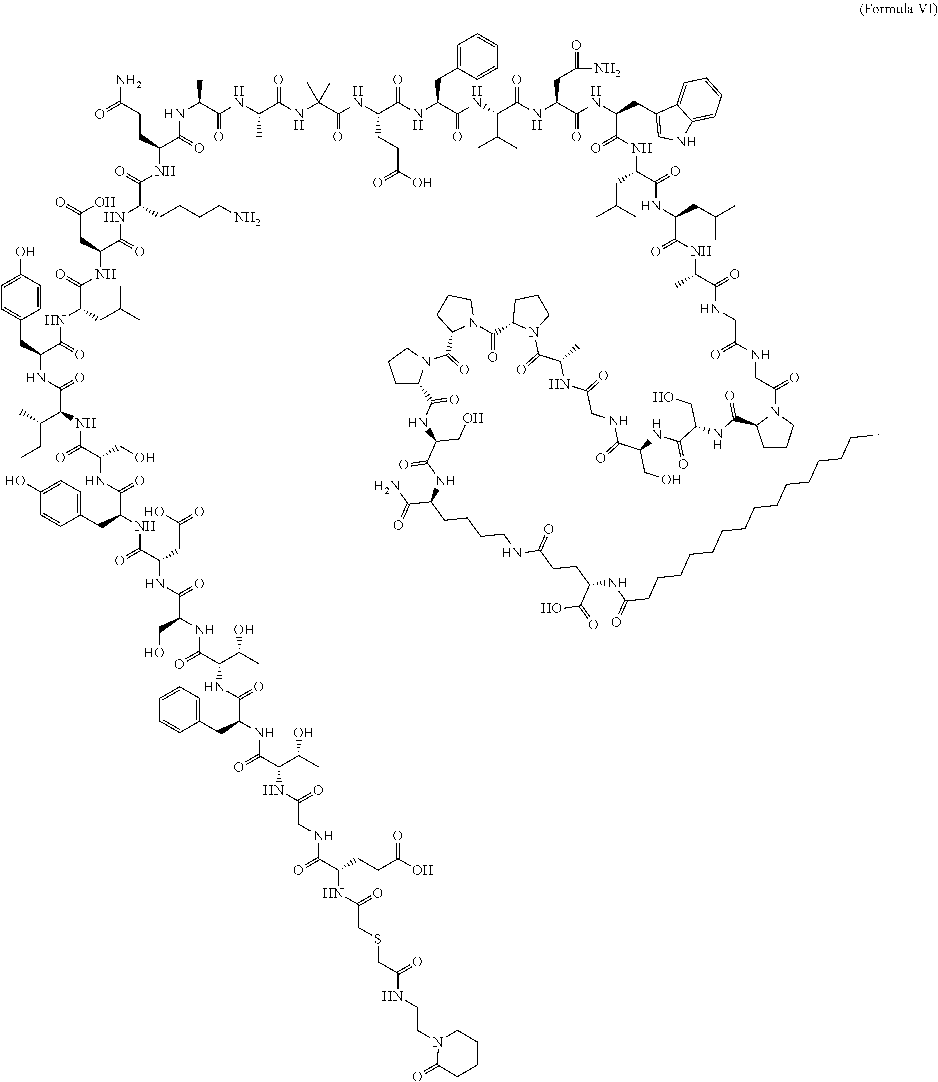

This compound is also referred to as CT-868 herein, and may be depicted as:

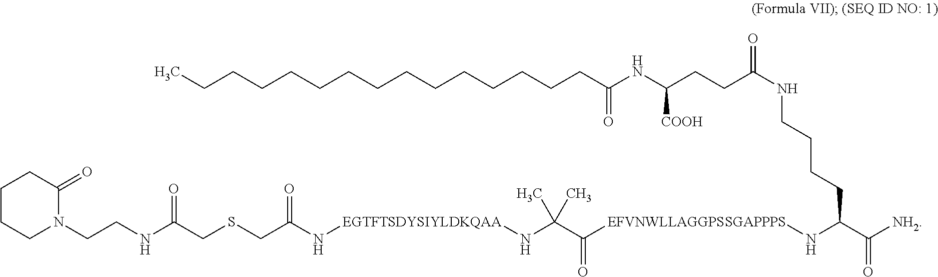

Pharmaceutically acceptable salts or esters of the above-illustrated compounds (e.g., of Formula I, IV, V, VI, or VII) may also be used in the presently described therapy (e.g., monotherapy or adjunctive therapy).

The dual GLP-1R/GIPR agonist herein, such as CT-859 and CT-868, is fully biased on both GLP-1R and GIPR with little or no β-arrestin coupling. The dual agonist may be well tolerated and cause fewer adverse effects than other incretin mimetics. For example, toxicology data show that CT-868 is well-tolerated at the highest dose tested, which exceeds the pharmacologically active dose by 100-fold. In non-human primate studies, there were no instances of vomiting, an adverse effect commonly reported with other incretin mimetics.

The potency of the present dual agonist in activating cAMP production may be measured by well-known cell-based assays. In some embodiments, the present dual agonist activates cAMP at human GLP-1R and GIPR with a potency of about 0.05 to about 5 nM. In some embodiments, the present dual agonist favors GLP-1R over GIPR at a ratio of about 1:5 to about 1:50 (e.g., about 1:10, about 1:20, about 1:30, or about 1:40). For example, CT-868 activates cAMP at human GLP-1R and GIPR with potencies of about 0.17 nM and about 3.3 nM, respectively, and favors GLP-1R over GIPR at a ratio of about 19.4.

### II. PHARMACEUTICAL COMPOSITIONS

The dual GLP-1R/GIPR agonist used in the present therapy may be provided in a pharmaceutical composition containing the agonist and pharmaceutically acceptable excipients. In some embodiments, the agonist (e.g., CT-868) is provided in a sterile aqueous solution suitable for subcutaneous injection. In some embodiments, the agonist (e.g., CT-868) is provided at a concentration of about 1 to about 100 mg/mL (e.g., about 5 to about 25 mg/mL, about 7.5 to about 20 mg/mL, or about 10 to about 15 mg/mL). As used herein, values intermediate to recited ranges and values are also intended to be part of this disclosure. In addition, ranges of values using a combination of any of recited values as upper and/or lower limits are intended to be included.

In some embodiments, the agonist (e.g., CT-868) is provided at a concentration of 15 mg/mL in a solution comprising 20 mM di-sodium hydrogen phosphate heptahydrate/sodium dihydrogen phosphate monobasic buffer solution, propylene glycol, and phenol at pH 7.0. The pharmaceutical composition may be administered to the patient with, for example, a syringe or an injector such as an injection pen (e.g., an auto-injector). The pharmaceutical composition may be clear or colorless. In some embodiments, the pharmaceutical composition is a clear liquid essentially free of visible particles. In some embodiments, the level of sub-visible particulate matter ≥10 μm is ≤6000 per container. In some embodiments, the level of sub-visible particulate matter is ≥25 μm is ≤600 per container. In some embodiments, no impurity is ≥5.0% Area. In some embodiments, the total impurities are ≤7.0% Area. In some embodiments, bacterial endotoxin is ≤5 EU/mL.

In some embodiments, the system for subcutaneous administration of CT-868 is a needle-based injection system with an integrated non-replaceable 3-mL Type 1 glass cartridge. The system may be referred to as a pen. In some embodiments, more than one dose of CT-868 is comprised within each pen. In some embodiments, a single dose of CT-868 is comprised within each pen. In some embodiments, five or more doses of CT-868 are comprised within each pen.

In some embodiments, a fixed dose of 1.1 mg CT-868 is delivered in 0.07 mL of the composition. In some embodiments, a fixed dose of 1.8 mg CT-868 is delivered in 0.12 mL of the pharmaceutical composition. In some embodiments, a fixed dose of 2.6 mg CT-868 is delivered in 0.17 mL of the pharmaceutical composition. In some embodiments, a fixed dose of 3.3 mg CT-868 is delivered in 0.22 mL of the pharmaceutical composition. In some embodiments, a fixed dose of 4.1 mg CT-868 is delivered in 0.27 mL of the pharmaceutical composition. Unless otherwise indicated, a CT-868 weight recited in the present disclosure is the weight of CT-868 free base (the active moiety).

Also provided herein are articles of manufacture and kits comprising the pharmaceutical composition or agonist. In such articles of manufacture and kits, the pharmaceutical composition or agonist may be provided in a system (e.g., a pen, vial, or needle-based injection system). The articles of manufacture and kits may also comprise instructions for use of the pharmaceutical composition or agonist in the method provided herein.

In some embodiments, the article of manufacture is an injection device, injector, syringe, or vial. The injection device, injector, syringe, or vial may be single-use, or may comprise a single dose unit of the pharmaceutical composition or agonist.

### III. DIABETES THERAPY

The pharmaceutical composition may be used as a monotherapy, or in a combination therapy adjunctive to insulin or other glycemic-controlling therapy, in T1DM or T2DM patients. The pharmaceutical composition may be injected subcutaneously at an interval deemed appropriate by a physician, for example, every day, every two days, every three days, every four days, every five days, every six days, or every week. In some embodiments, the pharmaceutical composition (e.g., containing CT-868 as the active pharmaceutical ingredient) is administered by injection (e.g., self-injection) at a body site (e.g., abdomen, upper arm, thighs, or hips) subcutaneously once daily (QD), for example, at about the same time each morning. In some embodiments, the pharmaceutical composition is administered irrespective of meals.

In some embodiments, the patient has a BMI of ≥25 kg/m², ≥27 kg/m², ≥30 kg/m², ≥35 kg/m², ≥40 kg/m², or ≥45 kg/m².

In some embodiments, the agonist herein (e.g., CT-868) is administered to a T1DM or T2DM patient at a flat dose of about 1 mg to about 30 mg, for example, about 1 mg to about 20 mg, about 1 mg to about 15 mg, about 1 mg to about 5 mg, about 2 mg to about 15 mg, about 5 mg to about 15 mg, or about 5 mg to 12 mg.

In some embodiments, a pharmaceutical composition comprising CT-868 is injected subcutaneously to a T1DM or T2DM patient at a daily dose of about 1.8 mg or about 2 mg CT-868.

In some embodiments, a pharmaceutical composition comprising CT-868 is injected subcutaneously to a T1DM or T2DM patient at a daily dose of about 2.5 mg or about 2.6 mg CT-868.

In some embodiments, a pharmaceutical composition comprising CT-868 is injected subcutaneously to a T1DM or T2DM patient at a daily dose of about 3 mg, about 3.3 mg, or about 3.5 mg CT-868.

In some embodiments, a pharmaceutical composition comprising CT-868 is injected subcutaneously to a T1DM or T2DM patient at a daily dose of about 4 mg, about 4.1 mg, or about 4.5 mg CT-868.

In some embodiments, a pharmaceutical composition comprising CT-868 is injected subcutaneously to a T1DM or T2DM patient at a daily dose of about 5 mg, about 5.2 mg, or about 5.5 mg CT-868. A daily dose of 5.2 mg CT-868 is administered as two doses of 2.6 mg each.

In some embodiments, a pharmaceutical composition comprising CT-868 is injected subcutaneously to a T1DM or T2DM patient at a daily dose of about 6 mg, about 6.5 mg, or about 6.6 mg CT-868. A daily dose of 6.6 mg CT-868 is administered as two doses of 3.3 mg each.

In some embodiments, a pharmaceutical composition comprising CT-868 is injected subcutaneously to a T1DM or T2DM patient at a daily dose of about 7 mg or about 7.5 mg CT-868.

In some embodiments, a pharmaceutical composition comprising CT-868 is injected subcutaneously to a T1DM or T2DM patient at a daily dose of about 8 mg or about 8.5 mg CT-868.

In some embodiments, a pharmaceutical composition comprising CT-868 is injected subcutaneously to a T1DM or T2DM patient at a daily dose of about 9 mg or about 9.5 mg CT-868.

In some embodiments, a pharmaceutical composition comprising CT-868 is injected subcutaneously to a T1DM or T2DM patient at a daily dose of about 10 mg or about 10.5 mg CT-868.

In some embodiments, a pharmaceutical composition comprising CT-868 is injected subcutaneously to a T1DM or T2DM patient at a daily dose of about 11 mg or about 11.5 mg CT-868.

In some embodiments, a pharmaceutical composition comprising CT-868 is injected subcutaneously to a T1DM or T2DM patient at a daily dose of about 12 mg or about 12.5 mg CT-868.

In some embodiments, a pharmaceutical composition comprising CT-868 is injected subcutaneously to a T1DM or T2DM patient at a daily dose of about 13 mg or about 13.5 mg CT-868.

In some embodiments, a pharmaceutical composition comprising CT-868 is injected subcutaneously to a T1DM or T2DM patient at a daily dose of about 14 mg or about 14.5 mg CT-868.

In some embodiments, a pharmaceutical composition comprising CT-868 is injected subcutaneously to a T1DM or T2DM patient at a daily dose of about 15 mg or about 15.5 mg CT-868.

In some embodiments, treatment with CT-868 is initiated on an up-titration schedule, i.e., starting treatment at a low dosing amount, and gradually increasing the dosing amount over a period of time to the higher or highest, tolerated dosing amount. In some embodiments, the titration occurs over a period of about 1, 2, 4, 6, 8, or 10 weeks, or longer; in further embodiments, during the titration period, the pharmaceutical composition containing CT-868 is injected QD. In some embodiments, the titration starts with a dosing amount (starting dose) of 1.1 mg, finally reaching a dosing amount (maintenance or maximum dose) that is between 1.8 and 6.6 mg, for example, 1.8, 4.1, 5.2, or 6.6 mg.

In some embodiments, the titration occurs according to the following regimen:
1.1 mg QD from days 1 to 7, and
1.8 mg QD from week 1 to week 2.

In some embodiments, the titration occurs according to the following regimen:
1.1 mg QD from days 1 to 7,
1.8 mg QD from week 1 to week 2,
2.6 mg QD from week 2 to week 4,
3.3 mg QD from week 4 to week 6, and
4.1 mg QD from week 6 to week 16.

In some embodiments, the titration occurs according to the following regimen:
1.1 mg QD from days 1 to 7,
1.8 mg QD from week 1 to week 2,
2.6 mg QD from week 2 to week 4,
3.3 mg QD from week 4 to week 6,
4.1 mg QD from week 6 to week 8,
5.2 mg QD from week 8 to week 10, and
6.6 mg QD from week 10 to week 16.

In some embodiments, the titration occurs according to the following regimen:
1.0 mg QD for weeks 1 and 2,
2.0 mg QD for weeks 3 and 4,
3.0 mg QD for weeks 5 and 6,
4.0 mg QD for weeks 7 and 8,
5.0 mg QD for weeks 9 and 10,
6.0 mg QD for weeks 11 and 12,
8.0 mg QD for weeks 12-15,
10.0 mg QD for weeks 16-20, and
12.0 mg QD for weeks 21-48.

In some embodiments, the titration occurs according to the following regimen:
1.0 mg QD for weeks 1 and 2,
2.0 mg QD for weeks 3 and 4,
3.0 mg QD for weeks 5 and 6,
4.0 mg QD for weeks 7 and 8,
5.0 mg QD for weeks 9 and 10,
6.0 mg QD for weeks 11 and 12,
7.0 mg QD for weeks 13 and 14, and
8.0 mg QD for weeks 15-48.

In some embodiments, the titration occurs according to the following regimen:
1.0 mg QD for weeks 1-4,
2.0 mg QD for weeks 5-8,
3.0 mg QD for weeks 9-12, and.
4.0 mg QD for weeks 13-48.

When titrating dose upwards, if a dose is not tolerated at any stage, the dosing may be reverted to the previous tolerated dose for the next dose (i.e., down-titration is allowed). In some embodiments, the patient may then attempt up-titration at the subsequent scheduled dosing time.

While receiving the present adjunctive therapy, a T1DM or T2DM patient may receive or continue to receive insulin therapy. Insulin doses may be given in a basal dose, which provides a steady amount of insulin delivered all day and night; and/or a bolus dose, which provides a dose of insulin at meals to help move absorbed sugar from the blood into muscle and fat. Bolus doses are also called nutritional or meal-time doses. Insulin therapy may entail use of rapid-acting or fast-acting insulin, regular or short-acting insulin, intermediate-acting insulin, long-acting insulin, premixed or mixed insulin, or inhaled insulin, as directed by a physician. The insulin dosing schedule may depend on the patient's body weight, blood sugar levels, and other health conditions, type of insulin, amount of food intake, and level of physical activity. The insulin may be administered with a syringe, a pump, a pen, an inhaler, or an injection port. For example, the insulin may be administered subcutaneously via multiple daily injections (MDIs), or continually via a pump (continuous subcutaneous insulin infusion, CSII) where the pumps may be stand-alone devices or hybrid closed-loop systems (e.g., coupled with a continuous glucose monitor or CGM) such that automated insulin delivery is provided based on sensing real-time blood glucose values. Injection may be performed at the abdomen, the upper arm, the thighs, and/or the hips. The present adjunctive therapy can be an adjunct to any and all types of insulin therapy. As used herein, the term “insulin-dependent” refers to a patient that requires insulin therapy.

The adjunctive therapy herein is expected to reduce needed insulin doses or dosing frequency, improve insulin sensitivity, and/or reduce excess body weight (e.g., by food intake suppression) in T1DM or T2DM patients. This is particularly advantageous in T1DM and T2DM patients who are prone to developing obesity and insulin resistance (double diabetes). Thus, use of an adjunctive non-insulin glucose-lowering agent such as CT-859 or CT-868 in combination with insulin may provide an improved diabetes therapy. For example, most T1DM patients require three or more injections of insulin daily, with doses adjusted on the basis of self-monitoring of blood glucose levels and carbohydrate intake. Administration of the agonist herein (e.g., CT-859 or CT-868) as an adjunctive therapy in such patients may reduce the number of daily insulin injections required to maintain normal or near-normal blood glucose levels in the patient. Administration of the agonist may also produce additional benefits such as decreased body weight, improved insulin sensitivity, and lowering of blood pressure and/or plasma lipids.

In some embodiments, the T1DM or T2DM patient receives another therapy for their diabetic condition. For example, the patient may be put on diet therapy and exercise therapy. In a T1DM patient, the agonist may be administered as an adjunct to insulin therapy. In a T2DM patient, the agonist may be administered as an adjunct to diet or exercise and to oral antidiabetic medications.

The term “adjunctive therapy” means that the present agonist is to be used in combination with another therapy. For example, in a T1DM patient, treatment with the present agonist may be an adjunct to insulin therapy. In a patient with T2DM and/or obesity or overweight, treatment with the present agonist may be an adjunct to, e.g., diet or exercise.

The present agonists may be administered as an adjunct to one or more additional therapies. As used herein, an “additional therapy” refers to a therapy that is carried out in addition to treatment with the present agonists. An additional therapy may be, for example, insulin therapy, diet therapy, exercise therapy, hypertension therapy, and/or blood lipid-lowering therapy.

In some embodiments, the above-described dosing regimens may also be used for weight management to cause the patient to lose excess body weight.

### IV. T1DM PATIENT CHARACTERISTICS

In some embodiments, a patient treated with the present methods is 18 years of age or older. In some embodiments, the patient has a documented diagnosis of T1DM for, e.g., at least one year prior to treatment. In some embodiments, the patient has a body mass index (BMI) that is greater than or equal to 25.0 kg/m². The patient may have a hemoglobin A1c (HbA1c) level between 7.0% and 9.5%, inclusive, prior to treatment.

In some embodiments, prior to receiving the present treatment methods, the patient has been treated with insulin either by means of an insulin pump for continuous subcutaneous insulin infusion (CSII), or by means of multiple daily injections (MDIs) of insulin for, e.g., six or more months prior to treatment. The patient may be on stable insulin formulation, dose (i.e., within 10% total daily dose), and devices (i.e., not switching from MDI to pump or vice versa) for two or more months prior to the treatment.

In some embodiments, the patient does not have type 2 diabetes mellitus (T2DM) or any other type of diabetes except for T1DM. In some embodiments, the patient has not experienced diabetic ketoacidosis (DKA) within, e.g., three months prior to treatment, or has not experienced severe hypoglycemia (Level 3 as defined in the ADA Standards of Medical Care in Diabetes (ADA 2022) within, e.g., three months prior to treatment. The patient may not have impaired awareness of hypoglycemia (i.e., a score 4 or more on the Clarke questionnaire (Clarke et al., Diabetes Care. (1995) 18 (4): 517-22)) prior to treatment. In some embodiments, the patient receives insulin through CSII or MDI. In some embodiments, the patient does not receive insulin through preparations other than CSII or MDI (e.g., inhaled insulin).

In some embodiments, the patient has not used (for, e.g., six or three months) any medication (except insulin) that could interfere with glycemic control (e.g., monoamine oxidase inhibitors, growth hormone); any adjunctive treatments for diabetes (e.g., pramlintide, metformin, glucagon-like peptide 1 [GLP-1] analogs [e.g., exenatide], GLP-1 receptor agonists (GLP-1RAs) [e.g., semaglutide, liraglutide, or dulaglutide], GLP-1/GIP RA (Mounjaro™), sodium-glucose cotransporter-2 inhibitors, sulfonylureas, or dipeptidyl peptidase 4 inhibitors); any prescription medication(s), and/or any over-the-counter product(s) including herbal and/or dietary supplements for weight loss (e.g., orlistat, lorcaserin, phentermine topiramate, naltrexone-bupropion, semaglutide, liraglutide, garcinia cambogia extract, Hydroxycut®, or glucomannan), or for weight gain (e.g., testosterone, growth hormone, anabolic steroids, or amino acid supplements); chronic (>14 consecutive days) systemic glucocorticoid therapy (excluding topical, intraocular, intranasal, intraarticular, or inhaled preparations) or have evidence of a significant, active autoimmune abnormality (e.g., lupus or rheumatoid arthritis) that has required (within the last 3 months) or is likely to require, in the opinion of the Investigator, concurrent treatment with systemic glucocorticoids (excluding topical, intraocular, intranasal, intra-articular or inhaled preparations) during the study participation; or any prescribed or non-prescribed drugs that are known to interfere with gut motility including, but not limited to chronic opioids, anticholinergics, antispasmodics, linaclotide, dopamine antagonists.

In some embodiments, the patient has not had any surgical treatment for obesity (of any type) or with a weight loss device or any other weight loss procedure (e.g., LAP-Band®, intragastric balloon, duodenal sleeve, resurfacing).

In some embodiments, the patient does not have obesity induced by other endocrinologic disorders (e.g., Cushing syndrome, acromegaly, or inadequately treated hypothyroidism) or diagnosis of monogenetic or syndromic forms of obesity (e.g., melanocortin 4 receptor deficiency or Prader Willi Syndrome). In some embodiments, the patient does not have a diagnosis and/or history of clinically significant gastroparesis, gastric motility, gastric emptying abnormalities, malabsorption disorders, chronic constipation, chronic diarrhea, inflammatory bowel disease, bowel resection, irritable bowel syndrome, or severe gastroesophageal reflux disease.

In some embodiments, the patient does not have myocardial infarction, unstable angina, coronary artery bypass graft, percutaneous coronary intervention (including participants who may have percutaneous transluminal coronary angioplasty [PTCA] planned and/or anticipated during the study period, such as participants with prior angiographic evidence that may require PTCA); transient ischemic attack, cerebrovascular accident, or hospitalization for congestive heart failure. The patient may not have current New York Heart Association Class III or IV heart failure.

In some embodiments, the patient does not have clinically significant electrocardiogram (ECG) findings (e.g., corrected QT interval using Fridericia's formula [QTcF] >450 msec for males, QTcF >470 msec for females, left bundle branch block), or any other ECG findings considered to be indicative of active cardiac disease or with abnormalities that are expected to interfere with the interpretation of ECG changes or may present a safety issue to the participant. In some embodiments, the patient does not have uncontrolled hypertension (i.e., mean seated systolic blood pressure of 160 mmHg or greater and/or mean seated diastolic blood pressure of 100 mmHg or greater).

In some embodiments, the patient does not have clinically significant proliferative retinopathy, macular edema, or other diabetes-related eye disease. In some embodiments, the patient does not have any hematologic conditions that may interfere with HbA1c measurement (e.g., hemolytic anemias, sickle cell disease, other hemoglobinopathies) or uncontrolled thyroid disease, defined as having active symptoms (e.g., palpitations, lethargy, weight gain, or weight loss) and/or having a thyroid-stimulating hormone (TSH) value outside the normal reference range.

In some embodiments, the patient does not have acute or chronic pancreatitis, or risk factors for pancreatitis, such as history of alcoholism, cholelithiasis (without cholecystectomy), hypercalcemia, or severe hypertriglyceridemia.

In some embodiments, the patient does not have alanine transaminase (ALT), or aspartate transaminase (AST), or gamma glutamyl transpeptidase (GGT) >3.0×the upper limit of normal (ULN) of the reference range; Alkaline phosphatase (ALP) >1.5×ULN of the reference range; total bilirubin >ULN of the reference range; Amylase or lipase level of >2×ULN of the reference range; fasting triglycerides level of >500 mg/dL; estimated glomerular filtration rate (eGFR) as calculated by the Modification of Diet in Renal Disease equation of <45 mL/min/1.73 m²; Calcitonin ≥20 ng/L, if eGFR is ≥60 mL/min/1.73 m²; or calcitonin ≥35 mg/L, if eGFR is <60 mL/min/1.73 m²; hepatitis (including hepatitis B or C); HIV; or hemoglobin level of <11 g/dL (male participants) or <10 g/dL (female participants).

In some embodiments, the T1DM patient is a patient with obesity. The patient may have a BMI of ≥30 to <40 kg/m². The patient may have HbA1c≥5.7% to ≤6.4%. In some embodiments, the present agonists may be used as an adjunct to a reduced calorie diet and increased physical activity for chronic weight management in adult patients with an initial body mass index (BMI) of 30 kg/m² or greater. In some embodiments, the patient has at least one weight-related comorbid condition (e.g., hypertension, type 2 diabetes mellitus, or dyslipidemia).

Body mass index (BMI) is a simple index of weight-for-height that is commonly used to classify overweight and obesity in adults. It is defined as a person's weight in kilograms divided by the square of his height in meters (kg/m²). For adults, WHO defines overweight as having a BMI of 25 to 29.9 kg/m², and obesity as having BMI ≥30 kg/m². Obesity is frequently subdivided into the following categories:
Obesity class I—BMI 30 to 34.9 kg/m²
Obesity class II—BMI 35 to 39.9 kg/m²; and
Obesity class III—BMI ≥40 kg/m² (also referred to as severe, extreme, or massive obesity)

In some embodiments, the definitions of overweight and obesity in Asian and South Asian populations may be as follows:
Overweight—BMI 23 to 24.9 kg/m²
Obesity—BMI ≥25 kg/m²

In some embodiments, the T1DM patient to be treated herein is overweight. In other embodiments, the patient to be treated herein is obese, with class I, II, or III obesity.

### V. T2DM PATIENT CHARACTERISTICS

In some embodiments, the patient has T2DM. In some embodiments, the patient does not have type 1 diabetes mellitus (T1DM). In some embodiments, the patient has BMI >27 to ≤45 kg/m². In some embodiments, the patient has HbA1c≤10.5% and FPG <250 mg/dL. The patient may be on a diet and exercise or stable therapy (≥3 months) with metformin monotherapy or metformin in combination with Sus prior to treatment. T2DM may be defined by the 2022 ADA standards of Medical Care in Diabetes (American Diabetes Association's (ADA), “Standards of Care in Diabetes,” Diabetes Care (2024) 47: S1-S321).

In some embodiments, the patient does not have any clinically significant active disease of the GI (e.g., peptic ulcers, severe gastroesophageal reflux disease (GERD), gastric emptying abnormality/gastroparesis, any malabsorption/motility disorders, chronic constipation, inflammatory bowel disease (IBD), or irritable bowel syndrome (IBS)), cardiovascular (e.g., arrhythmia, ischemic heart disease), hepatic, neurological, psychiatric, renal, immunological, dermatological, endocrine, genitourinary, or hematological systems. In some embodiments, the patient does not have hyperlipidemia, uncontrolled hypertension (BP) >140/90 mmHg (healthy participants), or a persistent BP systolic or diastolic >160/90 mmHg or <90/60 mmHg (participants with T2DM).

In some embodiments, the patient does not have acute or chronic pancreatitis or risk factors for pancreatitis (e.g., cholelithiasis (without cholecystectomy), hypercalcemia, or severe hypertriglyceridemia). In some embodiments, the patient does not have personal or family history of medullary thyroid carcinoma (MTC) or a genetic condition that predisposed to MTC (i.e., multiple endocrine neoplasia type 2).

In some embodiments, the patient does not have clinically significant physical or ECG findings (e.g., QTcF >450 msec for males, QTcF >470 msec for females, left bundle branch block [LBBB]). In some embodiments, the patient has not undergone a previous surgical treatment for obesity (any type of bariatric surgery) or any other GI surgery that may have induced malabsorption/motility issues, history of bowel resection >20 cm, or any GI procedure for weight loss (including LAP-BAND®).

In some embodiments, the patient does not use any prescribed or non-prescribed drugs that were known to interfere with glucose or insulin metabolism, including but not limited to systemic corticosteroids, testosterone, anabolic steroids, metformin, GLP-1 analogs/RAs, thiazolidinediones (TZDs), SU, dipeptidyl peptidase 4 (DPP-4) inhibitors, insulin therapy, monoamine oxidase (MAO) inhibitors, growth hormone, or other herbal/over the counter preparations, including amino acids.

In some embodiments, the patient does not use any prescribed or non-prescribed drugs that were known to interfere with gut motility including but not limited to chronic opioids, anticholinergics, antispasmodics, 5-hydroxytryptamine (5HT3) antagonists, or dopamine antagonists.

In some embodiments, the patient does not have clinically significant abnormal clinical laboratory values including transaminases (aspartate aminotransferase [AST], alanine aminotransferase [ALT] >1.5 (healthy) or ≥3 (with T2DM) times the upper limit of normal [ULN], total bilirubin >ULN, estimated glomerular filtration rate (eGFR)<60 mL/min/1.73 m² as estimated using the Modification of Diet in Renal Disease [MDRD] equation, and/or calcitonin levels >50 ng/L). Participants with T2DM were excluded if fasting serum triglycerides >500 mg/dL or laboratory values were suggestive of pancreatic impairment (e.g., amylase and/or lipase >3×ULN);

In some embodiments, the patient does not have hepatitis B, hepatitis C, or human immunodeficiency virus type 1 (HIV-1) or 2 (HIV-2).

In some embodiments, the T2DM patient is a patient with obesity. The patient may have a BMI of ≥30 to <40 kg/m². The patient may have HbA1c ≥5.7% to ≤6.4%. In some embodiments, the present agonists may be used as an adjunct to a reduced calorie diet and increased physical activity for chronic weight management in adult patients with an initial body mass index (BMI) of 30 kg/m² or greater. In some embodiments, the patient has at least one weight-related comorbid condition (e.g., hypertension, type 2 diabetes mellitus, or dyslipidemia).

In some embodiments, the patient to be treated herein is overweight (i.e., has a BMI of 25 to 29.9 kg/m²). In other embodiments, the patient to be treated herein is obese, with class I, II, or III obesity.

### VI. WEIGHT MANAGEMENT

In some embodiments, the patient does not have T1DM or T2DM but is overweight or obese. Patients may be 18-65 years of age. In some embodiments, the patient is a patient with obesity. The patient may have a BMI of ≥30 to <40 kg/m². The patient may have HbA1c≥5.7% to ≤6.4%. In some embodiments, the present agonists may be used as an adjunct to a reduced calorie diet and increased physical activity for chronic weight management in adult patients with an initial body mass index (BMI) of 30 kg/m² or greater. In some embodiments, the patient has at least one weight-related comorbid condition (e.g., hypertension, type 2 diabetes mellitus, or dyslipidemia).

In some embodiments, the patient to be treated herein is overweight. In other embodiments, the patient to be treated herein is obese, with class I, II, or III obesity.

Non-limiting examples of obesity (whether present alone or in combination with T1DM or T2DM) include symptomatic obesity, simple obesity, childhood obesity, morbid obesity and abdominal obesity (central obesity characterized by abdominal adiposity). Non-limiting examples of symptomatic obesity include endocrine obesity (e.g., Cushing syndrome, hypothyroidism, insulinoma, obese type II diabetes, pseudohypoparathyroidism, hypogonadism), hypothalamic obesity, hereditary obesity (e.g., Prader-Willi syndrome, Laurence-Moon-Biedl syndrome), and drug-induced obesity (e.g., steroid, phenothiazine, insulin, sulfonylurea agent, or β-blocker-induced obesity).

In addition to obesity, the patient may also have a condition, disease or disorder associated with obesity. Examples of such conditions, disease or disorders include, without limitation, glucose tolerance disorders, diabetes (e.g., type 2 diabetes, obese diabetes), lipid metabolism abnormality, hyperlipidemia, hypertension, cardiac failure, hyperuricemia, gout, fatty liver (including non-alcoholic steatohepatitis (NASH)), coronary heart disease (e.g., myocardial infarction, angina pectoris), cerebral infarction (e.g., brain thrombosis, transient cerebral ischemic attack), bone or articular disease (e.g., knee osteoarthritis, hip osteoarthritis, spondylitis deformans, lumbago), sleep apnea syndrome, obesity hypoventilation syndrome (Pickwickian syndrome), menstrual disorder (e.g., abnormal menstrual cycle, abnormality of menstrual flow and cycle, amenorrhea, abnormal catamenial symptom), visceral obesity syndrome, and metabolic syndrome. In certain embodiments, the chemical entities described herein can be used to treat subjects exhibiting symptoms of both obesity and insulin deficiency.

In some embodiments, a patient with obesity is treated with a dose of 0.1 to 15.0 mg of an agonist disclosed herein (e.g., CT-868). For example, patients may be treated with a dose of 0.5, 0.75, 1.5, 5.0, 7.5, or 11 mg CT-868. A patient with overweight or obesity may also be treated with a dose of 5.0 mg, 10.0 mg, or 15.0 mg of CT-868. In some embodiments, such patients are treated with the agonist daily.

In some embodiments, the patient does not have any clinically significant active disease of the GI (e.g., peptic ulcers, severe gastroesophageal reflux disease (GERD), gastric emptying abnormality/gastroparesis, any malabsorption/motility disorders, chronic constipation, inflammatory bowel disease (IBD), or irritable bowel syndrome (IBS)), cardiovascular (e.g., arrhythmia, ischemic heart disease), hepatic, neurological, psychiatric, renal, immunological, dermatological, endocrine, genitourinary, or hematological systems. In some embodiments, the patient does not have hyperlipidemia, uncontrolled hypertension (BP) >140/90 mmHg (healthy participants), or a persistent BP systolic or diastolic >160/90 mmHg or <90/60 mmHg (participants with T2DM).

In some embodiments, the patient does not have acute or chronic pancreatitis or risk factors for pancreatitis (e.g., cholelithiasis (without cholecystectomy), hypercalcemia, or severe hypertriglyceridemia). In some embodiments, the patient does not have personal or family history of medullary thyroid carcinoma (MTC) or a genetic condition that predisposed to MTC (i.e., multiple endocrine neoplasia type 2).

In some embodiments, the patient does not have clinically significant physical or ECG findings (e.g., QTcF >450 msec for males, QTcF >470 msec for females, left bundle branch block [LBBB]). In some embodiments, the patient has not undergone a previous surgical treatment for obesity (any type of bariatric surgery) or any other GI surgery that may have induced malabsorption/motility issues, history of bowel resection >20 cm, or any GI procedure for weight loss (including LAP-BAND®).

In some embodiments, the patient does not use any prescribed or non-prescribed drugs that were known to interfere with glucose or insulin metabolism, including but not limited to systemic corticosteroids, testosterone, anabolic steroids, metformin, GLP-1 analogs/RAs, thiazolidinediones (TZDs), SU, dipeptidyl peptidase 4 (DPP-4) inhibitors, insulin therapy, monoamine oxidase (MAO) inhibitors, growth hormone, or other herbal/over the counter preparations, including amino acids.

In some embodiments, the patient does not use any prescribed or non-prescribed drugs that were known to interfere with gut motility including but not limited to chronic opioids, anticholinergics, antispasmodics, 5-hydroxytryptamine (5HT3) antagonists, or dopamine antagonists.

In some embodiments, the patient does not have clinically significant abnormal clinical laboratory values including transaminases (aspartate aminotransferase [AST], alanine aminotransferase [ALT] >1.5 (healthy) or ≥3 (with T2DM) times the upper limit of normal [ULN], total bilirubin >ULN, estimated glomerular filtration rate (eGFR) <60 mL/min/1.73 m² as estimated using the Modification of Diet in Renal Disease [MDRD] equation, and/or calcitonin levels >50 ng/L). Participants with T2DM were excluded if fasting serum triglycerides >500 mg/dL or laboratory values were suggestive of pancreatic impairment (e.g., amylase and/or lipase >3×ULN);

In some embodiments, the patient does not have hepatitis B, hepatitis C, or human immunodeficiency virus type 1 (HIV-1) or 2 (HIV-2).

### VII. TREATMENT OUTCOMES

The present treatment methods may alter the insulin secretion rate; maximum glycemic excursion (Gₘₐₓ) AUC effect; fasting plasma glucose, insulin, C-peptide, GCC, or HOMA-IR; gastric emptying; glucose-stimulated insulin secretion (GSIS); glucose excursion; ambient glucose levels; HbA1c levels; or CGM metrics (TIR, time in hypo- and hyperglycemia, glycemic risk index) in a patient.

The present treatment methods may also decrease the incidence of level 3 hypoglycemic events or DKA in a patient.

The present treatment methods may reduce body weight in patients. In some embodiments, the methods result in weight loss of 5%, 10%, 15%, or 20%. In some embodiments, the present treatment methods alter total lean mass and total fat mass. Total lean mass and total fat mass may be measured by Dual energy X-ray absorptiometry (DEXA). DEXA scans assess body composition (fat and lean mass) and bone mineral density.

The present treatment methods may also result in changes in patient appetite, prospective food consumption, hunger, fullness, and satiety as measured by a VAS questionnaire. For the Satiety Questionnaire, the patients are requested to complete four questions using a 100 millimeter (mm) VAS. Based on the previous seven days, participants will be asked to rate their general Satiety/Satisfaction (100=completely satisfied, 0=not satisfied at all), Fullness (100-totally full, 0=not at all full), Hunger (100-never been more hungry, 0=not hungry at all), and Prospective Food Consumption (100=a lot, 0=nothing at all). The overall appetite score will be calculated as the average of the following 4 individual scores: [satiety+fullness+ (100-prospective food consumption)+ (100-hunger)]÷4.

The methods herein may also alter (e.g., decrease) food and caloric intake during an ad libitum meal.

The present treatment methods may also alter absolute and relative changes in basal, bolus, and total daily insulin use, as measured by units/day and units/kg/day.

### VIII. EXEMPLARY EMBODIMENTS

Non-limiting, exemplary embodiments are provided below to further illustrate the present disclosure.

1. A method of
adjunctive therapy for type 1 diabetes mellitus (T1DM),
improving glycemic control in a patient with T1DM,
increasing insulin sensitivity in a patient with T1DM,
increasing insulin-independent glucose disposal in a patient with T1DM,
reducing the need for therapeutic insulin in a patient with T1DM,
alleviating hypertension in a patient with T1DM,
reducing atherogenic lipids in a patient with T1DM, and/or
weight management in a patient with T1DM, comprising administering by subcutaneous injection to a patient in need thereof a dual agonist of glucagon-like peptide 1 (GLP-1) receptor and glucose-dependent insulinotropic polypeptide (GIP) receptor,
wherein the agonist comprises the following structure or a pharmaceutically acceptable salt or ester thereof:

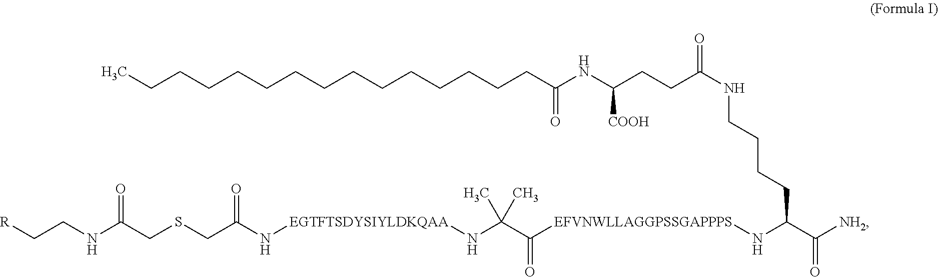

wherein R is a cycloalkyl or heterocyclic group that is 4-8 atoms in size, optionally substituted with a carbonyl, hydroxyl, methyl, phenyl, isopropyl, trifluoromethyl, or nitro group; and
wherein the agonist is administered at a body weight-independent dose of 1 mg to 20 mg.
   2. The method of embodiment 1, wherein the agonist comprises the following structure or a pharmaceutically acceptable salt or ester thereof:

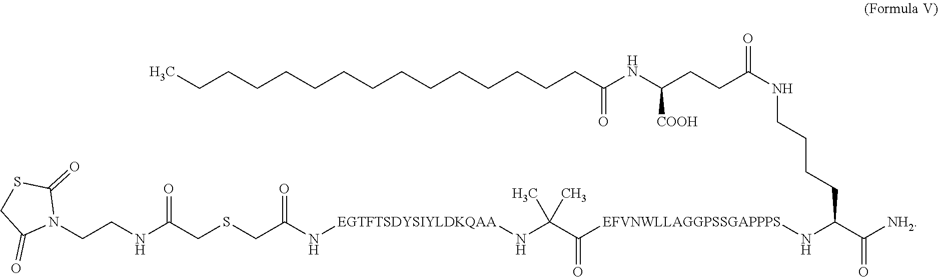

3. The method of embodiment 1, wherein the agonist comprises the following structure or a pharmaceutically acceptable salt or ester thereof:

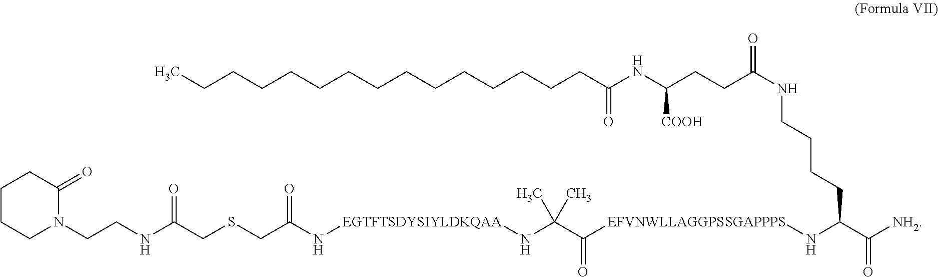

4. The method of any one of embodiments 1-3, wherein the administering step is repeated at an interval of one to seven days.
5. The method of embodiment 4, wherein the administering step is repeated once daily.
6. The method of any one of the preceding embodiments, wherein the dose is a dose that agonizes both GLP-1 receptor and GIP receptor.
7. The method of any one of the preceding embodiments, wherein the dose is about 1.8, 2, 3, 4, 5, 6, 7, 8, 9, 10, 11, 12, 13, 14, or 15 mg.
8. The method of any one of the preceding embodiments, wherein the patient has a BMI of 43 kg/m² or higher.
9. The method of any one of the preceding embodiments, wherein the patient receives an additional therapy for T1DM.
10. The method of embodiment 9, wherein the additional therapy is insulin therapy, diet therapy, exercise therapy, hypertension therapy, and/or blood lipid-lowering therapy.
11. A dual agonist of glucagon-like peptide 1 (GLP-1) receptor and glucose-dependent insulinotropic polypeptide (GIP) receptor for use in a method of any one of embodiments 1-10.
12. Use of a dual agonist of glucagon-like peptide 1 (GLP-1) receptor and glucose-dependent insulinotropic polypeptide (GIP) receptor for the manufacture of a medicament for use in a method of any one of embodiments 1-10.
An article of manufacture for use in a method of any one of embodiments 1-10, wherein the article of manufacture comprises one or more units of said dose.
13. The article of manufacture of embodiment 13, wherein the article of manufacture is a syringe or an injector, optionally a single-use syringe or injector.
14. A method of
adjunctive therapy for an insulin-dependent patient with type 1 diabetes mellitus (T1DM) or type 2 diabetes mellitus (T2DM),
treating T1DM or T2DM in a patient in need thereof,
improving glycemic control in a patient with T1DM or T2DM,
increasing insulin sensitivity in a patient with T1DM or T2DM,
increasing insulin-independent glucose disposal in a patient with T1DM or T2DM,
reducing the need for therapeutic insulin in a patient with T1DM or T2DM,
alleviating hypertension in a patient with T1DM or T2DM,
reducing atherogenic lipids in a patient with T1DM or T2DM, and/or
weight management in a patient with T1DM or T2DM, comprising administering by subcutaneous injection to a patient in need thereof a dual agonist of glucagon-like peptide 1 (GLP-1) receptor and glucose-dependent insulinotropic polypeptide (GIP) receptor,
wherein the agonist comprises the following structure or a pharmaceutically acceptable salt or ester thereof:

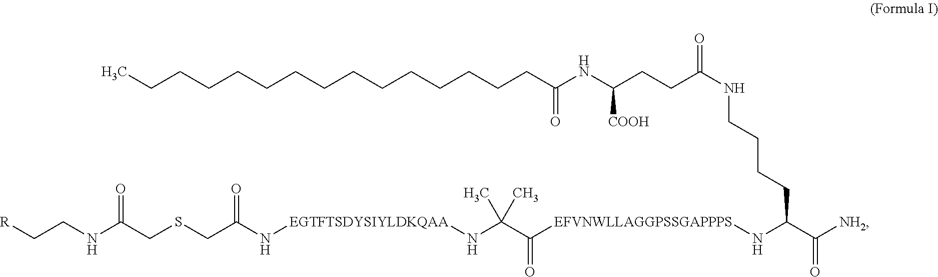

wherein R is a cycloalkyl or heterocyclic group that is 4-8 atoms in size, optionally substituted with a carbonyl, hydroxyl, methyl, phenyl, isopropyl, trifluoromethyl, or nitro group; and
wherein the agonist is administered at a body weight-independent dose of 1 mg to 20 mg.
   15. The method of embodiment 14, wherein the agonist comprises the following structure or a pharmaceutically acceptable salt or ester thereof:

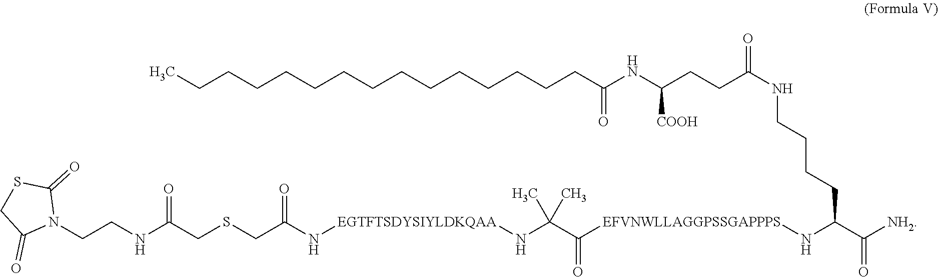

16. The method of embodiment 14, wherein the agonist comprises the following structure or a pharmaceutically acceptable salt or ester thereof:

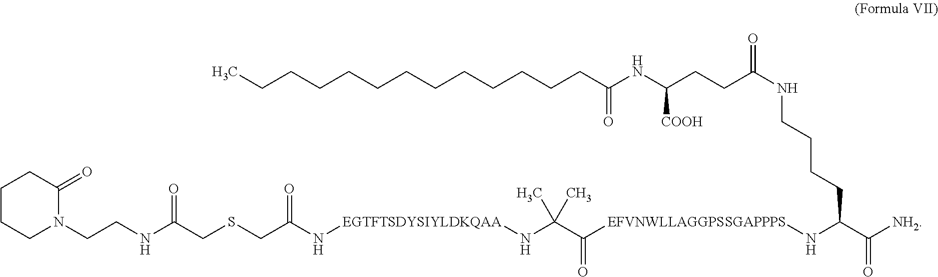

17. The method of any one of embodiments 14-16, wherein the administering step is repeated at an interval of one to seven days.
18. The method of embodiment 17, wherein the administering step is repeated once daily.
19. The method of any one of embodiments 14-18, wherein the dose is a dose that agonizes both GLP-1 receptor and GIP receptor.
20. The method of any one of embodiments 14-19, wherein the dose is about 1.8, 2, 3, 4, 5, 6, 7, 8, 9, 10, 11, 12, 13, 14, or 15 mg.
21. The method of any one of embodiments 14-20, wherein the patient has a BMI of 25 kg/m² or higher.
22. The method of any one of embodiments 14-21, wherein the patient receives an additional therapy for T1DM or T2DM.
23. The method of embodiment 22, wherein the additional therapy is insulin therapy, diet therapy, exercise therapy, hypertension therapy, and/or blood lipid-lowering therapy.
24. A dual agonist of glucagon-like peptide 1 (GLP-1) receptor and glucose-dependent insulinotropic polypeptide (GIP) receptor for use in a method of any one of embodiments 14-23.
25. Use of a dual agonist of glucagon-like peptide 1 (GLP-1) receptor and glucose-dependent insulinotropic polypeptide (GIP) receptor for the manufacture of a medicament for use in a method of any one of embodiments 14-23.
26. An article of manufacture for use in a method of embodiments 14-23, wherein the article of manufacture comprises one or more units of said dose.
27. The article of manufacture of embodiment 26, wherein the article of manufacture is a syringe or an injector, optionally a single-use syringe or injector.
28. A method of
adjunctive therapy for an insulin-dependent patient with type 1 diabetes mellitus (T1DM) or type 2 diabetes mellitus (T2DM),
treating T1DM or T2DM in a patient in need thereof,
improving glycemic control in a patient with T1DM or T2DM,
increasing insulin sensitivity in a patient with T1DM or T2DM,
increasing insulin-independent glucose disposal in a patient with T1DM or T2DM,
reducing the need for therapeutic insulin in a patient with T1DM or T2DM,
alleviating hypertension in a patient with T1DM or T2DM,
reducing atherogenic lipids in a patient with T1DM or T2DM, and/or
weight management in a patient with T1DM or T2DM, comprising administering by subcutaneous injection to a patient in need thereof a dual agonist of glucagon-like peptide 1 (GLP-1) receptor and glucose-dependent insulinotropic polypeptide (GIP) receptor,
wherein the agonist comprises the following structure or a pharmaceutically acceptable salt or ester thereof:

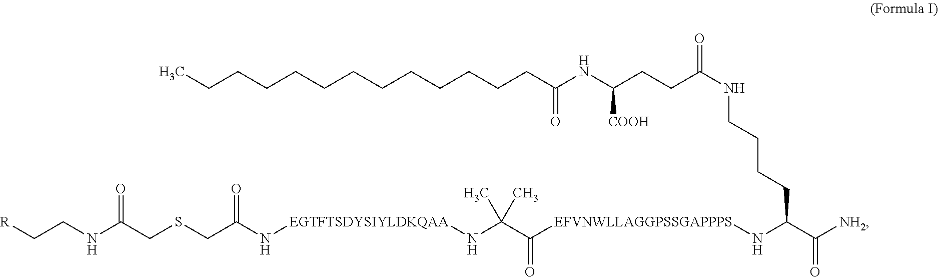

wherein R is a cycloalkyl or heterocyclic group that is 4-8 atoms in size, optionally substituted with a carbonyl, hydroxyl, methyl, phenyl, isopropyl, trifluoromethyl, or nitro group; and
wherein the agonist is administered at a body weight-independent dose of 1 mg to 20 mg.
   29. The method of embodiment 28, wherein the agonist comprises the following structure or a pharmaceutically acceptable salt or ester thereof:

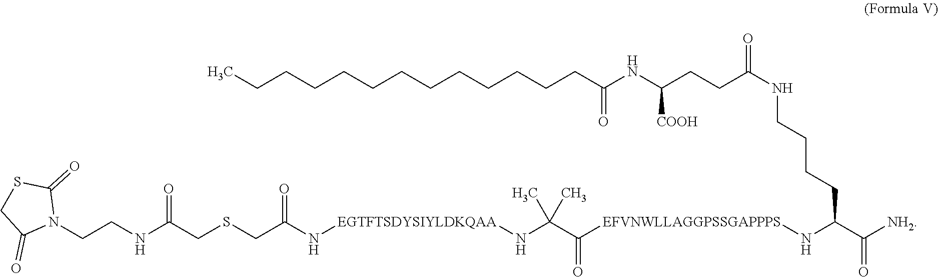

30. The method of embodiment 28, wherein the agonist comprises the following structure or a pharmaceutically acceptable salt or ester thereof:

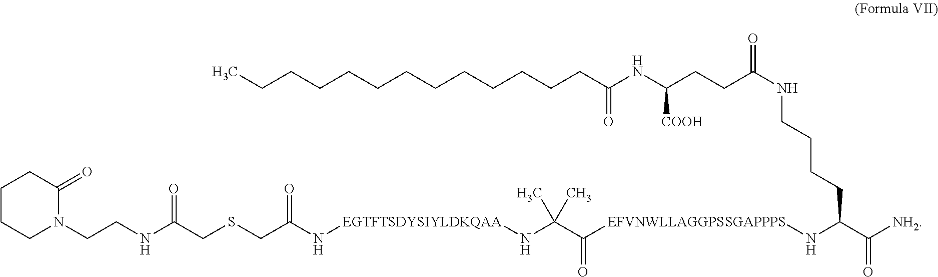

31. The method of any one of embodiments 28-30, wherein the administering step is repeated at an interval of one to seven days.
32. The method of embodiment 31, wherein the administering step is repeated once daily.
33. The method of any one of embodiments 28-32, wherein the dose is a dose that agonizes both GLP-1 receptor and GIP receptor.
34. The method of any one of embodiments 28-33, wherein the dose is about 1.5, 1.8, 2, 3, 4, 5, 6, 7, 8, 9, 10, 11, 12, 13, 14, 15, 16, 17, 18, 19, or 20 mg.
35. The method of any one of embodiments 28-34, wherein the patient has a BMI of 25 kg/m² or higher.
36. The method of any one of embodiments 28-35, wherein the patient receives another therapy for T1DM or T2DM.
37. The method of embodiment 36, wherein the other therapy is insulin therapy, diet therapy, exercise therapy, hypertension therapy, and/or blood lipid-lowering therapy.
38. A dual agonist of glucagon-like peptide 1 (GLP-1) receptor and glucose-dependent insulinotropic polypeptide (GIP) receptor for use in a method of any one of embodiments 28-37.
39. Use of a dual agonist of glucagon-like peptide 1 (GLP-1) receptor and glucose-dependent insulinotropic polypeptide (GIP) receptor for the manufacture of a medicament for use in a method of any one of embodiments 28-37.
40. An article of manufacture for use in a method of any one of embodiments 28-37, wherein the article of manufacture comprises one or more units of said dose.
41. The article of manufacture of embodiment 40, wherein the article of manufacture is a syringe or an injector, optionally a single-use syringe or injector.
42. A compound for use in treating type 1 diabetes mellitus (T1DM) in a patient in need thereof, wherein the compound comprises the following structure or a pharmaceutically acceptable salt or ester thereof:

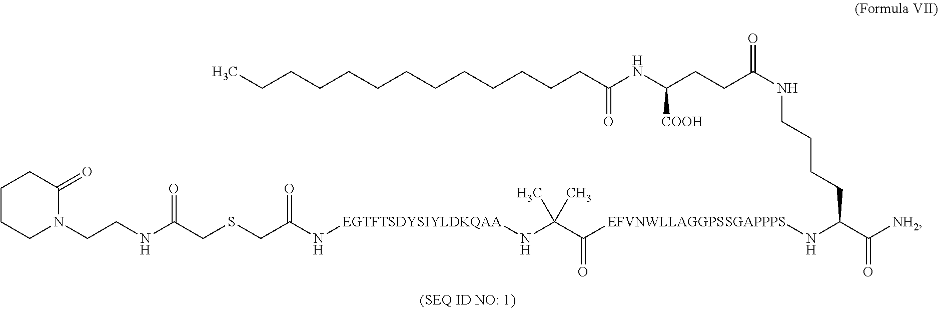

wherein the compound is to be administered by subcutaneous injection to the patient in need thereof at a body weight-independent dose of 1 mg to 20 mg.
43. A compound for use in treating type 2 diabetes mellitus (T2DM) in a patient in need thereof, wherein the compound comprises the following structure or a pharmaceutically acceptable salt or ester thereof:

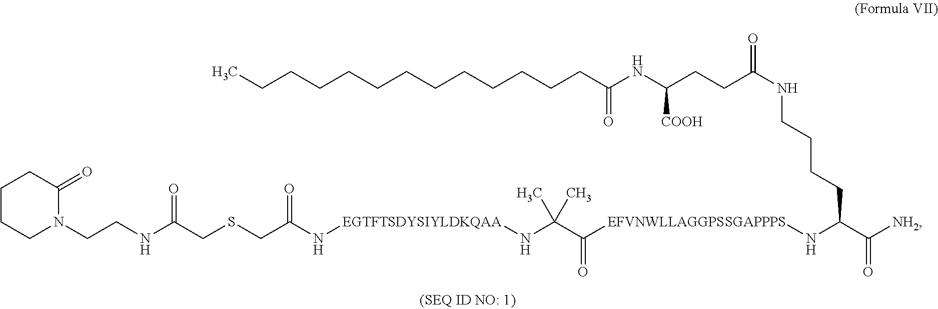

wherein the compound is to be administered by subcutaneous injection to the patient in need thereof at a body weight-independent dose of 1 mg to 20 mg.
44. The compound for use of embodiment 42 or 43, where the compound is for use to
improve glycemic control in the patient,
increase insulin sensitivity in the patient,
increase insulin-independent glucose disposal in the patient,
reduce the need for therapeutic insulin in the patient,
alleviate hypertension in the patient, and/or
reduce atherogenic lipids in the patient.
   45. The compound for use of any one of embodiments 42-44, wherein the patient is overweight or obese, and the method reduces body weight of the patient.
   46. The compound for use in of weight management in a patient in need thereof, wherein the compound comprises the following structure or a pharmaceutically acceptable salt or ester thereof:

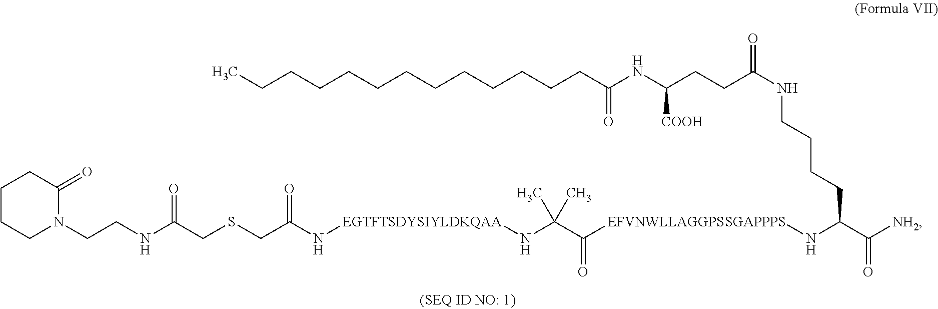

wherein the compound is to be administered by subcutaneous injection to the patient in need thereof at a body weight-independent dose of 1 mg to 20 mg.
47. The compound for use of embodiment 46, wherein the patient has type 1 or type 2 diabetes mellitus.
48. The compound for use of any one of embodiments 42-47, wherein the administration of the compound is an adjunct to one or more additional therapies.
49. The compound for use of embodiment 48, wherein the one or more additional therapies comprise insulin therapy, diet therapy, exercise therapy, hypertension therapy, and/or blood lipid-lowering therapy.
50. The compound for use of any one of embodiments 42-49, wherein the administering step is repeated at an interval of one to seven days.
51. The compound for use of any one of embodiments 42-49, wherein the administering step is repeated once daily.
52. The compound for use of any one of embodiments 42-51, wherein the compound is administered at a dose that agonizes both GLP-1 receptor and GIP receptor.
53. The compound for use of any one of embodiments 42-52, wherein the dose is about 1.5, 1.8, 2, 3, 4, 5, 6, 7, 8, 9, 10, 11, 12, 13, 14, 15, 16, 17, 18, 19, or 20 mg.
54. The compound for use of any one of embodiments 52-53, wherein the dose is 1.77, 3.25 mg, or 4 mg.
55. The compound for use of any one of embodiments 42-54, wherein the dose is 1.1, 1.8, 2.6, 3.3, 4.1, 5.2, or 6.6 mg.
56. The compound for use of any one of embodiments 42-55, wherein the patient has a BMI of 25 kg/m² or higher.
57. The compound for use of any one of embodiments 42-56, wherein the compound is to be administered to the patient in a pharmaceutical composition comprising:
the compound at a concentration of 15 mg/mL,
di-sodium hydrogen phosphate heptahydrate/sodium dihydrogen phosphate monobasic buffer solution, optionally at a concentration of 20 mM,
propylene glycol, and
phenol, further optionally wherein the pharmaceutical composition is at pH 7.0.
   58. A pharmaceutical composition comprising
(i) a compound comprising the following structure or a pharmaceutically acceptable salt or ester thereof, at a concentration of 15 mg/mL:

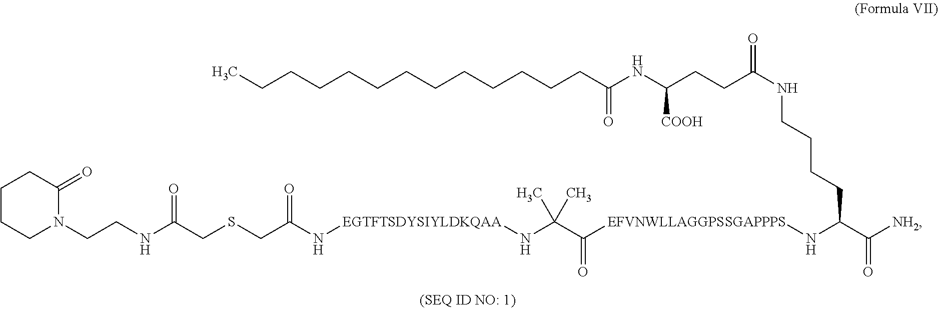

(ii) di-sodium hydrogen phosphate heptahydrate/sodium dihydrogen phosphate monobasic buffer solution, optionally at a concentration of 20 mM,
(iii) propylene glycol, and
(iv) phenol, further optionally wherein the pharmaceutical composition is at pH 7.0.
   59. The compound herein for use of any one of embodiments 42-57, provided in a pharmaceutical composition of claim 58
   60. An article of manufacture comprising the compound for use of any one of embodiments 42-57 and 59, or the pharmaceutical composition of embodiment 58, wherein the article of manufacture comprises one or more units of said dose, optionally a single dose unit.
   61. An article of manufacture comprising the compound for use of any one of embodiments 42-57 and 59, or the pharmaceutical composition of embodiment 58, wherein the article of manufacture comprises five units of said dose.
   62. The article of manufacture for use of embodiment 60 or 61, wherein the article of manufacture is a syringe, injection pen, or an injector, optionally wherein the article of manufacture is single-use.
   63. The article of manufacture for use of any one of embodiments 60-62, wherein the article of manufacture comprises a needle-based injection system with an integrated non-replaceable 3-mL Type 1 glass cartridge and a pharmaceutical composition comprising 3 mL of the agonist at a concentration of 15 mg/mL.

Unless otherwise defined herein, scientific and technical terms used in connection with the present disclosure shall have the meanings that are commonly understood by those of ordinary skill in the art. Exemplary methods and materials are described below, although methods and materials similar or equivalent to those described herein can also be used in the practice or testing of the present disclosure. In case of conflict, the present specification, including definitions, will control. Further, unless otherwise required by context, singular terms shall include pluralities and plural terms shall include the singular. Throughout this specification and embodiments, the words “have” and “comprise,” or variations such as “has,” “having,” “comprises,” or “comprising,” will be understood to imply the inclusion of a stated integer or group of integers but not the exclusion of any other integer or group of integers. All publications and other references mentioned herein are incorporated by reference in their entirety, as if each individual reference were specifically and individually indicated to be incorporated by reference in its entirety. Although a number of documents are cited herein, this citation does not constitute an admission that any of these documents forms part of the common general knowledge in the art. As used herein, the term “approximately” or “about” as applied to one or more values of interest refers to a value that is similar to a stated reference value. In certain embodiments, the term refers to a range of values that fall within 10%, 9%, 8%, 7%, 6%, 5%, 4%, 3%, 2%, 1%, or less in either direction (greater than or less than) of the stated reference value unless otherwise stated or otherwise evident from the context.

According to the present disclosure, back-references in the dependent claims are meant as short-hand writing for a direct and unambiguous disclosure of each and every combination of claims that is indicated by the back-reference. Any compound disclosed herein can be used in any of the treatment method here, wherein the individual to be treated is as defined anywhere herein. Further, headers herein are created for ease of organization and are not intended to limit the scope of the claimed invention in any manner.

In order that this invention may be better understood, the following examples are set forth. These examples are for purposes of illustration only and are not to be construed as limiting the scope of the invention in any manner.

### EXAMPLES

### Example 1: Biased GLP-1R and GIPR Agonists with No β-Arrestin Coupling Produce Sustained Glucose, Food Intake, and Body Weight Reduction in Rodent Models of Obesity

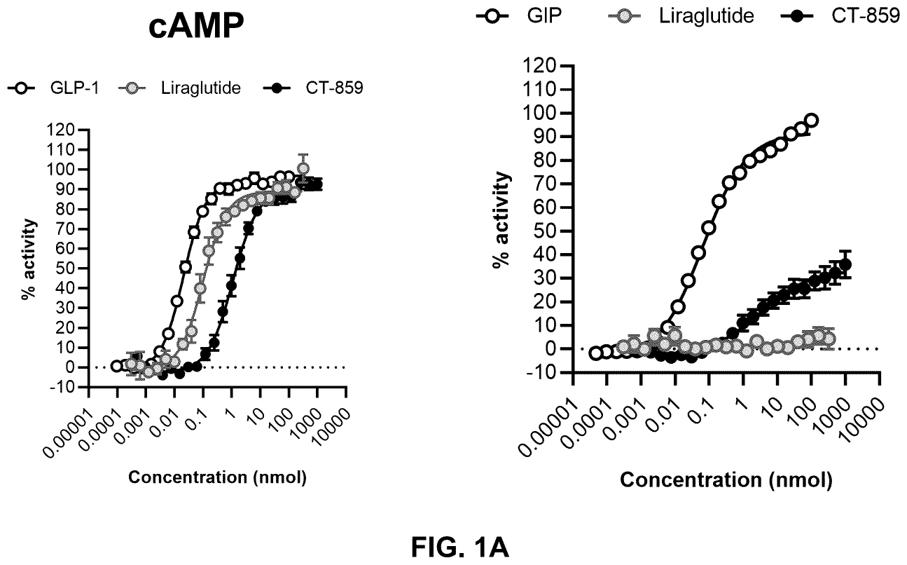

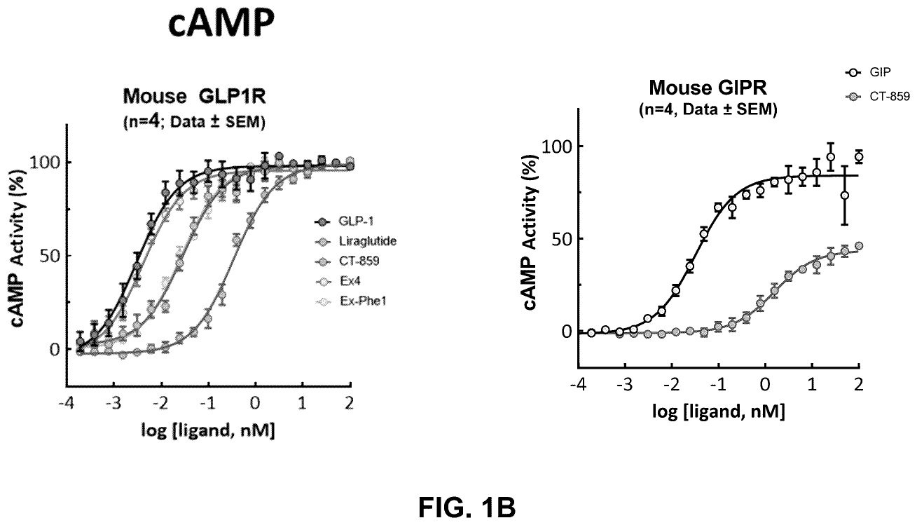

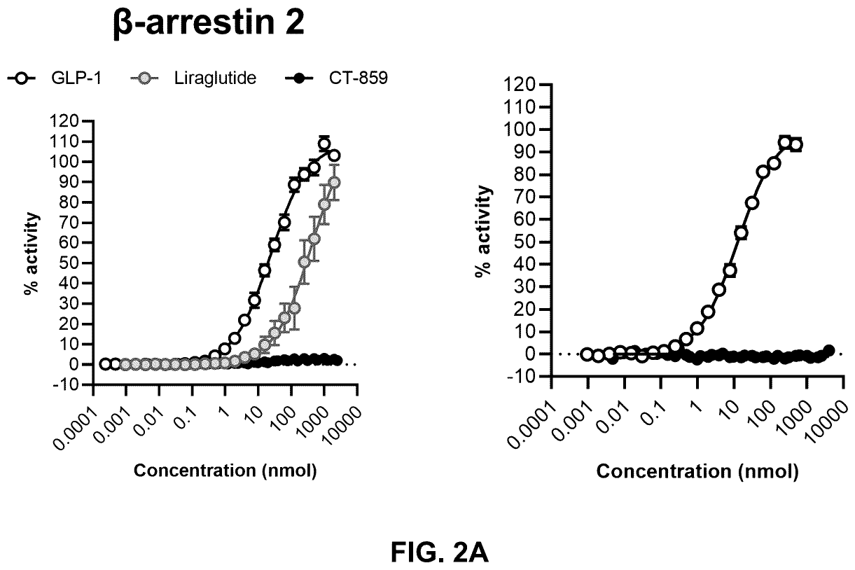

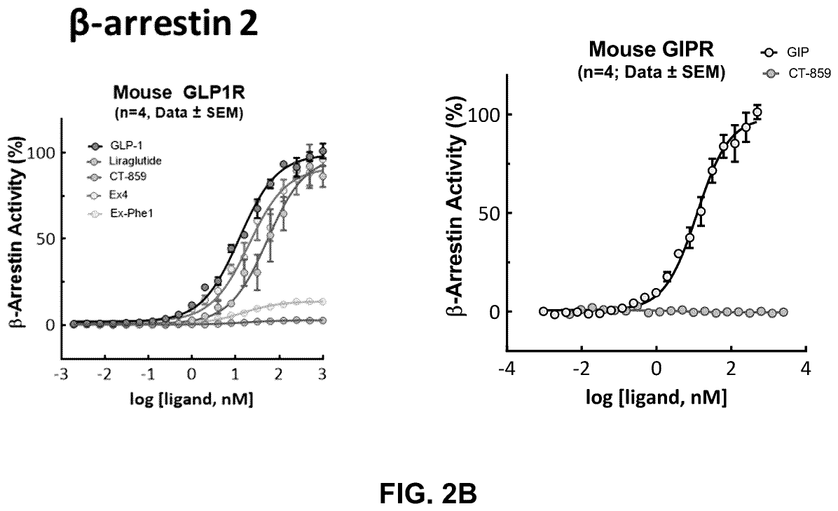

To elucidate the importance of signaling bias, CT-859, a fully cAMP-biased dual GLP-1/GIP receptor agonist (FIGS. 1A and 1B), was designed. CT-859 exhibits no β-arrestin coupling at either receptor (FIGS. 2A and 2B). The effects of CT-859 on glucose (GLUC) homeostasis, food intake (FI) regulation, and weight loss (WL) were evaluated in rodent models.

### Method

CAMP generation by GLP-1, GIP, liraglutide, CT-859, exendin-4, and exendin-Phe1 was measured using mammalian cell lines overexpressing either the GLP-IR or GIPR using the HitHunter® CAMP Assay for Small Molecules (Catalog number 90-0075SM) kit. For mouse receptors, stably transfected U2OS (GLP-IR-DiscoverX 95-0179C3) and CHO (GIPR-DiscoverX 95-0154C2) cells were used (FIGS. 1A and 1B). β-arrestin-2 recruitment by GLP-1, GIP, liraglutide, CT-859, exendin-4, and exendin-Phe1 was measured with a β-galactosidase complementation assay using the PathHunter® CHO-K1 GLP-IR β-arrestin (DiscoverX 93-0300C2), PathHunter® CHO-K1 GIPR β-Arrestin (DiscoverX 93-1095C2), HEK293 mGLPIR, and/or HEK293 mGIPR β-Arrestin cell lines with the PathHunter® Detection Kit (FIGS. 2A and 2B).

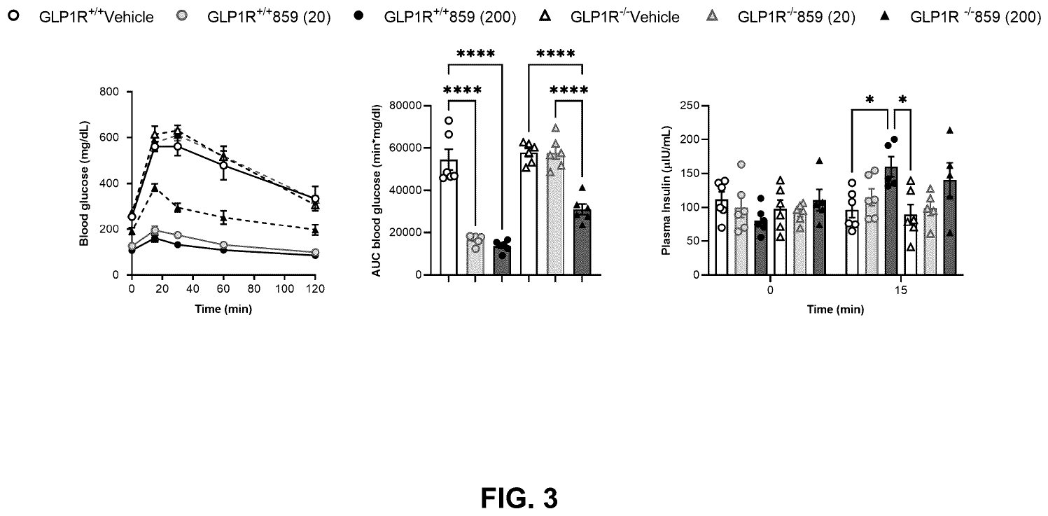

12-13-week-old male GLP-1R+/+ (WT) and GLP-IR−/− (GLP-1R-KO) mice from Taconic were individually housed and fed a regular chow diet. For the intraperitoneal glucose tolerance test (ipGTT), mice were randomized by body weight within their respective genotype to the following groups: WT and GLP-1R-KO Vehicle (phosphate buffer+Tween; n=6), WT and GLP-1R-KO CT-859 (20 nmol/kg; n=6), and WT and GLP-1R-KO CT-859 (200 nmol/kg; n=6). Intraperitoneal glucose tolerance tests (ipGTT) were performed after five hours of fasting and four hours after compound administration. After the fasting period glucose was measured, blood was collected, and glucose (2 g/kg) was administered by an intraperitoneal injection. After administration, blood glucose levels were measured and blood was collected for insulin measurements 15, 30, 60, and 120 minutes after the glucose injection. Insulin was measured using an MSD insulin kit. Results were analyzed using a two-way ANOVA and Holm-Sidak's test for multiple comparisons (FIG. 3).

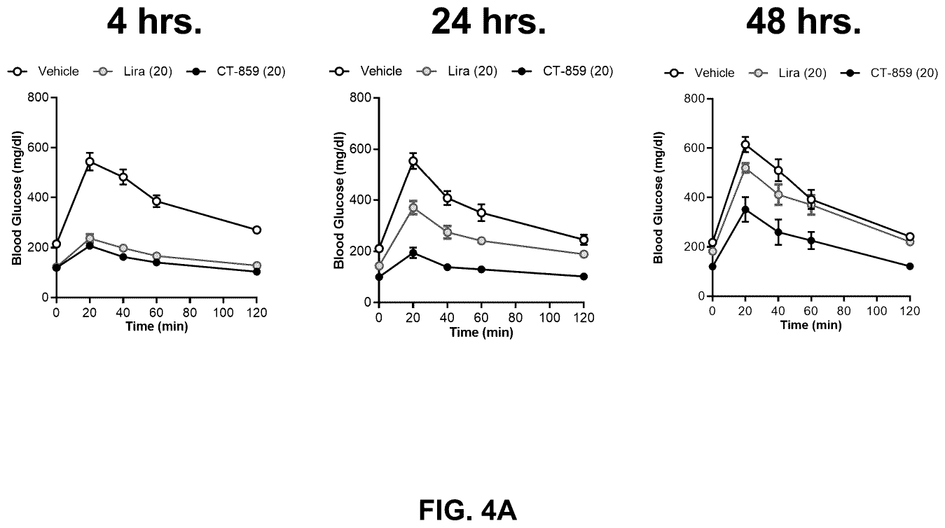

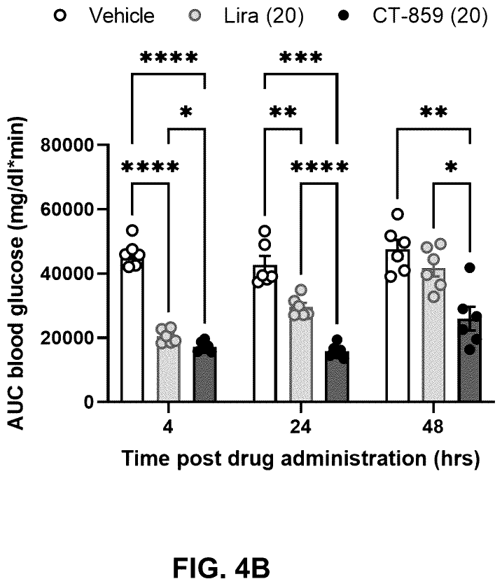

Naive 10-week-old male C57BL/6J mice from Jackson laboratory were housed individually and fed a regular chow diet. Mice were randomized by body weight to the following groups: vehicle (phosphate buffer+Tween; n=6 per time point), liraglutide (20 nmol/kg; n=6 per time point), and CT-859 (20 nmol/kg; n=6 per time point). Intraperitoneal glucose tolerance tests (ipGTT; 2 g/kg) were performed 4, 24, and 48 hours after compound administration. Mice were fasted for five hours before the ipGTT. After the fasting period glucose was measured and glucose (2 g/kg) was administered by an intraperitoneal injection. Blood glucose levels were measured 20, 40, 60, and 120 minutes after the glucose injection. Results were analyzed using a two-way ANOVA and Holm-Sidak's test for multiple comparisons (FIGS. 4A and 4B).

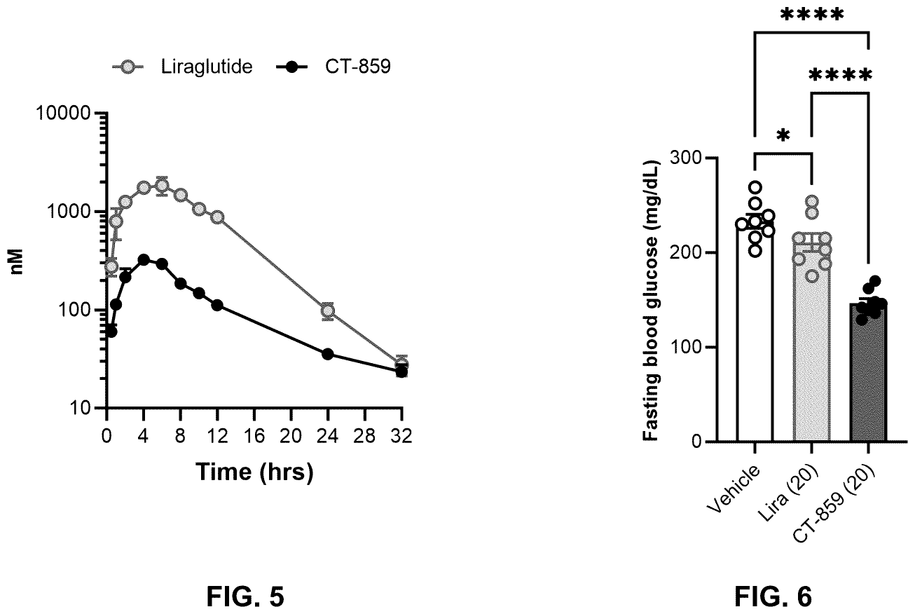

Pharmacokinetic studies and bioanalysis were performed by BioDuro Inc. and are briefly described here. 7- to 9-week-old male CD-1 mice received liraglutide (1 mg/kg) or CT-859 (1 mg/kg) by a single subcutaneous (SC) injection. Thereafter blood was collected in K2-EDTA Microvettes® from a saphenous vein puncture 0.5, 1, 2, 4, 6, 8, 10, 12, 24, and 32 after drug administration. Blood was kept on ice until centrifuged at 4600 rpm for five minutes at 4° C. Plasma was stored at −80° C. until analysis. Compounds were extracted from plasma and quantified against a standard curve using an AB Sciex 7500+LC-MS/MS system (FIG. 5).

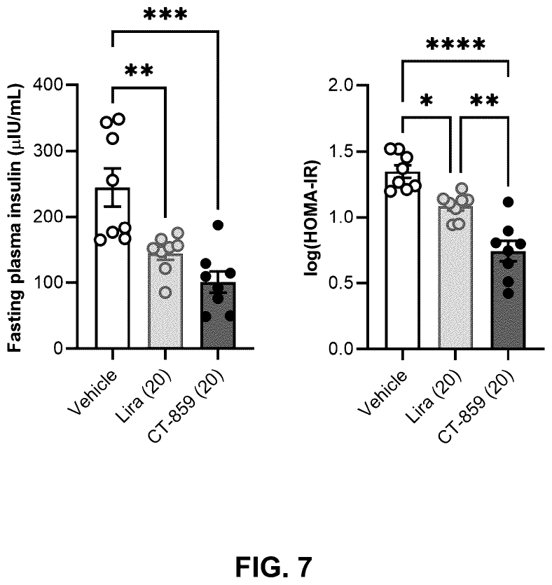

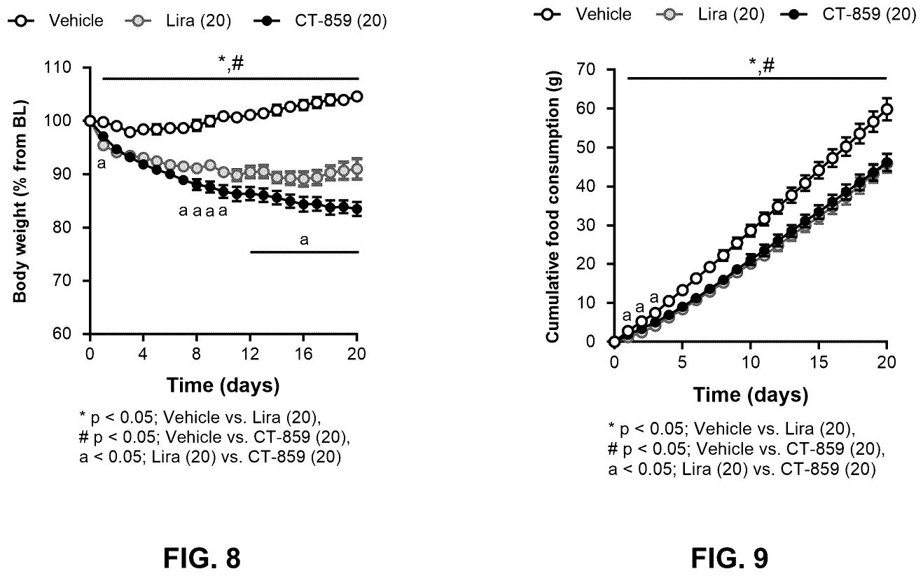

Naïve 24-week-old male DIO C57BL/6J mice from Jackson laboratory were individually housed and fed a 60% high fat diet. Mice were randomized by body weight to the following groups: vehicle (phosphate buffer+tween; n=8), liraglutide (20 nmol/kg; n=8), and CT-859 (20 nmol/kg; n=8). Compounds were administered daily by a single subcutaneous injection for 21 days. Body weight and food consumption were measured daily (FIGS. 8 and 9). On the last day of the study mice were fasted for 5 hours. At the end of the fasting period, glucose was measured (FIG. 6) and plasma was collected for insulin analysis. Plasma insulin was measured using an MSD kit. Fasting glucose and insulin were used to calculate the log (HOMA-IR) (FIG. 7). Body weight and cumulative food consumption were analyzed using a two-way ANOVA and Holm-Sidak's test for multiple comparisons. Fasting glucose, insulin and log (HOMA-IR) were analyzed using a one-way ANOVA and Holm-Sidak's test for multiple comparisons.

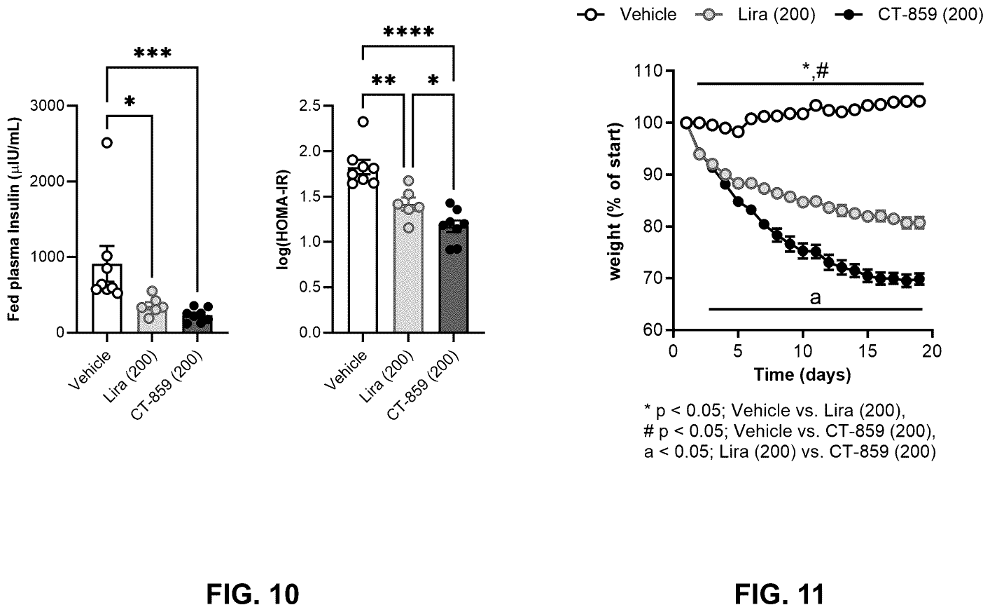

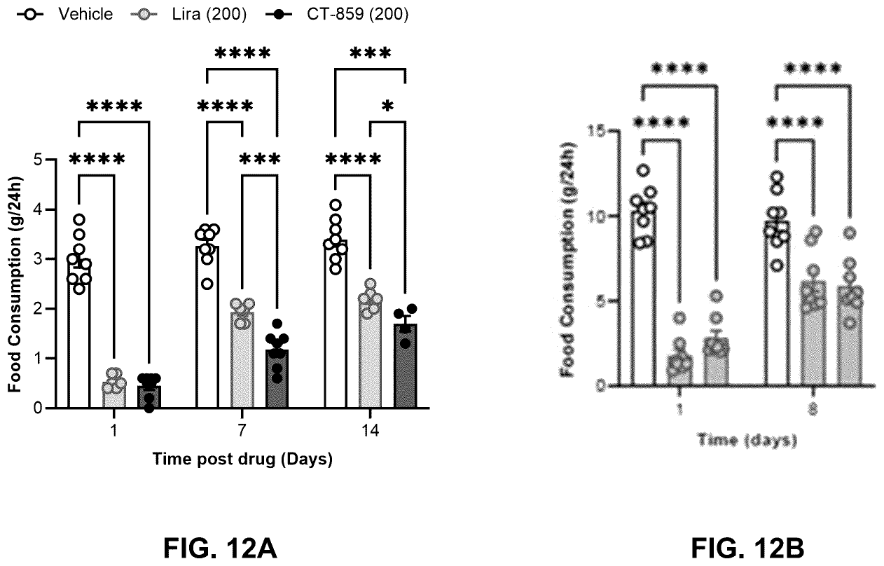

Naïve 23-week-old male DIO C57BL/6J mice from Jackson laboratory were individually housed and fed a 60% high fat diet. Mice were randomized by body weight to the following groups: vehicle (phosphate buffer+tween; n=8), liraglutide (200 nmol/kg; n=8), and CT-859 (200 nmol/kg; n=8). Compounds were administered daily by a single subcutaneous injection for 21 days. Body weight was measured daily (FIG. 11). Food consumption was measured either 1, 7, and 14 days (FIG. 12A) or 8 days (FIG. 12B) after the start of the treatments. On the last day of the study, glucose was measured, and plasma was collected for insulin analysis. Plasma insulin was measured using an MSD kit. Glucose and insulin were used to calculate log (HOMA-IR) (FIG. 10). Body weight and food consumption were analyzed using a two-way ANOVA and Holm-Sidak's test for multiple comparisons. Plasma insulin and log (HOMA-IR) were analyzed using a one-way ANOVA and Holm-Sidak's test for multiple comparisons.

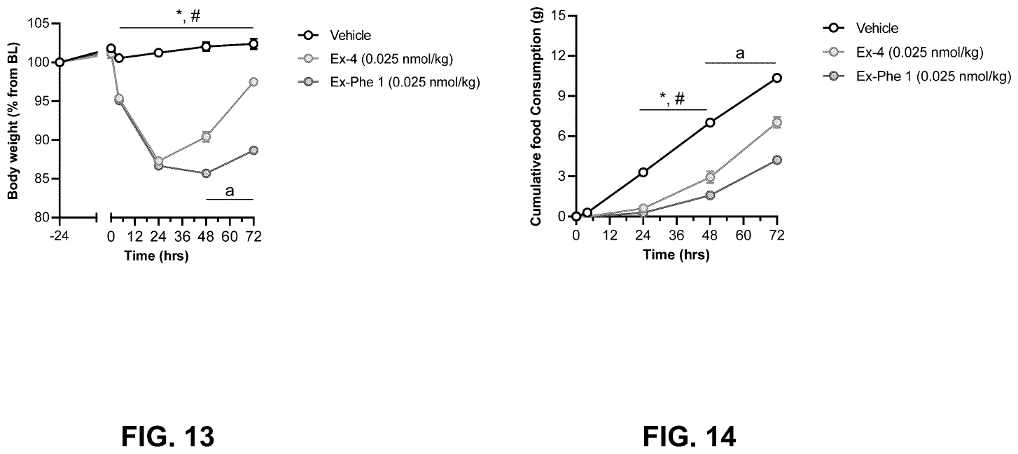

WT mice were randomized by body weight to the following groups: Vehicle (phosphate buffer+Tween; n=9), exendin-4 (0.025 nmol/kg; n=8), and exendin-Phe1 (0.025 nmol/kg; n=10). Compounds were administered only once by an intracerebroventricular (ICV) injection, and body weight and food consumption were measured at 4-hours, 24-hours, 48-hours, and 72-hours thereafter (FIGS. 13 and 14). Body weight and cumulative food consumption were analyzed using a two-way ANOVA and Holm-Sidak's test for multiple comparisons.

### Results

In GLP-1R knockout mice, a 20 nmol/kg dose of CT-859 was established as a GLP-1R-only engaging dose as it had no effect on GLUC AUC during intraperitoneal glucose tolerance test (IPGTT) and was similar to that of vehicle (veh), indicating a lack of GIPR activation at this dose (FIG. 3). In lean mice, a 20 nmol/kg dose of CT-859 led to an acute reduction of glucose (GLUC) AUC that was similar to that achieved with a 20 nmol/kg dose of an unbiased GLP-IRA (liraglutide), four hours after drug administration (FIGS. 4A and 4B) despite CT-859 having 82% lower plasma exposure four hours after drug administration (FIG. 5). Remarkably, CT-859 remained efficacious up to 48 hours (45% reduction in GLUC AUC vs. veh, p<0.001), whereas liraglutide-treated mice were indistinguishable from vehicle-treated mice (FIGS. 4A and 4B). Diet-induced obese (DIO) mice dosed for 20 days with CT-859 at 20 nmol/kg exhibited decreased fasting GLUC (146.5 vs. 210.6 mg/dL; p<0.0001) (FIG. 6) and insulin resistance (log (HOMA-IR); 0.74 vs. 1.08; p=0.001) (FIG. 7), and achieved greater weight loss (WL) (−16.5 vs. −9.1%; p=0.001) (FIG. 8) without significant difference in cumulative food intake (FI) (FIG. 9) compared to DIO mice dosed for 20 days with liraglutide at 20 nmol/kg. In contrast, DIO mice dosed for 20 days with a GIPR-engaging 200 nmol/kg dose of CT-859, exhibited decreased insulin resistance (nonfasted log (HOMA-IR); 1.17 vs. 1.42, p<0.05) (FIG. 10), produced more WL (−30.1 vs. −19.2%; p<0.0001) (FIG. 11), and induced significant FI suppression (day 7:1.2 vs. 1.9 g; p=0.0003; day 14:1.7 vs. 2.2 g; p<0.05) (FIG. 12A) compared to DIO mice dosed for 20 days with a 200 nmol/kg dose of liraglutide, suggesting these differences may be a consequence of central GIPR activation.

In lean mice, administration of exendin-4 and exendin-Phe1 via ICV injection led to significant reduction of cumulative food consumption, which was associated with reduction of body weight. However, administration of exendin-Phe1 led to greater reduction of food intake and much more prolonged body weight reduction as compared to exendin-4 (FIGS. 13 and 14).

Together, these findings demonstrate that CT-859, a fully biased dual GLP-1/GIP R agonist (GLP-1/GIPRA) with imbalanced activity in favor of GLP-IR, can produce both a sustained and higher magnitude of GLUC reduction, FI suppression, and WL beyond that achieved by liraglutide, an unbiased GLP-IR agonist (GLP-1RA).

### Example 2: Biased Signaling Enhances the Glucose-Lowering and Weight-Loss Effects of a Dual GLP-1R/GIPR Agonist

GLP-1 and GIP are hormones that promote insulin secretion in a glucose-dependent manner and suppress food intake. This Example describes CT-859, a biased dual GLP-1/GIPR agonist that exhibits inverse receptor internalization agonism. At lower concentrations, CT-859 lowers glucose levels for up to 48 hours through GLP-IR activation, whereas the effects of liraglutide last only 24 hours. Moreover, CT-859 decreased body weight more than liraglutide. We also demonstrated that a biased GIPR agonist was more effective in reducing glucose levels than an unbiased agonist. Central administration of biased GLP-1R and GIPR agonists results in greater food consumption and body weight reduction than unbiased agonists, and their combination is more efficacious than combining a biased GLP-IR agonist and an unbiased GIPR agonist. These findings demonstrate that incretin-based therapies that stimulate biased signaling are more effective than those that are unbiased and that bias signaling should be considered when designing new incretin-based therapies.

### Methods

The sources for select reagents and resources are shown in Table 1 below.

TABLE 1
Sources for Select Reagents and Resources

| | | |
|---|---|---|
| Reagent or Resource | Source | Identifier |

Chemicals, peptides, and recombinant proteins

| | | |
|---|---|---|
| IBMX | Sigma | 45-I7018-1G-EA |
| Human GLP-1 (7-36) | Anaspec | AS-22463 |
| Human GIP (1-42) | Anaspec | AS-61226-1) |
| Liraglutide | SelleckChem | S8256 |
| Exendin-4 | Santa Cruz Biotech | Sc-474611 |
| Potassium Phosphate Monobasic | Sigma-Aldrich | P8709-1L |
| solution | | |
| Potassium phosphate dibasic solution | Sigma-Aldrich | P8584-1L |
| TWEEN ® 20, PROTEIN GRADE | EMD Millipore Corp. | 655206-50 mL |
| Water, Optima LC/MS Grade | Fisher Scientific | W64 |
| Saline | Pfizer | 00409488810 |

Critical commercial assays

| | | |
|---|---|---|
| HitHunter ® cAMP Assay for | Eurofins DiscoverX | 90-0075LM |
| Biologics | | |
| Nano-Glo ® Live Cell Substrate | Promega | N2012 |
| Nano-Glo ® HiBiT Extracellular | Promega | N2421 |
| Detection System | | |
| Fugene 6 | Promega | E2692 |
| U-PLEX Mouse Insulin Assay | Meso Scale Discovery | K1526HK-2 |

Experimental models: Cell lines

| | | |
|---|---|---|
| HEK293 | ATCC | CRL-1573 |
| CHO-K1 | ATCC | CCL-61 |
| CHO-K1 Snap-human GLP-1R Cell | Carmot | |
| Line | | |
| CHO-K1 Snap-human GIPR Cell Line | Carmot | |
| CHO-K1 murine GIPR Gs Cell Line | Eurofins DiscoverX | 95-0154C2 |
| U2OS murine GLP-1R Gs Cell Line | Eurofins DiscoverX | 95-0179C3 |
| HEK293 human GLP-1R-LgBiT | Carmot | |
| SmBiT-human-b-arrestin-2 | | |
| HEK293 human GIPR-LgBiT SmBIT- | Carmot | |
| human-b-arrestin-2 | | |
| HEK293 mouse GLP-1R-LgBiT | Carmot | |
| SmBiT-mouse-b-arrestin-2 | | |
| HEK293 mouse GIPR-LgBiT SmBIT- | Carmot | |
| mouse-b-arrestin-2 | | |

Experimental models: Organisms/strains

| | | |
|---|---|---|
| C57BL/6J | The Jackson Laboratory | 000664 |
| C57BL/6J DIO | The Jackson Laboratory | 380050 |
| C57BL/6-Glp1r^{em1}_1Cgen | Taconic Biosciences | |
| C57BL/6- | Taconic Biosciences | |
| Gt(ROSA)26Sor^{tm16(cre)Arte} | | |
| Gipr^{em1.2Cgen} | | |

Recombinant DNA

| | |
|---|---|
| SNAP-human GLP-1R | Cisbio |
| SNAP-human GIPR | Cisbio |
| (Rattus norvegicus)-GLP-1R | Genscript |
| pcDNA3.1/Zeo | |
| (Rattus norvegicus)-GIPR | Genscript |
| pcDNA3.1/Zeo | |
| cynomolgus monkey GLP-1R | Genscript |
| pcDNA3.1/Zeo | |
| cynomolgus monkey GIPR | Genscript |
| pcDNA3.1/Zeo | |
| Human GLP-1R-LgBiT | Promega |
| Human GIPR-LgBiT | Promega |
| SmBiT- (human)-β-arrestin-2 | Promega |
| Mouse GLPIR-LgBiT | Promega |
| Mouse GIPR-LgBiT | Promega |
| smBIT_(mouse)- β-arrestin-2 | Promega |
| pcDNA3.1 (empty) | Genscript |
| HiBiT-(human)-GLP-1R | Promega |
| HiBiT-(human)-GIPR | Promega |

Software and algorithms

| | |
|---|---|
| Prism | GraphPad |
| Excel | Microsoft |

Other

| | | |
|---|---|---|
| Guide cannula | Protech International Inc. | C315GAS-5/SPC |
| Dummy cannula | Protech International Inc. | C315DCS-5/SPC |
| Internal cannula | Protech International Inc. | C315IAS-5/SPC |
| Stainless-steel screw | Protech International Inc. | |
| Dental cement | Stoelting | 51458 |
| Syringes | Hamilton Company, US | 203073 |
| Glucose meter | Data Sciences International | 51073 |
| Glucose strips | Data Sciences International | 42214-100 |
| Alpha Trak 3 Starter Kit | Zoetis | MWI 119405 |
| AlphaTRAK 3 Test Strips | Zoetis | MWI 119409 |
| Chow diet | | |
| 60% HFD | Research Diets, Inc | D12492 Q#1 |
| 50% dextrose | | |

### Mice

C57BL/6J and diet induced obese (DIO) C57BL/6J male mice were obtained from The Jackson Laboratory. GLP-1R^{+/+}, GLP-1R^{-/-}, GIPR^{+/+}, and GIPR^{-/-} male mice were obtained from Taconic Biosciences. Mice were singly housed under standard environmental conditions (22° C., 12 h: 12 h light: dark cycle), with ad libitum access to water and regular chow (C57BL/6J; Teklad rodent diet 2920x, Inotiv) or HFD (DIO C57BL/6J; 60% kcal from fat, Research Diets #D12492) unless otherwise specified. Lean mice were used between 8-10 weeks of age. DIO mice were maintained on HFD for at least 18 weeks before experimentation. All studies were approved by FibroGen and Explora BioLabs' Institutional Animal Care and Use Committee.

### Peptide Synthesis

Peptides were synthesized by microwave-assisted solid-phase peptide synthesis (SPPS) techniques using an Fmoc/t-Bu strategy on a Liberty Blue Microwave Peptide Synthesizer (CEM Corporation). Fmoc Deprotections were carried out using 20% piperidine in 0.1 M Oxyma/DMF solution. Amino acid couplings were performed using a five-fold excess of reagent; Fmoc-amino acids (0.2 M solution in DMF), DIC (0.5 or 1.0 M solution in DMF) and Oxyma (0.5 or 1.0 M solution in DMF) were employed on 0.05 or 0.1 mmol scale on Rink Amide ProTide Resin (LL) or preloaded Wang (LL) resin.

Cleavage and Work-up Conditions: side chain protecting group removal with concomitant cleavage from the resin and was carried out in a TFA/TIS/H2O/PhOH (88:2:5:5 v/v/v/v) solution (10 mL/0.05 mmol) for 3 hours at room temperature. Cold diethyl ether (30 mL/0.05 mmol) was used to precipitate the peptide, which was isolated by centrifugation (3000 rpm, 10 min).

Purification Conditions: crude peptide was iteratively purified by RP-HPLC until >95% purity was obtained. Purification conditions are listed in Tables 2-5 as follows; suitable fractions were pooled and lyophilized. Peptide purity was determined by analytical RP-HPLC, and identity was confirmed using LCMS.

TABLE 2
Purification Procedure P1

| Column: Phenomenex Jupiter Proteo 90Å 4 μm, 250 × 21.2 mm | | |
|---|---|---|
| Solvent A = H₂O with 0.1% TFA | | |
| Solvent B = MeCN with 0.1% TFA | | |
| Flow rate = 15 mL/min | | |
| Column temperature: 20-25° C. | | |
| Gradient: 0-100% B over 30 min | | |
| Time (min) | % A | % B |
| 0 | 100 | 0 |
| 3 | 100 | 0 |
| 33 | 0 | 100 |
| 33.1 | 0 | 100 |
| 43 | 0 | 100 |
| 43.1 | 100 | 0 |
| 48 | 100 | 0 |

TABLE 3
Purification Procedure P2
Column: Phenomenex Aeris 5 μm Peptide XB-C18 250 × 21.2 mm
AXIA packed
Solvent A = H₂O with 20 mM NH₄HCO₃
Solvent B = MeCN
Flow rate = 15 mL/min
Column temperature: 20-25° C.
Gradient: 20-50% B over 45 min

| Time (min) | % A | % B |
|---|---|---|
| 0 | 100 | 0 |
| 0.1 | 95 | 5 |
| 2 | 80 | 20 |
| 47 | 50 | 50 |
| 47.1 | 0 | 100 |
| 57 | 0 | 100 |
| 57.1 | 100 | 0 |
| 61 | 100 | 0 |

TABLE 4
Purification Procedure P3
Column: Phenomenex Aeris 5 μm Peptide XB-C18 250 × 21.2 mm
AXIA packed
Solvent A = H₂O with 0.1% TFA
Solvent B = MeCN with 0.1% TFA
Flow rate = 25 mL/min
Column temperature: 21° C.
Gradient: 20-40% B over 25 min

| Time (min) | % A | % B |
|---|---|---|
| 0 | 100 | 0 |
| 0.25 | 95 | 5 |
| 2.50 | 80 | 20 |
| 27.50 | 60 | 40 |
| 27.51 | 0 | 100 |
| 32.50 | 0 | 100 |
| 32.51 | 100 | 0 |
| 35.00 | 100 | 0 |

TABLE 5
Purification Procedure P4
Column: Phenomenex Aeris 5 μm Peptide XB-C18 250 × 21.2 mm
AXIA packed
Solvent A = H₂O with 20 mM NH₄HCO₃
Solvent B = MeCN
Flow rate = 25 mL/min
Column temperature: 21° C.
Gradient: 20-40% B over 25 min

| Time (min) | % A | % B |
|---|---|---|
| 0 | 100 | 0 |
| 0.25 | 95 | 5 |
| 2.50 | 80 | 20 |
| 27.50 | 60 | 40 |
| 27.51 | 0 | 100 |
| 32.50 | 0 | 100 |
| 32.51 | 100 | 0 |
| 35.00 | 100 | 0 |

Analytical HPLC Conditions: The purity of peptides was examined by analytical RP-HPLC, and identity confirmed using LCMS with the conditions shown in Tables 6-8.

TABLE 6
Analytical RP-HPLC Condition A1
Column: Waters XSelect ® CSH ™ Prep C18 5 μm 3.0 × 150 mm
Solvent A = H₂O with 0.1% TFA
Solvent B = MeCN with 0.1% TFA
Flow rate = 1 mL/min
Column temperature: 21° C.
Wavelength: 214 nm

| Time (min) | % A | % B |
|---|---|---|
| 0 | 95 | 5 |
| 3.00 | 70 | 30 |
| 23.00 | 45 | 55 |
| 24.00 | 5 | 95 |
| 33.00 | 5 | 95 |
| 33.10 | 95 | 5 |
| 35.00 | 95 | 5 |

TABLE 7
Analytical RP-HPLC Condition A2
Column: Waters XSelect ® CSH ™ Prep C18 5 μm 3.0 × 150 mm
Solvent A = H₂O with 0.1% TFA
Solvent B = MeCN with 0.1% TFA
Flow rate = 1 mL/min
Column temperature: 21° C.
Wavelength: 214 nm

| Time (min) | % A | % B |
|---|---|---|
| 0 | 95 | 5 |
| 3.00 | 70 | 30 |
| 23.00 | 50 | 50 |
| 24.00 | 5 | 95 |
| 33.00 | 5 | 95 |
| 33.10 | 95 | 5 |
| 35.00 | 95 | 5 |

TABLE 8
LC-MS Conditions
Column: Phenomenex Gemini ® 3 μm C19 110 Å LC Column 30 × 2 mm
Solvent A = H₂O with 0.1% Formic Acid
Solvent B = MeCN with 0.1% Formic Acid
Flow rate = 0.9 mL/min
Column temperature: 21° C.
Mass Range: 900-1500 m/z

| Time (min) | % A | % B |
|---|---|---|
| 0.10 | 95 | 5 |
| 0.11 | 75 | 25 |
| 1.50 | 0 | 100 |
| 1.80 | 0 | 100 |
| 1.81 | 95 | 5 |
| 2.00 | 95 | 5 |

Preparation of CT-859: The sold-phase preparation of CT-859 is shown below:

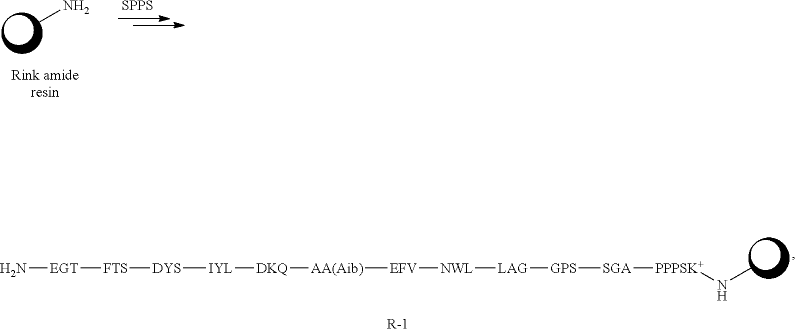

wherein K* is:

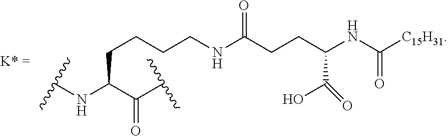

The N-terminal modification of CT-859 was prepared according to the following synthesis:

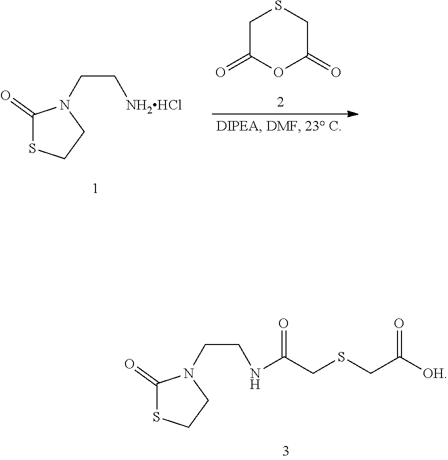

R-1 was coupled to carboxylic acid 3 above and subsequently cleaved from the resin according to the following procedure:

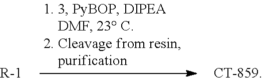

Resin-bound peptide intermediate R-1 was prepared from Rink amide resin by standard solid phase peptide synthesis methods using Fmoc chemistry with α-Fmoc and sidechain protected canonical amino acid building blocks. Fmoc-(α-aminoisobutyric acid) and N2-(((9H-fluoren-9-yl) methoxy) carbonyl)-N6-((S)-5-(tert-butoxy)-5-oxo-4-palmitamidopentanoyl)-L-lysine were also employed in addition to standard amino acid building blocks.

To a slurry of hydrochloride salt 1 (9.83 g, 50 mmol) and thiodiglycolic anhydride 2 (6.61 g, 50 mmol, 1.0 equiv.) in DMF (150 mL) was added DIPEA (34.8 mL, 4.0 equiv.) at 23° C. The reaction mixture was stirred at ambient temperature for 30 minutes, at which point the reaction was deemed complete by HPLC analysis. PyBOP (26.0 g, 1.0 equiv.) was added followed by additional DIPEA (17.4 mL, 2.0 equiv.). After stirring for five minutes at ambient temperature, the reaction mixture was added to resin R-1 (14 mmol). The slurry was agitated for 4 hours at ambient temperature and filtered. The resin was washed with DMF (3×), IPA (3×) and isopropyl ether (2×) and dried in vacuo to afford 128.6 g of product resin. The resin was treated with TFA/TIS/DOT/H2O (90/5/2.5/2.5 v/v/v/v) for 3 hours and the reaction mixture was filtered. The resin was washed with TFA and the filtrate and washes were combined and diluted with pre-cooled isopropyl ether. The precipitated solids were collected by filtration and dried in vacuo to afford 44 g of the crude peptide.

The crude peptide was purified using a 2″ C18 column with a gradient of 0.05M AcOH in H₂O/MeCN (flow rate=100 mL/min), followed by salt exchange to the hydrochloride salt with 0.01N HCl buffer (flow rate=100 mL/min) on a 2″ C18 column. CT-859 hydrochloride (6 g) was afforded as a white powder (97.0% purity by analytical HPLC), ESI-MS found 1543.9. C₂₁₂H₃₂₀N₄₇O₆₅S₂ [M+3H]³⁺ requires 1543.9.

### Preparation of Ex-Phe1

The sequence of Ex-Phe1 is:

(SEQ ID NO: 2)
FG-EGT-FTS-DLS-KQM-EEE-AVR-LFI-EWL-LAG-GPS-SGA-
PPPS-NH₂.

Exendin-Phe1 (Ex-Phe1) was prepared on 0.15 mmol scale in two batches (0.1 mmol and 0.05 mmol) by standard Fmoc-SPPS methods. The resin from the 0.1 mmol scale batch was subjected to cleavage conditions using 6 mL of TFA/TIS/PhOH/H2O (88:2:5:5 v/v/v/v) for three hours. The resin was filtered and washed with TFA (2×2 mL). The filtrate and washes were combined and diluted with ice cold Et20 (30 mL) to precipitate the peptide. The slurry was centrifuged at 2000 rpm for 10 minutes and the supernatant was drained. The crude peptide was dissolved in 10 mL of DMSO/AcOH (1:1 v/v). The resin from the 0.05 mmol scale batch was also subjected to cleavage conditions using TFA/TIS/DODT/H2O (90:5:2.5:2.5 v/v/v/v) for three hours and processed as described for the earlier batch. The solutions of crude peptide from both batches were combined for HPLC purification. Initial purification was carried out using HPLC method P1, followed by a second purification using HPLC method P2 to afford 27.7 mg of Ex-Phe1 (96.0% purity, 214 nm) by analytical HPLC (condition A2). ESI-MS found 1050.0, C₁₈₇H₂₈₈N₄₈O₆₀S (M+4H)⁴⁺ requires 1050.2.

### CAMP Production

Cells were maintained in cell-specific media in a 37° C. incubator at 5% CO₂. CAMP generation was measured using the HitHunter® CAMP Assay kit. For both GLP-1R and GIPR, 10,000 cells per well were plated 24 hours prior in assay complete cell plating reagent 2 (GIPR) or 5 (GLP-1R) in 384 well low-volume tissue culture treated plates. Before starting the assay, media was replaced with κ mL 1:2 ratio of anti-cAMP antibody: 1×HBSS/10 mM HEPES/625 mM IBMX. Compound dilutions were made 1:1 in DMSO, and then 5 nL was transferred to wells using an ECHO acoustic liquid handler (Labcyte). Plated cells were incubated for 30 minutes at 37° C., and cells in suspension were incubated for 30 minutes at room temperature while shaking. cAMP detection reagents were added according to the manufacturer's specifications, and luminescence was measured after the indicated incubation times.

β-arrestin 2 Recruitment

To measure GLP-1R and GIPR-mediated β-arrestin recruitment, we utilized Promega's NanoBiT® technology or NanoLuc® Binary Technology. The NanoLuc luciferase is split into two subunits, called LgBiT and SmBIT, which are expressed in HEK293 cells as fusion proteins at the C-terminus of GLP-IR or GIPR and the N-terminus of β-arrestin-2. Twenty-four hours before β-arrestin NanoBiT® assays, cells were lifted with Cell Dissociation media and plated at 10,000 cells per well in TC-treated 384 low-volume plates. The next day, the media was removed and replaced with 10 mL 1:100 dilution of Nano-Glo® Live Cell Substrate in Opti-MEM™ and equilibrated to room temperature for ten minutes. Background luminescence was measured before 10 nL compound was added using an ECHO Acoustic Liquid Handler. Luminescence was measured at 1.5-minute intervals for 30 minutes using an En Vision Multimode Plate Reader (PerkinElmer). All assays were set up such that each row of a 384 well plate contained a single dilution series and, for normalization purposes, a single low control well (vehicle-treated) and a single high control well (GLP-1 or GIP-treated).

### GLP-1R Internalization

GLP-1R and GIPR internalization were measured using Promega's Nano-Glo® HiBit extracellular detection system (Promega Corporation; Madison, WI). Hek293 cells were transiently transfected with HiBiT-tagged hGLP-1R or hGIPR plasmids (Promega Corporation; Madison, WI). Cells were lifted using the TrypLE express enzyme (ThermoFisher Scientific) and plated at 80,000-100,000 cells per well in 96 well plates. Cells were then treated with Nano-Glo HibiT extracellular buffer containing LgBiT protein (1:100), and the Nano-Glo HiBiT extracellular substrate (1:50). Next, test compounds in DMSO were added to cells, and plates were read on an EnVision multimode plate reader (Perkin Elmer; Waltham, MA), for 120 minutes at two-minute intervals.

### Intraperitoneal Glucose Tolerance Test

Liraglutide and CT-859 were administered by a single subcutaneous injection (SC) at the doses provided in each FIG. 4—48 hours before the intraperitoneal glucose tolerance test (ipGTT). On the day of the ipGTT, mice were fasted for five hours. Baseline blood glucose was determined using an AlphaTrakII® glucose meter from whole blood collected from the tip of the tail. After that, glucose 2 g/kg dextrose (as a 20% solution in saline) was injected by a single intraperitoneal injection. Blood glucose was determined at the specified time intervals in each figure. When plasma insulin concentration was desired, mice were warmed under a heat lamp, blood glucose concentration was determined, and ˜20 μL whole blood was collected in K2-EDTA microvettes (Sarstedt). The area under the curve (AUC) analysis was performed using GraphPad Prism software with the baseline set at Y=0.

### Food Consumption

Acute food intake: Mice were weighed, glucose levels were measured using a Nova Biomedical glucose meter (Data Sciences International), and treatments were administered by a single SC injection or an intracerebroventricular injection into the lateral ventricle. Mice were returned to their cage with a pre-weighed amount of food on the cage floor. Body weight, food consumption, and glucose levels were measured 24, 48, and up to 72 hours post-treatment administration.

### Intracerebroventricular Cannulations

Male C57BL/6J, GLP-1RKO, wild type litter mates, and C57BL/6J DIO were implanted with a 26-gauge guide cannula with a 5 mm pedestal, and the cannula cut 1.6 mm below the pedestal using the following coordinates: anterior-posterior (AP); −0.4 mm, R/L; 1 mm, dorsal-ventral (DV); −1.3 mm. Mice were allowed to recover for one week. After one week, mice were manually restrained and injected with vehicle or compounds using a 33-gauge internal cannula injector with a 0.8 mm projection.

### Weight Loss

DIO mice were acclimated to daily weighing and handling for approximately 1 week until their weight had stabilized. Mice were injected subcutaneously once daily with the indicated dose of peptide, or vehicle, based on current body weight. Injections were performed six hours before the dark cycle started to approximate reaching Tmax when the dark cycle started. Where indicated, 24-hour food consumption studies were performed as described above. Body weight as a percentage of initial weight was calculated daily by dividing the daily body weight by the body weight taken before the first dose of peptide, multiplied by 100%. Blood glucose was determined at study termination, and blood was collected to quantify plasma insulin concentrations. Mice were sedated by isoflurane and euthanized by decapitation. Whole trunk blood was collected in K2-EDTA microvettes and was kept on ice until centrifugation at 5000 rcf for 10 minutes at 4° C. Plasma was stored at −80° C. until analysis. Subcutaneous and inguinal fat and the liver were removed and weighed.

### Pharmacokinetics

Pharmacokinetic (PK) analysis for CT-859 and liraglutide were performed in CD-1 male mice by BioDuro Inc.

### Plasma Insulin

Whole blood collected in K2-EDTA microvettes was kept on ice until centrifugation at 5000 rcf for ten minutes at 4° C. Plasma was removed and stored at −80° C. until analyzed. Plasma insulin concentrations were determined using the U-PLEX Mouse Insulin Assay (Meso Scale Discovery).

### Statistics

Results are presented as means (±standard error). A one-way ANOVA with Bonferroni's test for multiple comparisons was used to detect differences in glucose tolerance. A 2-way ANOVA with group and time as between-subject factors was used for everything else. Multiple comparisons were carried out using a Bonferroni correction. Statistical significance was set at p<0.05. Statistical analyses were performed with GraphPad Prism 9.2.0 (GraphPad Software, Boston, MA).

### Results

CT-859 is a biased dual GLP-1R/GIPR agonist.

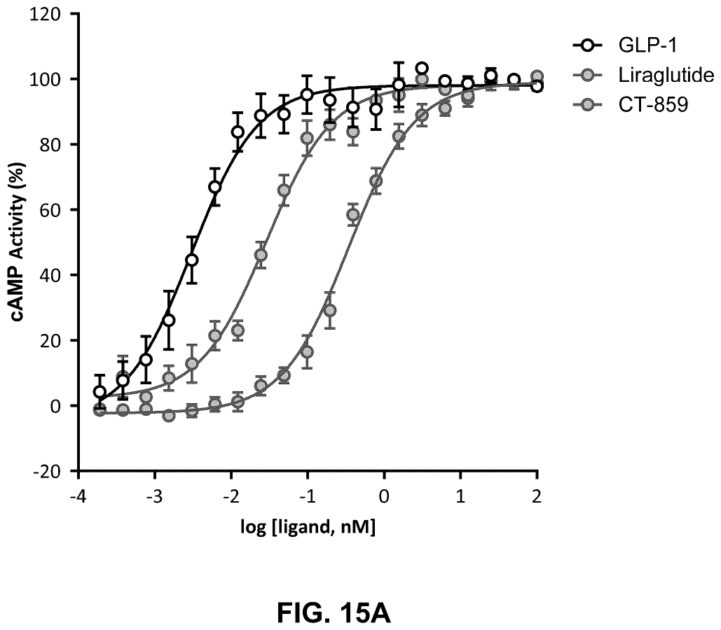

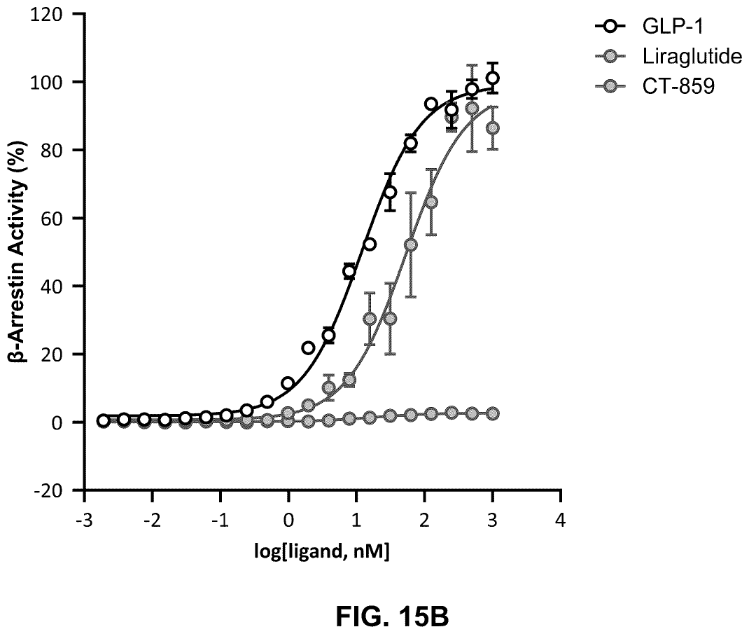

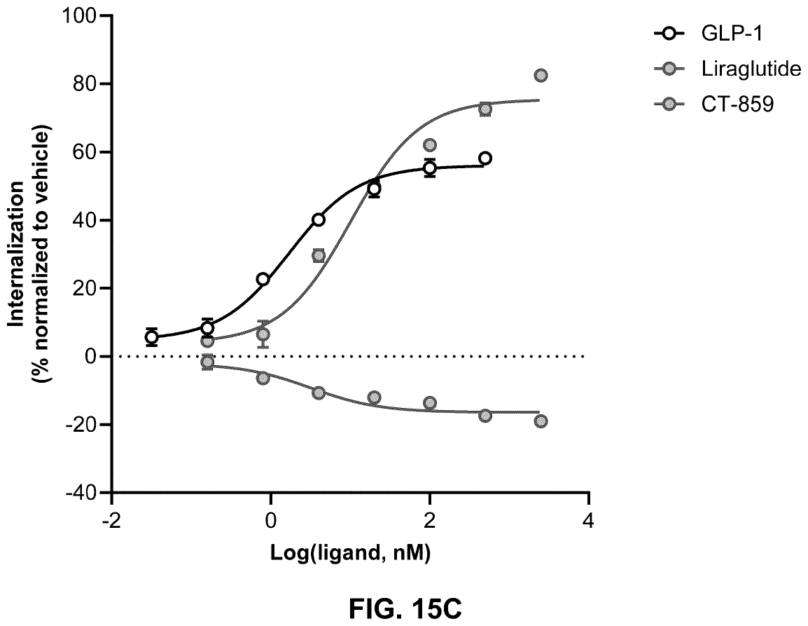

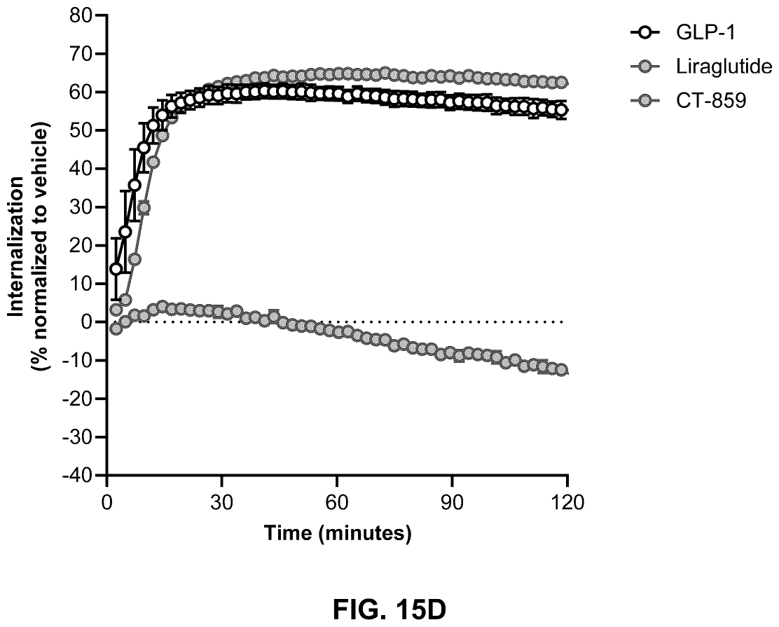

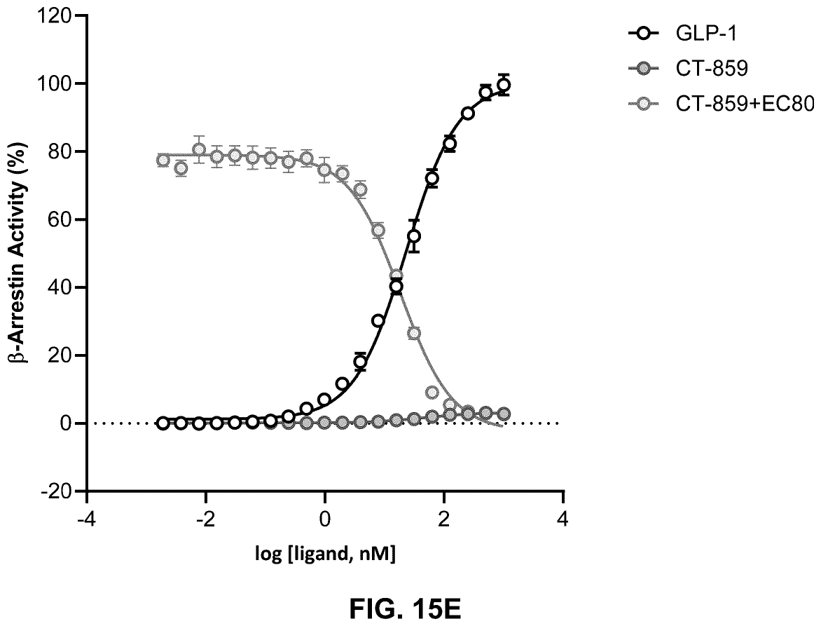

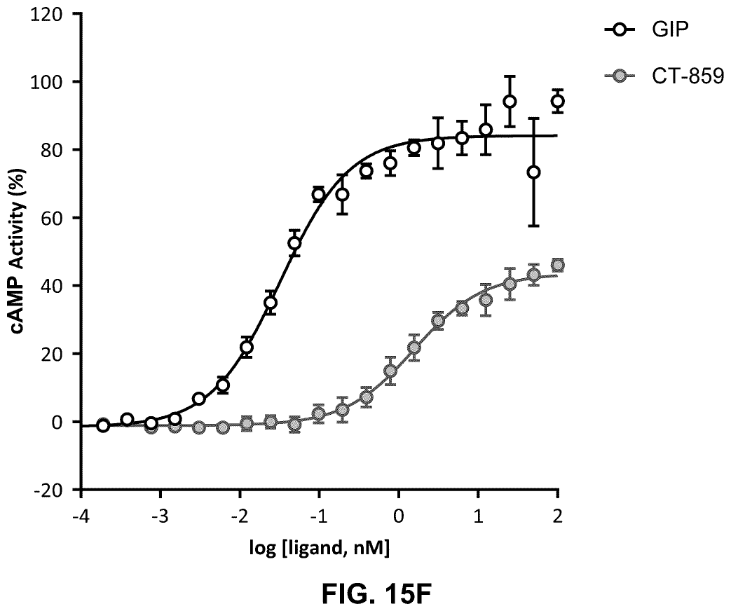

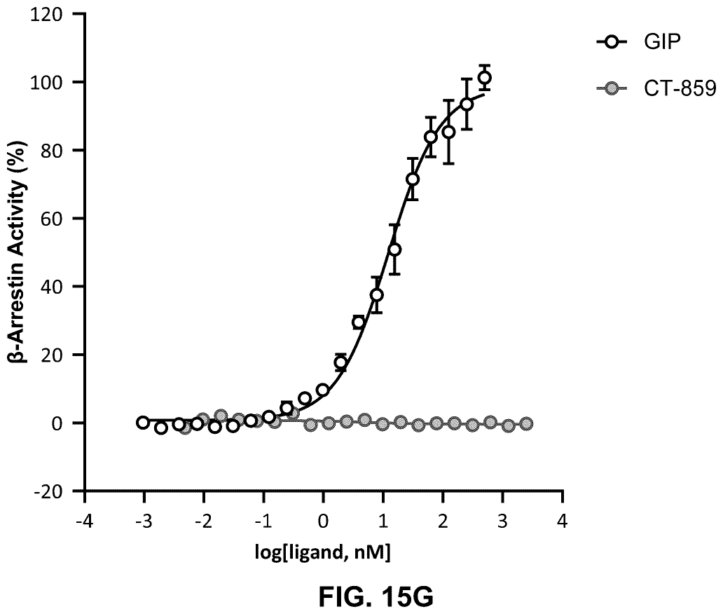

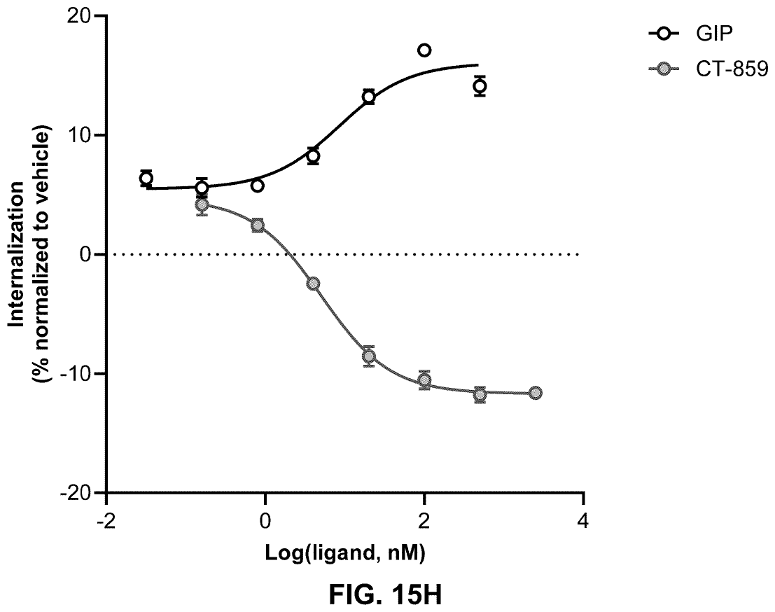

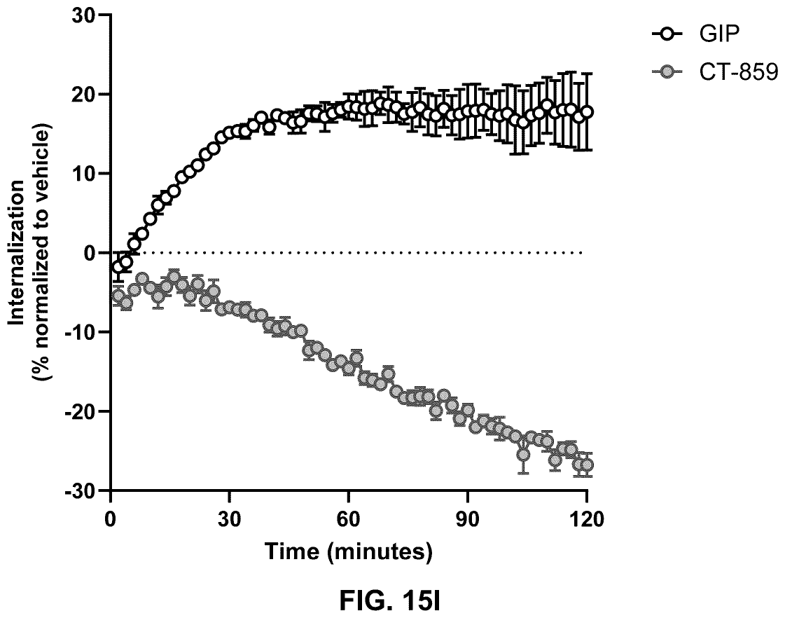

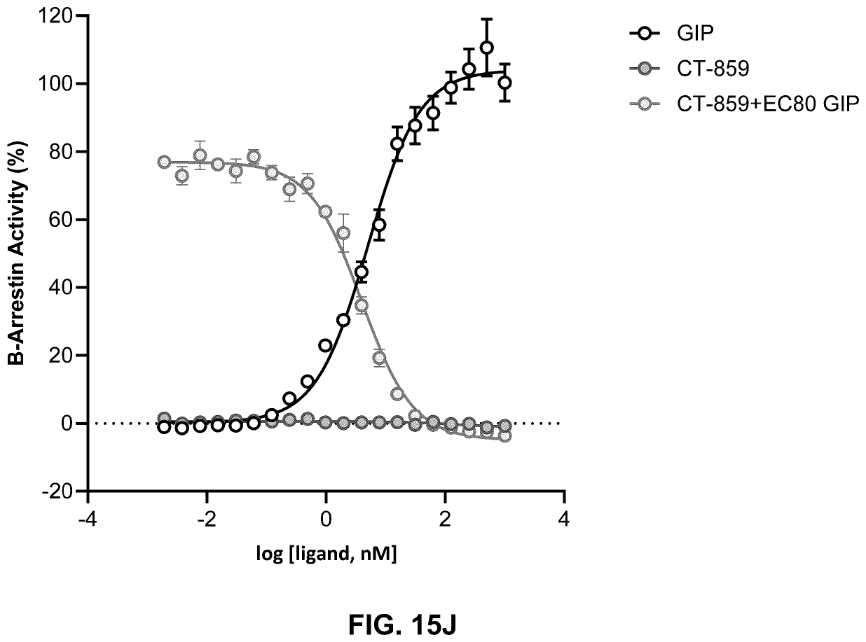

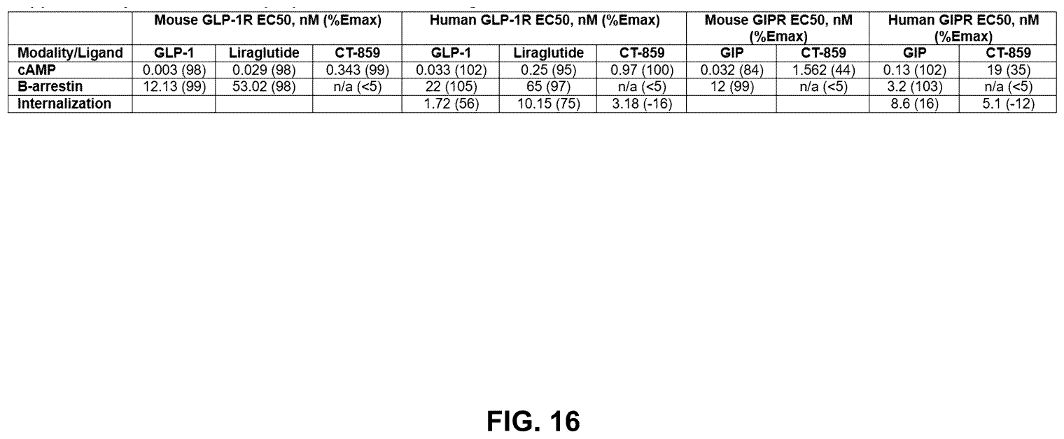

To investigate cAMP accumulation, β-arrestin coupling, and internalization properties of CT-859 and relevant tool compounds, recombinant cell lines expressing GLP-1R or GIPR were utilized. CT-859 is a full agonist for cAMP accumulation at mouse GLP-1R and is 113 times less potent than native ligand GLP-1 and 12 times less potent than Liraglutide (EC50: GLP-1 3 pM, Liraglutide 29 pM, CT-859 343 pM, FIG. 15A). However, it exhibits modality bias, as it elicits negligible (<5%) recruitment of β-arrestin-2 to mouse GLP-1R vs GLP-1 (99%) and Liraglutide (98%) (FIG. 15B). At mouse GIPR, CT-859 is a partial agonist (44% Emax) for cAMP accumulation, with 48 times lower potency than GIP to drive cAMP accumulation (1.6 nM vs 32 pM respectively, FIG. 15F). Similar to the effects of CT-859 at GLP-1R, it also has negligible (<5%) recruitment of β-arrestin-2 to mouse GIPR vs GIP (FIG. 15G). Next, the effects on internalization at human receptors, which likely mimics internalization properties at mouse receptors as ligand cAMP and β-arrestin coupling properties are similar at both receptors (FIG. 16), was investigated. These receptors are highly conserved across species (human and mouse GLP-1R are 92% identical, 94% similar and human and mouse GIPR are 81% identical, 85% similar). In contrast to GLP-1 and liraglutide, which drives human GLP-IR internalization (EC50 1.7 and 10.2 nM, Emax 56% and 75% respectively), CT-859 did not cause receptor internalization (Emax—16%; FIGS. 15C and 15D). To the contrary, after two hours of exposure to CT-859, GLP-IR expression was higher versus vehicle-treated cells (FIG. 15D). CT-859 had a similar effect at human GIPR, stimulating greater surface expression than vehicle (FIGS. 15H and 15I). This would suggest that CT-859 is acting as an antagonist of β-arrestin coupling resulting in inverse agonism for receptor internalization. Indeed, CT-859 inhibited the native ligand mediated recruitment of β-arrestin-2 at the GLP-IR and GIPR (FIGS. 15E and 15J).

### CT-859 Normalizes Postprandial Glucose Levels Primarily Through GLP-1R Activation

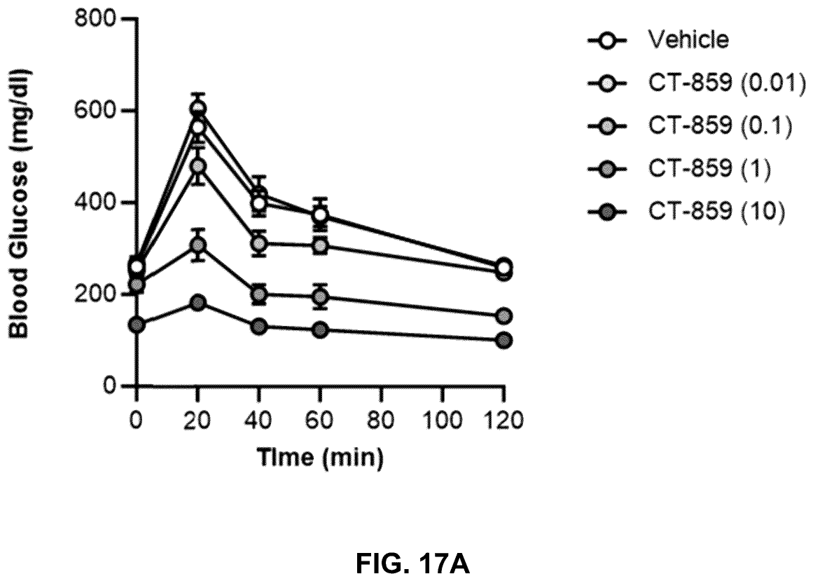

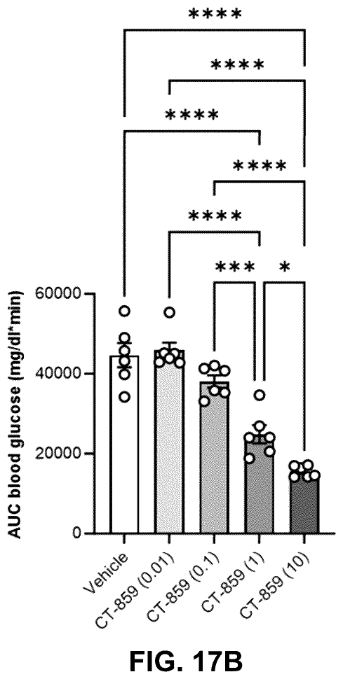

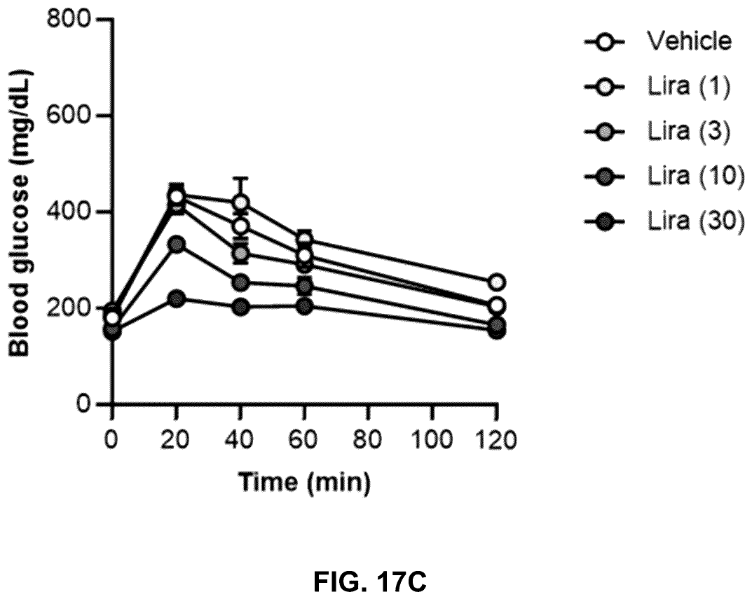

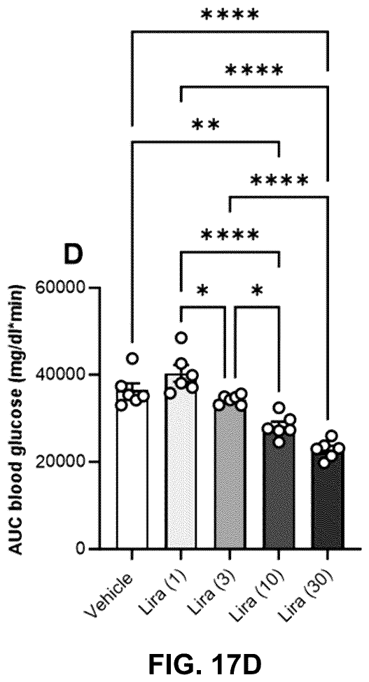

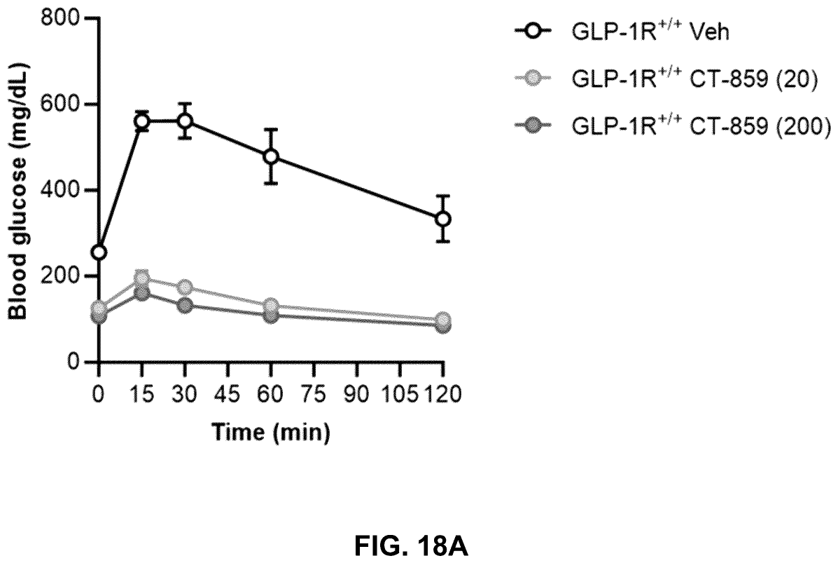

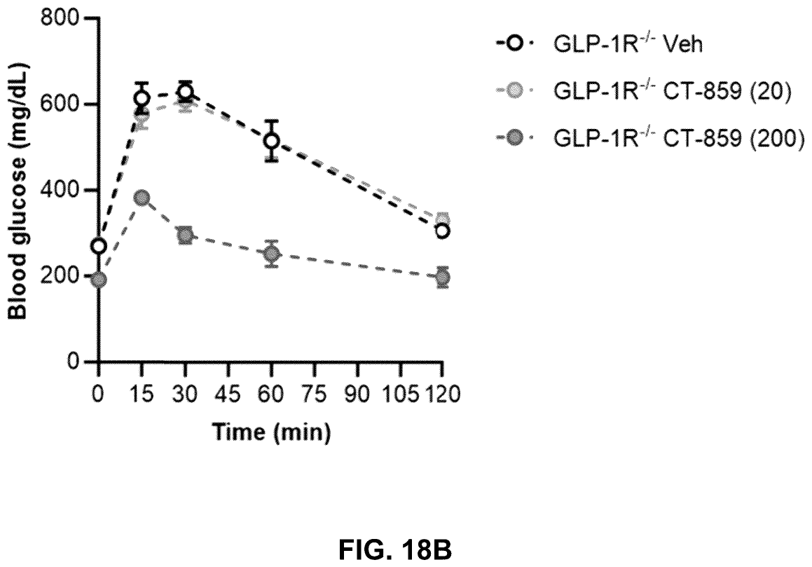

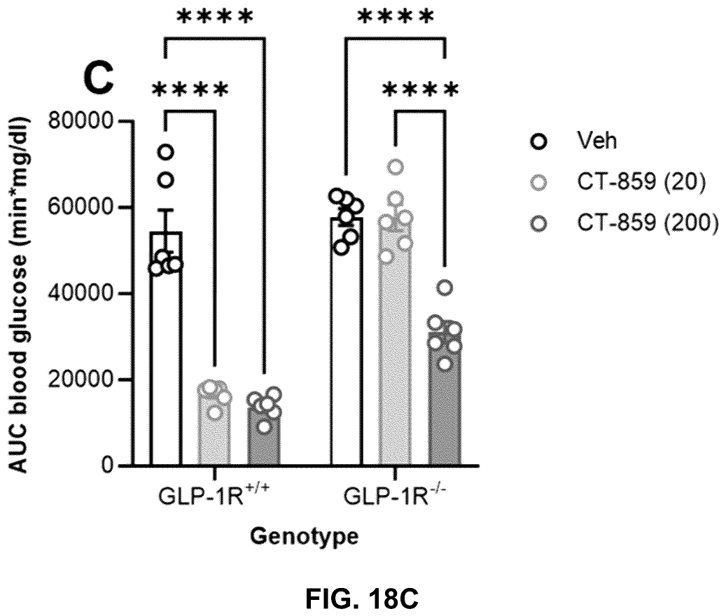

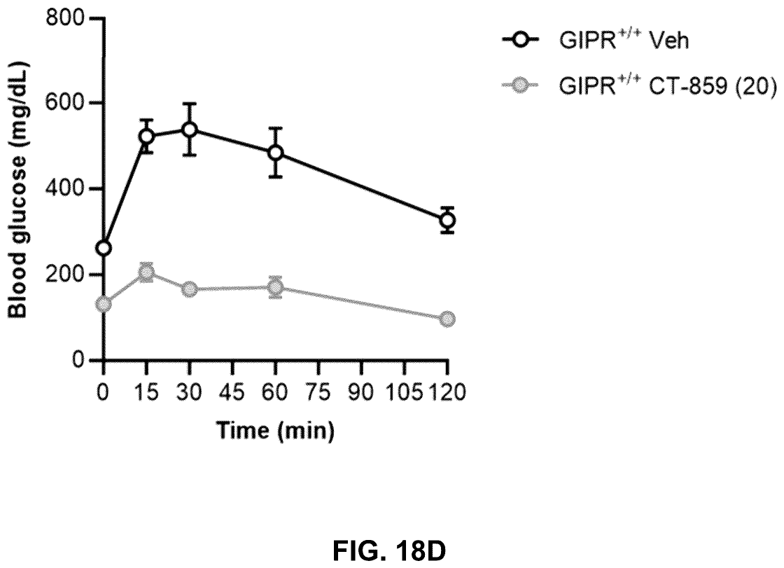

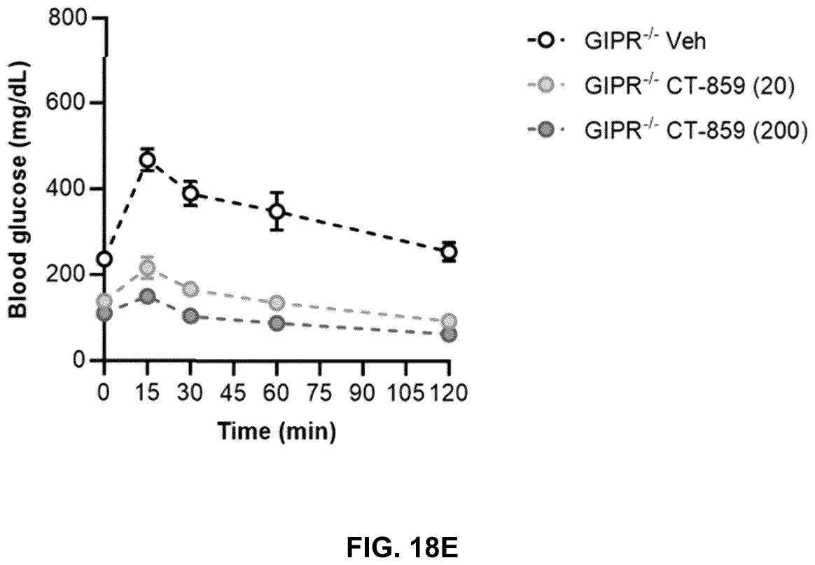

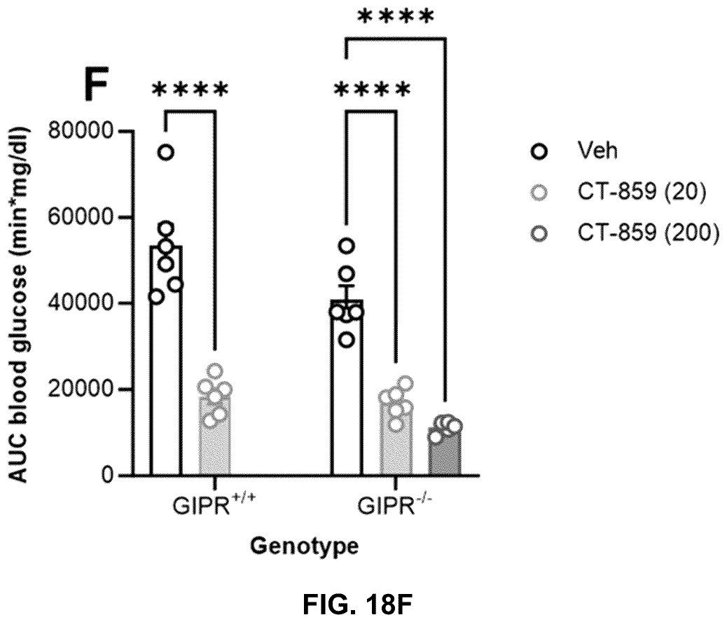

The glucose-lowering effects of CT-859 were characterized in lean C57BL/6J mice. CT-859 dose-dependently improved post-prandial glucose during an intraperitoneal glucose tolerance test (ipGTT) with an ED50 of 0.46 nmol/kg (FIGS. 17A and 17B), whereas liraglutide improved it with an ED50 of 7.3 nmol/kg (FIGS. 17C and 17D). As CT-859 is a dual GLP-1/GIPR agonist, the contribution of each receptor to the glucose-lowering effects of CT-859 was assessed. To do so, ipGTTs in GLP-IRKO, GIPRKO mice, and their wild-type (+/+) littermates were performed. In GLP-IR^{+/+} mice, CT-859 (20 and 200 nmol/kg) lowered the post-prandial glucose levels equally and significantly compared to vehicle (FIGS. 18A and 18C). On the other hand, in GLP-IR^{-/-} mice, CT-859 at 20 nmol/kg was ineffective at lowering glucose levels, while a dose of 200 nmol/kg lowered glucose levels by 46% compared to vehicle, whereas CT-859 (200 nmol/kg) lowered glucose levels by 75% in GLP-1R+/+ mice (FIGS. 18B and 18C). This indicates that the glucose-lowering effect at 20 nmol/kg was mainly mediated through GLP-IR, while at 200 nmol/kg, GIPR was likely involved. In GIPR^{-/-} mice, both doses of CT-859 (20 and 200 nmol/kg) were efficacious at lowering glucose levels. Compared to GIPR^{+/+}, the glucose improvement with 20 nmol/kg of CT-859 was maintained in GIPR^{-/-} mice (FIGS. 18D, 18E, and 18F). These results demonstrate that the glucose-lowering effects of CT-859 at 20 nmol/kg are dominated by GLP-1R activation and do not require the engagement of GIPR. This dose is referred to as a “a non-GIPR-engaging dose.”

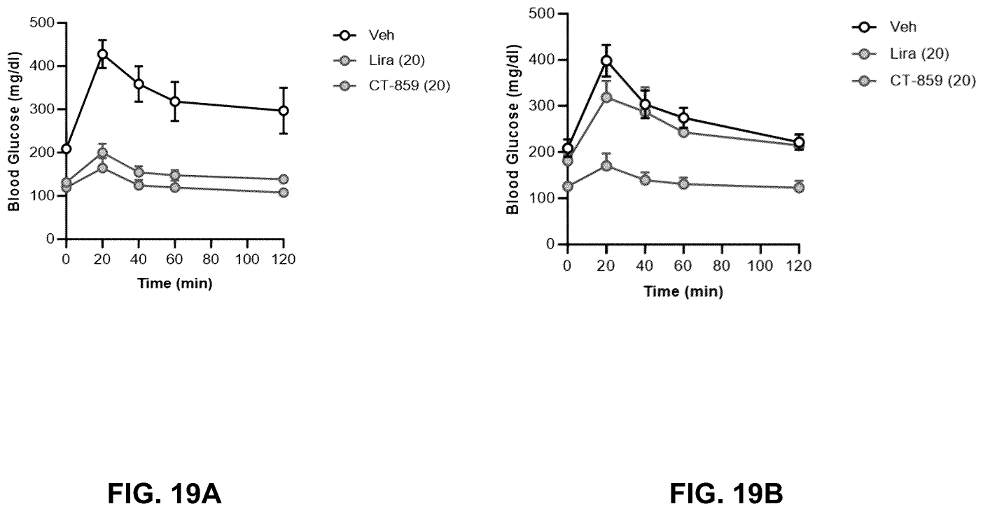

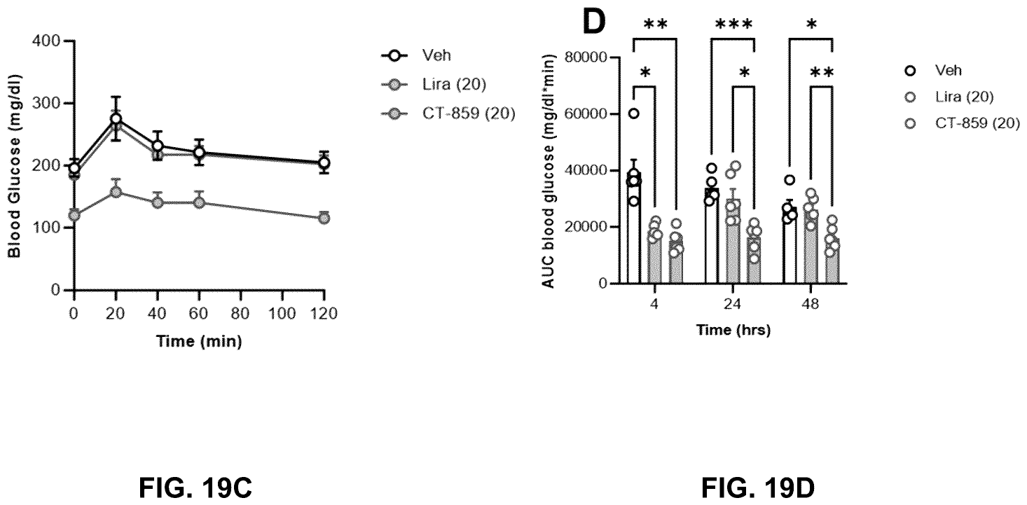

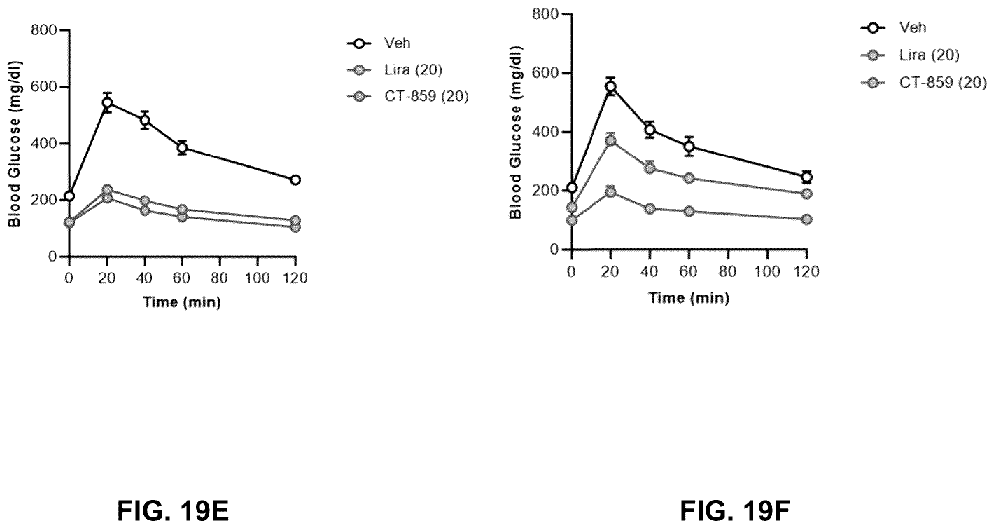

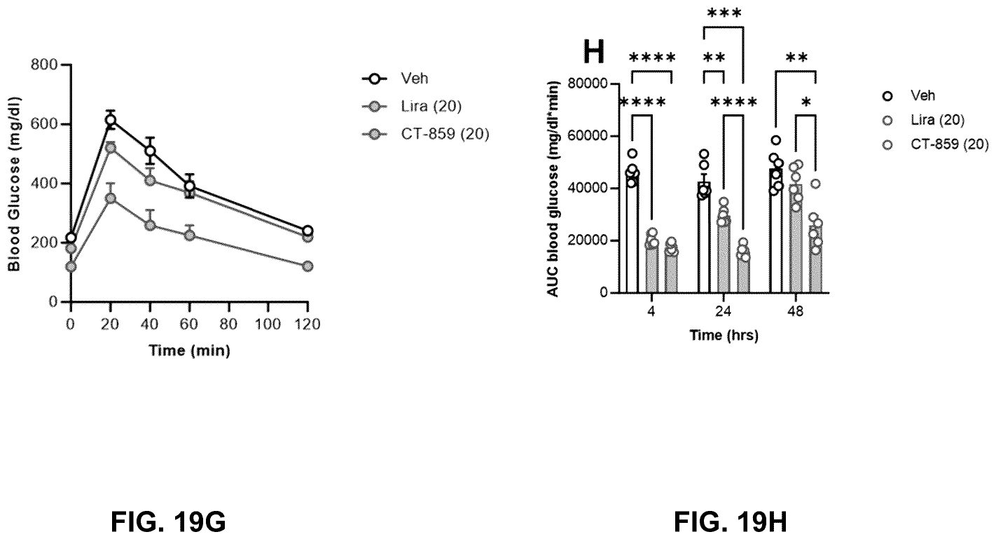

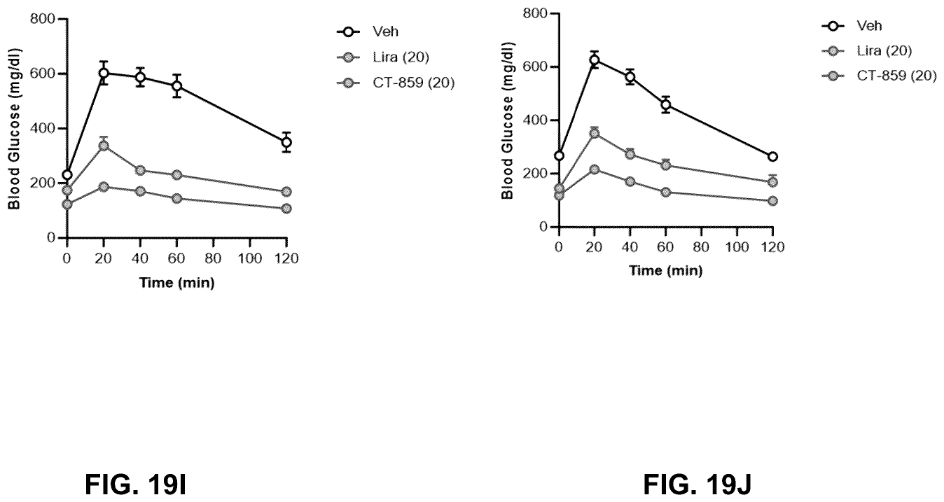

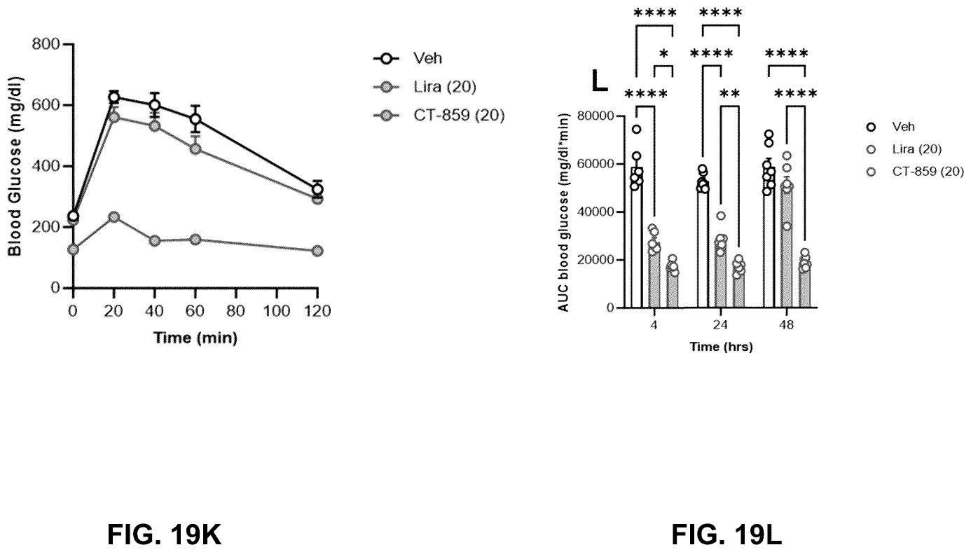

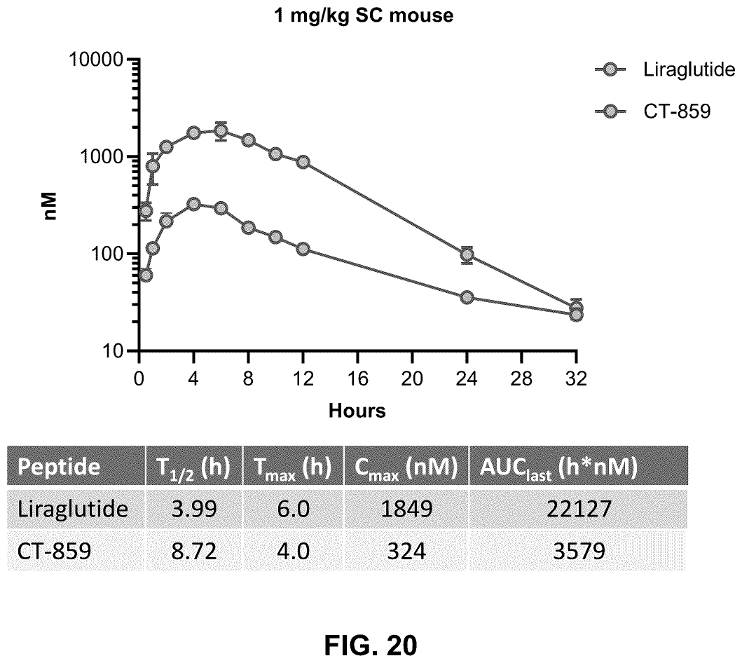

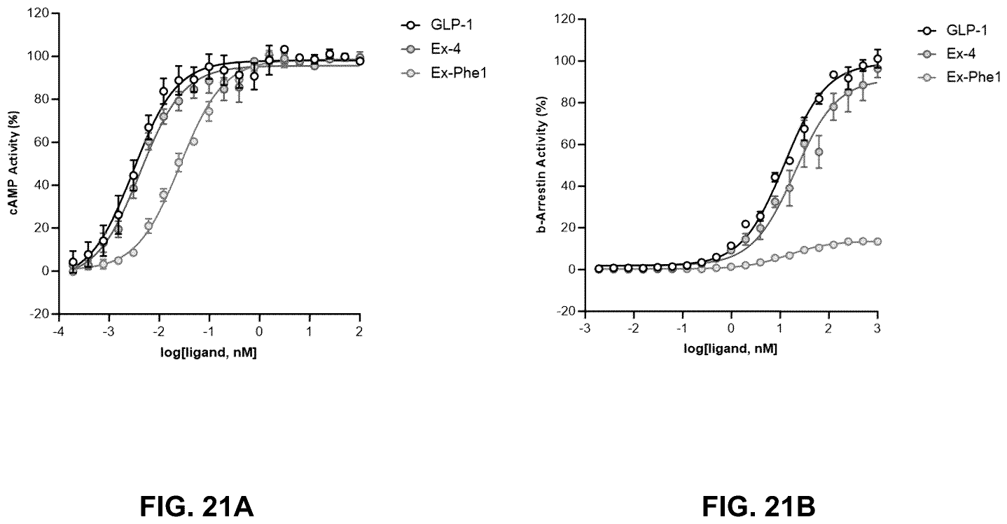

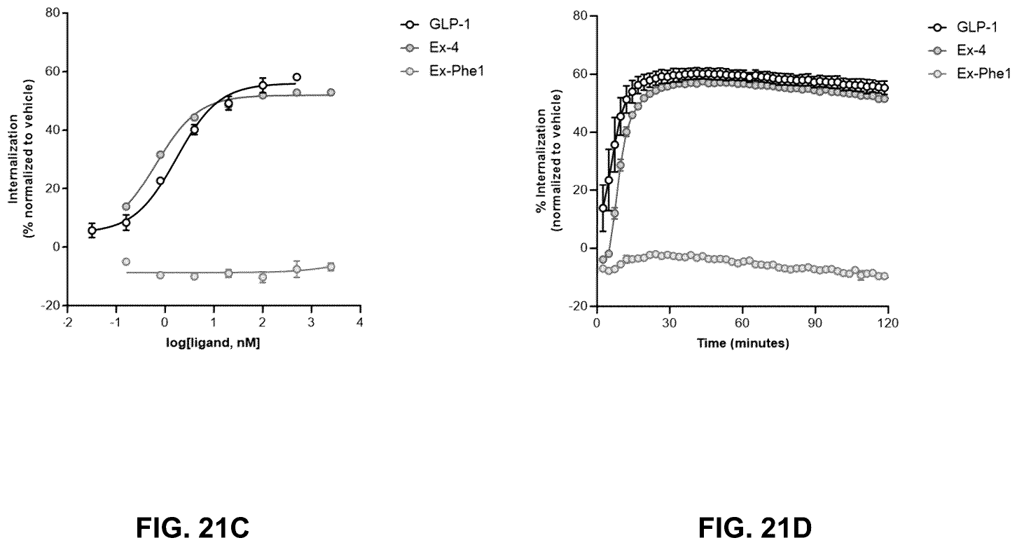

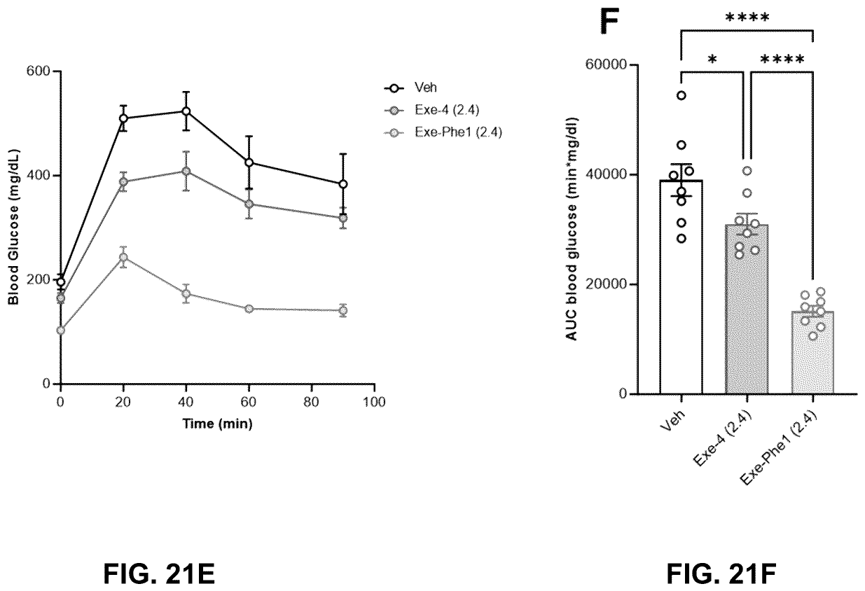

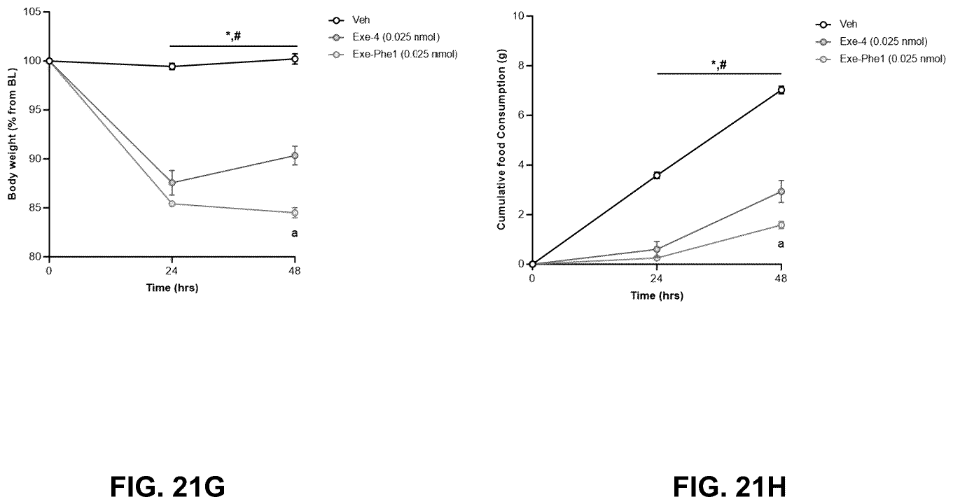

At a non-GIPR-engaging dose, CT-859 demonstrated a prolonged glucose-lowering effect compared to liraglutide. To assess whether there is a difference in glucose-lowering effects between biased and unbiased GLP-1R agonism, ipGTTs using a maximum glucose lowering dose of CT-859 (20 nmol/kg), a non-GIPR engaging dose, and liraglutide (20 nmol/kg) in GIPR^{-/-} mice were performed. CT-859 and liraglutide improved post-prandial glucose 4 hours following a single dose. However, liraglutide no longer differed from vehicle 24 hours after treatment, while CT-859 maintained its activity at 24 and 48 hours (FIGS. 19A-19D). Similar studies were conducted in lean C57BL/6J mice and diet-induced obese (DIO) mice to confirm the observations in GIPR^{-/-} mice. The results were for the most part consistent with the results in the GIPR^{-/-} mice. There was no difference in the glucose levels 4 hours after drug administration between CT-859 and liraglutide as shown by the area under the curve (AUC) in lean C57BL/6J mice (FIGS. 19E and 19H); nevertheless, CT-859 was more efficacious than liraglutide in DIO mice (FIGS. 191 and 19L). Consistent with the results in GIPR^{-/-} mice, it was found that CT-859 remained efficacious up to 48 hours post-administration, whereas liraglutide was no longer different from vehicle in both lean C57BL/6J and DIO mice (FIGS. 19F, 19G, 19H, 19J, 19K, and 19L). To exclude the possibility that prolonged glucose lowering is due to differences in drug exposure, the pharmacokinetics of CT-859 and liraglutide in mice were compared. Though CT-859 has a longer half-life, liraglutide has 6 times more exposure than CT-859 (FIG. 20). These results suggest that the observed difference in glucose lowering is intrinsic to CT-859 and may result from biased signaling at the GLP-IR. To provide further evidence, Exendin-Phe1 (Ex-Phe1) was characterized and confirmed as a cAMP-biased agonist that prevents receptor internalization (FIGS. 21A-21D). A single dose of Ex-Phe-1 showed that the biased GLP-1R agonist remained efficacious in an ipGTT eight hours post-drug administration, while the unbiased GLP-1R agonist Exendin-4 (Ex-4) was no longer effective (FIGS. 21E and 21F). These findings are consistent with observations by Jones et al.

Central Administration of CT-859 at a Non-GIPR Engaging Dose Decreases Body Weight More than Liraglutide

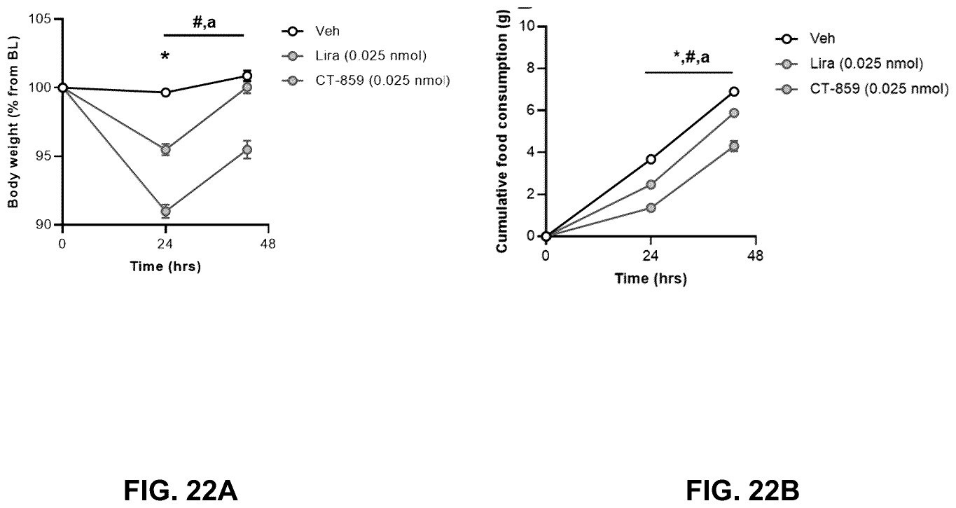

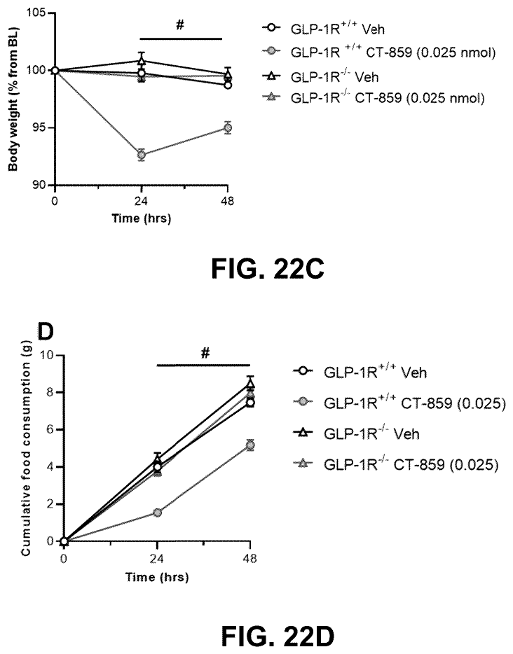

Studies have indicated that GLP-IR agonists suppress food intake through a centrally mediated mechanism. To characterize the central effects of biased and unbiased GLP-1R activation on food consumption and weight loss, compounds were administered to the lateral ventricle by intracerebroventricular (ICV) injection. In lean C57BL/6J mice, ICV administration of CT-859 (0.025 nmol) lowered food consumption by 63% and 38% after 24- and 48-hours post-treatment, respectively, compared to vehicle, whereas liraglutide (0.025 nmol) lowered it by 33% and 15% after 24- and 48-hours post-treatment, respectively, compared to vehicle. CT-859 lowered food consumption by 45% and 27% after 24- and 48-hours post-treatment, respectively, compared to liraglutide (FIG. 22B). Consistent with the decrease in food consumption, CT-859 decreased body weight by 9% and 5% after 24- and 48-hours post-treatment, respectively. Liraglutide decreased body weight by 4% after 24 hours post-treatment. CT-859 decreased body weight by 5% and 5% after 24- and 48-hours post-treatment, respectively, compared to liraglutide (FIG. 22A). Next, ICV injections of CT-859 in GLP-1R^{-/-} mice and their WT littermates were evaluated to delineate how each receptor contributes to the observed effects in lean C57BL/6J mice. CT-859 (0.025 nmol) suppressed food consumption and reduced body weight in the GLP-IR^{+/+} mice while it had no effect in GLP-IR−/− mice (FIGS. 22C and 22D), suggesting that at this dose, CT-859 suppresses food consumption and reduces body weight through activation of GLP-1R and does not engage GIPR at this dose. To confirm that these effects were mediated by biased signaling exclusively at the GLP-IR, the effects of ICV administration of Ex-phe1 and Ex-4 in lean C57BL/6J mice were assessed. ICV administration of Ex-4 and Ex-Phe1 lowered food intake and body weight equally 24-hours post-treatment (FIGS. 21G and 21H). However, Ex-4 started to lose efficacy by 48 hours, whereas Ex-phe1 remained efficacious (FIGS. 21G and 21H). The data show that a single dose of ICV administered biased GLP-1 is more efficacious at suppressing food consumption and reducing body weight than unbiased GLP-1.

Chronic CT-859 Decreases Body Weight More than Liraglutide.

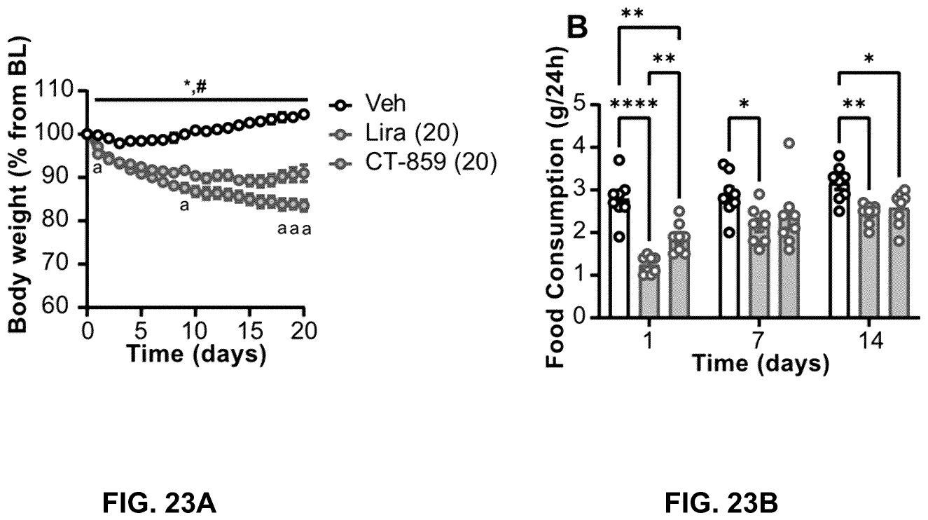

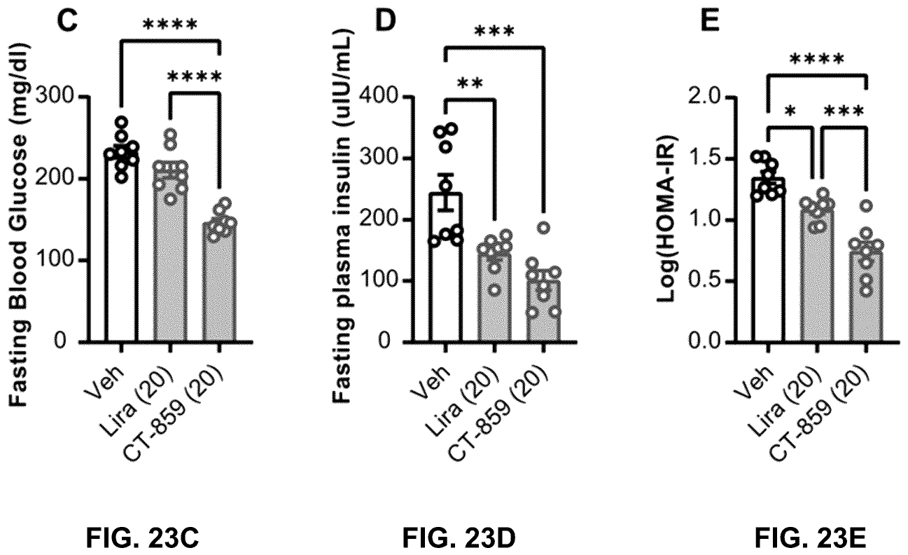

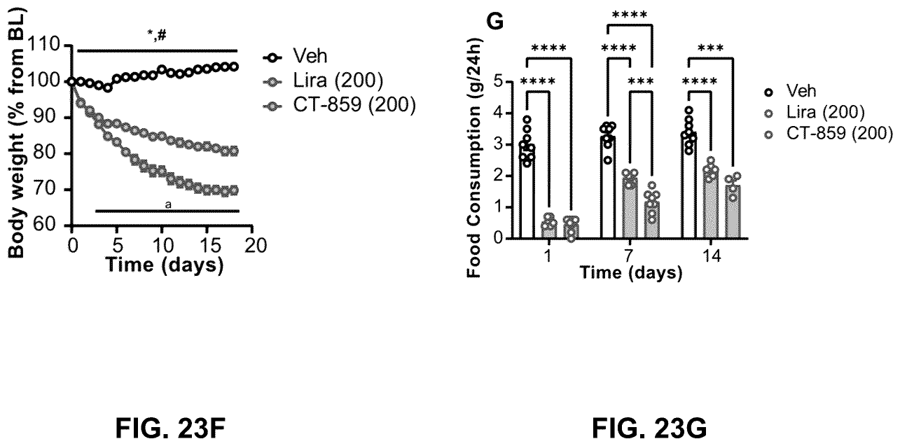

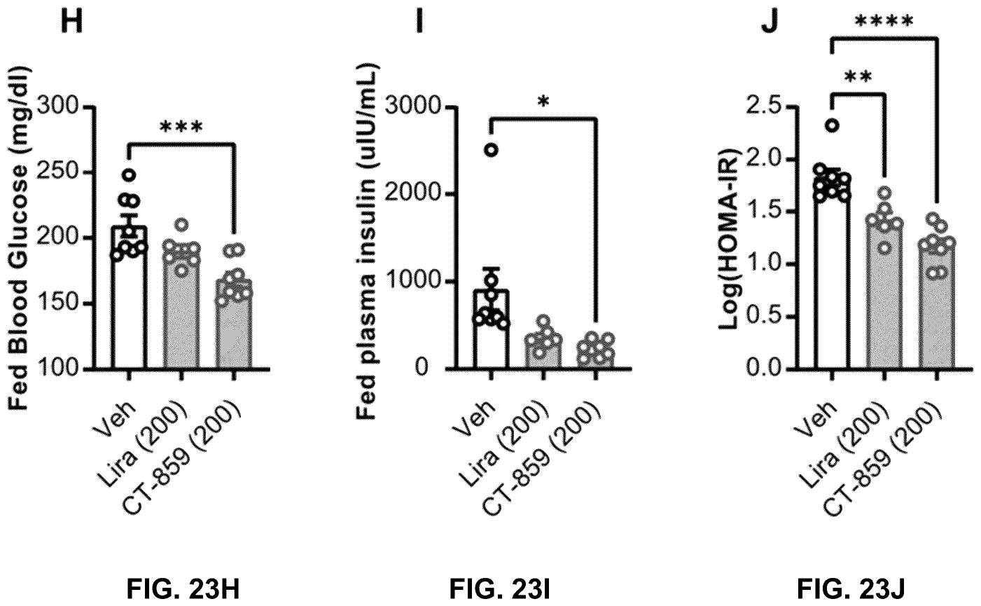

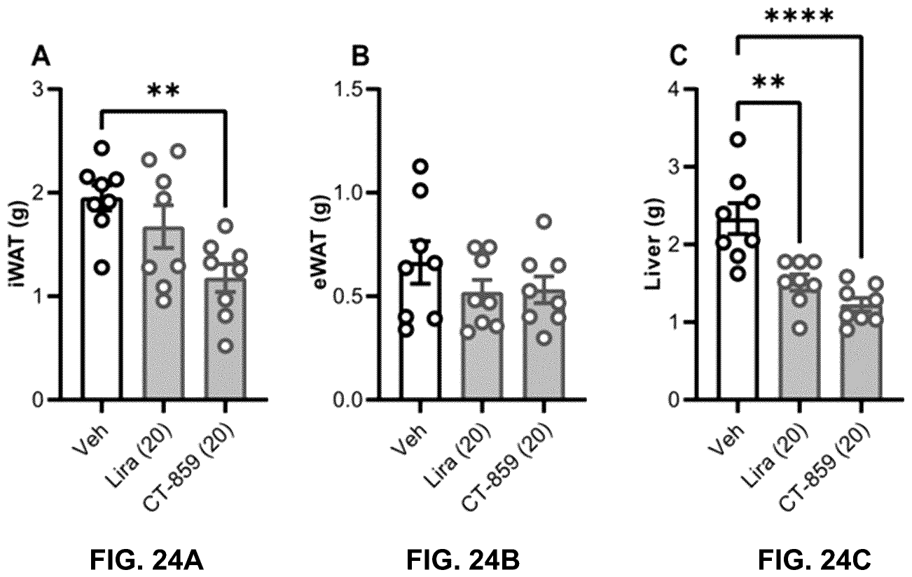

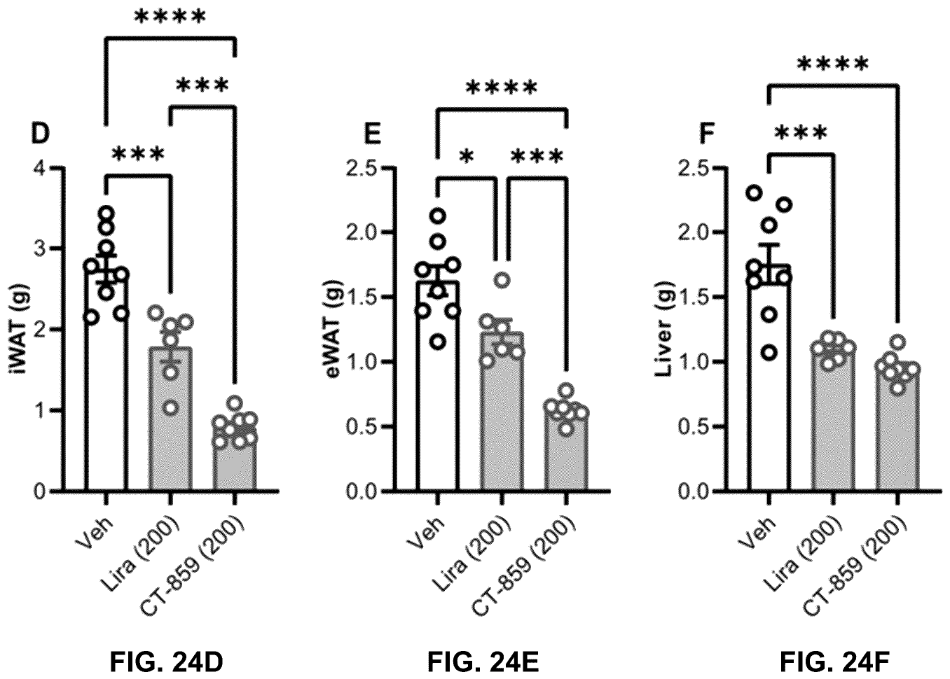

To further assess the difference between a biased and unbiased GLP-IR agonist, chronic studies were performed in C57BL/6J DIO mice, a model of obesity and hyperglycemia. First, CT-859 (20 nmol/kg), a non-GIPR-engaging dose, was compared to liraglutide (20 nmol/kg). CT-859 and liraglutide reduced body weight similarly at first. Nevertheless, CT-859 started to separate from liraglutide after a week of dosing. At the end of the study, CT-859 decreased body weight by 20% compared to vehicle. Liraglutide decreased body weight by 13% compared to vehicle. Moreover, CT-859 decreased body weight by 8% compared to liraglutide (FIG. 23A). Interestingly, there was no difference between CT-859 and liraglutide on food intake (FIG. 23B), indicating the potential of additional mechanisms involved in weight loss with biased GLP-IR activation. At the end of the study, body composition was checked to determine the source of the weight loss. Inguinal adipose weight was 39% lower in the CT-859 group than in the vehicle; however, liraglutide did not differ from the vehicle (FIG. 24A). There was no difference in epididymal adipose weight between the groups (FIG. 24B). Both liraglutide and CT-859 lowered the weight of the liver compared to the vehicle (FIG. 24C). CT-859 lowered fasting glucose more than liraglutide; however, both treatments lowered plasma insulin levels more than vehicle (FIGS. 23C and 23D); as a result, the Log (HOMA-IR) was lower with CT-859 compared to liraglutide (FIG. 23E). Next, CT-859 (200 nmol/kg), a GIPR-engaging dose, and liraglutide (200 nmol/kg) were administered for 19 days in DIO mice to assess the full pharmacological potential on weight loss. Following a similar pattern to the non-GIPR engaging dose, CT-859 and liraglutide initially decreased body weight equally. However, CT-859 started to separate from liraglutide after 3 days of dosing, and at the end of the study, CT-859 decreased body weight by 33% compared to vehicle. Liraglutide decreased body weight by 22% compared to vehicle. Furthermore, CT-859 decreased body weight by 14% compared to liraglutide (FIG. 23F). In contrast to the non-GIPR-engaging dose, CT-859 lowered food consumption by 39% compared to liraglutide after 7 days of dosing (FIG. 23G). At this dose GIPR engagement may have altered the dynamic effects on food intake and the mechanisms involved in weight loss with CT-859. At the end of the study, CT-859 decreased inguinal and epididymal adipose weight more than liraglutide, and both decreased liver weights equally (FIGS. 24D-24F). CT-859 decreased fed glucose levels more than the vehicle, whereas liraglutide had no effect (FIG. 23H). CT-859 decreased fed insulin levels more than vehicle (FIG. 23I). CT-859 and liraglutide lowered the Log (HOMA-IR) compared to the vehicle (FIG. 23J). These data show that, while CT-859 is more efficacious than liraglutide at non-GIP engaging doses, the superior efficacy of CT-859 is more pronounced at GIP-engaging doses.

### Discussion

In the present study CT-859, a GLP-1R/GIPR agonist biased for G protein coupling at both receptors, was engineered. CT-859 does not induce receptor internalization at either receptor; and it inhibits β-arrestin coupling by the native ligands GLP-1 and GIP. These characteristics seemed to translate into improved in vivo efficacy, as demonstrated by the observation that CT-859 improved glucose tolerance with an ED₅₀ of 0.46 nmol/kg whereas, liraglutide's ED₅₀ was 7.3 nmol/kg. These results are surprising as CT-859 is more than one hundred times less potent than the native ligand GLP-1 and more than ten times less potent than liraglutide on GLP-IR CAMP accumulation assays. When liraglutide and CT-859 were tested at a maximum glucose lowering dose, CT-859 was demonstrated to have a prolonged glucose tolerance improvement in lean and DIO mice compared to liraglutide. This prolonged efficacy did not result from the additional engagement of GIPR as the dose tested does not engage the GIPR, and the circulating drug concentrations do not explain it, as liraglutide has six times more drug exposure than CT-859. These results may imply that the improved efficacy of CT-859 is possibly mediated through biased signaling at the GLP-1R. In support of this studies have demonstrated that loss of β-arrestin-2 acutely decreases the insulin secretion response to GLP-IR agonists (Bitsi et al., Science Advances. (2023) 9: eadf7737). Nevertheless, prolonged exposure to GLP-IR agonists leads to better glucose reduction in these mice, suggesting that loss of β-arrestin-2 signaling acutely impairs the insulin response by GLP-IR agonists. However, prolonged exposure to these agonists increases their effectiveness at reducing glucose levels by increasing insulin secretion (Bitsi, supra). Additionally, studies have demonstrated that biased GLP-IR agonists are more efficacious than unbiased GLP-IR agonists on glucose regulation (Pickford et al., British Journal of Pharmacology. (2020) 177:3905-23; Lucey et al., Molecular Metabolism. (2020) 37:100991; Jones et al., Nat Comm. (2018) 9:1602; Zhang et al., Nat Comm. (2015) 6:8918). Taken together, these results suggest that lack of β-arrestin coupling increases the efficacy of GLP-1 receptor agonists on glucose lowering.

The acute observations translated into better efficacy in chronic studies. CT-859, at a non-GIPR-engaging dose, was compared to liraglutide in a 3-week study in DIO mice; CT-859 lowered fasting BG and improved HOMA-IR more than liraglutide. More importantly, treatment with CT-859 led to more weight loss than liraglutide. In this study, both treatments suppressed food consumption equally. These results suggest that appetite suppression may contribute to the efficacy of both treatments, but additional mechanisms may be responsible for the observed difference in weight loss between CT-859 and liraglutide. In agreement, GLP-IR agonists have been demonstrated to stimulate brown adipose tissue (BAT) thermogenesis and adipocyte browning independent of nutrient intake in mice. Furthermore, a longitudinal study involving obese T2D patients demonstrated that chronic treatment with liraglutide increased energy expenditure (Beiroa et al., Diabetes. (2014) 63:3346-58).

On the contrary to peripheral administration, central administration of CT-859 suppressed food consumption more than liraglutide. The assessment of biased GLP-IR agonists has mostly been done following peripheral administration, and enhanced efficacy on glucose regulation has been consistently observed. However, the effects on appetite and body weight regulation were inconsistent with different biased GLP-IR agonists (Jones et al., Nat Comm. (2018) 9:1602; Zhang et al., Nat Comm. (2015) 6:8918).

One potential explanation is the different bioavailability of these agonists in the brain. GLP-IR agonist peptides are typically derivatized by half-life extending lipid modifications on lysine residues to extend their durability. These lipid modifications can alter the bioavailability of GLP-1R agonist in the brain as the length of such lipid modifications aids in the ability of these agonists to cross the blood-brain barrier (BBB). Short-acting GLP-IR agonists such as Ex-4 are mainly detectable outside the BBB; however, GLP-1R agonists with half-life extending modifications are detected in areas of the brain beyond the BBB, with the spread being associated with the length of the lipid modification (Skovbjerg et al., Neuropharmacology. (2023) 238:109637). The ability of GLP-1R agonists to cross the BBB is essential because studies that inhibited or knocked down/out neuronal GLP-1R demonstrated that the anorectic and weight loss effects of GLP-1R agonists are mediated mainly by the central nervous system (CNS) (Sisley et al., J Clin Investigation. (2014) 124:2456-463; Secher et al., J Clin Investigation. (2014) 124:4473-88; Fortin et al., Sci Trans Med. (2020) 12: eaay8071. Many factors can alter the bioavailability of GLP-1R agonists in the brain such as GLP-IR expression, glycemia, and obesity (Imbernon et al., Cell Metabolism. (2022) 34:1054-63.e1057; Bakker et al., Cell Reports. (2022) 41). Therefore, central administration is more appropriate to assess the effects of biased and unbiased GLP-1R agonists on food intake suppression and weight loss. Indeed, ICV administration of a non-GIPR-engaging dose of CT-859 suppressed food intake and decreased body weight more than liraglutide in lean mice. To confirm the results with CT-859 and liraglutide, Ex-phe1 and Ex-4 were administered by ICV and demonstrated that Ex-phe1 suppressed food intake and decreased body weight more than Ex-4. These effects were not observed with peripheral administration (Jones et al., Nat Comm. (2018) 9:1602; Pickford et al., British Journal of Pharmacology. (2020) 177:3905-3923). Access to CNS GLP-1Rs is likely a key, which may also explain the appetite suppression discrepancy between central and peripheral administration of CT-859. Our data demonstrate that in reference to unbiased GLP-1R agonists (liraglutide and Ex-4), superior efficacy on glucose and body weight regulation can be obtained with biased GLP-IR agonists (CT-859 and Ex-phe1).

Next, the efficacy of CT-859 at a GIPR-engaging dose and compared it to an equimolar dose of liraglutide was assessed. Treatment with both molecules led to significant weight loss, but treatment with CT-859 led to greater weight loss, a ˜35% reduction versus vehicle, where liraglutide was ˜25%. This additional 10% weight loss with CT-859 is likely a result of biased GLP-IR agonism and activation of GIPR through suppression of food intake. At this dose, CT-859 suppressed food intake more than liraglutide. The results demonstrate that activation of GIPR regulates energy consumption and weight loss, and that enhanced efficacy can be obtained with biased GIPR agonists. Regarding synergism between GLP-1 and GIP, biased GIPR agonists combine best with biased GLP-IR agonists to achieve the best efficacy. As CT-859 is a biased dual GLP-1R/GIPR agonist, it has all the characteristics to be a highly efficacious molecule. Indeed, when a GIPR-engaging dose was given, superior efficacy on food intake suppression and weight loss was obtained.

### Conclusion

In the present study, CT-859 is described as a dual GLP-1R/GIPR agonist that is CAMP biased at both receptors. CT-859 was used as a tool compound to examine the importance of biased signaling in glycemic control, food intake, and body weight. A biased dual GLP-1R/GIPR agonist is demonstrated to be more efficacious than an unbiased GLP-1R agonist on glycemic regulation, food intake suppression, and weight loss. The better efficacy of the biased dual GLP-1R/GIPR agonist is demonstrated to result from the fact that individual-biased GLP-1R and GIPR agonists are more efficacious in glycemic control, food intake suppression, and weight loss than their unbiased counterparts, and that there is positive cooperativity with combining the two biased agonists. These results suggest that biased signaling should be considered when designing more efficacious incretin receptor agonists.

### Example 3: Biased GLP-1 Improves Weight Loss with Additional Benefits on Glucose Homeostasis via Biased GIP in Diabetic Rodent Models

CT-868 and CT-859 are biased dual GLP-1 and GIP receptor modulators exhibiting no β-arrestin coupling on either receptor. Effects of both compounds on weight loss (WL) and glucose (GLUC) homeostasis were assessed in relevant rodent models.

### Methods

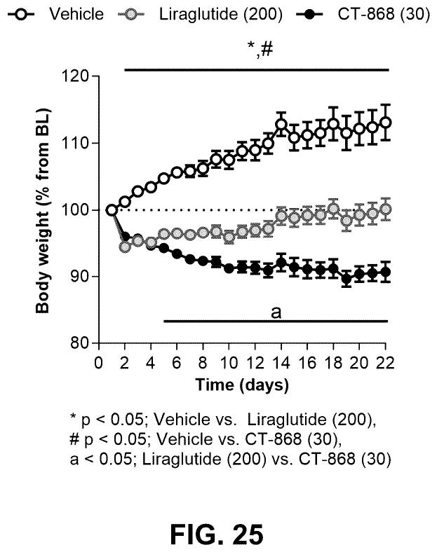

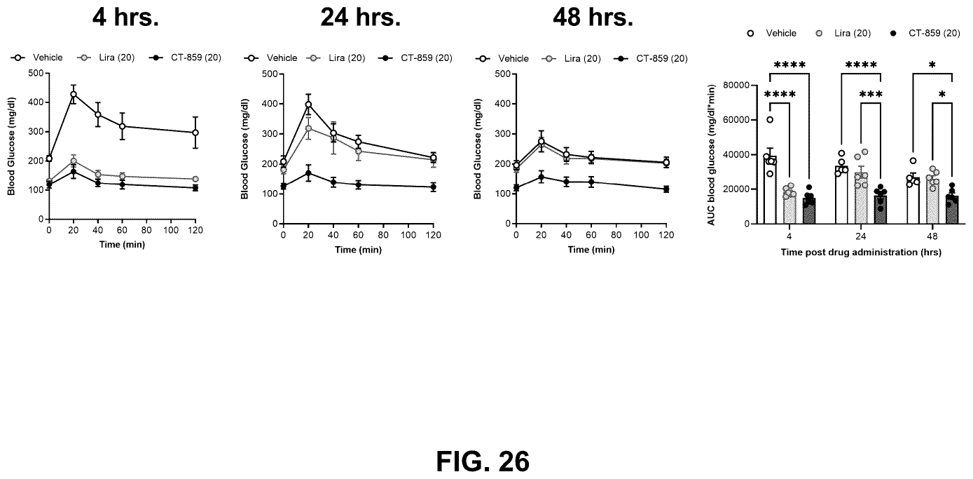

Naïve 9-week-old male Lep^{ob} (ob/ob) mice from Jackson laboratory were individually housed and fed a regular chow diet. Mice were randomized by body weight to the following groups: Vehicle (phosphate buffer+Tween; n=8), liraglutide (200 nmol/kg; n=8), and CT-868 (30 nmol/kg; n=8). Compounds were administered daily by a single subcutaneous injection. Results were analyzed using a two-way ANOVA and Holm-Sidak's test for multiple comparisons (FIG. 25).

Non-naïve 11-15-week-old male GIPR-KO mice from Taconic were housed individually and fed a regular chow diet. Mice were randomized by body weight to the following groups: Vehicle (phosphate buffer+Tween; n=5/6), liraglutide (20 nmol/kg; n=6), and CT-859 (20 nmol/kg; n=6). Intraperitoneal glucose tolerance tests (ipGTT; 2 g/kg) were performed after 5 hours of fasting and 4 hours after compound administration. After one week of washout, compounds were administered by subcutaneous injection. After that, an ipGTT was performed in the same mice 24 and 48 hours after compound administration. Results were analyzed using a two-way ANOVA and Holm-Sidak's test for multiple comparisons (FIG. 26).

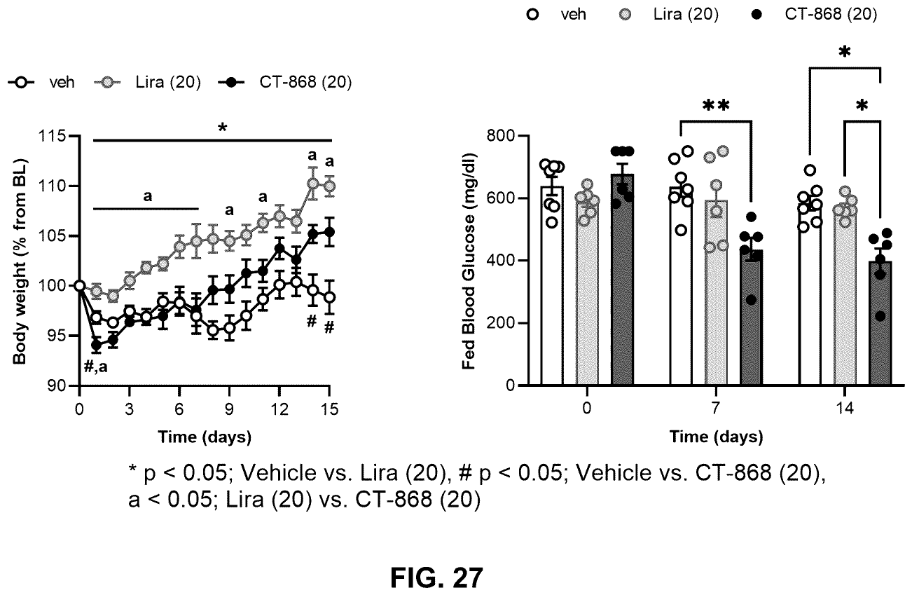

Seven-week-old male C57BL/6-Ins2^{Akita}/J (Akita) mice from Jackson laboratory were individually housed and fed a regular chow diet. Mice were randomized by body weight to the following groups: Vehicle (phosphate buffer+Tween; n=7), liraglutide (20 nmol/kg; n=6), and CT-868 (20 nmol/kg; n=6). Compounds were administered daily by a single intraperitoneal injection. Body mass was recorded daily, and fed glucose was measured at baseline, 7, and 14 days after the start of the treatments. Results were analyzed using a two-way ANOVA and Holm-Sidak's test for multiple comparisons (FIG. 27).

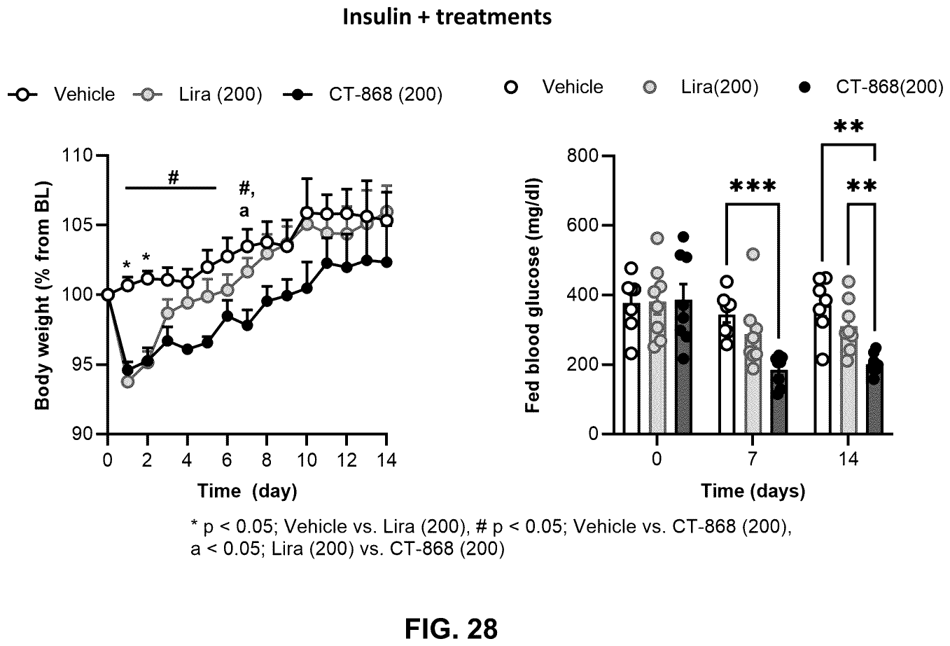

9 to 10-week-old male C57BL/6-Ins2^{Akita}/J (Akita) mice from Jackson laboratory were individually housed and fed a regular chow diet. Approximately 2 weeks before the liraglutide and CT-868 treatments started, mice were implanted with half a pellet of LinBit insulin. Mice were randomized by fed blood glucose to the following groups: Vehicle (phosphate buffer+Tween; n=7), liraglutide (200 nmol/kg; n=8), and CT-868 (200 nmol/kg; n=8). Compounds were administered daily by a single intraperitoneal injection. Body mass was recorded daily, and fed glucose was measured at baseline, 7, and 14 days after the start of the treatments. Results were analyzed using a two-way ANOVA and Holm-Sidak's test for multiple comparisons (FIG. 28).

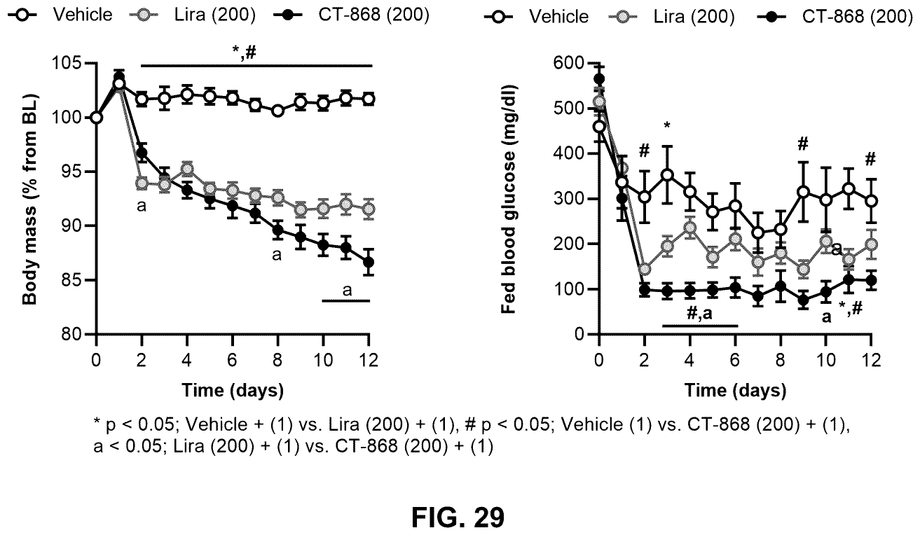

24-week-old C57BL/6J diet-induced obese (DIO) mice received daily intraperitoneal injections with streptozotocin (STZ; 50 mg/kg) for κ days at Jackson laboratory. Mice were received from Jackson's laboratory at 27 weeks of age. Mice were implanted with 1 LinBit insulin pellet and monitored for 18 days. Next, before the Liraglutide and CT-868 treatments started, mice were implanted with a second pellet of LinBit insulin. Mice were then randomized by body weight to the following groups: Vehicle (phosphate buffer+Tween; n=7), liraglutide (200 nmol/kg; n=7), and CT-868 (200 nmol/kg; n=7). Compounds were administered daily by a single intraperitoneal injection. Body weight and fed blood glucose were recorded daily. Results were analyzed using a two-way ANOVA and Holm-Sidak's test for multiple comparisons (FIG. 29).

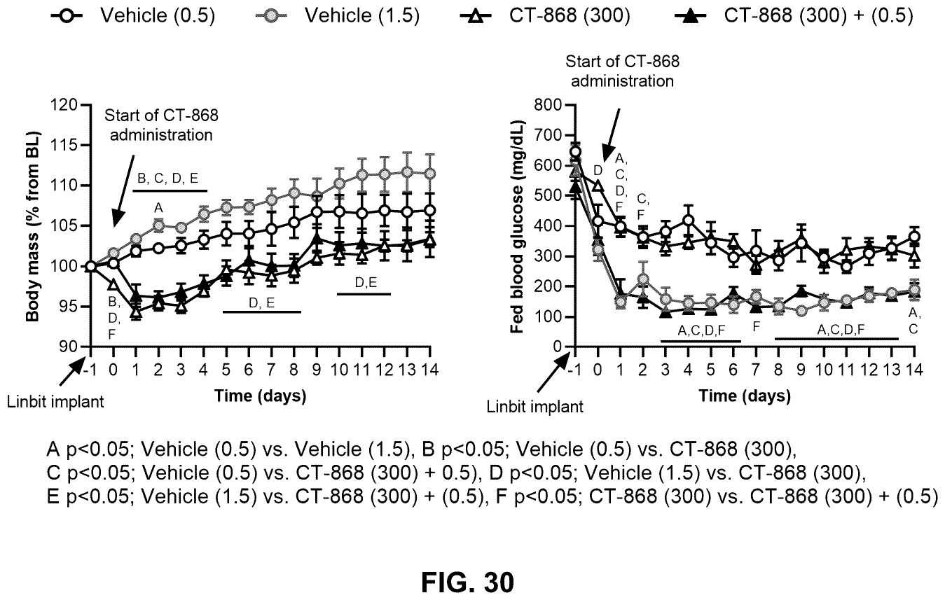

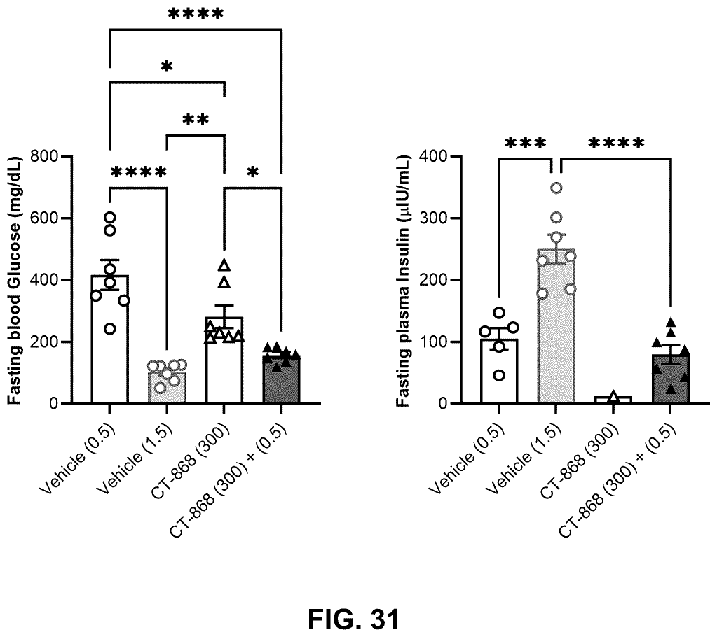

8-week-old male C57BL/6-Ins2^{Akita}/J (Akita) mice from Jackson laboratory were individually housed and fed a regular chow diet. Mice were randomized by body weight to receive insulin or a blank pellet. Mice were anesthetized by isoflurane, and a blank pellet or LinBit insulin pellet/pellets were implanted under the skin in the interscapular area. The following day after the pellet implantations, mice were again randomized to the following groups: Vehicle (phosphate buffer+tween)+0.5 LinBit insulin pellets (Low insulin; n=7), vehicle+1.5 LinBit insulin pellets (High insulin; n=7), CT-868 (300 nmol/kg)+blank pellet (n=7), and CT-868 (300 nmol/kg)+0.5 LinBit insulin pellets (CT-868+Low insulin; n=7). CT-868 was administered daily by a single intraperitoneal injection for 14 days. Body weight and fed blood glucose were recorded daily. On the last day of the study, mice were fasted for 4 hours; blood glucose was measured after that, and blood was collected for insulin. Plasma insulin was measured using an MSD kit. Results were analyzed using a two-way ANOVA and Holm-Sidak's test for multiple comparisons (FIGS. 30 and 31).

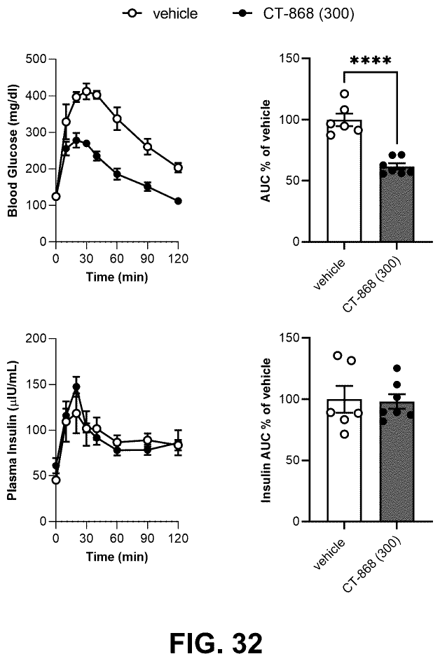

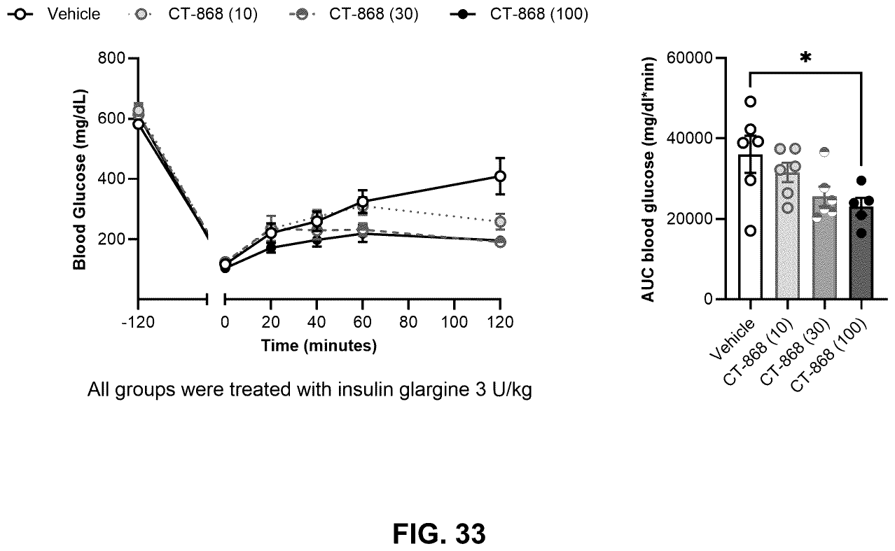

26-27-week-old male GLP-1R-KO mice from Taconic were individually housed and fed a regular chow diet. For the intraperitoneal glucose tolerance test (ipGTT), mice were randomized by body weight to the following groups: Vehicle (PBS; n=6) and CT-868 (300 nmol/kg; n=7). Mice were fasted overnight (18 hours). Following the overnight fast, compounds were administered by subcutaneous injection. One hour after compound administration, glucose was measured, blood was collected, and glucose (2 g/kg) was administered by an intraperitoneal injection. After that, blood glucose levels were measured, and blood was collected for insulin measurements 10, 20, 30, 40, 60, and 120 minutes after the glucose injection. Insulin was measured using an MSD insulin kit. Results were analyzed using an unpaired t-test (FIG. 32). Non-naïve 7-week-old male C57BL/6-Ins2^{Akita}/J (Akita) mice from Jackson laboratory were individually housed and fed a regular chow diet. Mice were randomized by body weight to the following groups: Vehicle (phosphate buffer+Tween)+INS glargine (3 U/kg; n=6), CT-868 (10 nmol/kg)+INS glargine (3 U/kg; n=6), CT-868 (30 nmol/kg)+INS glargine (3 U/kg; n=6), and CT-868 (100 nmol/kg)+INS glargine (3 U/kg; n=5). Mice were fasted for κ hours before the pyruvate tolerance test. After 3 hours of fasting, insulin glargine and CT-868 were administered using separate subcutaneous injections. At the end of the fast, glucose was measured, and pyruvate (1 g/kg) was administered by intraperitoneal injection. After that, blood glucose levels were measured 20, 40, 60, and 120 minutes after the pyruvate injection. Results were analyzed using a one-way ANOVA and Holm-Sidak's test for multiple comparisons (FIG. 33).

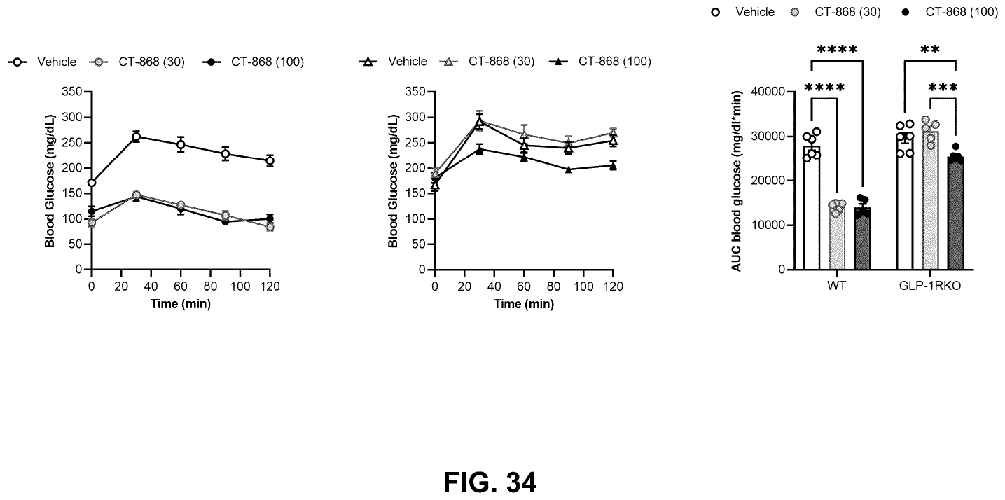

Non-naïve 15-week-old male GLP-1R-WT (WT) and GLP-1R-KO (KO) mice from Taconic were individually housed and fed a regular chow diet. Mice were randomized by body weight into the following groups: WT-Vehicle, WT-CT-868 (30 nmol/kg), WT-CT-868 (100 nmol/kg), KO-Vehicle, KO-CT-868 (30 nmol/kg), KO-CT-868 (100 nmol/kg). Mice were fasted overnight (17 hours). Following the overnight fast, glucose was measured, and compounds were administered by subcutaneous injection. Next, glucose was measured 2 hours after compound administration, and pyruvate (1 g/kg) was administered by intraperitoneal injection. After that, blood glucose levels were measured 20, 40, 60, and 120 minutes after the pyruvate injection. Results were analyzed using a two-way ANOVA and Holm-Sidak's test for multiple comparisons (FIG. 34).

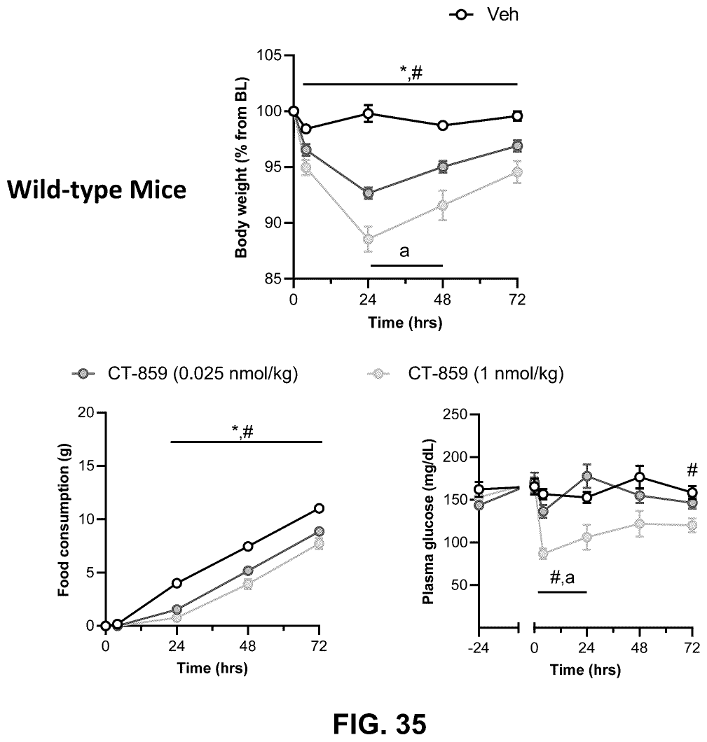

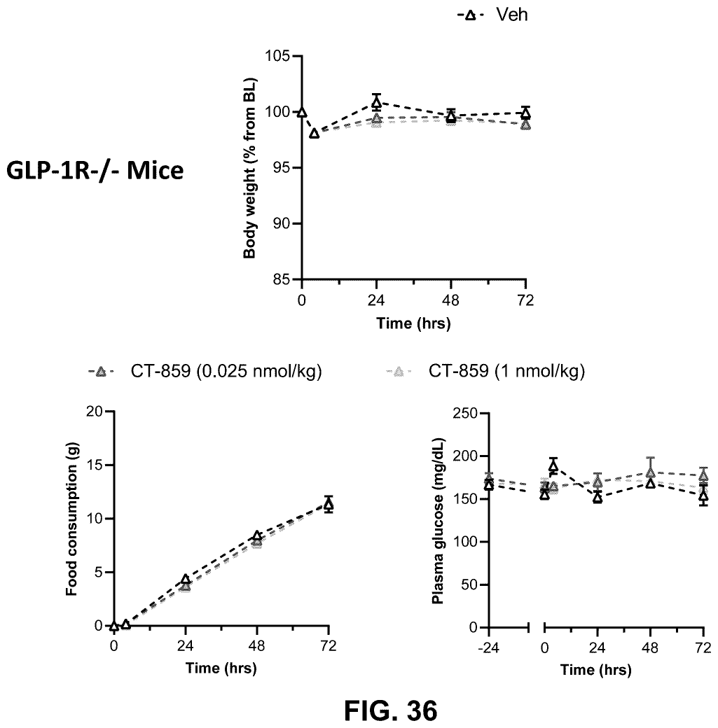

GLP-1R-WT (Wildtype) and GLP-1R-KO (GLP-1R−/−) mice from Taconic were individually housed and fed a regular chow diet. Mice were randomized by body weight into the following groups: Vehicle (phosphate buffer+Tween; n=6), CT-859 (0.025 nmol/kg; n=6), and CT-859 (1 nmol/kg; n=6). Compounds were administered by a single ICV injection. Body weight (BW), total food consumption (F), and plasma glucose were measured at 24-hour intervals up to 72 hours post drug administration (FIGS. 35 and 36). Results were analyzed using a two-way ANOVA and Holm-Sidak's test for multiple comparisons.

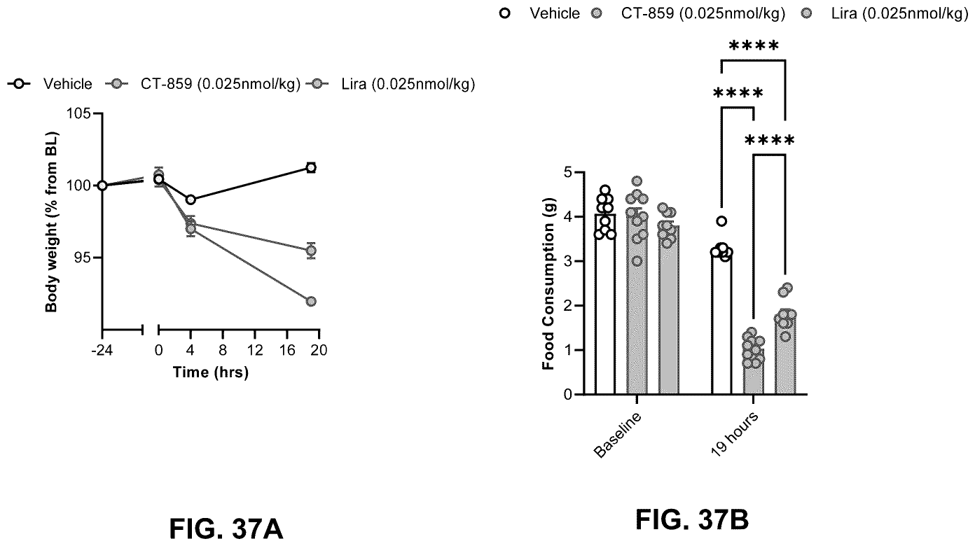

GLP-1R-WT (Wildtype) mice from Taconic were individually housed and fed a regular chow diet. Mice were randomized by body weight into the following groups: Vehicle (phosphate buffer+Tween; n=9), CT-859 (0.025 nmol/kg; n=10), and liraglutide (0.025 nmol/kg; n=9). Compounds were administered by a single ICV injection. Body weight (BW) and total food consumption (F) were measured up to 19 hours post drug administration (FIGS. 37A and 37B). Results were analyzed using a two-way ANOVA and Holm-Sidak's test for multiple comparisons.

### Results

At non-GIPR engaging doses in ob/ob mice, a 30 nmol/kg dose of CT-868 achieved greater weight loss as compared to a 200 nmol/kg dose of liraglutide, an unbiased GLP-1 receptor agonist (19.8% vs 11.5%, p=0.002), indicating biased GLP-1 improves weight loss vs unbiased GLP-1 (FIG. 25). GLUC tolerance test (GTT) in GIPR−/− mice showed similar reduction in GLUC AUC (53% vs 62%, p=NS) 4 h after dosing with 20 nmol/kg doses of liraglutide and CT-859, but only CT-859 maintained significant reduction after 24 h and 48 h, indicating biased GLP-1 induces a sustained GLUC lowering effect (FIG. 26).

After 14 days of dosing in Akita mice with non-GIPR engaging 20 nmol/kg doses of CT-868, body weight (BW) decreased 4% and blood glucose (BG) decreased 30% as compared to a 20 nmol/kg dose of liraglutide (p=0.01), indicating enhanced effect of biased GLP-1 on glycemic control (FIG. 27). At 200 nmol/kg GIPR engaging doses of CT-868 on background of insulin (INS), BG was significantly lowered as compared to a 200 nmol/kg dose of liraglutide in Akita (↓35%, p=0.009) (FIG. 28) and DIO-STZ (↓32%, p<0.0001) (FIG. 29) mice. Additionally, CT-868 adjunctive to insulin lowered BW more than liraglutide in DIO-STZ mice (FIG. 29).

Akita mice treated for 14 days with a 300 nmol/kg dose of CT-868 in combination with low INS (0.5 pellets LinBit INS) normalized BG to same extent as vehicle+high INS (1.5 pellets LinBit INS), but with 68% lower INS levels (p<0.0001) (FIGS. 30 and 31).

In GLP-1R−/− mice, a 300 nmol/kg dose of CT-868 lowered GLUC AUC 38% vs vehicle (p<0.0001) without concomitant INS excursion or hyperinsulinemia, showing GIP enhances INS independent of GLUC disposal or INS sensitivity (FIG. 32).

In response to pyruvate challenge, a 100 nmol/kg dose of CT-868 suppressed GLUC AUC 36% (p<0.05) and 14% (p=0.006) on a background of INS treatment in Akita (FIG. 33) and GLP-1R^{-/-} (FIG. 34) mice, respectively, suggesting GIPR activation may contribute to suppression of endogenous glucose production.

In GLP-1R-WT mice, administration of CT-859 via ICV injection led to a dose dependent reduction of cumulative food consumption, which was associated with a dose dependent reduction of body weight as compared to vehicle control. The reduction of body weight lasted up to 72 hours. The higher dose of CT-859 was also associated with a reduction of blood glucose (FIG. 35). By contrast, administration of CT-859 via ICV injection to GLP-1R-KO mice did not have any significant effects on food consumption, body weight, or blood glucose as compared to vehicle control (FIG. 36).

In a head-to head comparison study, administration of CT-859 via ICV injection led to a significantly greater reduction of food intake and body weight compared to an equal dose of liraglutide (FIGS. 37A and 37B).

Together, these data demonstrate the importance of biased cAMP signaling of GLP-1RA in the central nervous system for prolonged suppression of food intake and weight loss in the mouse model. These data also demonstrate that CT-859 and CT-868 provide improved weight loss via biased GLP-1 with enhanced GLUC homeostasis via biased GIP relative to an unbiased GLP-1 receptor agonist such as liraglutide.

### Example 4: Weight-Independent Effects of CT-868 on Glucose Homeostasis in Overweight and Obese Diabetic Adults

### Methods

CT-868 is a biased dual GLP-1 and GIP receptor modulator that exhibits no β-arrestin coupling or receptor internalization at either receptor. Its effects on insulin (INS) secretion via graded glucose infusion (GGI), glucose (GLUC) homeostasis in response to a mixed meal tolerance test (MMTT), gastric emptying (GE), and food intake (FI) were investigated in a randomized, double-blind crossover study in 20 overweight and obese adults with T2DM. In a previous Phase 1 study, CT-868 was tested up to 11 mg as a single dose and up to 5 mg/day for 14-days without any up-titrations in healthy and overweight/obese participants and found to be safe and well tolerated.

The subjects were split into two groups: CT-868 vs. placebo, n=7; and CT-868 vs placebo vs liraglutide, n=13; the latter as a fixed 3^{rd} period. Mean age and BMI were 52.2 years and 32.7 kg/m², respectively, with 55% males. Treatments were administered for 4 days to minimize weight loss with 14 days washout between periods.

### Objectives

The primary objective of this study was to assess the weight-independent effects of CT-868 on insulin secretion rate (ISR) and glucose homeostasis compared to placebo and liraglutide in overweight and obese adults with T2DM.

### Study Design

This was a Phase 1, double-blind, placebo-controlled, randomized, single-center, cross-over trial of 20 adults of 18-65 years of age diagnosed with T2DM:
a) On diet and exercise only, or on stable therapy (≥3 months) with metformin monotherapy or metformin in combination with sulfonylurea (SU)≥3 months prior to screening. SUs were washed out ≥7 days prior to randomization.
b) Body Mass index (BMI) >27 and →45 kg/m²
c) Baseline HbA1c≤10.5%; and fasting plasma glucose <250 mg/dL

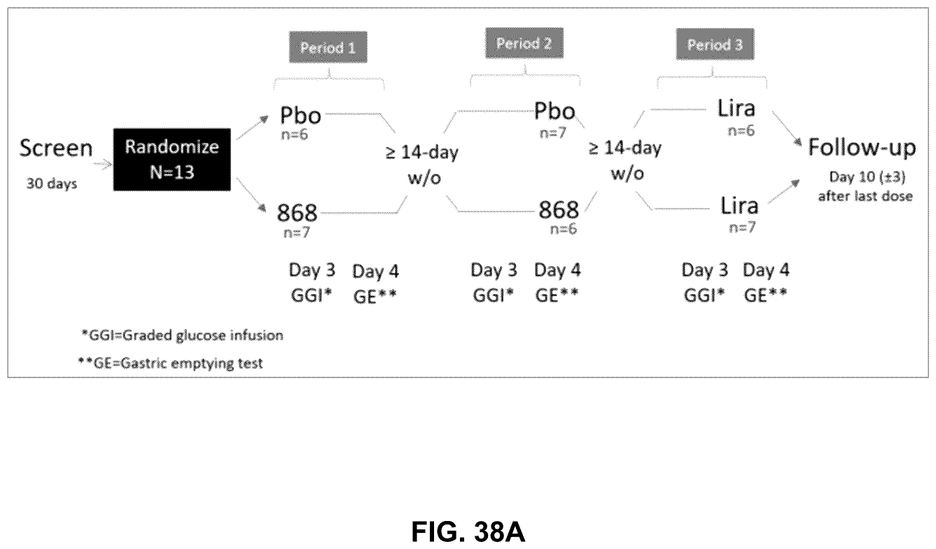

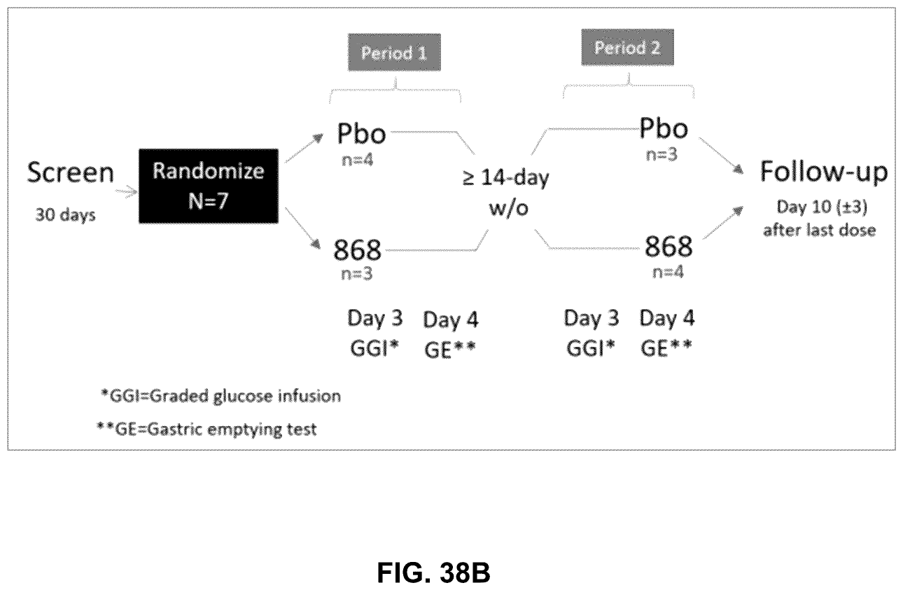

Group 1 (n=13) included a three-way crossover design to assess CT-868 (5 mg, 3.25 mg, and 3.25 mg doses), placebo, and liraglutide (0.6 mg, 1.2 mg, and 1.2 mg doses) as active comparator, assessed during three in-house periods (FIG. 38A). Group 2 (n=7) included a two-way crossover design to assess CT-868 (5 mg and 3.25 mg doses) and placebo during two in-house periods (FIG. 38B).

During each period, subjects received randomized study drug via subcutaneous injection (SC) on Days 1, 2, and 3; pharmacokinetic (PK) and pharmacodynamic (PD) blood samples were collected. Assessments included a Graded Glucose Infusion (GGI) on Day 3; Mixed Meal Tolerance Test (MMTT), Gastric Emptying (GE), and ad libitum food intake assessments on Day 4; and appetite & satiety ratings via Visual Analogue Scales (VAS) on Days 1, 2, 3, and 4.

### Endpoints

The primary endpoint of this study was to evaluate insulin secretory rate (ISR) relative to ambient glucose (ISR/G).

The secondary endpoints of this study were:
To assess changes in glucose, insulin, C-peptide, and glucagon levels during a mixed meal tolerance test (MMTT);
To assess gastric emptying via acetaminophen absorption;
To assess appetite, hunger, and satiety by visual analog scales, and ad libitum food intake; and
To assess plasma exposure of CT-868 [maximum plasma concentration (Cₘₐₓ); time to maximum plasma concentration (tₘₐₓ); area under the concentration-time curve over a dosing interval (AUC₀₋ₜₐᵤ); terminal half-life (t_{1/2})].

Safety assessments of this study included treatment-emergent adverse events (TEAEs), serious adverse events (SAEs), adverse events of special interest (AESI), vital signs, 12-lead electrocardiogram (ECG), clinical lab evaluations, and physical exam.

### Results

A total of 20 participants with T2DM were randomized in the study. Subject demographics are shown in Table 9 below.

TABLE 9
Demographics and Baseline Characteristics

| Characteristics | T2DM (N = 20) |
|---|---|
| Gender-Male [n (%)] | 11 (55.0) |
| Age (years) at Treatment Start-Mean | 52.2 |
| Ethnicity-Hispanic or Latino [n (%)] | 16 (80.0) |
| Weight (kg)-Mean | 93.0 |
| BMI (kg/m²)-Mean | 32.7 |
| Duration (years) of Diabetes-Mean | 6.8 |
| HbA1c (%)-Mean | 7.3 |

Graded Glucose Infusion (GGI) endpoints are shown in Table 10 below.

TABLE 10
PD-Graded Glucose Infusion (GGI) Endpoints

| | Slope of the ISR/G | | |
|---|---|---|---|
| | CT-868 | Placebo | Liraglutide |
| Parameter | (N = 20) | (N = 18) | (N = 10) |
| LS Mean | 2.43 | 0.47 | 2.4 |
| LS Mean (Compared to CT-868) | | 1.92* | 0.02 |
| 95% Confidence Interval | | [1.23, 2.61] | [−0.99, 1.03] |

*Significant compared to CT-868; LS, least squares

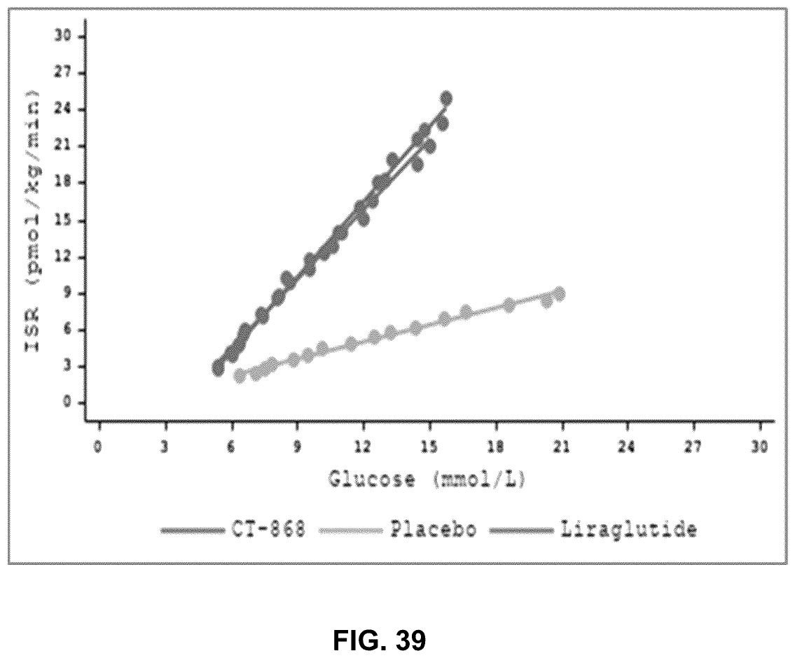

GGI data showed robust insulin secretion for CT-868 vs. placebo [change in ISR/GLUC at GIRₘₐₓ (pmol/kg/min): CT-868 1.1 (0.1), placebo 0.1 (0.2), liraglutide 1.0 (0.3)]. The data demonstrate robust insulin secretory responses in T2DM patients treated with liraglutide or CT-868 as compared to placebo-treated participants (FIG. 39).

MMTT data and insulin incursion data are shown in Table 11 below.

TABLE 11
MMTT and Insulin Excursions

| Incremental | CT-868 | Placebo | Liraglutide |
|---|---|---|---|
| AUC₀₋₂₄₀ₘᵢₙ | (N = 20) | (N = 18) | (N = 10) |
| Glucose, mmol/L*min | 72** | 392 | 187 |
| Insulin, mU/L*min | 1178*^{,‡} | 5832 | 4613 |
| Insulin x Glucose | 84,378 | 2,286,347 | 862,910 |
| | 27x lower vs. placebo | | 2.6x lower vs. |
| | 10x lower vs. Lira | | placebo |

*p < 0.05 compared to placebo;
^{‡}p < 0.05 compared to Lira

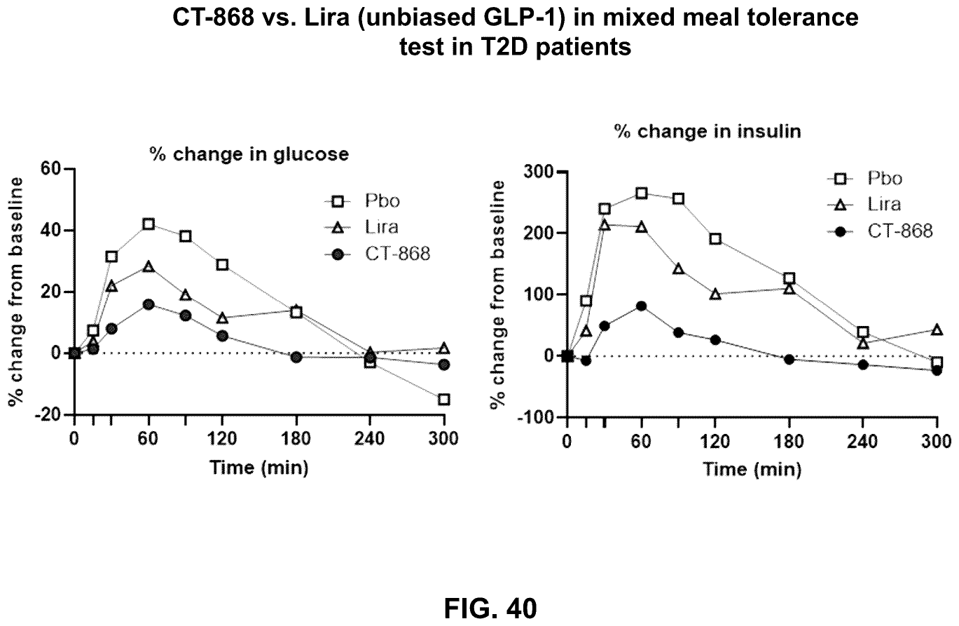

These data show that MMTT based GLUC iAUC₀₋₂₄₀ was significantly reduced for CT-868 vs. placebo [72(46) vs 392(48) mmol/L*min] and numerically lower vs. liraglutide [187(55) mmol/L*min] accompanied by significantly decreased INS iAUC₀₋₂₄₀ VS both placebo and liraglutide: CT-868 1178(846), placebo 5833(873), liraglutide 4613(1253) mU/L*min (FIG. 40). Thus, CT-868 lowers glucose with significantly less insulin excursions as compared to liraglutide during mixed meal tolerance test (MMTT) in T2DM patients.

As both CT-868 and liraglutide had similar delayed gastric emptying (GE) as compared to placebo, the reduced glucose (GLUC) and INS excursion seen with CT-868 suggests improved INS sensitivity or enhanced INS-independent GLUC disposal. CT-868 also demonstrated minimal suppression of glucagon during MMTT as compared to liraglutide that showed glucagon lowering. Food intake (FI) was lower for CT-868 as compared to placebo, accompanied by reduction in hunger and appetite as assessed by VAS. There were no significant body weight changes in any treatment period. GI side effects were mostly mild and transient.

According to these data, concomitantly reduced plasma glucose and insulin excursions could be a consequence of enhanced glucose disposal, e.g., facilitated by insulin sensitizing mechanisms. Disposal of glucose with less/minimal suppression of glucagon could potentially lower hypoglycemic risk for CT-868 as compared to liraglutide. These data support a robust weight-independent effect of CT-868 (vs. unbiased liraglutide) on glucose disposal with minimal suppression of glucagon.

Further, CT-868 tends to lower appetite and hunger scores which translates to a significant suppression of food intake (absolute amounts and calories consumed) during ad libitum meal, as shown in Table 12 below.

TABLE 12
Body Weight, Appetite/Hunger Scores, and Food Intake

| | CT-868 | Placebo (Pbo) | liraglutide |
|---|---|---|---|
| Parameters | (N = 12) | (N = 11) | (N = 4) |
| Body weight (kg) | | | |
| Pre-dose | 93.3 | 94.3 | 94.6 |
| Day 4 | 93.4 | 94.3 | 94.2 |
| Change | 0.09 | 0.00 | -0.39 |
| Appetite (0-100) | | | |
| Pre-dose | 70.8 | 51.5 | 59.2 |
| Day 4 | 51.5 | 66.3 | 50.8 |
| Change | ↓19 points | ↑15 points | ↓8 points |
| Hunger (0-100) | | | |
| Pre-dose | 61.5 | 37.9 | 54.5 |
| Day 4 | 53.0 | 51.1 | 52.0 |
| Change | ↓9 points | ↑13 points | ↓3 points |
| Food Intake (g) | 591.4* | 745.7 | 680.2 |
| | ↓ 21% vs. Pbo | | ↓ 9% vs. Pbo |
| Total Calories consumed | 733.7* | 1078.1 | 959.3 |
| (kcal) | ↓32% vs. Pbo | | ↓11% vs. Pbo |

*Significantly less food (total amount and calories) was consumed after CT-868 treatment vs. Placebo (p < 0.05), but the difference between liraglutide and Placebo was not significant.

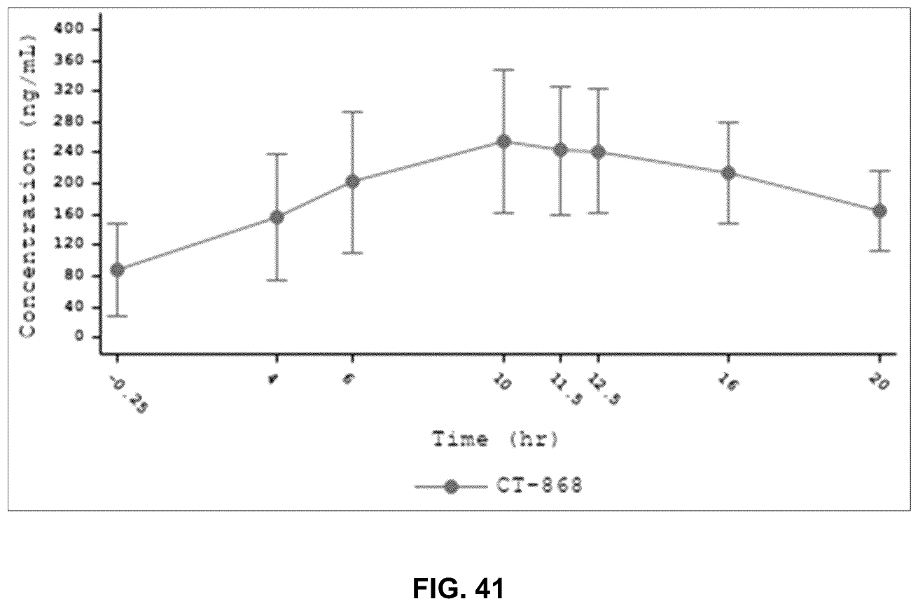

Plasma exposure of CT-868 in T2DM patients is shown in FIG. 41 and Table 13 below. These data confirm that the key pharmacodynamic assessments (i.e., GGI, MMTT, and GE) were generally performed at the time of maximal plasma concentration of CT-868.

TABLE 13
Plasma Exposure of CT-868 in T2DM Patients

| | T2DM |
|---|---|
| Key Parameters (mean) | (N = 20) |
| Maximum Concentration of CT-868 (ng/ml) | 273.9 |
| Time of Maximum Concentration of CT-868 (minutes/hours) | 655.8/10.9 |
| Average Concentration of CT-868 (ng/ml) | 211.9 |
| AUC (0-tau) (ng/ml*min) | 231699 |

Safety studies, summarized in Table 14 below, indicated that:
1. CT-868 was well tolerated;
2. Most TEAEs were Grade 1 (mild) in severity;
3. GI TEAEs for nausea, vomiting, and constipation were of Grade 1 severity, transient, and self-resolved without the need for any concomitant medications;
4. Grade 1 hypoglycemia events (which mostly occurred during the GGI procedure) were observed in 7 participants following CT-868 administration, and in 2 and 3 participants after placebo and liraglutide dosing, respectively; and
5. One subject discontinued (Period 1/Day 4 prior to discharge) for injection site reaction of Grade 1 for RUQ ecchymosis (4 cm×3 cm).

TABLE 14
Safety Data-AE Overview

| | AE Overview | | |
|---|---|---|---|
| | CT-868 | Placebo | Liraglutide |
| | (N = 20), | (N = 18), | (N = 10), |
| Reported AE (at least one) | n (%) | n (%) | n (%) |
| At Least 1TEAE | 14 (70.0) | 9 (50.0) | 7 (70.0) |
| At Least 1 Study Drug Related TEAE | 9 (45.0) | 7 (38.9) | 3 (30.0) |
| Treatment Discontinuation | 1 (5.0)* | 1 (5.6)** | 0 |
| Injection Site Reaction | 1 (5.0) | 0 | 0 |
| AEs of Special Interest (AESI)^{Φ} Total | 10 (50.0) | 2 (11.1) | 4 (40.0) |
| Hypoglycemia^{£} | 7 (35.0) | 2 (11.1) | 3 (30.0) |
| Nausea | 6 (30.0) | 1 (5.6) | 2 (20.0) |
| Vomiting | 2 (10.0) | 0 | 0 |
| Constipation | 1 (5.0) | 0 | 0 |

*Anemia (mild);
**Abnormal coagulation test (mild)
^{Φ}All AESIs were Grade 1;
^{£}reported during GGI procedure

### Conclusions

The results in this study show: body weight was not significantly changed following any of the treatments during the study periods; gastric emptying was delayed by both CT-868 and liraglutide relative to placebo; CT-868 demonstrated a robust insulin secretion response from beta cells in patients with T2DM relative to placebo; CT-868 treatment lowered appetite and hunger scores accompanied by significantly decreased food intake relative to placebo; during the MMTT, patients with T2DM treated with CT-868 demonstrated lower blood glucose accompanied by significantly less insulin excursion as compared to both placebo and liraglutide. The concomitantly reduced glucose and insulin excursions suggests enhanced insulin sensitivity and/or enhanced insulin independent glucose disposal induced by CT-868, independent of weight loss. Finally, the study shows that CT-868 was well tolerated with no significant adverse effects in patients with T2DM.

### Example 5: Pancreatic and Extra-Pancreatic Effects of CT-868, a Signaling-Biased Dual GLP-1/GIP Receptor Agonist, on Metabolic Homeostasis in Pre-Clinical Models and in Participants with Overweight/Obesity with or without Type 2 Diabetes

CT-868, a dual GLP-1/GIP (glucagon-like peptide-1/glucose-dependent insulinotropic polypeptide) receptor agonist, shows selectivity for the GLP-1 receptor (GLP-1R) over the GIP receptor (GIPR) and full cAMP signaling bias on both receptors. CT-868 possesses a once-a-day dosing pharmacokinetic (PK) profile, potently enhancing glucose-stimulated insulin secretion in mice. In mouse obesity models, CT-868 positively influences glucose metabolism and reduces food intake and body weight, showing potential superiority over signal unbiased GLP-IR agonists like liraglutide.

Results from two Phase 1 clinical studies indicate that single (0.1-11 mg) and multiple doses (up to 5.0 mg/day) of CT-868 without up-titration are generally safe and well-tolerated in participants with overweight/obesity with and without type 2 diabetes mellitus. Compared with placebo and liraglutide, CT-868 demonstrates robust glucose lowering with less insulin excursion and preserved glucagon secretion, and induces dose-dependent body weight reduction. These results support further evaluation of CT-868's long-term effects on glucose levels, body weight, and related parameters in individuals with excess weight and/or diabetes.

### Methods

The sources for key reagents and resources are shown in Table 15.

TABLE 15
Sources of Select Resources

| | | |
|---|---|---|
| Reagent or Resource | Source | Identifier |

Chemicals, peptides, and recombinant proteins

| | | |
|---|---|---|
| IBMX | Sigma | 45-17018-1G-EA |
| Human GLP-1 (7-36) | Anaspec | AS-22463 |
| Human GIP (1-42) | Anaspec | AS-61226-1) |
| Liraglutide | SelleckChem | S8256 |
| Tirzepatide | MedChemExpress | HY-P1731B |
| Potassium Phosphate | Sigma-Aldrich | P8709-1L |
| Monobasic | | |
| solution | | |
| Potassium phosphate | Sigma-Aldrich | P8584-1L |
| dibasic solution | | |
| TWEEN ® 20, PROTEIN | EMD Millipore | 655206-50 mL |
| GRADE | Corp. | |
| Water, Optima LC/MS Grade | Fisher Scientific | W64 |
| Saline | Pfizer | 00409488810 |

Critical commercial assays

| | | |
|---|---|---|
| HitHunter ® CAMP Assay for | Eurofins | 90-0075LM |
| Biologics | DiscoverX | |
| Nano-Glo ® Live | Promega | N2012 |
| Cell Substrate | | |
| Nano-Glo ® HiBiT | Promega | N2421 |
| Extracellular | | |
| Detection System | | |
| Fugene 6 | Promega | E2692 |
| U-PLEX Mouse Insulin | Meso Scale | K1526HK-2 |
| Assay | Discovery | |

Experimental models: Cell lines

| | | |
|---|---|---|
| HEK293 | ATCC | CRL-1573 |
| CHO-K1 | ATCC | CCL-61 |
| CHO-K1 Snap-human | Carmot | |
| GLP-1R Cell Line | | |
| CHO-K1 Snap-human | Carmot | |
| GIPR Cell Line | | |
| CHO-K1 murine | Eurofins DiscoverX | 95-0154C2 |
| GIPR Gs Cell Line | | |
| U2OS murine GLP-1R | Eurofins DiscoverX | 95-0179C3 |
| Gs Cell Line | | |
| HEK293 human | Carmot | |
| GLP-1R-LgBiT | | |
| SmBiT-human-β-arrestin-2 | | |
| HEK293 human GIPR-LgBiT | Carmot | |
| SmBiT-human-β-arrestin-2 | | |
| HEK293 mouse | Carmot | |
| GLP-1R-LgBiT | | |
| SmBiT-mouse-β-arrestin-2 | | |
| HEK293 mouse GIPR-LgBiT | Carmot | |
| SmBiT-mouse-β-arrestin-2 | | |

Experimental models: Organisms/strains

| | | |
|---|---|---|
| C57BL/6J | The Jackson | 000664 |
| | Laboratory | |
| C57BL/6J DIO | The Jackson | 380050 |
| | Laboratory | |
| C57BL/6-Glp1r^{em1}_1Cgen | Taconic Biosciences | |

Recombinant DNA

| | |
|---|---|
| SNAP-human GLP-1R | Cisbio |
| SNAP-human GIPR | Cisbio |
| Human GLP-1R-LgBiT | Promega |
| Human GIPR-LgBiT | Promega |
| SmBiT-(human)-β-arrestin-2 | Promega |
| Mouse GLPIR-LgBiT | Promega |
| Mouse GIPR-LgBiT | Promega |
| smBIT (mouse)-β-arrestin-2 | Promega |
| pcDNA3.1 (empty) | Genscript |
| HiBiT-(human)-GLP-1R | Promega |
| HiBiT-(human)-GIPR | Promega |

Software and algorithms

Prism
Excel 3

Other

| | | |
|---|---|---|
| Alpha Trak 3 Starter Kit | Zoetis | MWI 119405 |
| AlphaTRAK 3 Test Strips | Zoetis | MWI 119409 |
| Chow diet | Teklad | 2920x |
| 60% High-fat diet | Research Diets, Inc | D12492 Q#1 |
| High-fat diet | Research Diets, Inc | D12451 |
| 50% dextrose | Vet one | NDC 13985-067-00 |
| Ensure | Abbott nutrition | 57263 |

### Animal Experiments

All rodent experiments were conducted with the necessary approvals from FibroGen and Explora BioLabs' Institutional Animal Care and Use Committee.

### Drug Substance and Structure

CT-868 DS is a novel synthetic hydrochloride salt manufactured as a lyophilized white powder. The structure, molecular formula, and molecular weight of CT-868 are as follows:

CT-868 Structure:
(SEQ ID NO: 3)
2-(2-Piperidon-1-yl)-ethylcarbamoylmethylthio-
acetyl-Glu-Gly-ThrPhe-Thr-Ser-Asp-Tyr-Ser-Ile-
Tyr-Leu-Asp-Lys-Gln-Ala-Ala-Aib-Glu-Phe-Val-Asn-
Trp-Leu-Leu-Ala-Gly-Gly-Pro-Ser-Ser-Gly-Ala-Pro-
Pro-Pro-Ser-Lys(γ-Glu-palmitoyl)-NH₂
hydrochloride salt

Molecular Formula: C₂₁₄H₃₂₃N₄₇K₆₅S (free base)
Molecular Weight: 4610.3 g/mol (free base)

### Cell Lines and Maintenance

Cells were maintained using standard lab practices and in cell-specific media in a 37° C. incubator at 5% CO₂. For human GLP-1R and GIPR cAMP assays, SNAP-human GLP-1R and SNAP-human GIPR CHO-K1 stable clones made in-house were used. For mouse GLP-1R and GIPR CAMP assays, DiscoverX Hithunter U2OS and CHO-K1 cells were used, respectively. For β-arrestin-2 NanoBiT® assays, equal amounts of indicated GPCR-LgBit and SmBiT-β-arrestin-2 plasmids were co-transfected into HEK293 cells and assayed either 48 hours post-transfection or after antibiotic selection for stable plasmid DNA expression. For internalization assays, 6-12 ng of plasmid DNA containing N-terminally HiBiT-tagged human GLP-1R or GIPR open-reading frames plus 6-12 μg carrier DNA were co-transfected. All transfections were carried out using 6-12 ug DNA, a 1:3 ratio of Fugene 6, and 10-12.5 million HEK293 cells in a T75 flask. Assays were conducted at 48 hours post-transfection.

### CAMP Production

The HitHunter® CAMP Assay for Biologics kit was used to measure cAMP production in cells. For human receptors, cells were lifted with cell dissociation media, counted, spun down, and resuspended in 1:2 ratio of anti-cAMP antibody: 1×HBSS/10 mM HEPES/625 μM IBMX. 10,000 cells in 5 uL were added to each well of a 384-well low-volume assay plate. For mouse GLP-1R and GIPR cAMP assays, 3-10,000 cells per well were plated 24 hours prior in Assay Complete Cell Plating reagent 2 (murine GIPR cells) or 5 (murine GLP-1R cells) in 384 well low-volume tissue culture treated plates. Before beginning the assay, media was replaced with 5 μL 1:2 ratio of anti-cAMP antibody: 1×HBSS/10 mM HEPES/625 mM IBMX. Compound dilutions were made 1:1 in DMSO, and then 5 μL was transferred to wells using an ECHO Acoustic Liquid Handler (Labcyte). After 30 minutes of incubation, cAMP detection reagents were added according to the manufacturer's specifications, and after suggested incubation times, luminescence was measured.

### β-Arrestin 2 Recruitment

To measure GLP-1R and GIPR-mediated β-arrestin recruitment, Promega's NanoBiT® technology or NanoLuc® Binary Technology were utilized. Both allow for the detection and quantification of protein: protein interactions in live cells (Samms et al., Trends Endocrinol Metab 31:410-21). The NanoLuc luciferase is split into two subunits, called LgBiT and SmBIT, which are expressed in HEK293 cells as fusion proteins at the C-terminus of GLP-1R or GIPR and the N-terminus of β-arrestin-2, respectively. Twenty-four hours before assays, cells were lifted with Cell Dissociation media and plated at 10,000 cells per well in TC-treated 384 low-volume plates. The next day, media was removed and replaced with 10 μL 1:100 dilution of Nano-Glo® Live Cell Substrate in Optimem and equilibrated to room temperature for ten minutes. Background luminescence was measured before 10 nL compound was added using an ECHO Acoustic Liquid Handler. Luminescence was measured at 1.5-minute intervals for 30 minutes.

### GLP-1R Internalization

GLP-1R and GIPR internalization was measured using Promega's Nano-Glo® HiBit extracellular detection system (Promega Corporation; Madison, WI). HEK293 cells were transiently transfected with low quantities of HiBiT-tagged hGLP-1R or hGIPR plasmids (Promega Corporation; Madison, WI). The next day, cells were lifted using TrypLE express enzyme (ThermoFisher Scientific) and plated at 80,000-100,000 cells per well in 96 well plates. After 48 hours transfection, media was replaced with Nano-Glo HiBIT extracellular buffer containing LgBiT protein (1:100), and the Nano-Glo HiBiT extracellular substrate (1:50). After 15′ equilibration and reading background luminescence, test compounds were added to cells, and plates were read on an EnVision multimode plate reader (Perkin Elmer; Waltham, MA), for 120 minutes at two-minute intervals.

### Equipment and Data Analysis

Luminescence was measured using an En Vision Multimode Plate Reader (PerkinElmer). All assays were set up such that each row of a 384 well plate contained a single dilution series and, for normalization purposes, a single low control well (vehicle-treated) and a single high control well (GLP-1 or GIP-treated). GraphPad Prism version 8.4.3 was used to normalize data, generate nonlinear regression curves, and generate graphs. Each dilution series was normalized to the adjacent high (100%) and low (0%) wells on the plate. Dose-response data was fitted to a curve using nonlinear regression analysis using the “log (agonist) vs response-variable slope” setting, where Y=Bottom+ (Top-Bottom)/(1+10\hat{ }((LogEC₅₀−X)*HillSlope)) and the Hillslope was constrained to 1. Compound potency (EC₅₀) and efficacy (Eₘₐₓ) were extracted from this regression analysis. When maximal activity was less than 10%, a curve was not fitted. For NanoBiT® assays, a single timepoint during the 30-minute activity measurements was selected for maximum luminescence response. Then, each well at this time point was normalized to the background signal and to the low (DMSO) and high (500 nM GLP-1/GIP) controls in every row.

### Mice

Lean C57BL/6J, diet-induced obese (DIO) C57BL/6J, and Leptin deficient mice ob/ob male mice were obtained from the Jackson Laboratory. Mice were singly housed under standard environmental conditions (22° C., 12 h: 12 h light: dark cycle), with ad libitum access to water and regular chow (C57BL/6NJ and ob/ob; laboratory rodent diet 5001, LabDiet) or HFD (DIO C57BL/6J; 60% kcal from fat, Research Diets #D12492) unless otherwise specified. Lean mice were used at eight weeks of age. DIO mice were maintained on HFD for at least 18 weeks before experimentation and were used when they were approximately 23 weeks of age. Ob/ob mice were used when they were nine weeks of age. RenaSci Ltd performed the respiratory exchange ratio study. For this study, C57BL/6J mice were ordered from Charles River, UK, at seven to eight weeks of age. Mice were group-housed (n=3 per cage) and fed a high-fat diet (D12451 diet, 45% kcal as fat; Research Diets) for 21 weeks.

### Intraperitoneal Glucose Tolerance Test

Intraperitoneal glucose tolerance tests (ipGTT) were performed in lean C57BL/6J. Vehicle and CT-868 at the doses provided in each figure were administered by a single subcutaneous injection (SC) five hours before the ipGTT. On the day of the ipGTT, mice were fasted for five hours. Baseline blood glucose was determined using an AlphaTrak glucose meter from whole blood collected from the tip of the tail. After that, glucose 2 g/kg (dextrose as a 20% solution in saline) was administered by a single intraperitoneal injection. Blood glucose was determined at the specified time intervals in each figure. Additionally, whole blood was collected in K2-EDTA microvettes (Sarstedt) and kept on ice until centrifugation at 5000 ref for ten minutes at 4° C. Plasma was removed and stored at −80° C. until insulin was analyzed.

### Meal Tolerance Test

The mixed meal tolerance test (MMTT) was performed in C57BL/6J DIO mice at 23 weeks of age. Vehicle, liraglutide (30 nmol/kg), and CT-868 (30 nmol/kg) were administered by a SC injection (10 mL/kg) 24 hours before the MMTT. Mice were fasted for 16 hours. At the end of the fasting period, blood glucose was measured using an AlphaTrak glucose meter from the tip of the tail. After that, a liquid meal (Ensure Plus; Abbott Nutrition #64905) was administered by oral gavage (10 mL/kg). Blood glucose was determined at the specified time intervals in each figure, and whole blood was collected in K2-EDTA microvettes (Sarstedt) and kept on ice until centrifugation at 5000 ref for ten minutes at 4° C. Plasma was removed and stored at −80° C. until insulin was analyzed.

### Weight Loss

DIO and ob/ob mice were acclimated to daily weighing and handling for approximately one week until weight had stabilized. Mice were injected subcutaneously once daily with the indicated dose of CT-868, liraglutide, or vehicle, based on current body weight. Injections were performed six hours before the start of the dark cycle to approximate reaching Tₘₐₓ when the dark cycle started. In ob/ob, where indicated, 24-hour food consumption studies were performed by adding a pre-weighed amount of food on the cage floor and measuring the amount of food remaining the following day. In both the DIO and ob/ob weight loss studies, body weight as a percentage of initial weight was calculated daily by dividing the daily body weight by the body weight taken before the first dose of peptide, multiplied by 100%. Blood glucose was determined at study termination, and blood was collected to quantify plasma insulin concentrations. Mice were sedated by isoflurane and euthanized by decapitation. Whole trunk blood was collected in K2-EDTA microvettes and was kept on ice until centrifugation at 5000 ref for 10 minutes at 4° C. Plasma was stored at −80° C. until insulin was analyzed. Subcutaneous and inguinal fat and the liver were removed and weighed.

### Respiratory Exchange Ratio

DIO mice were acclimated to being individually housed for two weeks before being placed in the PhenoMaster system (TSE Systems, Bad Homburg, Germany). Baseline parameters were recorded for three days before the start of dosing with CT-868. Mice were allocated to treatment using body weight, food intake, water intake, oxygen consumption (VO₂), carbon dioxide production (VCO₂), and locomotor activity as variables. Vehicle and CT-868 (20 nmol/kg) were dosed daily by a single subcutaneous injection at approximately 15:55 for two weeks. BW was recorded daily during dosing, whereas food intake, water intake, VO2, CO₂, and locomotor activity were recorded by the PhenoMaster system. Results are presented as daily means for the light and dark cycles.

### Plasma Insulin

Plasma insulin concentrations were determined using the U-PLEX mouse insulin assay or mouse/rat insulin assay (Meso Scale Discovery) and read on a MESO Quickplex Q 60 MM instrument.

### Pharmacokinetics

PK of CT-868 was measured in CD-1 male mice. Blood samples were collected following a single SC injection through 32 hours Plasma concentrations were measured using an API 6500 LC-MS/MS system.

### Quantification and Statistical Analysis

Results are presented as means (±standard error). For detecting differences between groups in glucose homeostasis a one-way ANOVA with Tukey's test for multiple comparisons was used. For everything else a 2-way ANOVA with group and time as between-subject factors was used. When significant differences were observed, multiple comparisons were carried out using a Tukey correction. Statistical significance was set at p<0.05. Statistical analyses were performed with GraphPad Prism 9.2.0 (GraphPad Software, Boston, MA).

First-In-Human Study on Safety and Tolerability of Ct-868 in Healthy Participants and Participants with Obesity (Single Ascending Dose and Multiple Ascending Dose Study)

This was a randomized, double-blind, placebo-controlled, dose escalation first-in-human (FIH) conducted from December 2018 to January 2020 at a study center in Victoria, Australia in accordance with principles of the Declaration of Helsinki (Ethical Principles for Medical Research Involving Human Subjects), and the NHMRC National Statement on Ethical Conduct in Human Research 2007 (updated 2018). (Registered on the Australian New Zealand Clinical Trials Registry [ANZCTR] under the identifier: ACTRN12618001988246).

Eligible participants were aged 18 to 65 years (inclusive), with BMI 27 to 45 kg/m² or 20 to 25 kg/m² (‘Lean’ cohort), waist circumference ≥102 cm (males) or ≥88 cm (females) for Part 2 participants only, ≤5% weight loss within the preceding 3 months, normal blood pressure or well managed hypertension (only if dose of blood pressure medication(s) was stable for ≥2 months), normal lipid profile or well managed dyslipidemia (only if dose of lipid-lowering medication(s) was stable for ≥2 months), fasting plasma glucose ≤100 mg/dL (6.0 mmol/L), certified as healthy by comprehensive clinical assessment (detailed medical history, complete physical examination. Comorbidities of higher weight (e.g., mild impaired glucose tolerance, mild hypertension, mild hyperlipidemia) were permitted), clinical laboratory parameters within normal range or deemed not clinically significant; however, serum creatinine, ALP, hepatic enzymes (AST, ALT), and total bilirubin (unless participant has Gilbert syndrome) could not exceed ≥2 (i.e., AST, ALT or ALP) or ≥1.5×the upper limit of normal (ULN) (total bilirubin) and no history of cardiovascular disease over the preceding three years or any other major disease other than well managed hypertension or dyslipidemia. All participants provided written informed consent before any study-specific tests or procedures were performed.

### Trial Design

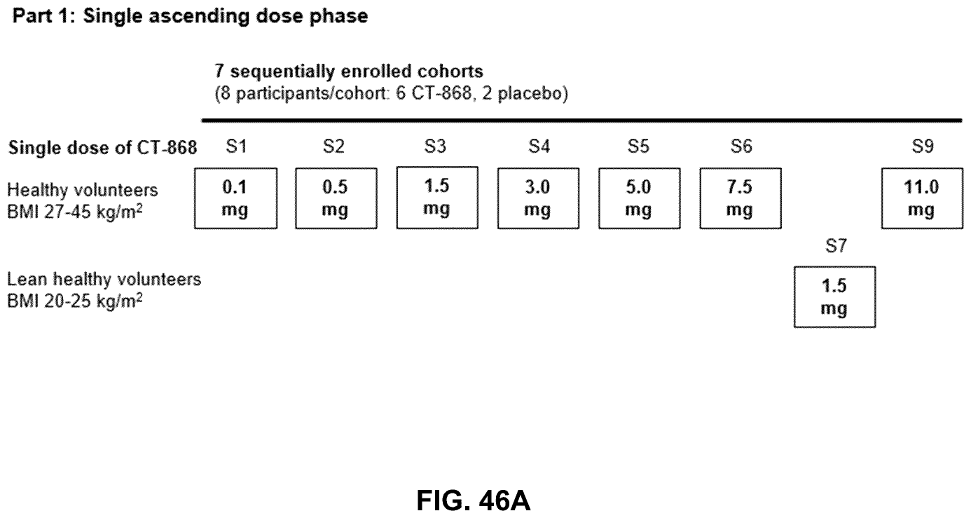

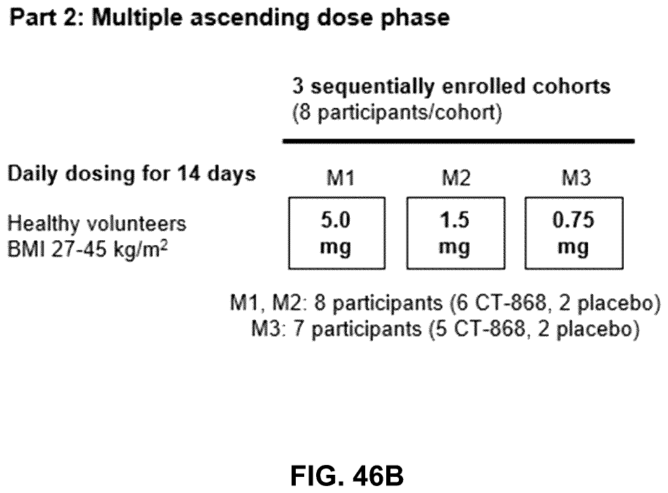

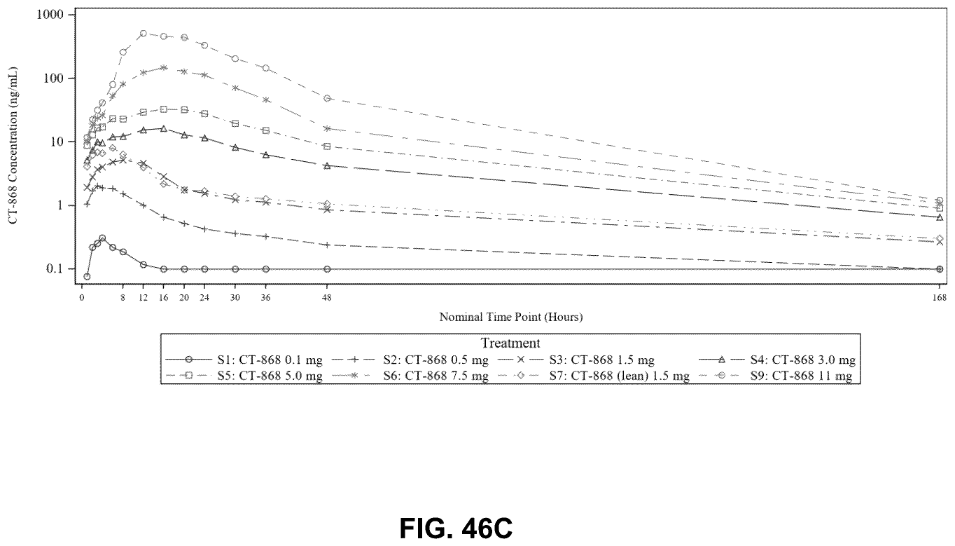

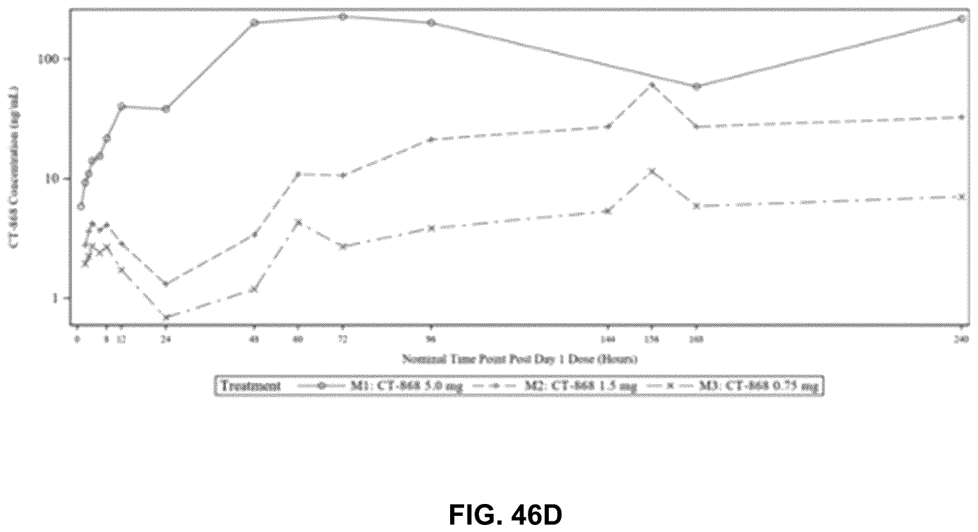

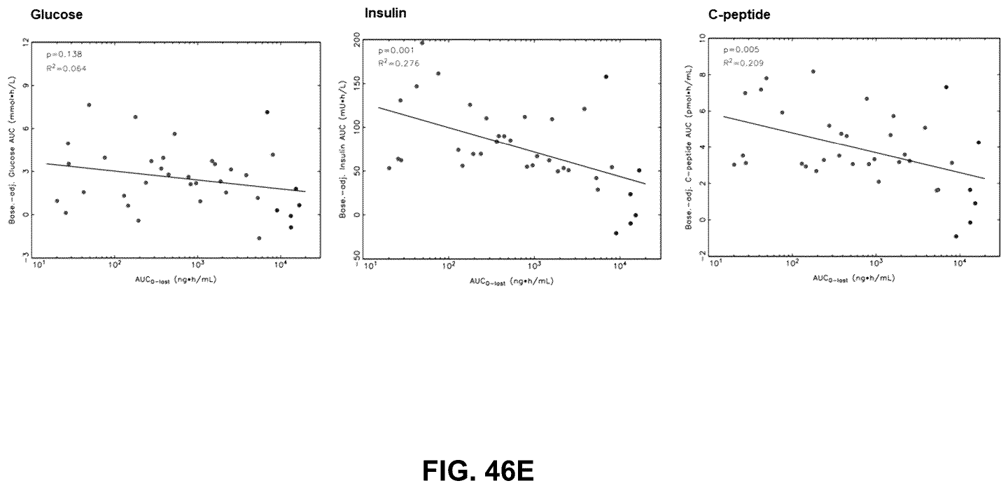

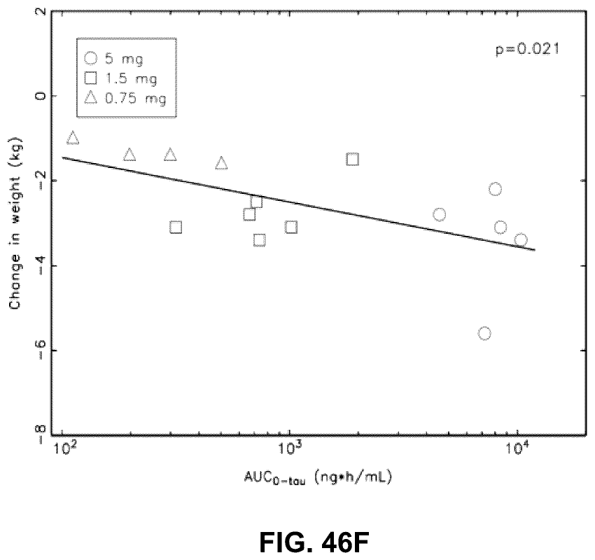

This study was designed to systematically assess the safety, tolerability, PK, and PD of CT-868 when administered as single (SAD) and multiple ascending doses (MAD) in healthy participants and participants with obesity. In Part 1 (SAD), participants were randomized on the morning of Day 1, two sentinel participants received their first dose of study drug (one received CT-868, one received placebo) on Day 1. In the absence of clinically significant safety signals in sentinel participants over this period, the remaining participants received a single SC injection of study drug (CT-868 or matching placebo) except for Cohort S6 and S9, as their dose was above 5.0 mg, received two (S6 participants) or three (S9 participants) SC injections to achieve the specified dose level (FIG. 46A).

In Part 2 (MAD) of the study, participants were randomized on the morning of Day 1 to receive CT-868 or placebo by SC injection. Daily doses were given on Day 2 to Day 14. Cohort M1 was dosed with the highest dose level, determined based on the safety and PK data from the preceding Part 1 (SAD), and doses were decreased sequentially for Cohorts M2 and M3 (FIG. 46B).

### Trial Procedures

Part 1 (SAD) participants were admitted to the treatment unit on Day −1 and were confined and discharged after the completion of all Day 3 assessments and in the absence of clinically significant safety signals. Similarly, Part 2 (MAD) participants were admitted on Day −2 and remained confined until the completion of all Day 16 assessments.

Following study drug administration, Part 1 (SAD) participants underwent safety and other assessments on Day 3 and Day 8 (±1 day, outpatient) and were followed for at least 30 days after the last administered dose of study drug. Part 2 (MAD) participants underwent safety and other assessments and discharged in the absence of clinically significant safety signals, following completion of all Day 16 assessments. All participants were followed for at least 30 days after the last administered dose of study drug. Participants were follow-up for safety and other assessments on Day 21 (±2 days, outpatient) and Day 44 (±3 days, teleconference).

### Primary Endpoints

The primary endpoints were the incidence, nature and severity of AEs, safety laboratory analytes, vital signs, electrocardiograms (ECGs), and incidence of events of special interest (AESI).

### Secondary Endpoints

Characterization of PK profile: PK parameters included maximum concentration (Cₘₐₓ), time to maximum concentration (Tₘₐₓ), area under the plasma concentration-time curve (AUC). Blood samples were collected within two hours prior to dosing for pre-dose samples and within ±10 minutes of the nominal/planned time post-dose. For Part 1 (SAD) participants, samples were taken at −2 to 0 hr (pre-dose), 1, 2, 3, 4, 6, 8, 12, 16, 20 hr (Day 1), 24, 30, 36 hr (Day 2), 48 hr (Day 3) and on Day 8 (±1 day, follow-up) post-dose. For Part 2 (MAD) participants, collection occurred −2 to 0 hr (pre-dose), 2, 3, 4, 6, 8, 12 hr (Day 1), 24 hr (Day 2), 48, 60 hr (Day 3), 72 hr (Day 4), 96 hr (Day 5). For Days 7, 8, 11 and 14, samples were taken −2 to 0 hr (pre-dose), 12 hr on Day 7, 2, 4, 6, 8, 12 hr on Day 14, 24, 36 hr on Day 15, 48 hr on Day 16 and on Day 21 (+1 day, follow-up) post-dose.

Characterization of PD profile: In Part 1 (SAD) participants, plasma glucose was assessed at −2, −1 hr Day −1 (fasting), −2 to 0 hr (pre-dose), 1, 2, 3, 4, 6, 8, 10, 12, 16, 20 hr (Day 1), 24 hr (Day 2), 48 hr (Day 3) and Day 8 (+1 day, follow-up) post-dose. Part 2 (MAD) participants were assessed at 0, 1 hr (Day −1, fasting), −2 to 0 (pre-dose), 1, 2, 3, 4, 6, 8, 12, 16 hr (Day 1); pre-dose (fasting), and before lunch and dinner at 24 hr (Day 2), 48 hr (Day 3), 72 hr (Day 4), 96 hr (Day 5) and −2 to 0 hr pre-dose (fasting) on Days 6, 8, 10 and 12; pre-dose, and before lunch and dinner on Days 6-13; pre-dose, and at 1, 2, 3, 4, 6, 8, 12, 16 hr (Day 14), 24 hr (Day 15) and Day 21 (+1 day, follow-up).

For Part 1 (SAD, Cohorts S2 onwards), participants underwent a meal tolerance test (MTT) at 0 hr (Day −1) and 24 hr (Day 2). Participants ingested 237 mL of a liquid, standardized meal (Ensure PlusR), within 5 minutes, served chilled or at room temperature as desired. Samples were collected 60 minutes prior to the MTT and after start of test meal ingestion, at 20 and 40 minutes (±2 minutes), and at 1, 1.5, 2, 2.5, 3, 3.5, and 4 hours (±10 minutes). Following completion of the MTT, blood samples were collected for the measurement of endpoints (Cavg and AUC for glucose, insulin, and C-peptide). Part 2 (MAD) participants were assessed at 2 hr on Day −1 and 3 hr (post-dose) on Days 7 and 14. In addition, data on their weight, waist circumference, total low-density lipoprotein (LDL)-cholesterol, high-density lipoprotein (HDL)-cholesterol and triglycerides were also collected.

Exploratory outcome measures included insulin, HbA1c, GLP-1, gastric inhibitory polypeptide [GIP], glucagon, adiponectin, fibroblast growth factor 21 [FGF-21], C-terminal telopeptide of type I collagen [CTX] also referred to as C-terminal marker of cross links of collagen, leptin, and ghrelin). For SAD cohort S2 onwards, PD biomarkers were collected on Day 1 at −2 to 0 hr (pre-dose) and four hr post-dose. MAD cohorts had their biomarkers were collected on Day 1 at −2 to 0 hr (pre-dose) and three hr, Day 3 at 48 hr (pre-dose) and 51 hr, Day 5 at 96 hr (pre-dose) and 99 hr, and Day 14, at −2 to 0 hr (pre-dose) and three hr post-dose.

### Quantification and Statistical Analysis

The sample size chosen for the study was selected without statistical justification but was considered adequate for assessing the study objectives. The ITT population comprises all randomized participants. The Safety population comprises all randomized participants who received any amount of study drug (CT-868 or placebo). The PK population comprises all randomized participants who received any amount of active study drug (CT-868) with sufficient plasma concentration-time data to determine at least one PK parameter. The PD population comprises all randomized participants who received any amount of study drug (CT-868 or placebo), with results from baseline and from ≥1 post-baseline PD assessment.

Adverse events were coded using the Medical Dictionary for Regulatory Activities (MedDRAR Version 21.1). The number of participants experiencing treatment-emergent AEs (TEAEs) and number of individual TEAEs are summarized by treatment, SOC, and PT. TEAEs are also summarized by severity and by relationship to study drug. AESIs are listed and summarized by treatment group. Listings of AEs leading to discontinuation of the study, SAEs and deaths, are provided as applicable.

Laboratory evaluations, vital sign assessments, and ECG parameters are listed by participant and summarized by treatment and protocol specified collection time point. A summary of change from baseline and counts of the number of values out of normal range at each protocol specified time point by treatment is also presented for laboratory data. Physical examination abnormalities, including incidence of ISRs, are summarized by treatment and time point and overall.

### PK-PD Modeling

Individual and mean CT-868 concentration-time data are tabulated by cohort/dose level. PK parameters were computed from the individual plasma concentrations of CT-868 using a non-compartmental approach. Estimates for PK parameters are tabulated and summarized by descriptive statistics (mean, standard deviation [SD], median, minimum, and maximum, coefficient of variation [CV %], geometric mean and geometric CV %). Dose proportionality was analyzed using the power model with log-transformed PK parameter values and log-transformed dose. The power model was used to estimate the slope parameter and the 90% confidence intervals (CIs) for the slope. Dose proportionality was generally concluded if the 90% CIs around the slope estimate include the value of 1.

All individual PD and exploratory biomarker data are presented in data listings and summarized by nominal sampling time point, and treatment group with descriptive statistics presented by CT-868 dose, all CT-868 doses combined and combined placebo group. Observed change from baseline and percent changes from baseline for PD biomarker parameters are summarized at each protocol specified time point, by treatment. A generalized linear mixed model approach was used. Where endpoints are a summary statistic over time, for example C_{avg} or AUC for glucose, insulin or C-peptide measures during the meal tolerance test, the model included treatment as a fixed factor and the baseline value as a covariate (i.e., by means of an Analysis of Covariance [ANCOVA] model). Where endpoints were the results from the changes from baseline to each time point, the model included treatment, time point and the interaction between treatment and time point as fixed factors, the baseline value as a covariate and participant identification as a random or repeated factor to cater for the repeated measures nature of the data. Estimates and 95% CIs for the treatment means overall and at each time point and the differences between treatments are presented. P-values of <0.05 will be declared significant.

Placebo- and Comparator-Controlled Crossover Study of CT-868 in Participants with Obesity and Participants with T2DM.

The clinical trial design for the two-part Phase 1 clinical study (Clinicaltrials.gov ID: NCT04973111) was conducted at one center in the United States to evaluate the pharmacokinetics (PK), pharmacodynamics (PD) and safety of CT-868 in participants with obesity (“participants with obesity”) and participants with T2DM (“participants with T2DM”) in accordance with the Protocol, the International Conference on Harmonization (ICH), Guideline for Good Clinical Practice (GCP): Consolidated Guidance (E6) and applicable regulatory requirements including clinical research guidelines established by the Basic Principles defined in the U.S. 21 CFR Parts 50, 56, and 312 and principles of the Declaration of Helsinki (revised version Fortaleza 2013); whichever provided greater protection for the individual.

### Patient Eligibility Criteria

All participants were aged 18-65 years of age at randomization. Participants with obesity had BMI of ≥30 to <40 kg/m² and HbA1c ≥5.7% to ≤6.4% and no history of any type of diabetes. Participants with T2DM were diagnosed with T2DM (but not type 1 diabetes mellitus) with duration ≥6 months based on disease specific diagnostic criteria (World Health Organization [WHO] Classification of Diabetes) and had BMI >27 to ≤45 kg/m², HbA1c≤10.5% and FPG <250 mg/dL. They had to be on a diet and exercise only or stable therapy (≥3 months) with metformin monotherapy or metformin in combination with SUs prior to screening. Participants were fully naïve to GLP-1 analogs/GLP-1 RAs, and DPP-4 inhibitor therapies. All participants were non-smokers (for >6 months prior to the start of the study). Male participants agreed to practice true abstinence or use a condom plus effective contraception and female participants of childbearing potential (WOCBP), appropriate contraception. If postmenopausal (no menses >12 months), status was confirmed through testing of FSH levels outside the normal range for amenorrheic participants <60 years of age.

The exclusion criteria included:
any clinically significant active disease of the GI (peptic ulcers, severe gastroesophageal reflux disease (GERD), gastric emptying abnormality/gastroparesis, any malabsorption/motility disorders, chronic constipation, inflammatory bowel disease (IBD), or irritable bowel syndrome (IBS)), cardiovascular (including a history of arrhythmia, ischemic heart disease), hepatic, neurological, psychiatric, renal, immunological, dermatological, endocrine, genitourinary, or hematological systems, uncontrolled hyperlipidemia, or uncontrolled hypertension (BP) >140/90 mmHg (healthy participants) or a persistent BP systolic or diastolic >160/90 mmHg or <90/60 mmHg (participants with T2DM);
history or current diagnosis of acute or chronic pancreatitis or risk factors for pancreatitis, such as a history of cholelithiasis (without cholecystectomy), hypercalcemia, or severe hypertriglyceridemia;
personal or family history of medullary thyroid carcinoma (MTC) or a genetic condition that predisposed to MTC (i.e., multiple endocrine neoplasia type 2);
clinically significant physical or ECG findings (e.g., QTcF >450 msec for males, QTcF >470 msec for females, left bundle branch block [LBBB]) at screening that may have interfere with any aspect of study conduct or interpretation of results, or present a safety issue to that particular participant;
a history of any weight control treatment, including use of approved weight-lowering pharmacotherapy and/or dietary supplements within the three months of screening;
a previous surgical treatment for obesity (any type of bariatric surgery) or any other GI surgery that may have induced malabsorption/motility issues, history of bowel resection >20 cm, or any GI procedure for weight loss (including LAP-BAND®);
history of, or recent change in body weight ≥±5% within 30 days (by self-report or medical records);
current use of any prescribed or non-prescribed drugs that were known to interfere with glucose or insulin metabolism, including but not limited to systemic corticosteroids, testosterone, anabolic steroids, metformin, GLP-1 analogs/RAs, thiazolidinediones (TZDs), SU, dipeptidyl peptidase 4 (DPP-4) inhibitors, insulin therapy, monoamine oxidase (MAO) inhibitors, growth hormone, or other herbal/over the counter preparations, including amino acids. For participants with current T2DM treatment, SUs was washed out ≥7 days prior to first dose and were on metformin monotherapy only for the duration of the study (participants were fully naïve to GLP-1 analogs/GLP-1 RAs, and DPP-4 inhibitor therapies) and those on stable (≥2 months) therapy of lipid lowering drugs and/or antihypertensive medication, up to 2 different antihypertensive medications were allowed;
current use of any prescribed or non-prescribed drugs that were known to interfere with gut motility including but not limited to chronic opioids, anticholinergics, antispasmodics, 5-hydroxytryptamine (5HT3) antagonists, or dopamine antagonists;
clinically significant abnormal clinical laboratory values including transaminases (aspartate aminotransferase [AST], alanine aminotransferase [ALT] >1.5 (healthy) or ≥3 (with T2DM) times the upper limit of normal [ULN], total bilirubin >ULN, estimated glomerular filtration rate (eGFR)<60 mL/min/1.73 m² as estimated using the Modification of Diet in Renal Disease [MDRD] equation, and/or calcitonin levels >50 ng/L). Participants with T2DM were excluded if fasting serum triglycerides >500 mg/dL or laboratory values were suggestive of pancreatic impairment (e.g., amylase and/or lipase >3×ULN);
a history of any serious adverse reaction or hypersensitivity to any of the study drug components or medicinal products with similar chemical structure, a history of significant multiple and/or severe allergies or had had an anaphylactic reaction or significant intolerability to prescription or non-prescription drugs or food;
a history or positive test of hepatitis B surface antigen (HBsAg), or positive test for hepatitis C, or presence of human immunodeficiency virus type 1 (HIV-1) or 2 (HIV-2) antibodies;
any active or clinically significant infection, including Coronavirus disease (COVID-19) within 15 days prior to screening, or administration of COVID-19 vaccine within 5 days prior to any dosing periods, or if scheduled for such vaccination during any domiciled (in-house treatment) periods. Vaccination for COVID-19 was allowed during the study provided a minimum of 5 days had elapsed after the vaccine administration before any dosing, and if participant was asymptomatic for at least 48 hours after the vaccination. Participants followed site specific COVID-19 protocols during the entire study;
history of alcohol abuse within 1 year; or history of illicit drug abuse, including marijuana, within approximately 3 months, or evidence of current use or positive drug test, including marijuana, at screening; and
participated in an investigational drug/device study within 30 days or 5 half-lives within the last dose of any IP, whichever was longer, donated or suffered a loss of >500 mL of blood within 56 days or had any major surgery within 6 months.

In participants with T2DM, the exclusion criteria additionally included evidence of significant active neuropsychiatric disease, or chronic seizures, or major neurological disorders were excluded. Participants that were stable and controlled by stable doses of selective serotonin reuptake inhibitors (SSRIs), serotonin norepinephrine reuptake inhibitors (SNRIs), antipsychotics and lithium for ≥6 months prior to screening may have been allowed. In addition, those with a history of osteoporosis, osteopenia, recurrent bone fractures, anemia, or hemoglobinopathies; acute proliferative retinopathy or maculopathy, and/or severe neuropathy, especially autonomic neuropathy; recurrent major hypoglycemia, or hypoglycemic unawareness, or recent ketoacidosis; heart disease, defined as symptomatic heart failure (New York Heart Association class III or IV), myocardial infarction, coronary artery bypass graft surgery, or angioplasty, unstable angina requiring medication, transient ischemic attack, cerebral infarct, or cerebral hemorrhage; significant liver disease, other than fatty liver disease; neoplastic disease within the past 5 years (exceptions: participants with adequately treated non-melanomatous skin carcinoma or other malignancies which had been successfully treated ≥10 years prior to the screening visit and were highly unlikely to sustain a recurrence for the duration of the study).

### Trial Design

Participants with obesity were assigned to Group 1 or 2 based on their order of enrollment. In Group 1, participants were randomized to a crossover drug sequence of CT-868, placebo, or liraglutide in a ratio of 1:1 (n=3 on CT-868: placebo: liraglutide sequence and n=3 on placebo: CT-868: liraglutide sequence). Liraglutide was administered in an open labeled fashion. In Group 2, participants were randomized to CT-868 or placebo in a 1:1 ratio (FIG. 46B).

Participants with T2DM were also assigned to Group 1 or 2 based on their order of enrollment. In Group 1, participants were randomized to a crossover drug sequence of CT-868, placebo or liraglutide in a ratio of n=7 on CT-868: placebo: liraglutide sequence and n=6 on placebo: CT868: liraglutide sequence. Liraglutide was administered in an open labeled fashion. In Group 2, participants were randomized to a crossover drug sequence of CT-868 or placebo in a ratio of n=3 on CT-868: placebo sequence and n=4 on placebo: CT-868 sequence.

### Trial Procedures

Participants with obesity had a screening visit (Day −30 to Day 1 prior to dosing), 2 (Group 1) or 3 (Group 2) 4-day in-house treatment periods (Day −1 to Day 3), a washout period(s) of ≥14 days (maximum of 21 days allowed) between the in-house treatment periods, and a follow-up (F/U, Day 10±3 after last dose) visit. In the evening of Days 1 and 2 of treatment period, participants received SC administration of randomized study drug (see Medication dispensing chart). All participants were released home in the evening on Day 3.

Participants with T2DM had a screening visit (Day −30 to Day 1 prior to dosing), 2 (Group 1) or 3 (Group 2) 5-day in-house treatment periods (Day −1 to Day 4), a washout period(s) of ≥14 days (maximum of 21 days allowed) between the in-house treatment periods, and a follow-up (F/U, Day 10±3 after last dose) visit. Participants on metformin and sulfonylureas (SU) combination therapy may have had a run-in period where they were washed off their SUs for at least 7 days prior to In-house Period 1 and remained on metformin monotherapy for the entire study. During treatment, participants received SC administration of randomized study drug in the evenings of Days 1, 2, and 3 (see Medication dispensing chart). Participants in Group 2 were released home in the afternoon/evening of Day 4. Group 1 participants continued to In-house Period 3.

TABLE 16
Medication Dispensing Chart

| In-house | | | | | | |
|---|---|---|---|---|---|---|
| Treatment | | | | | | |
| Period | Group 1 | | | Group 2 | | |
| Assignment | Day 1 | Day 2 | Day 3 | Day 1 | Day 2 | Day 3 |

Participants with obesity

| | | | | | | |
|---|---|---|---|---|---|---|
| CT-868 | 2 injections | 1 injection | n.a. | 2 injections | 1 injection | n.a. |
| | (CT-868 | (CT-868 | | (CT-868 | (CT-868 | |
| | total of | total of | | total of | total of | |
| | 5 mg) | 3.25 mg) | | 5 mg) | 3.25 mg) | |
| Placebo | 2 injections | 1 injection | n.a. | 2 injections | 1 injection | n.a. |
| | (matching | (matching | | (matching | (matching | |
| | placebo | placebo for | | placebo for | placebo for | |
| | for CT-868) | CT-868) | | CT-868) | CT-868) | |
| Liraglutide | 1 injection | 1 injection | n.a. | n.a. | n.a. | n.a. |
| | 0.6 mg | 1.2 mg | | | | |

Participants with T2DM

| | | | | | | |
|---|---|---|---|---|---|---|
| CT-868 | 2 injections | 1 injection | 1 injection* | 2 injections | 1 injection | 1 injection* |
| | (CT-868 | (CT-868 | (CT-868 | (CT-868 | (CT-868 | (CT-868 |
| | total of | total of | total of | total of 5 | total of | total of |
| | 5 mg) | 3.25 mg) | 3.25 mg) | mg) | 3.25 mg) | 3.25 mg) |
| Placebo | 2 injections | 1 injection | 1 injection* | 2 injections | 1 injection | 1 injection* |
| | (matching | (matching | (matching | (matching | (matching | (matching |
| | placebo for | placebo for | placebo for | placebo for | placebo for | placebo for |
| | CT-868) | CT-868) | CT-868) | CT-868) | CT-868) | CT-868) |
| Liraglutide | 1 injection | 1 injection | 1 injection* | n.a. | n.a. | n.a. |
| | 0.6 mg | 1.2 mg | 1.2 mg | | | |

*On Day 3, CT-868/Placebo and/or liraglutide doses may have been adjusted downward to 2.5 mg and 0.6 mg, respectively, based on tolerability as judged by the Investigator.
Abbreviation: n.a. = not applicable

Primary endpoint: The primary endpoint was the insulin secretion rate (ISR) relative to glucose (G). The relationship of ISR at any given level of glycemia was assessed after acute administration of CT-868 by performing a graded glucose infusion (GGI) procedure following two days (Day 3) of dosing to adequately investigate the glucometabolic effects of CT-868 relative to placebo and liraglutide before its weight loss inducing effects had manifested.

Secondary endpoints: Secondary endpoints included maximum glycemic excursion (Gₘₐₓ), Area under the effect curve (AUEC [0-t]) for ISR over the 150-min time interval during GGI, estimated from C-peptide levels, AUEC (0-t) for ISR×G and ISR/G over the 150-min time interval during GGI, estimated from C-peptide levels and AUEC (0-t) and time-averaged PD response (AUEC [0-t]/t) for insulin, C-peptide, glucose and GLG during GGI.

Blood samples, pre- and post-dose, were collected during the in-house treatment periods for the assessment of PD and PK endpoints. In healthy participants, samples were collected on Days 2 and 3 for PK parameters (maximum plasma concentration [Cₘₐₓ], time to maximum plasma concentration [Tₘₐₓ], AUC over a dosing interval [AUC₀₋ₜₐᵤ], total and incremental AUCs, e.g., AUC₍₀₋₂₀ₕ₎ and terminal half-life [t_{1/2}]); and PD parameters (glucose, homeostatic model assessment for insulin resistance [HOMA-IR], insulin, C-peptide, GCG). In addition, fasting plasma glucose (FPG), insulin, C-peptide, GCG and HOMA-IR after ≥10 hours overnight fast and VAS assessments were done on Days 1, 2 and 3.

In participants with T2DM, blood samples were collected on Days 2, 3 and 4 for assessing PK profiles and for PD profiles, on Days 2 and 3. Fasting plasma glucose (FPG), insulin, C-peptide, GCG and HOMA-IR after ≥10 hours overnight fast on Days 1, 2, 3 and 4. A mixed meal tolerance test (MMTT) and prospective meal consumption (ad libitum meal) was administered on Day 4. Appetite and food intake were assessed using a VAS questionnaire (appetite, hunger and satiety) on Days 1, 2, 3 and 4. A gastric emptying/acetaminophen test was also administered on Day 4 and the effect of CT-868 on gastric emptying was assessed through parameters AUC_{0-60 min}, AUC_{0-300 min}, average concentration of acetaminophen, Cₘₐₓ, and Tₘₐₓ.

Safety assessments which occurred throughout the entire study, included monitoring of incidence and severity of AEs, treatment-emergent AEs (TEAEs) and AEs of special interest (AESIs) (e.g., GI events, hypoglycemia), clinical laboratory abnormalities (ECGs, chemistry, hematology, and urinalysis), and changes in vital sign measurements (BP, heart rate, respiratory rate, and aural temperature).

### Quantification and Statistical Analysis

The Enrolled Set was defined as all participants who signed informed consent and was used for the disposition analysis. The Safety Set was defined as all randomized participants who received at least one dose of study drug, determined overall and separately for each of the in-house treatment periods. The Safety Set was used for all safety analyses, demographic and baseline characteristics, prior/concomitant medications, protocol deviations. The PK Set used for all PK analyses was defined as all participants in the Safety Set who received the study drug with evaluable PK data that are appropriate for the evaluation of the PK profile within each in-house treatment period, determined overall and separately for each of the in-house treatment periods. The PD Set used for all PD analyses was defined as all participants in the Safety Set who received the study drug, with evaluable PD data that are appropriate for the evaluation of PD parameters within each in-house treatment period, determined overall and separately for each of the in-house treatment periods.

The pre-hepatic ISR was calculated based on deconvolution of peripheral C-peptide concentrations during GGI. The assessment of the effect of CT-868 on the relationship of ISR and G (ISR×G and ISR/G), compared to placebo and liraglutide were assessed using the PD population with two mixed-effect models, for each part and each comparison due to the design of the study. The model for the comparison of CT-868 versus placebo included treatment group, treatment sequence, and in-house treatment period as fixed effects, and participant-within-sequence as a random effect. The model for the comparison of CT-868 versus liraglutide included treatment group, treatment sequence, and treatment group-by-sequence interaction as fixed effects, and participant-within-sequence as a random effect. The response variables were the slope of the ISR×G curve as well as the slope of ISR/G within two independent models. The least-squares (LS) means of each treatment group and the LS mean differences between CT-868 and placebo and between CT-868 and liraglutide were calculated and associated 95% confidence intervals (CIs) constructed. Kenward-Roger's approximation for the denominator degrees of freedom was used. Timepoints were defined as intervals of 10 minutes from time 0 minutes to 150 minutes.

Except for whether an ad libitum meal was consumed, each PD exploratory endpoint used the same analysis method (mixed-effect model) as the primary endpoint analysis using the applicable timepoints described in each section. All exploratory PD and PK parameters were to be summarized with descriptive statistics (n, mean, SD, Coefficient of variation [% CV], min, quartile 1, median, quartile 3, and max) per parameter by study part, treatment, and timepoint, by study part for GGI, or by treatment for Part 2.

All statistical analyses were 2-tailed and assessed at the 5% significance level. Tabulations were produced for appropriate demographic, baseline, and safety parameters. For categorical variables, summary tabulations of the number and percentage of participants within each category (with a category for missing data) of the parameter were presented. For continuous variables, the number of participants (n), mean, standard deviation (SD), median, minimum (min), and maximum (max) were presented. Coefficient of variation were presented for certain analyses.

AE terms recorded by the Investigator were coded to system organ class (SOC) and preferred term (PT) using the Medical Dictionary for Regulatory Activities (MedDRA) version 24.0. Severity was collected using the Common Terminology Criteria for AEs (CTCAE) version 5.0 Toxicity Grade. Events with missing severity were summarized as Grade 3. TEAEs summarized by severity counted each participant once for the maximum severity within each SOC and PT within each in-house treatment period and overall. TEAEs marked with a relationship to study treatment as definitely, probable, possible, or missing were considered as related TEAEs. Events with missing relationship to study treatment were summarized as related. The number and percentage of participants with TEAEs were summarized by MedDRA-coded SOCs and PTs.

### Results

### Chemical Structure of CT-868

CT-868 is a modified 39 amino acid peptide. While the hybrid GLP-1/GIP peptide sequence affords optimal activity against both GLP-1R and GIPR, the N-terminal modification with a 2-((2-oxo-2-((2-(2-oxopiperidin-1-yl)ethyl)amino)ethyl)thio) acetyl group is designed to stabilize the molecule toward dipeptidyl peptidase-4 cleavage, imparting bias for G-protein coupling over β-arrestin recruitment. Acylation of the C-terminal lysine side chain with a γ-glutamylpalmitoyl group extends the circulating half-life of the molecule via albumin binding to facilitate once daily dosing.

### Pre-Clinical Models

CT-868 is a cAMP-biased dual agonist for GLP-1/GIP receptors with imbalanced selectivity for GLP-1R.

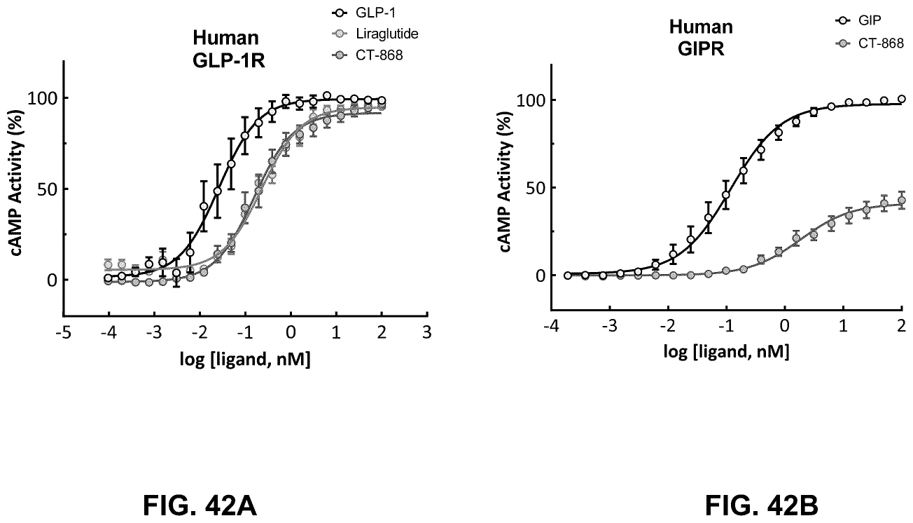

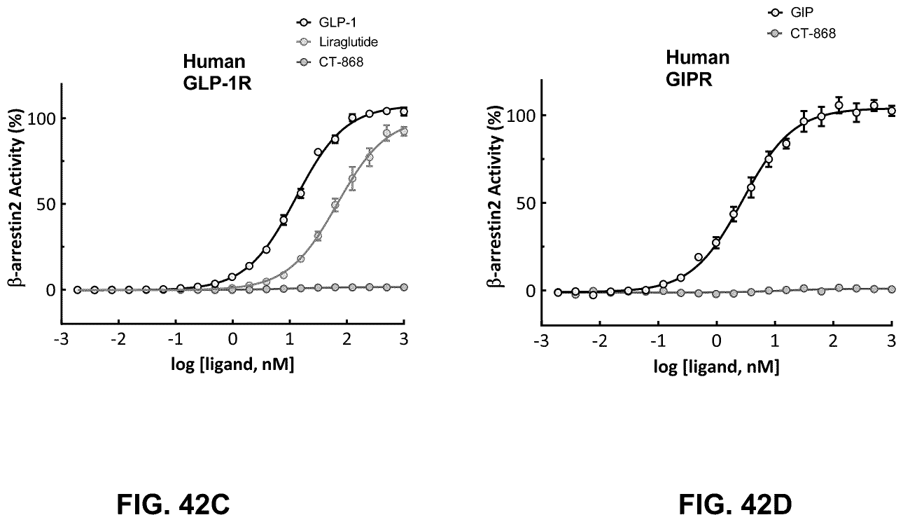

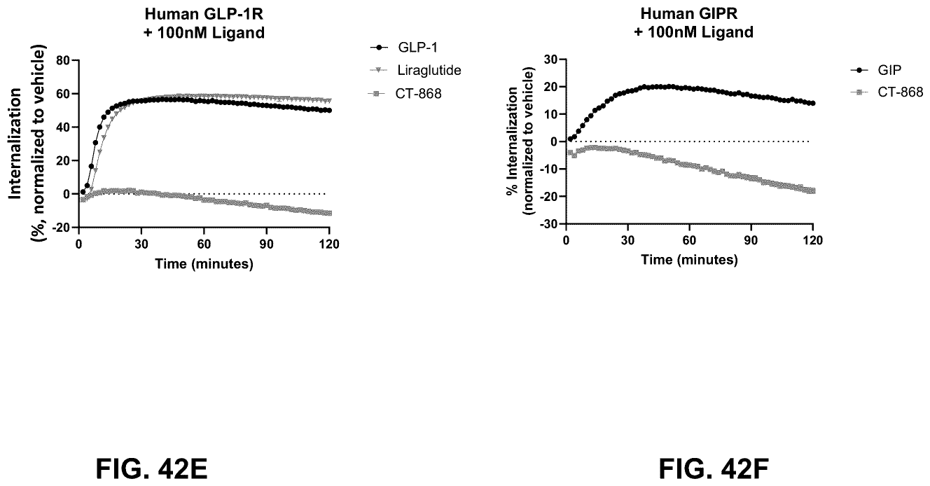

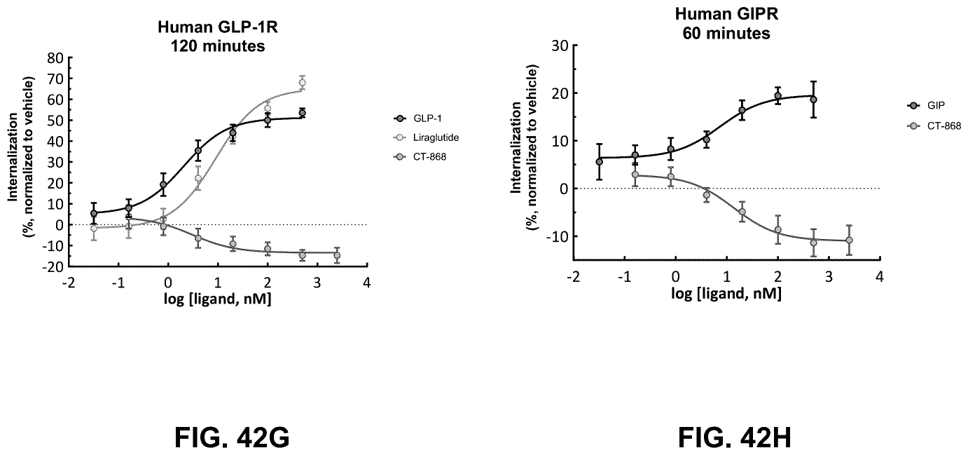

In vitro experiments using recombinantly expressed receptors were conducted to evaluate the potency, efficacy, and signaling characteristics of CT-868 at GLP-1R and GIPR. At human GLP-1R, CT-868 acts as a potent, full agonist of cAMP accumulation. CT-868 is six-fold less potent than the native ligand GLP-1, similar to liraglutide (FIG. 42A and Table 17).

TABLE 17
The Intrinsic Potency of CT-868 in GLP-1R or GIPR Expressing Cells

| | GLP-1/GIP | CT-868 | Liraglutide |
|---|---|---|---|
| hGLP-1R CAMP (-alb) | 0.026 (99, 6) | 0.156 (92, 8) | 0.230 (95,8) |
| EC₅₀, nM (% Eₘₐₓ, N) | | | |
| hGIPR CAMP (-alb) | 0.118 (98, 6) | 1.9 (41, 6) | n.t. |
| EC₅₀, nM | | | |
| (% Eₘₐₓ, N) | | | |
| hGIPR/hGLP-IR | 5 | 12 | n.t. |
| CAMP ratio | | | |
| hGLP-1R | 13 (108, 6) | 11 (1.5, 6) | 68 (101, 6) |
| β-arrestin-2 EC₅₀, nM | | | |
| (% Eₘₐₓ, N) | | | |
| hGIPR β-arrestin-2 | 2.8 (104, 6) | 13 (1, 6) | n.t. |
| EC₅₀, nM | | | |
| (% Eₘₐₓ, N) | | | |
| hGLP-IR | 2.1 (51, 6) | 3.1 (−13, 6) | 9.1 (65, 6) |
| Internalization EC₅₀, | | | |
| nM (% Eₘₐₓ, N) | | | |
| hGiPR Internalization | 7.4 (20, 12) | 14 (−11, 12) | n.t. |
| EC₅₀, nM | | | |
| (% Eₘₐₓ, N) | | | |

The intrinsic potency of CT-868 in mouse GLP-1R or
GIPR expressing cells

| EC₅₀, nM (E ₘₐₓ%) | GLP-1/GIP | CT-868 | Lira |
|---|---|---|---|
| mGLP-1R cAMP (-alb) | 0.007 (96, 6) | 0.164 (95, 6) | 0.028 (92, 6) |
| EC₅₀, nM (% Eₘₐₓ, N) | | | |
| mGIPR CAMP (-alb) | 0.022 (89, 6) | 1.03 (36, 6) | n.t. |
| EC₅₀, nM (% Eₘₐₓ, N) | | | |
| mGIPR/mGLP-IR | 3 | 6 | — |
| CAMP ratio | | | |
| mGLP-1R β-arrestin- | 23.13 | 21.05 (4.69, 6) | 132.7 (80.68, 6) |
| 2 EC₅₀, nM (% Eₘₐₓ, N) | (101.2, 6) | | |
| mGIPR β-arrestin- | 13.79 | 6.211 (−1.92, 6) | — |
| 2 EC₅₀, nM (% Eₘₐₓ, N) | (98.97, 6) | | |

In contrast, at human GIPR, CT-868 is 16-fold less potent than the native ligand GIP, and is a partial agonist of cAMP accumulation (FIG. 24B and Table 17). These data illustrate the selectivity of CT-868 for GLP-1R over GIPR, contrasting with the dual agonist tirzepatide's GIPR selectivity (El et al., Nat Metab. (2023) 5:945-54). However, unlike the native ligands, or liraglutide and tirzepatide, there is a complete absence of β-arrestin recruitment in response to CT-868 stimulation at either receptor (FIGS. 24C-D, Table 17).

Further analyses showed that, whereas the native ligands promote receptor internalization, CT-868 effectively suppresses GLP-1R and GIPR internalization, as evidenced by the greater surface expression after 60 to 120 minutes compared with vehicle-treated controls (FIGS. 42E-F and Table 17). This is consistent with CT-868's limited ability to recruit β-arrestin. These results show that CT-868 is selective toward GLP-1R and fully biased in engaging cAMP signaling over β-arrestin recruitment and internalization. Supporting its use in pre-clinical studies, CT-868 displayed comparable potency and efficacy in mouse GLP-1R and GIPR expressing cells, as validated by cAMP accumulation assays (Table 17).

### CT-868 Enhances Glucose Homeostasis Through GLP-1R- and GIPR-Mediated Mechanisms.

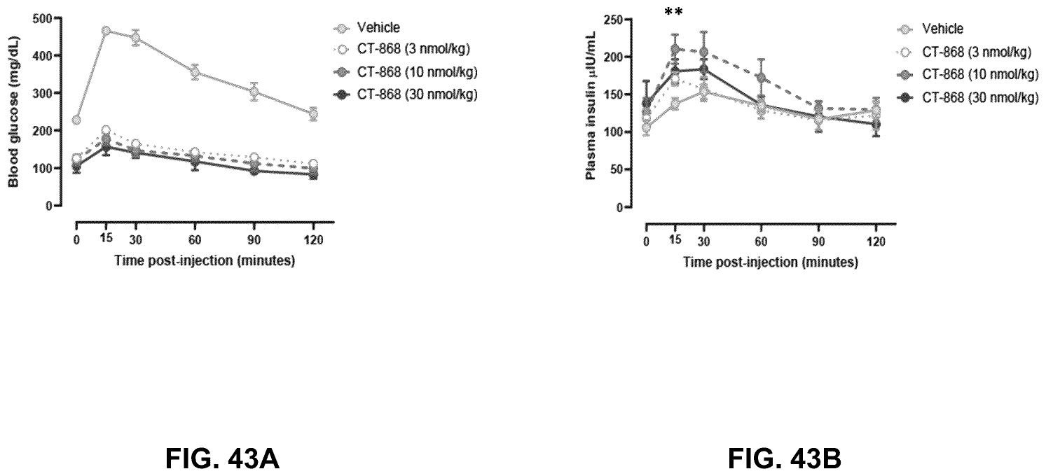

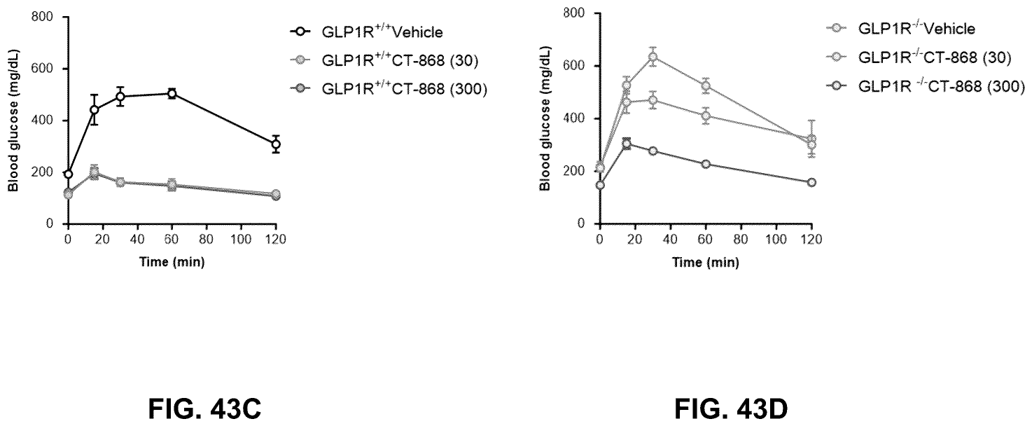

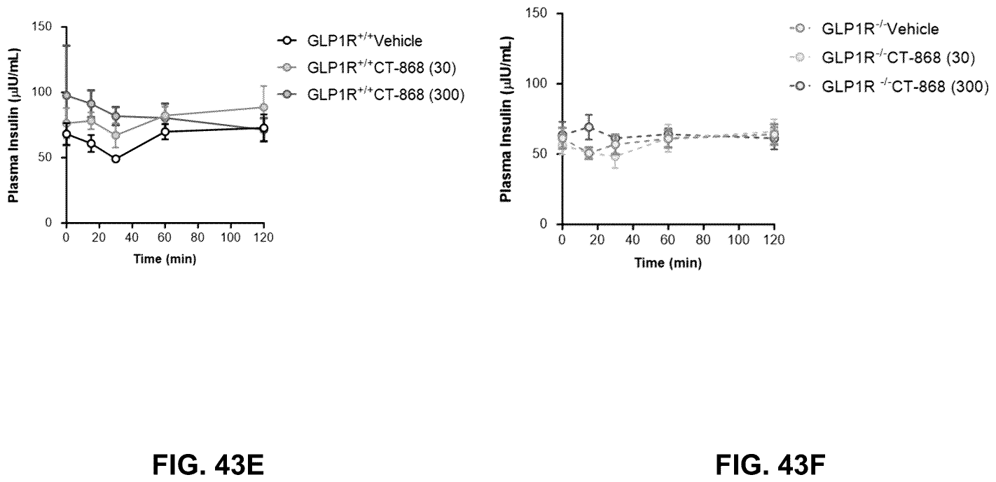

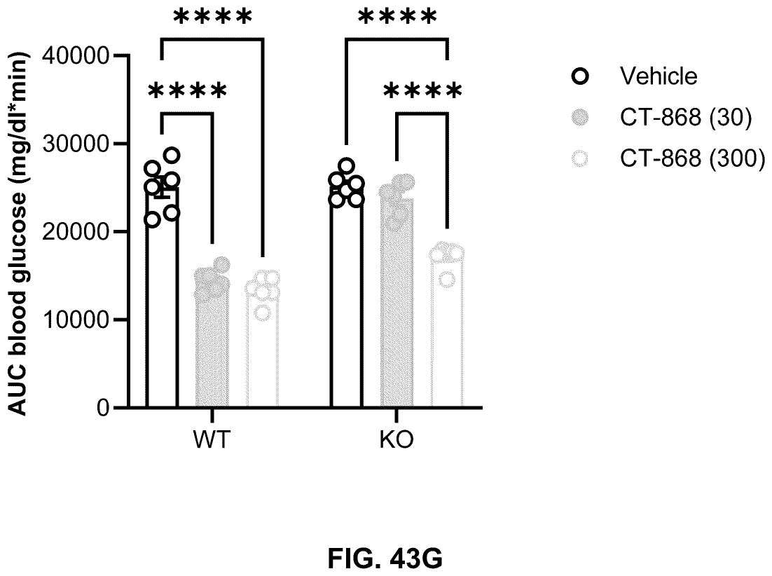

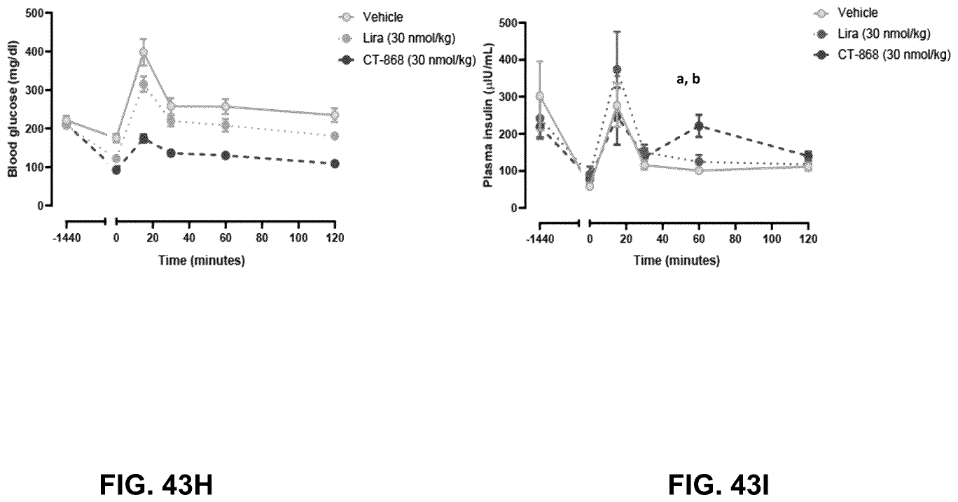

Glucose tolerance tests (GTT) were conducted in mice to assess the effects of CT-868 on glucose-stimulated insulin secretion (GSIS) and glucose excursion. During intraperitoneal glucose tolerance tests (IPGTTs), a single subcutaneous injection of CT-868, even at the lowest dose tested (3 nmol/kg), substantially and significantly suppressed glucose excursion compared with the vehicle group (FIG. 43A). Area under the curve (AUC) analyses for plasma insulin revealed no significant differences between the vehicle and the administered doses of CT-868; however, post-prandial plasma insulin levels at 15 min were significantly higher with CT-868 (10 nmol/kg) than with vehicle treatment (FIG. 43B).

To assess GIPR-mediated activity on glucose control, CT-868 was administered to GLP-1R knock-out (KO) mice prior to IPGTT. At a dose of 30 nmol/kg, CT-868 exhibits limited suppression of glucose through GIPR; however, significantly greater glucose-lowering effects are observed at higher doses (i.e., 300 nmol/kg) (FIG. 43D). At the higher GIPR-engaging dose, reduction in blood glucose with CT-868 is achieved without increased insulin secretion. These results suggest that the glucose-lowering effects at this dose are insulin-independent. Therefore, a pyruvate tolerance test (PTT) was performed to determine if suppression of endogenous glucose production contributes to the observed effects. CT-868 at 30 nmol/kg and 300 nmol/kg decreased glucose levels during the PTT in wild-type mice; however, in GLP-IR KO mice, only the 300 nmol/kg dose lowered glucose levels during the PTT (FIGS. 43C-D), suggesting that GIPR activation may contribute to glucose lowering by suppressing endogenous glucose production.

Previous studies reported improved efficacy of a biased GLP-IR agonist (Willard et al., JCI Insight (2020) 5). To confirm this finding, the glucose-lowering effects of CT-868 were investigated at a minimal-GIPR-engaging dose compared with liraglutide, an unbiased GLP-1R agonist, in diet-induced obese (DIO) mice. To this end, a mixed meal tolerance test (MMTT) was performed 24 h after equimolar dosing (30 nmol/kg) of either CT-868, liraglutide, or vehicle. The AUC analysis indicated that both liraglutide and CT-868 significantly reduced blood glucose levels compared to the vehicle-treated group. Furthermore, CT-868 at a minimal-GIPR-engaging dose exhibited greater efficacy than liraglutide in suppressing glucose excursion following the meal challenge (FIG. 43H). CT-868 treated mice had higher insulin levels at 60 minutes post-treatment; however, the AUC analysis indicated only a non-significant trend toward higher insulin levels compared with vehicle and liraglutide (FIG. 43I).

To better interpret these results and to help determine the dosing regimen of CT-868 in pre-clinical studies, pharmacokinetic (PK) studies in mice were conducted. The PK studies revealed that the exposure of liraglutide is three-fold higher than that of CT-868, with an AUC_{0-Inf} of 22286 and 7921 h*nM/L, and circulating half-life of 4 h and 7 h, respectively. This suggests that the improved glucose handling as a result of CT-868 treatment compared with liraglutide treatment in the previous study was likely not due to a difference in PK properties but rather to the unique signaling properties of CT-868. These PK properties support once-a-day dosing of CT-868 in mice.

### Chronic CT-868 Administration Dose-Dependently Reduced Body Weight

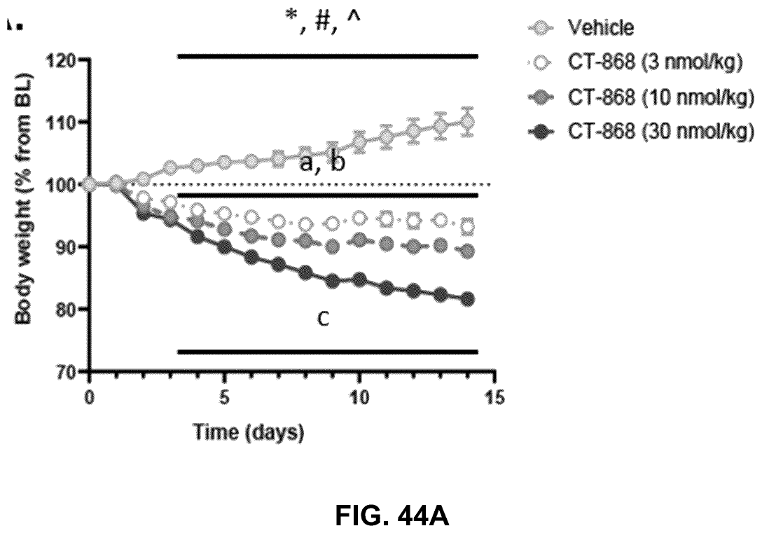

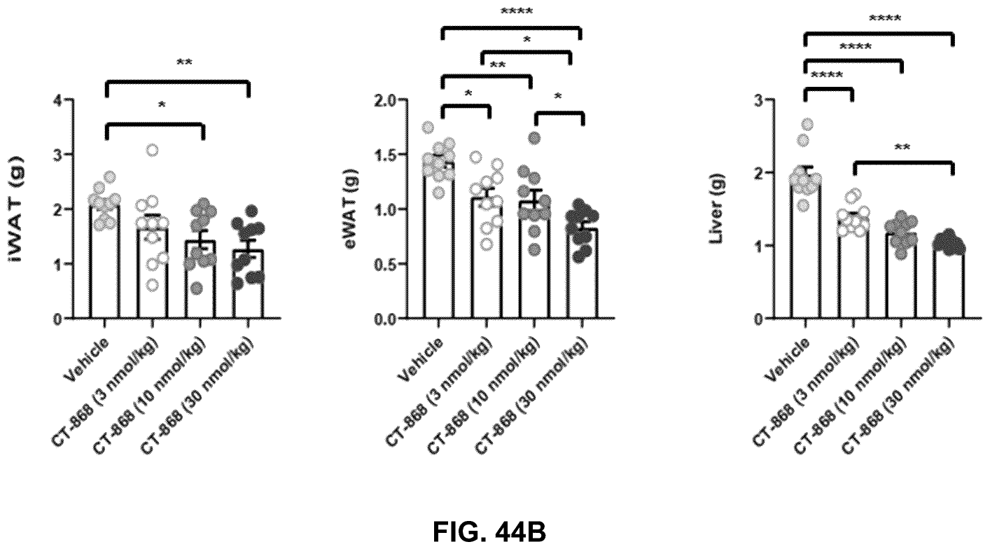

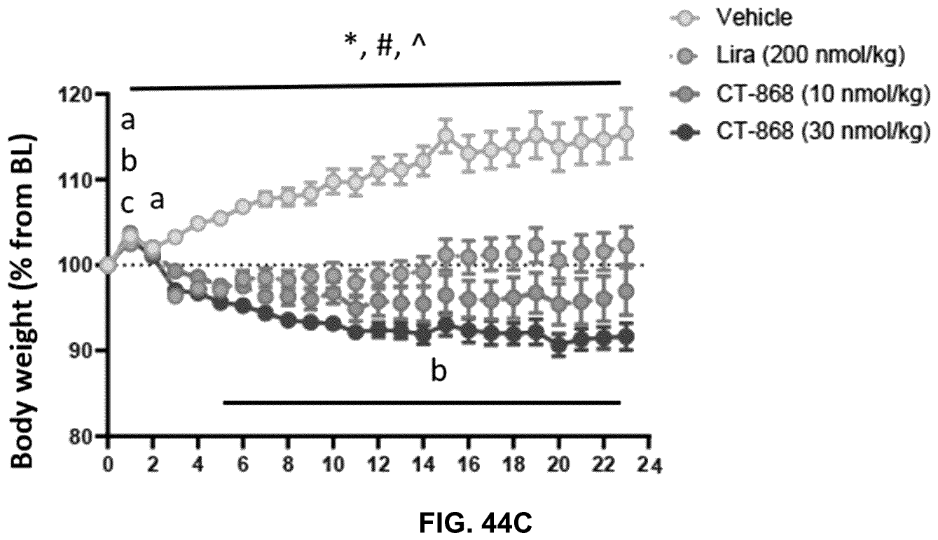

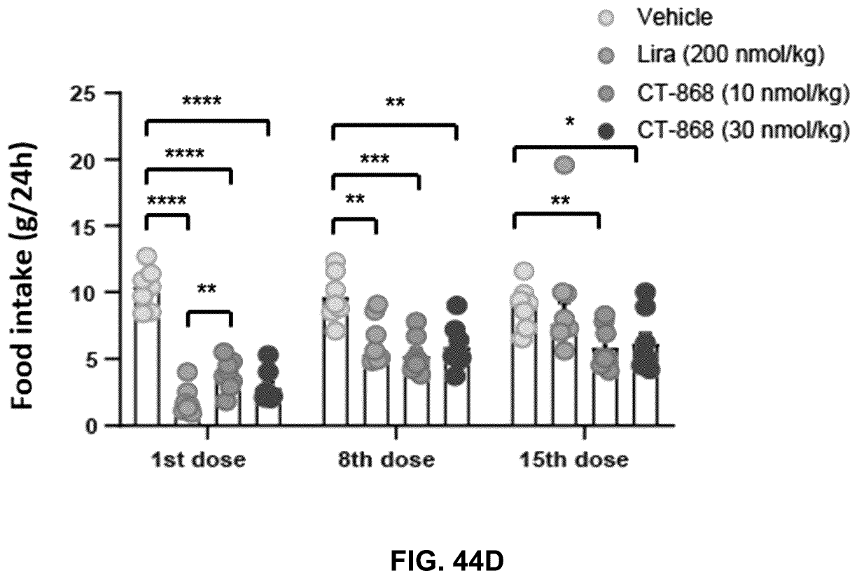

A series of in vivo studies were conducted to investigate the effects of chronic CT-868 treatment on body weight and body composition in mice. DIO mice receiving daily subcutaneous injections of CT-868 (3, 10, or 30 nmol/kg) exhibited a dose-dependent decrease in body weight over 14 days, with mean reductions of 6.8±1.2% (SD), 10.7±0.6% (SD), and 18.4±1.0% (SD) from their baseline weights, respectively (FIG. 44A). In contrast, vehicle-treated mice gained on average 10.0±2.2% (SD) of their baseline body weight over the same treatment period.

Body composition was analyzed at the end of the study. CT-868 treatment resulted in a dose-dependent reduction in inguinal white adipose tissue (iWAT), epididymal white adipose tissue (eWAT), and liver weight compared to vehicle-treated controls (FIG. 44B). Specifically, at 30 nmol/kg, CT-868 resulted in an impressive 61%, 58%, and 52% reduction in iWAT, eWAT, and liver weight, respectively, compared with vehicle-treated controls. This demonstrates that the weight loss observed upon CT-868 treatment is largely due to the reduction of fat mass.

In a subsequent set of in vivo chronic administration studies, it was determined whether CT-868 would also be effective in a mouse model of genetic obesity. Leptin-deficient (Lep^{ob/ob}, hereafter referred to as ob/ob) mice were treated with CT-868 (10 or 30 nmol/kg), liraglutide (200 nmol/kg), or vehicle. After 24 days of daily dosing, mice receiving 10 and 30 nmol/kg of CT-868 exhibited significant dose-dependent weight loss (3.1±2.8% (SD) and 8.4±1.6% (SD), respectively) compared with liraglutide-treated and vehicle-treated counterparts (FIG. 44C). In contrast, liraglutide-treated and vehicle-treated mice displayed gains of 2.2%±2.2% (SD) and 15.4%±2.9% (SD) of their baseline body weight, respectively, over the same treatment period. These results show that CT-868 is more effective than liraglutide in reducing body weight and achieves its maximal effects at substantially lower doses.

All treatment groups showed significantly reduced food intake relative to vehicle-treated controls after the 1st dose (Day 1) and the 8th dose (Day 8). However, only the CT-868 group continued to show lower food intake at Day 15, after the 15th dose (FIG. 3D). These findings showed that CT-868 treatment was associated with a consistent and sustained reduction in food consumption, whereas the effects of liraglutide treatment on food consumption diminished by the final measurement.

Having established the weight-reducing effects of CT-868, the potential mechanisms driving these changes were next investigated. To gain insight into the respiratory exchange ratio (RER), a key indicator of fuel utilization and energy expenditure, indirect calorimetry was performed. DIO mice received daily subcutaneous injections of either CT-868 (20 nmol/kg) or vehicle. Oxygen consumption (VO₂), carbon dioxide production (VCO₂), and ambulatory activity were monitored twice daily for 14 days to capture potential diurnal variations. The RER (VCO₂/VO₂ ratio) was similar across treatment groups during the baseline period and ranged from 0.81-0.86 (left side of red dividing line in Supplemental Figure S1A). A value of 0.8 suggests mixed utilization of carbohydrate and fat, which is typical, and the RER in both groups tended to be slightly higher during the dark phase.

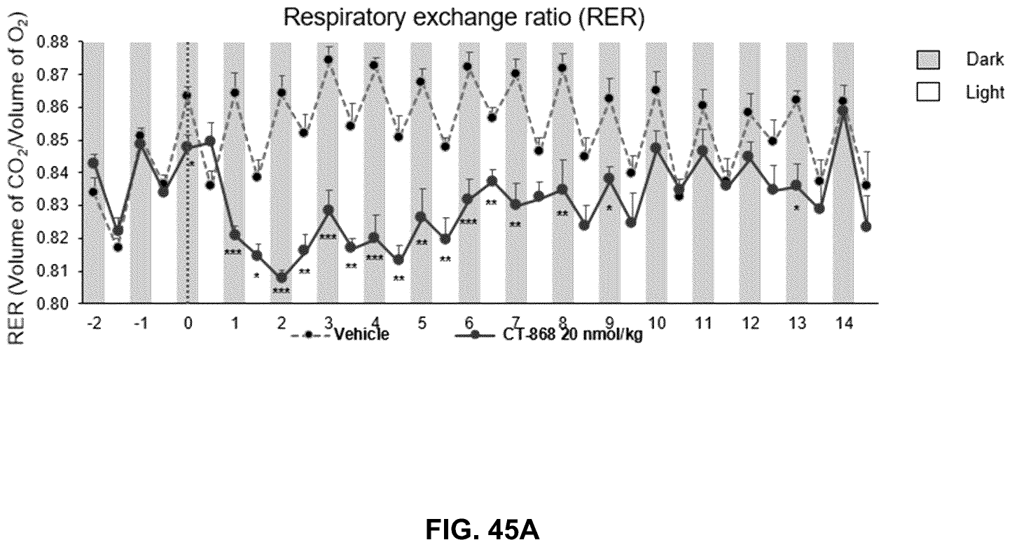

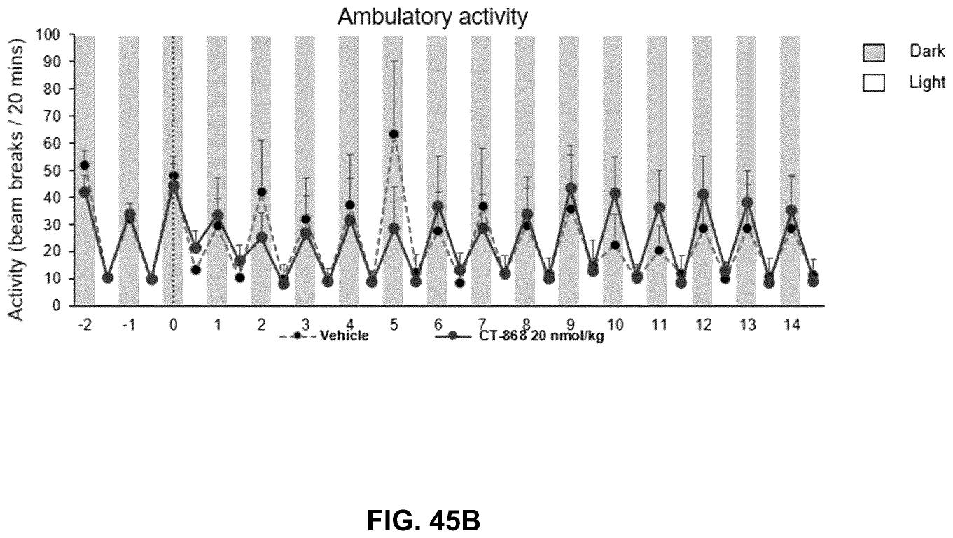

Notably, the RER of CT-868 treated animals was significantly lower than that of the vehicle-treated group in the first nine days of the test phase (right side of red dividing line in FIG. 45A). This reduction in RER suggests a shift toward increased utilization of fat as an energy source following CT-868 administration. This is unlikely to be due to increased physical activity, as there were no differences between the two groups in ambulatory activity after CT-868 administration (right side of red dividing line in FIG. 45B).

Findings from these in vitro and animal studies suggested that further exploration of CT-868 was warranted in clinical studies.

### Phase 1 Single and Multiple Ascending Dose Clinical Study of CT-868 Safety, Tolerability, and Pharmacokinetics

A double-blind, randomized, placebo-controlled Phase 1 clinical study was performed to evaluate the safety, tolerability, and PK of CT-868 when administered as single or multiple doses. In the single ascending dose (SAD) phase, 56 participants with overweight/obesity (BMI 27-45 kg/m²) were sequentially enrolled into Cohorts S1-7 and 8 lean participants (BMI 20-25 kg/m²) were enrolled into Cohort S8 (FIG. 46A). Of eight participants in each cohort, two received placebo and six received CT868. In the multiple ascending dose (MAD) phase, 23 participants with overweight/obesity were enrolled into Cohorts M1-M3 and were randomized (1:3) to receive placebo or CT-868 (FIG. 46B). All 64 participants completed the SAD study, and 22 participants completed the MAD study.

Pharmacokinetic profile of CT-868

Following a single dose of CT-868, the mean maximum plasma concentration (Cₘₐₓ) increased with increasing dose level (FIG. 46C) and the median time of maximum concentration (Tₘₐₓ) ranged from 3 h (Cohort S2: 0.5 mg CT-868) to 20 h (S6: 7.5 mg CT-868) but was predominantly between 3 h-12 h. Median t_{1/2} decreased with increasing CT-868 dose level and ranged from 77.3 h (S3: 1.5 mg CT-868) to 19.7 h (S9: 11 mg CT-868).

At the 1.5 mg dose of CT-868 (S3 and S7 [lean participants]), Cₘₐₓ was reached earlier in lean participants, with a median Tₘₐₓ of 6.0 h for lean participants and 8.0 h for participants with overweight/obesity. Geometric mean (% CV) Cₘₐₓ was higher in lean participants (7.79 [42.4%] ng/ml) than in participants with overweight/obesity (5.62 [28.1%] ng/ml). Similarly, CT-868 AUC trended higher in lean participants (AUC0-t: 167.2 [67.5%] h*ng/mL [lean] vs 154.2 [45.7%] h*ng/ml; AUC_{0-inf}: 209.2 [50.5%] h*ng/mL [lean] vs 184.9 [47.6%] h*ng/ml). Other PK parameters (t_{1/2}, CL/F, or Vz/F) did not differ markedly between lean participants and participants with overweight/obesity.

With repeated daily dosing (MAD phase), plasma CT-868 concentrations were detectable from 2 h post-dose (Day 1) until 240 h post-dose (Day 11) for all doses (0.75-5.0 mg), and reached a plateau after a few days of dosing (FIG. 46D). The plasma concentrations reached after 14 days of dosing were dose-related but not dose-proportional. The pharmacokinetic profile supports once daily dosing in humans.

CT-868 Demonstrated Exposure-Dependent Effects on Glycemic Parameters and Body Weight Reduction in Participants with Overweight/Obesity.

Meal tolerance tests (MTTs) in the SAD cohorts showed a trend toward lower mean AUC values for glucose, insulin, and C-peptide as a function of drug dose. The AUC values for insulin and C-peptide reached significance when they were instead analyzed as a function of CT-868 exposure (FIG. 46E).

Weight loss occurred in all MAD cohorts and was maintained until the end of dosing in all cohorts. The mean change in weight from baseline at Day 13 was −2.62% in the 1.5 mg cohort and −3.37% in the 5.0 mg cohort (Table 18). CT-868 concentration (AUC_{0 τ}) and weight reduction at Day 14 were significantly correlated (FIG. 46F).

TABLE 18
Mean Change in Weight

| | Baseline BW (kg) | D13 BW Change (%) | D21 BW Change (%) |
|---|---|---|---|
| Placebo | 108.03 | −1.90 (−1.76%) | −0.27 (−0.25%) |
| 0.75 mg | 101.06 | −0.84 (−0.83%) | 0.50 (0.50%) |
| 1.5 mg | 99.33 | −2.62 (−2.62%) | −1.90 (−1.91%) |
| 5.0 mg | 99.82 | −3.37 (−3.38%) | −0.92(−0.92%) |

CT-868 was Safe and Well Tolerated at Single Doses of 0.1 mg to 7.5 mg or Multiple Doses of 0.75 mg to 1.5 mg without Up-Titration.

CT-868 was generally safe and well tolerated in lean participants and participants with overweight/obesity at single doses of up to 7.5 mg, and up to 1.5 mg/day for repeated dosing up to 14 days, without undertaking any up-titrations. There were no serious adverse events (SAEs) or drug-induced hypoglycemia events observed during either phase of the study. In the SAD phase, the most common treatment-emergent adverse events (TEAEs) were gastrointestinal symptoms, which occurred with the highest frequency in the highest dose cohort (11.0 mg); twelve events were reported in six (100%) participants, and all were considered drug-related. At 7.5 mg, considered the maximum tolerated single dose, three participants (50%) reported three drug-related TEAEs (two events of nausea and one of diarrhea).

In the MAD phase, all 23 (100%) participants reported ≥1 TEAE and ≥1 drug-related TEAE. Of the 84 TEAEs reported, 58 (69%) were considered related to the study drug. One participant in the highest dose cohort (5.0 mg) reported two drug-related TEAEs (mild nausea and vomiting) that led to study drug withdrawal and subsequently to discontinuation from the study; at this dose level, mild to moderate gastrointestinal symptoms were reported by all six participants. In the 1.5 mg dose cohort, 4/6 participants reported gastrointestinal symptoms, including constipation (4/6), nausea (2/6), vomiting (2/6), abdominal pain (1/6), and diarrhea (1/6). In the lowest dose cohort (0.75 mg), 2/6 participants reported only nausea.

Phase 1 Placebo- and Comparator-Controlled Crossover Clinical Study of CT-868 in Participants with Overweight/Obesity with and without T2DM (CT-868-003 Study)

A second Phase 1 study was conducted to evaluate the pharmacokinetic and

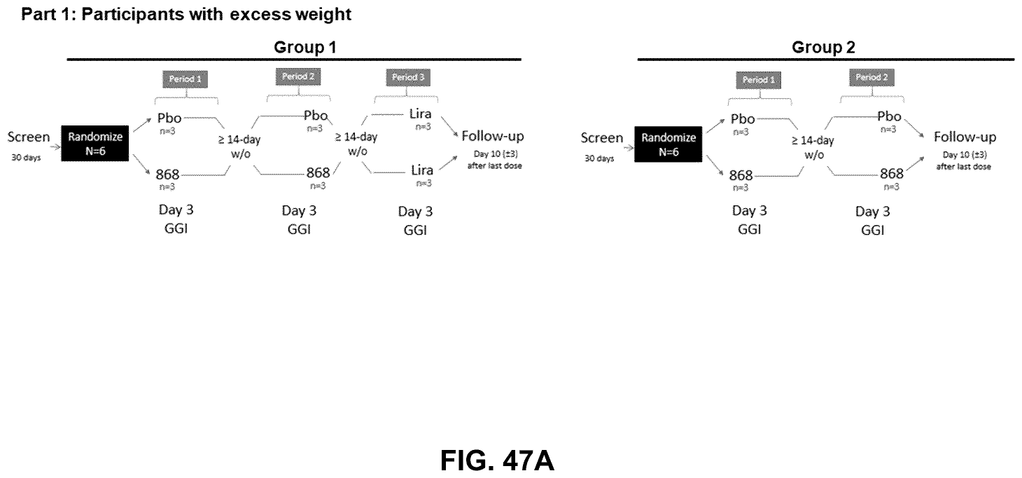

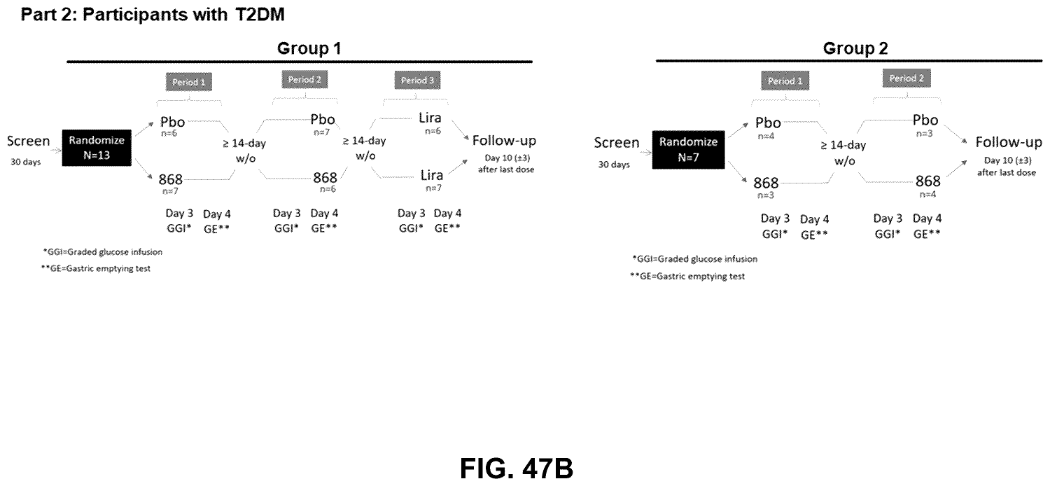

pharmacodynamic effects of CT-868 on the relationship of insulin secretion rate and ambient glucose levels in twelve male participants with obesity but without T2DM (“participants with obesity”; Part 1) and 20 participants with overweight/obesity and T2DM (“participants with T2DM”; Part 2), compared with placebo and with an active comparator, liraglutide. This was a single-center controlled crossover study (FIGS. 47A-B). The demographics and baseline characteristics of the participants in the CT-868-003 study are shown in Table 19.

TABLE 19
Demographics and Baseline Characteristics of Participants in Placebo- and
Comparator-Controlled Crossover Study in Participants with Excess Weight and
Participants with T2DM (CT-868-003 study)

| | Participants with excess weight | | | Participants with T2DM | | |
|---|---|---|---|---|---|---|
| | Group 1 | Group 2 | | Group 1 | Group 2 | |
| | (CT-868/ | (CT-868/ | | (CT-868/ | (CT-868/ | |
| | Placebo/ | Placebo) | | Placebo/ | Placebo) | |
| | Liraglutide) | Sub- | | Liraglutide) | Sub- | |
| | Sub-Total | Total | Total | Sub-Total | Total | Total |
| | (N = 6) | (N = 6) | (N = 12) | (N = 13) | (N = 7) | (N = 20) |
| Age (years), | 40.7 (7.2) | 49.0 (8.3) | 44.8 (8.6) | 52.2 (8.5) | 52.3 (6.0) | 52.2 (7.5) |
| mean (SD) | | | | | | |
| Gender | | | | | | |
| Male | 6 (100%) | 6 (100%) | 12 (100%) | 8 (61.5%) | 3 (42.9%) | 11 (55.0%) |
| Female | 0 | 0 | 0 | 5 (38.5%) | 4 (57.1%) | 9 (45.0%) |
| Ethnicity | | | | | | |
| Hispanic or | 1 (16.7%) | 3 (50.0%) | 4 (33.3%) | 9 (69.2%) | 7 (100%) | 16 (80.0%) |
| Latino | | | | | | |
| Not Hispanic | 5 (83.3%) | 3 (50.0%) | 8 (66.7%) | 4 (30.8%) | 0 | 4 (20.0%) |
| or Latino | | | | | | |
| Race | | | | | | |
| American | 0 | 0 | 0 | 0 | 0 | 0 |
| Indian or | | | | | | |
| Alaska Native | | | | | | |
| Asian | 1 (16.7%) | 0 | 1 (8.3%) | 0 | 0 | 0 |
| Black or | 4 (66.7%) | 1 (16.7%) | 5 (41.7%) | 2 (15.4%) | 0 | 2 (10.0%) |
| African | | | | | | |
| American | | | | | | |
| Native | 0 | 0 | 0 | 0 | 0 | 0 |
| Hawaiian or | | | | | | |
| Other Pacific | | | | | | |
| Islander | | | | | | |
| White | 1 (16.7%) | 5 (83.3%) | 6 (50.0%) | 11 (84.6%) | 7 (100%) | 18 (90.0%) |
| Baseline weight | 96.8 (7.0) | 99.6 (6.6) | 98.2 (6.6) | 93.6 (17.9) | 91.9 (15.8) | 93.0 (16.8) |
| (kg), mean (SD) | | | | | | |
| Baseline BMI | 32.1 (2.2) | 32.4 (2.1) | 32.3 (2.1) | 32.7 (3.89) | 33.5 (4.97) | 32.7 (4.21) |
| (kg/m²), mean | | | | | | |
| (SD)^{b} | | | | | | |
| Diagnosed with | | | | | | |
| T2DM | | | | | | |
| Yes | 0 | 0 | 0 | 13 (100%) | 7 (100%) | 20 (100%) |
| No | 6 (100%) | 6 (100%) | 12 (100%) | 0 | 0 | 0 |
| Duration of | NA | NA | NA | 8.2 (6.0) | 4.1 (1.9) | 6.8 (5.3) |
| diabetes (years), | | | | | | |
| mean (SD) | | | | | | |
| Baseline HbA1c, | 5.9 (0.2) | 5.9 (0.2) | 5.9 (0.2) | 6.7 (1.0) | 8.4 (1.7) | 7.3 (1.5) |
| mean (SD) | | | | | | |
| Baseline C- | 1.1 (0.4) | 1.3 (0.2) | 1.2 (0.3) | 1.1 (0.3) | 1.3 (0.3) | 1.1 (0.3) |
| peptide, mean | | | | | | |
| (SD | | | | | | |

Abbreviations: BMI = Body Mass Index; NA = not applicable; T2DM = type 2 diabetes mellitus.
^{a}Percentages are based on the number of non-missing values in each column.
^{b}Baseline was considered the last value prior to any study treatment.

For Parts 1 and 2, participants in Group 1 received crossover treatment with CT-868, placebo, and liraglutide for two days each, with a 14-day washout period between treatments. In Group 2, participants received crossover treatment with CT-868 and placebo for two days each, with a 14-day washout in between (FIGS. 47A-B). In Part 1, two (16.7%) participants withdrew from the study and the remaining 10 (83.3%) participants completed the study. In Part 2, 16 (80.0%) participants completed the study. Of the four (20.0%) participants who discontinued, two participants discontinued due to AEs, one due to protocol violation and one for other reasons.

In total, 12 participants with obesity completed the CT-868 treatment period (Groups 1 and 2), ten completed the placebo period (Groups 1 and 2) and four completed the liraglutide period (Group 1 only). For participants with T2DM, 20 participants completed the CT-868 treatment period, and 18 and 10 completed the placebo and liraglutide treatment periods, respectively. Given the crossover design, differences in demographics and baseline characteristics between groups and treatment sequences were not expected to affect the interpretation of the results. Data for each participant group were combined for analysis purposes based on the treatment they received.

Endpoints evaluated in this study included the acute effects of CT-868 on the relationship between insulin secretion rate (ISR) and ambient glucose levels, PK and glycemic parameters, gastric emptying rates, ad libitum food intake, and hunger, appetite, and satiety parameters in an MMTT. These acute-phase assessments were performed to thoroughly investigate the weight reduction-independent glucometabolic effects of CT-868, i.e., before the onset of any weight reduction effects.

CT-868 Pharmacokinetics in Participants with Obesity and Participants with T2DM

Participants with obesity and participants with T2DM showed similar CT-868 plasma concentration-time profiles following two days of dosing with CT-868. The key PK parameters for participants with obesity and for participants with T2DM, i.e., Cₘₐₓ (247.0 vs 273.8 ng/ml respectively), Tₘₐₓ (732.8 vs 655.8 minutes), AUC (209,126 vs 231,699 ng/ml*min), incremental AUC (135.840 vs 128,267 ng/ml*min) and terminal half-life (t1/2, 1,190 vs 1,190 minutes), appeared similar. CT-868 demonstrated robust insulin secretory response in participants with obesity and participants with T2DM.

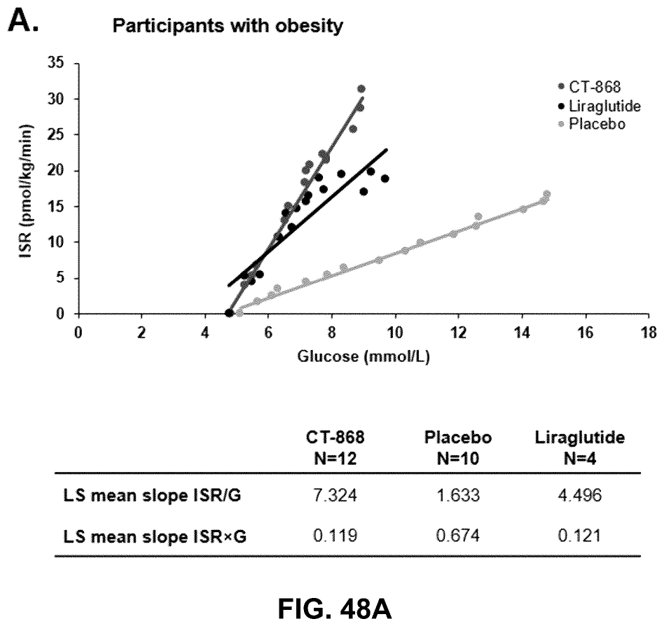

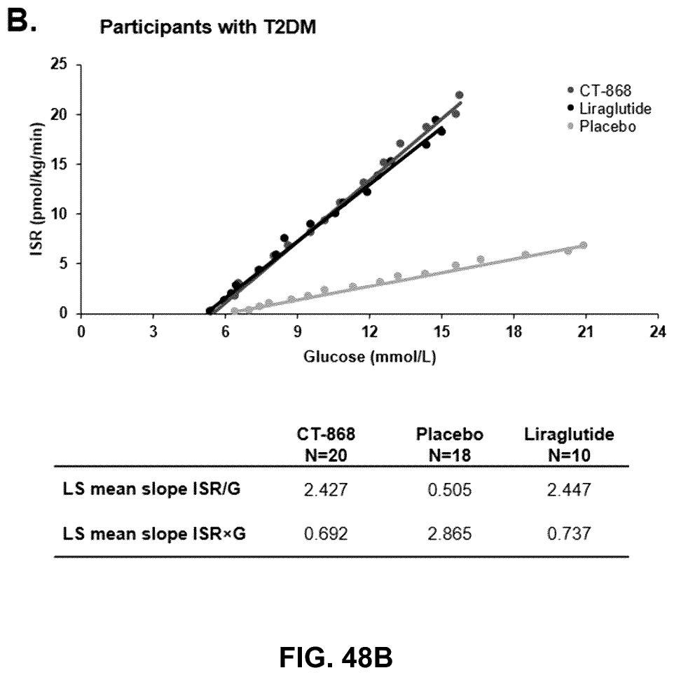

The relationship between insulin secretion rate [ISR] and plasma glucose levels (ISR/G and ISR× G) serves as an indicator of B-cell health and was therefore assessed during a graded glucose infusion (GGI) procedure following two days of dosing with CT-868, liraglutide, or placebo. CT-868 treatment increased B-cell responsiveness to glucose (as indicated by the greater steepness of ISR/G slope) in both participants with obesity and participants with T2DM (FIGS. 48A-B). In participants with obesity, the slope of ISR/G for participants receiving CT-868 was steeper compared with participants receiving placebo (least-squares [LS] mean difference in ISR/G slope: 5.691 [95% CI: 4.227, 7.154]) and those receiving liraglutide (2.750 [95% CI: −0.368, 5.869]) (FIG. 48A). For participants with T2DM, the slope of ISR/G for participants receiving CT-868 was also steeper than for participants receiving placebo (LS mean difference in ISR/G slope: 1.922 [95% CI: 1.232, 2.612]) (FIG. 48B). There was no difference between the ISR/G slopes for CT-868 and liraglutide in participants with T2DM.

CT-868 Lowered Glucose with Less Insulin Excursion than Liraglutide.

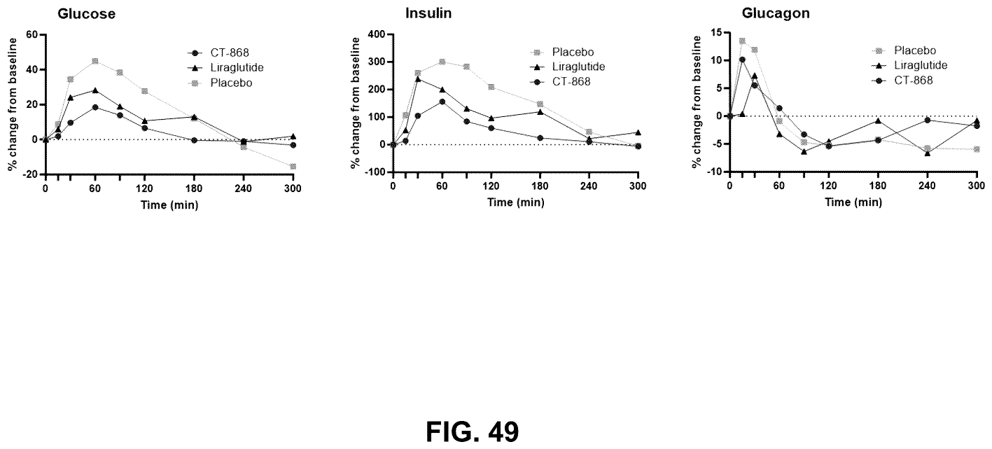

The effects of CT-868 on post-prandial glucose, insulin, and glucagon in participants with T2DM were evaluated in MMTTs (FIG. 49, Table X). Compared with placebo, CT-868 and liraglutide both showed robust glucose-lowering effects. CT-868 was associated with smaller insulin excursions than liraglutide and placebo. Glucagon response was similar between CT-868 and placebo, whereas liraglutide delayed glucagon secretion with lower levels at 15 and 60 minutes post-MMTT start.

During the MMTT, the LS mean insulin iAUC_{0-240 min} was significantly lower with CT-868 versus placebo or liraglutide (FIG. 49, Table 20). For glucose, the LS mean iAUC_{0-240 min} was significantly lower for CT-868 versus placebo (LS mean difference: −320.40 [95% CI: −455.41, −185.39]), but the difference between CT-868 and liraglutide was not significant (LS mean difference: −110.60 [95% CI: −241.54, 20.34]). For glucagon, there was no significant difference in LS mean iAUC_{0-240 min} between CT-868 and liraglutide (LS mean difference: 351.349 [95% CI: −673.85, 1376.55]), or between CT-868 and placebo (LS mean difference: −799.29 [95% CI: −2482.44, 883.85]). For CT-868, the product of insulin and glucose (I×G) was ten times lower than for liraglutide and 27 times lower than for placebo, and for liraglutide, the I×G was 2.6 times lower than for placebo (data not shown).

Table 20 shows mean incremental AUC_{0-240 min} LS mean differences between CT-868 and placebo or liraglutide. Least squares (LS) means were derived using two mixed-effect models. The first model, which compared only CT-868 and placebo, included treatment group, sequence, and period as fixed effects, and participant-within-sequence as a random effect. The second model, which compared only CT-868 and liraglutide, included treatment group, sequence, and group-by-sequence interaction as fixed effects, and participant-within-sequence as a random effect. Denominator degrees of freedom used the Kenward-Roger approximation, and the random effect used a variance component covariance structure. Abbreviations: iAUC=incremental area under the curve; LS=least squares; T2DM=type 2 diabetes mellitus.

TABLE 20
Mean Incremental AUC₀₋₂₄₀ₘᵢₙ LS Mean Differences Between CT-868 and
Placebo or Liraglutide

| | | | |
|---|---|---|---|
| | CT-868 | Placebo | Liraglutide |
| Parameter | N = 19 | N = 18 | N = 9 |

Insulin iAUC₀₋₂₄₀ₘᵢₙ (mU/L*min)

| | | | |
|---|---|---|---|
| Mean (SD) | 1187.48 (3873.08) | 5786.18 (3264.23) | 4477.417 (4777.53) |
| LS mean (SE) | 1178.46 (845.99) | 5832.56 (873.37) | 4613.358 (1252.65) |
| LS mean difference vs. | — | −4654.11 [−7127.92, | −2701.72 [−5311.22, |
| CT-868 [95% CI] | | −2180.30] | −92.23] |

Glucose iAUC₀₋₂₄₀ₘᵢₙ (mmol/L*min)

| | | | |
|---|---|---|---|
| Mean (SD) | 72.29 (214.92) | 404.28 (208.50) | 187.06 (147.68) |
| LS mean (SE) | 71.60 (46.24) | 391.997 (47.90) | 187.05 (55.13) |
| LS mean difference vs. | — | −320.40 [−455.41, | −110.60 [−241.54, 20.34] |
| CT-868 [95% CI] | | −185.39] | |

Glucagon iAUC₀₋₂₄₀ₘᵢₙ (pmol/L*min)

| | | | |
|---|---|---|---|
| Mean (SD) | −200.28 (1196.66) | 512.46 (3349.17) | −344.79 (979.94) |
| LS mean (SE) | −208.42 (575.60) | 590.88 (594.23) | −408.63 (413.72) |
| LS mean difference vs. | — | −799.29 [−2482.44, | 351.349 [−673.85, |
| CT-868 [95% CI] | | 883.85] | 1376.55] |

CT-868 and Liraglutide Demonstrated Similar Gastric Emptying Delay Versus Placebo in Participants with T2DM.

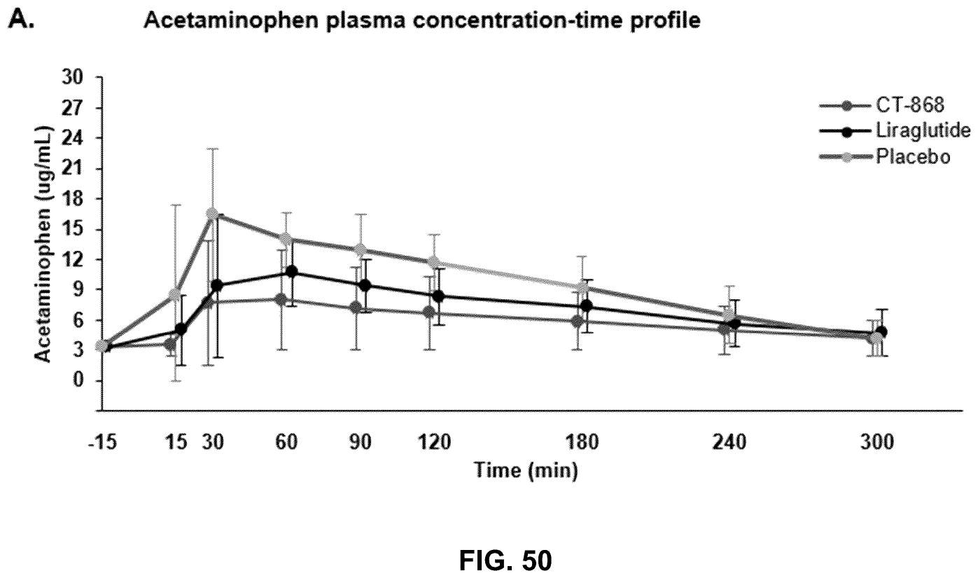

The effect of CT-868 on gastric emptying rates was assessed via an acetaminophen absorption test. Dosing with CT-868 and liraglutide resulted in similar gastric emptying delay versus placebo, based on acetaminophen plasma concentration-time profiles (FIG. 50, Table 21).

TABLE 21
Acetaminophen Plasma Concentration-Time Profiles

| | | | |
|---|---|---|---|
| | CT-868 | Placebo | Liraglutide |
| Parameter | (N = 20) | (N = 18) | (N = 10) |

Acetaminophen AUC₀₋₃₀₀ₘᵢₙ (mg/L*min)

| | | | |
|---|---|---|---|
| Mean (SD) | 1785.2 (838.63) | 2915.2 (747.55) | 2217.0 (655.84) |
| % CV | 46.98 | 25.64 | 29.58 |

Acetaminophen AUC₀₋₆₀ₘᵢₙ (mg/L*min)

| | | | |
|---|---|---|---|
| Mean (SD) | 371.4 (209.11) | 728.1 (267.16) | 472.8 (239.02) |
| % CV | 56.30 | 36.69 | 50.55 |

Acetaminophen Cₘₐₓ (mg/L), 0-300 min

| | | | |
|---|---|---|---|
| Mean (SD) | 9.2 (6.31) | 18.2 (6.79) | 12.9 (4.79) |
| % CV | 68.42 | 37.36 | 37.17 |

Acetaminophen Tₘₐₓ (mg/L), 0-300 min

| | | | |
|---|---|---|---|
| Mean (SD) | 42.2 (29.52) | 45.9 (28.28) | 60.0 (30.00) |
| % CV | 69.92 | 61.55 | 50.00 |

Time-weighted average acetaminophen AUC₀₋₃₀₀ₘᵢₙ (mg/L*min)

| | | | |
|---|---|---|---|
| Mean (SD) | 6.0 (2.80) | 9.7 (2.50) | 7.4 (2.19) |
| % CV | 46.98 | 25.77 | 29.58 |

CT-868 Reduced Food Intake Versus Placebo in Participants with T2DM.

Food intake and total calorie intake during an ad libitum meal were assessed in participants with T2DM after three days of dosing with CT-868, liraglutide, or placebo. Mean food intake and total calories consumed were similar in participants administered CT-868 or liraglutide (LS mean difference −48.7 g [95% CI: −142.6, 45.3] for food intake, −202.3 kcal [95% CI: −478.1, 73.6] for total calories), and were significantly different between CT-868 and placebo (Table 22). There were no significant differences between CT-868, liraglutide, and placebo in body weight change after three days of dosing (Day 4; Table 22). Compared with Day 1 (prior to the first CT-868 dose), pre-breakfast hunger and appetite scores were lower at Day 4 (after 3 days of dosing) in the CT-868 and liraglutide groups, and higher in the placebo group (Table 22).

TABLE 22
Effects of CT-868 on Hunger and Appetite Scores Before and After Three
Days of Dosing, and on Food Intake and Total Calorie Intake During
an Ad Libitum Meal in Participants with T2DM (CT-868-003 study)

| | | | |
|---|---|---|---|
| | CT-868 | Placebo | Liraglutide |
| Parameters | (N = 20) | (N = 18) | (N = 10) |

Appetite score (VAS: 0-100 points)^{a}

| | | | |
|---|---|---|---|
| Day 1^{b}, LS mean | 69.2 [59.5, | 48.6 [37.6, | 60.1 [38.9, |
| [95% CI] | 78.8] | 59.6] | 81.3] |
| Day 4^{b}, LS mean | 45.1 [33.9, | 60.6 [51.6, | 50.4 [29.0, |
| [95% CI] | 56.3] | 69.5] | 71.9] |
| LS mean change from | −24.1 [−33.6,− | 11.9 [−0.8, | −9.7 [−20.5, |
| Day 1 to Day 4 | 14.5] | 24.7] | 1.2] |
| [95% CI] | | | |

Hunger score (VAS: 0-100 points)^{a}

| | | | |
|---|---|---|---|
| Day 1^{b}, LS mean | 60.3 [50.1, | 37.9 | 59.7 [37.0, |
| [95% CI] | 70.51 | | 82.3] |
| Day 4^{b}, LS mean | 46.8 [34.6, | 42.9 | 50.7 [29.0, |
| [95% CI] | 59.0] | | 72.3] |
| LS mean change from | −13.5 [−24.6,− | 5.0 | −9.0 [−20.4, |
| Day 1 to Day 4 | 2.4] | | 2.4] |
| [95% CI] | | | |

Body weight, kg

| | | | |
|---|---|---|---|
| Day 1, LS mean | 93.3 [85.4, | 94.3 [85.9, | 94.6 [81.8, |
| [95% CI] | 101.2] | 102.7] | 107.4] |
| Day 4, LS mean | 93.4 [85.6, | 94.3 [86.0, | 94.2 [81.61, |
| [95% CI] | 101.2] | 102.6] | 106.8] |
| LS mean change from | 0.09 [−0.47, | 0 [−0.38, 0.38] | −0.39 [−0.92, |
| Day 1 to Day 4 | 0.65] | | 0.14] |
| [95% CI] | | | |

Food intake during ad libitum meal on Day 4, g

| | | | |
|---|---|---|---|
| Mean (SD) | 591.4 (170.1) | 745.7 (199.6) | 680.2 (220.7) |
| LS mean difference vs. | NA | −147.9 [−222.1, | −48.7 [−142.6, |
| CT-868 [95% CI]^{c} | | −73.8] | 45.3] |

Caloric intake during ad libitum meal on Day 4, kcal

| | | | |
|---|---|---|---|
| Mean (SD) | 733.7 (410.8) | 1078.1 (389.0) | 959.3 (504.4) |
| LS mean difference vs. | NA | −328.6 [−536.1, | −202.3 [−478.1, |
| CT-868 [95% CI]^{c} | | −121.1] | 73.6] |

Abbreviations: LS = least-squares; NA = not applicable; T2DM = type 2 diabetes mellitus.
ªAppetite and hunger VAS scores were recorded 15 minutes prior to administration of CT-868 on Day 1 and Day 4.
^{b}CT-868 was administered daily, with the first dose administered on Day 1.
^{c}Least-squares means were derived using two mixed-effect models. The first model, which compared only CT-868 and placebo, included treatment group, sequence, and period as fixed effects, and participant-within-sequence as a random effect. The second model, which compared only CT-868 and liraglutide, included treatment group, sequence, and group-by-sequence interaction as fixed effects, and participant-within-sequence as a random effect. Denominator degrees of freedom used the Kenward-Roger approximation, and the random effect used a variance component covariance structure.

CT-868 was safe and well tolerated in participants with obesity and participants with T2DM.

In the crossover study, CT-868 overall showed a favorable tolerability and safety profile (Table 23).

TABLE 23
Treatment-Emergent Adverse Events in Placebo- and Comparator-Controlled
Crossover Study (CT-868-003 study)

| | Part 1 (participants with obesity) | | | Part 2 (participants with T2DM) | | |
|---|---|---|---|---|---|---|
| Category, n (%) | CT-868 | Placebo | Liraglutide | CT-868 | Placebo | Liraglutide |
| Severity, n (%) | (N = 12) | (N = 11) | (N = 4) | (N = 20) | (N = 18) | (N = 10) |
| Any TEAEs | 8 (66.7%) | 4 (36.4%) | 3 (75.0%) | 14 (70.0%) | 9 (50.0%) | 7 (70.0%) |
| Grade 1 | 5 (41.7%) | 4 (36.4%) | 2 (50.0%) | 14 (70.0%) | 8 (44.4%) | 7 (70.0%) |
| Grade 2 | 3 (25.0%) | 0 | 1 (25.0%) | 0 | 1 (5.6%) | 0 |
| Drug-related | 4 (33.3%) | 0 | 0 | 9 (45.0%) | 7 (38.9%) | 3 (30.0%) |
| TEAEs | | | | | | |
| SAEs | 0 | 0 | 0 | 0 | 0 | 0 |
| TEAEs leading | 0 | 0 | 0 | 1 (5.0%) | 1 (5.6%) | 0 |
| to drug | | | | | | |
| discontinuation | | | | | | |
| Injection site | 0 | 0 | 0 | 1 (5.0%) | 0 | 0 |
| reactions | | | | | | |
| Any AESIs | 6 (50.0%) | 1 (9.1%) | 2 (50.0%) | 10 (50.0%) | 2 (11.1%) | 4 (40.0%) |
| Grade 1 | 5 (41.7%) | 1 (9.1%) | 1 (25.0%) | 10 (50.0%) | 2 (11.1%) | 4 (40.0%) |
| Grade 2 | 1 (8.3%) | 0 | 1 (25.0%) | | | |

Abbreviations: AE = adverse events; AESI = AEs of special interest; SAE = serious adverse events; TEAE = treatment-emergent adverse events; T2DM = type 2 diabetes mellitus.
Percentages are based on the number of participants in the Safety Set in each treatment group.
Drug-related AEs were those with possible, probable, definitely, or missing relationship to the study drug.
For hypoglycemia AEs, severity was graded as follows: Grade 1 = Glucose <70 mg/dL and glucose ≥54 mg/dL; Grade 2 = Glucose <54 mg/dL; Grade 3 = A severe event characterized by altered mental and/or physical status requiring assistance. AEs with unknown severity were included in Grade 3.

In Part 2 (participants with T2DM), 14/20 (70.0%) participants administered CT-868 reported >1 TEAE, and all TEAEs were considered mild (Grade 1), comparable with participants administered liraglutide. One participant reported TEAEs leading to discontinuation of treatment. TEAEs related to CT-868 were reported by 9/20 participants (45.0%). Most of these were gastrointestinal symptoms (nausea, gastroesophageal reflux 5 disease and vomiting). TEAEs were reported for 7/18 participants (38.9%) on placebo and 3/10 participants (30.0%) on liraglutide (Table 24).

TABLE 24
Summary of TEAEs by System Organ Class and Preferred Term
(CT-868-003 study)

| System Organ | Part 1 (participants with obesity) | | | Part 2 (participants with T2DM) | | |
|---|---|---|---|---|---|---|
| Class, n (%) | CT-868 | Placebo | Liraglutide | CT-868 | Placebo | Liraglutide |
| Preferred Term, n (%) | (N = 12) | (N = 11) | (N = 4) | (N = 20) | (N = 18) | (N = 10) |
| At least one TEAE, | 4 (33.3%) | 0 | 0 | 14 (70.0%) | 9 (50.0%) | 7 (70.0%) |
| n (%) | | | | | | |
| Blood and | 0 | 0 | 0 | 1 (5.0%) | 0 | 0 |
| lymphatic system | | | | | | |
| disorders | | | | | | |
| Anaemia | 0 | 0 | 0 | 1 (5.0%) | 0 | 0 |
| GI disorders | 1 (8.3%) | 0 | 0 | 10 (50.0%) | 6 (33.3%) | 3 (30.0%) |
| Abdominal | 0 | 0 | 0 | 1 (5.0%) | 0 | 1 (10.0%) |
| distension | | | | | | |
| Abdominal pain | 0 | 0 | 0 | 1 (5.0%) | 1 (5.6%) | 0 |
| Constipation | 0 | 0 | 0 | 1 (5.0%) | 0 | 0 |
| Diarrhoea | 0 | 0 | 0 | 1 (5.0%) | 1 (5.6%) | 0 |
| Dyspepsia | 1 (8.3%) | 0 | 0 | 1 (5.0%) | 3 (16.7%) | 1 (10.0%) |
| Eructation | 0 | 0 | 0 | 1 (5.0%) | 0 | 0 |
| Gastroesophageal | 0 | 0 | 0 | 3 (15.0%) | 0 | 0 |
| reflux disease | | | | | | |
| Nausea | 0 | 0 | 0 | 6 (30.0%) | 1 (5.6%) | 2 (20.0%) |
| Vomiting | 0 | 0 | 0 | 2 (10.0%) | 0 | 0 |
| Metabolism and | 1 (8.3%) | 0 | 0 | 7 (35.0%) | 3 (16.7%) | 3 (30.0%) |
| nutrition disorders | | | | | | |
| Decreased appetite | 1 (8.3%) | 0 | 0 | 0 | 1 (5.6%) | 0 |
| Hypoglycaemia | 0 | 0 | 0 | 7 (35.0%) | 2 (11.1%) | 3 (30.0%) |
| Nervous system | 2 (16.7%) | 0 | 0 | 4 (20.0%) | 2 (11.1%) | 0 |
| disorders | | | | | | |
| Dizziness | 0 | 0 | 0 | 1 (5.0%) | 0 | 0 |
| Headache | 2 (16.7%) | 0 | 0 | 3 (15.0%) | 2 (11.1%) | 0 |
| General disorders | 0 | 0 | 0 | 3 (15.0%) | 0 | 0 |
| and administration | | | | | | |
| site conditions | | | | | | |
| Chills | 0 | 0 | 0 | 1 (5.0%) | 0 | 0 |
| Injection site | 0 | 0 | 0 | 1 (5.0%) | 0 | 0 |
| haemorrhage | | | | | | |
| Peripheral | 0 | 0 | 0 | 1 (5.0%) | 0 | 0 |
| swelling | | | | | | |
| Skin and | 0 | 0 | 0 | 2 (10.0%) | 0 | 1 (10.0%) |
| subcutaneous tissue | | | | | | |
| disorders | | | | | | |
| Dermatitis | 0 | 0 | 0 | 0 | 0 | 1 (10.0%) |
| Rash | 0 | 0 | 0 | 1 (5.0%) | 0 | 0 |
| Skin | 0 | 0 | 0 | 1 (5.0%) | 0 | 0 |
| hyperpigmentation | | | | | | |
| Infections and | 0 | 0 | 0 | 0 | 1 (5.6%) | 0 |
| infestations | | | | | | |
| Urinary tract | 0 | 0 | 0 | 0 | 1 (5.6%) | 0 |
| infection | | | | | | |
| Investigations | 0 | 0 | 0 | 0 | 1 (5.6%) | 0 |
| Coagulation test | 0 | 0 | 0 | 0 | 1 (5.6%) | 0 |
| abnormal | | | | | | |
| Musculoskeletal and | 0 | 0 | 0 | 0 | 2 (11.1%) | 0 |
| connective tissue | | | | | | |
| disorders | | | | | | |
| Arthralgia | 0 | 0 | 0 | 0 | 1 (5.6%) | 0 |
| Back pain | 0 | 0 | 0 | 0 | 1 (5.6%) | 0 |
| Renal and urinary | 1 (8.3%) | 0 | 0 | 0 | 1 (5.6%) | 1 (10.0%) |
| disorders | | | | | | |
| Pollakiuria | 1 (8.3%) | 0 | 0 | 0 | 1 (5.6%) | 1 (10.0%) |

Abbreviations: TEAE = treatment-emergent adverse events; T2DM = type 2 diabetes mellitus.
^{a}All investigator-recorded AE terms were coded using MedDRA dictionary version 24.0.
Note:
Percentages are based on the number of participants in the Safety Set in each column. Participants were counted once within each system organ class and each preferred term within each column.

Occurrence of hypoglycemia during the GGI was similar in the CT-868 and liraglutide groups. No participants reported any SAEs. There were no clinically significant CT-868-related changes in vital signs, ECG parameters, or laboratory values reported.

### Discussion

The effects of CT-868, a signaling-biased dual receptor agonist engineered for enhanced signaling efficacy at both GLP-1R and GIPR, on insulin sensitivity, glucose homeostasis, and body weight were characterized in in vitro and animal models and two Phase 1 clinical studies.

In vitro studies showed that CT-868 functions as a dual GLP-1/GIP receptor agonist and exhibits full signaling bias at both receptors, prioritizing cAMP accumulation without β-arrestin recruitment or receptor internalization (FIGS. 42A-H). The physiological relevance of these properties was assessed in animal studies, wherein CT-868 at minimal-GIPR-engaging doses reduced blood glucose excursion in response to a meal challenge more effectively than a signal unbiased GLP-1R mono-agonist, liraglutide (FIGS. 43A-I). Moreover, these improvements in blood glucose control were not the consequence of increased insulin secretion with CT-868, but rather mediated through GIP signaling, as demonstrated in experiments with GLP-1R^{-/-} mice.

CT-868 not only avoids receptor internalization but may also act as an inverse agonist, maintaining receptors at the cell surface. Studies of cAMP biased GLP-IR agonists have demonstrated enhanced insulinotropic and weight loss effects relative to unbiased GLP-IR agonists. Studies of CAMP biased GLP-IR agonists have demonstrated enhanced insulinotropic and weight loss effects relative to unbiased GLP-IR agonists. Animal studies in this application support these observations, wherein CT-868, at dose levels that showed limited activation of GIPR, reduced postprandial blood glucose levels and induced weight loss more effectively than a signal-unbiased GLP-1R mono-agonist, liraglutide (FIGS. 44A-D). Unlike liraglutide, the initial weight reduction with CT-868 was maintained beyond the dosing period, and attributable to a sustained reduction in food intake, without a decrease in the animals' activity levels. Additionally, there were metabolic changes (VO₂, VCO₂, RER) suggestive of increased utilization of fat as an energy source. The extent to which these effects can be attributed primarily to biased cAMP signaling, rather than contributions from GIPR activation, warrants further investigation. Nonetheless, it is evident that at higher doses (e.g., 300 nmol/kg), there is additional weight loss, presumably resulting from GIPR activation.

In the first-in-human Phase 1 study, CT-868 was tested at single doses of up to 11.0 mg and multiple doses up to 5.0 mg/day for 14 days without stepwise up-titration in participants with normal weight and overweight/obesity and was found to be safe and generally well tolerated. Plasma CT-868 exposure was dose-dependent, supporting once-daily administration. In participants receiving multiple doses of CT-868, the PK profiles showed sustained systemic exposure to CT-868 throughout the 14-day dosing period, with maximum plasma concentrations reached within 48-96 h after the first dose. In the crossover study, PK analyses in participants with obesity and participants with T2DM showed that maximum plasma concentrations were attained at 11-12 h post-dose and the terminal half-life was approximately 20 h. This further supported a once-daily dosing regimen for CT-868 and confirmed that key pharmacodynamic assessments (GGI, MMTT, gastric emptying tests) were performed at times when exposure to CT-868 was maximal. The highest multiple dose (5.0 mg/day) was not associated with any SAEs, drug-induced hypoglycemia or severe TEAEs, but was considered non-tolerated in one participant due to TEAEs leading to drug withdrawal and study discontinuation in that individual. In both clinical studies, most TEAEs were mild and GI-related (nausea, vomiting), largely as expected for drugs that target GLP-1R. No severe or serious TEAEs or drug-related hypoglycemia was reported with CT-868 in this ascending dose study.

The pancreatic and extra-pancreatic effects of CT-868 in the context of T2DM and

obesity were investigated in a placebo- and comparator-controlled crossover study. In participants with T2DM, CT-868 robustly enhanced glucose-stimulated insulin response, similar to liraglutide. This effect was more pronounced in participants with obesity (without T2DM), in part due to preserved β-cell function, as indicated by higher baseline C-peptide levels in this group compared to those with T2DM. Among participants with obesity, CT-868 appeared to stimulate insulin secretion more strongly than liraglutide, which could be due to the additional GIPR agonism of CT-868 or to GLP-IR desensitization with liraglutide. Although CT-868 and liraglutide showed similarly robust glucose-lowering effects following a mixed meal, CT-868 was associated with less insulin excursion (iAUC_{0-240 min}) and did not suppress glucagon. These effects were not driven by significant differences in gastric emptying or weight loss, which were largely similar over the CT-868 and liraglutide treatment periods. Rather, CT-868 appears to promote enhanced insulin sensitivity and/or insulin-independent glucose disposal (relative to liraglutide), likely through modulation of GIPR signaling (Hammoud et al., Nat Rev Endocrinol (2023) 19:201-16). GIPR may also influence energy metabolism through other insulin-independent mechanisms, such as regulating lipid storage via its activity in adipose tissue (Samms, supra). In type 1 diabetes or advanced type 2 diabetes, wherein β-cells fail to produce the necessary insulin, the ability to target insulin-independent mechanisms for glucose control, such as GIP stimulation of peripheral glucose uptake, may assume greater therapeutic importance.

Across these studies, CT-868 at minimal-GIPR-engaging doses generally showed larger effects than the unbiased mono-agonist liraglutide in terms of both glycemic and body weight reduction, underscoring the physiological relevance of CT868's signaling bias. Additionally, compared with mono-agonists, the added GIPR agonism of CT-868 may contribute to enhanced glucose lowering, as reported for two other dual GLP-1R/GIPR agonists (Finan et al., Sci Transl Med (2013) 5: 209ra151). The results suggest that some of the observed GIPR-mediated effects of CT-868 may be insulin-independent. It has also been proposed that simultaneous targeting of GIPR may help mitigate the pro-emetic effects of GLP-1R activation (Borner et al., Diabetes (2021) 70:2545-53), thus improving treatment tolerability. Results from a recently completed Phase 2 study (ClinicalTrials ID: NCT05110846) will provide additional insight into optimizing CT-868 dosing and up-titration to support maximal weight reduction and glycemic improvement with acceptable tolerability.

### Conclusion

These findings shed additional light on the effects of dual GLP-1R/GIPR agonism and how the balance of GLP-1R/GIPR activation and cAMP signaling bias simultaneously at both the GLP-1R and GIPR can favorably influence metabolic homeostasis. In the two Phase 1 clinical studies, the signaling-biased dual GLP-1/GIP receptor agonist CT-868 was observed to be safe and well-tolerated. Independent of body weight changes, CT-868 demonstrated robust glucose-lowering effects but with reduced insulin excursion and preserved glucagon secretion relative to liraglutide. These results support further evaluation of CT-868's longer term effects on glucose, body weight and other parameters in people with overweight/obesity and type 1 or type 2 diabetes mellitus.

### Example 6: A Phase 2, Double-Blind, Randomized, Placebo-Controlled Study to Evaluate the Efficacy, Safety, Tolerability, and Pharmacokinetics of CT-868 Administered for 16 Weeks to Overweight and Obese Adult Participants with Type 1 Diabetes Mellitus

This Example outlines the protocol for a Phase 2 study to evaluate the efficacy, safety, tolerability, and pharmacokinetics of CT-868 in overweight or obese adult participants with a documented diagnosis of T1DM for ≥1 year, treated with insulin via continuous subcutaneous insulin infusion (CSII) or by means of multiple daily injections (MDIs) of insulin for ≥6 months.

### Objectives and Endpoints

The primary objective of this study is to compare the effect of CT-868 titrated up to maximum of 6.6 mg treatment group (Arm 4) versus placebo (Arm 1) on change in HbA1c (%) from baseline (Day 1) to Week 16, and the primary endpoint was mean change in HbA1c (%) from baseline at Day 1 to Week 16 in Arm 4 compared with Arm 1.

Key secondary objectives of this study are:
to compare the effect of CT-868 titrated to 4.1 mg treatment group (Arm 3) versus placebo (Arm 1) on change in HbA1c (%) from baseline (Day 1) to Week 16; and
to compare the effect of CT-868 titrated to 1.8 mg treatment group (Arm 2) versus placebo (Arm 1) on change in HbA1c (%) from baseline (Day 1) to Week 16.

Key secondary endpoints of this study are:
mean change in HbA1c (%) from baseline at Day 1 to Week 16 in Arm 3 compared with Arm 1; and
mean change in HbA1c (%) from baseline at Day 1 to Week 16 in Arm 2 compared with Arm 1.

Other secondary objectives of this study are:
To assess the effect of CT-868 in each treatment arm relative to placebo on the following parameters:
   CGM metrics (TIR, time in hypo- and hyperglycemia, glycemic risk index);
   Percentage of participants achieving HbA1c of <7.0%, ≤6.5%, and <5.7%;
   Change in body weight;
   Percentage of participants achieving ≥5%, ≥10%, ≥15%, and ≥20% weight loss;
   Number of Level 3 hypoglycemic events;
   Incidence of DKA; and/or
   solute and relative changes in basal, bolus, and total daily insulin use (units/day and units/kg/day).
To assess the safety and tolerability of CT-868 administered via an injection pen in each treatment arm versus placebo over 16 weeks in overweight or obese participants with T1DM.
To evaluate CT-868 plasma concentration.

Other secondary endpoints of this study are:
Change from baseline at Day 1 to Weeks 8 and 16 in each treatment arm in the following CGM parameters:
   mean sensor glucose (mg/dL) and its standard deviation and % CV [both intraday (i.e., within 24 hours) and interday (i.e., over multiple days)];
   percentage of CGM values that are in range (TIR 70-180 mg/dL);
   percentage of participants with TIR 70-180 mg/dL with ≥5% and ≥10% points improvement;
   percentage of CGM values <70-54 mg/dL (Level 1 hypoglycemia) and <54 mg/dL (Level 2 hypoglycemia);
   percentage of CGM values >180-250 mg/dL (Level 1 hyperglycemia) and >250 mg/dL (Level 2 hyperglycemia);
   time (hours/minutes) spent in range (70-180 mg/dL) per 24 hours during the CGM wear period;
   time (hours/minutes) spent in hypoglycemia Level 1 (<70-54 mg/dL) and Level 2 (<54 mg/dL) per 24 hours during the CGM wear period;
   time (hours/minutes) spent in hyperglycemia Level 1 (>180-250 mg/dL) and Level 2 (>250 mg/dL) per 24 hours during the CGM wear period;
   number of events with sensor glucose <70 mg/dL lasting at least 120 minutes (event ends when glucose returns to ≥70 mg/dL for ≥15 minutes);
   number of events with sensor glucose >250 mg/dL lasting at least 120 minutes (event ends when glucose returns to ≤180 mg/dL for ≥15 minutes);
   proportion of participants with TIR 70-180 mg/dL for >70% of each day;
   proportion of participants with TBR <70 mg/dL for <4% of each day;
   proportion of participants with TBR <54 mg/dL for <1% of each day; and/or
   glycemia Risk Index.
HbA1c
   change in HbA1c (%) from baseline at Day 1 to Weeks 4, 8, and 12; and/or
   percentage of participants achieving HbA1c of <7.0%, ≤6.5%, and <5.7% at Week 16.
Body weight
   percent (%) change in body weight from baseline at Day 1 to Weeks 4, 8, 12, and 16;
   absolute change in body weight (kg) from baseline at Day 1 to Weeks 4, 8, 12, and 16; and/or
   percentage of participants achieving ≥5%, ≥10%, ≥15%, and >20% weight loss at Week 16.
Number of Level 3 hypoglycemic events over the period of 16 weeks in each treatment arm.
Number of “definite” DKA events (listed below) (Kitabchi et al., Diabetes Care (2009) 32 (7): 1335-43).
Number of DKA events that led to hospitalization or emergency room visits.
Change (absolute and percentage change) in basal, bolus, and total daily insulin use (units/day and units/kg/day) over the period of 16 weeks in each treatment arm.
AEs/ADEs, physical examinations, vital signs (temperature, blood pressure, and heart rate), ECGs, and safety laboratory measurements (urinalysis, hematology, chemistry, BHB, amylase, lipase, calcitonin) over the period of 16 weeks in each treatment arm.
Plasma drug concentrations at the Weeks 2, 4, 6, 8, 12, and 16 visits.

Discontinuation of the study drug may be considered when a study participant meets any of the following criteria:
Any event that is highly suggestive of severe drug-induced liver injury. Examples may include injury with any of the following findings:
   Clinical manifestations (e.g., liver-related hospitalization, diagnosis of acute liver failure).
   Concerning symptoms consistent with the diagnosis of acute drug-induced liver injury (e.g., abdominal pain, vomiting, jaundice).
   Biochemical manifestations such as those described below based on the FDA guidance regarding individual participant stopping criteria for drug-induced liver injury (CDER 2009):
      ALT or AST >8×ULN
      ALT or AST >5×ULN for >2 weeks
      ALT or AST >3×ULN and total bilirubin >2×ULN or international normalized ratio >1.5×ULN
      ALT or AST >3×ULN with the appearance of fatigue, nausea, vomiting, right upper quadrant pain or tenderness, fever, rash, and/or eosinophilia (>5%)
      ALP >3×ULN
      ALP >2.5×ULN and total bilirubin >×ULN or
      ALP >2.5×ULN with the appearance of fatigue, nausea, vomiting, right quadrant pain or tenderness, fever, rash, and/or eosinophilia (>5%).
Unable to tolerate a minimum of 1.8, 3.3, or 4.1 mg/day of CT-868 in Arm 2, Arm 3, or Arm 4, respectively.
Any prescription or over-the-counter medication for weight loss is given for more than 1 week.
Any prohibited anti-hyperglycemic agent(s) for more than 1 week.
Calcitonin ≥100 ng/L.
eGFR <15 mL/min.
Suspicion of pancreatitis.
Experiences any of the following clinically significant hypoglycemic events in the absence of any confounding conditions that lower blood glucose:
   At any time during the study, participants experience Level 3 hypoglycemia, defined as a severe event characterized by impaired mental (cognitive) and/or physical functioning that requires external assistance from another person for recovery; or
   At any time during the study, CGM values are <54 mg/dL (3.0 mmol/L) with or without symptoms for a consecutive ≥120 minutes (extended Level 2 hypoglycemia).
Experiences a “definite” DKA episode [defined as the presence of acidosis (blood pH<7.3, serum bicarbonate levels <18 mEq/L), and accompanied by symptoms/signs per the ADA consensus statement on diagnosis of DKA, or if the DKA episode results in a hospital or emergency room stay.

Exploratory Objectives of this study are:
To assess the effect of CT-868 in each treatment arm relative to placebo on composite response parameters at Week 16.
To assess the effect of CT-868 in each treatment arm relative to placebo during MMTT and on gastric emptying (via acetaminophen absorption).
To assess the effect of CT-868 in each treatment arm relative to placebo on blood biomarkers of metabolism, inflammation, and endothelial health.
To assess the effect of CT-868 in each treatment arm relative to placebo on total lean mass and total fat mass by DEXA.
To assess the effect of CT-868 in each treatment arm relative to placebo on quality of life and treatment satisfaction using PRO questionnaires.
To assess the effect of CT-868 in each treatment arm relative to placebo on VAS ratings of hunger, satiety, fullness, and prospective food consumption.
To assess the percentage of participants at the 4.1 mg, 5.2 mg, and 6.6 mg doses at the end of Week 16 and the time to achieve their final dose (Arm 4 only).
To determine correlation between time to attain the highest CT-868 dose and change in HbA1c and % body weight change from baseline to Week 16 (Arm 4 only).
To assess the percentage of participants who from randomization at Day 1 to Week 16 have permanently discontinued study drug.
To assess the time (weeks) to permanent discontinuation of study drug.

Exploratory endpoints of this study are:
Percentage of participants in each treatment arm at Week 16 compared with corresponding Day 1 baseline with:
   Improvement in HbA1c ≥0.5% points and <1% TBR <54 mg/dL;
   ≥70% TIR 70-180 mg/dL and <4% TBR <70 mg/dL;
   ≥70% TIR 70-180 mg/dL and <1% TBR <54 mg/dL; and/or
   Either HbA1c improvement >0.5% points or ≥70% TIR 70-180 mg/dL, with <1% TBR <54 mg/dL, and without requiring significant increase in daily insulin use [i.e., >10% increase in total (bolus+basal) insulin (units/kg/day)].
Change from baseline at Day 1 to Week 16 in fasting (i.e., immediately before start of MMTT) plasma glucose, insulin, C-peptide, glucagon, GLP-1 (total and active), GIP, and FFA in each treatment arm.
Change from baseline at Day 1 to Week 16 in the following MMTT parameters in each treatment arm:
   Glucose iAUC_{0-240 min};
   Insulin iAUC_{0-240 min};
   C-peptide iAUC_{0-240 min};
   Glucagon iAUC_{0-240 min};
   GLP-1 (total and active) iAUC_{0-240 min};
   GIP iAUC_{0-240 min};
   FFA iAUC_{0-240 min};
   HOMA-IR, QUICKI, Matsuda index, HOMA-B, disposition index; and/or
   Acetaminophen Cₘₐₓ, Tₘₐₓ, AUC_{0-240 min}, TWA_{0-240 min}.
Change from baseline at Day 1 to Week 16 in each treatment arm in fasting blood biomarkers: lipid profile (triglycerides, total cholesterol, LDL-C, HDL-C, VLDL, apoB), adiponectin, leptin, hsCRP, PAI-1, CTX-1, MCP-1, IL-6, endothelin-1, E-selectin, VCAM-1, 3-nitrotyrosine, and UACR.
Change from baseline at Day 1 to Week 16 in each treatment arm in total lean mass and total fat mass as determined by DEXA.
Change in each treatment arm on diabetes-related quality of life as assessed using ADDQOL. Baseline for these assessments is the end of the Screening Period and before they experience the study provided CGM device (i.e., at start of the discretionary Insulin Adjustment Period or before Placebo Run-in Period).
Change in each treatment arm on diabetes treatment satisfaction as assessed using DTSQs and DTSQc. Baseline for DTSQs is the end of the Screening Period and before they experience the study provided CGM device (i.e., at start of the discretionary Insulin Adjustment Period or before Placebo Run-in Period).
Change from baseline at Day 1 to Week 8 and Week 16 in each treatment arm on Composite Appetite Score, as assessed by VAS ratings of hunger, satiety, fullness, and prospective food consumption.
Number of participants maintained at the 4.1 mg, 5.2 mg, and 6.6 mg doses at the end of Week 16 (Arm 4 only).
Time (weeks) to achieve the final dose (Arm 4 only).
Correlation between the time to attain the highest CT-868 dose and change in HbA1c and % body weight change from baseline Day 1 to Week 16 (Arm 4 only).
Percentage of participants who from randomization at Day 1 to Week 16 have permanently discontinued study drug.
Time (weeks) to permanent discontinuation of study drug.

Objectives related to pharmacokinetics & immunogenicity are:
To assess the CT-868 time-concentration profile over 24 hours (in a subset of the overall study population).
To evaluate correlation between CT-868 plasma exposure and relevant efficacy parameters
To evaluate treatment-emergent anti-drug antibodies to CT-868 (Arms 2, 3, 4 only).

Endpoints related to pharmacokinetics & immunogenicity are:
CT-868 PK parameters (Cₘₐₓ, Tₘₐₓ, C_{trough}, Cₗₐₛₜ, Tₗₐₛₜ, AUC₀₋₂₄, AUC₀₋ₗₐₛₜ, and t_{1/2});
Correlation between CT-868 plasma exposure (C_{trough}, Cₘₐₓ, AUC) and HbA1c/Body weight changes; and/or
Treatment-emergent anti-drug antibodies to CT-868 on Day 1 and Week 16 (Arms 2, 3, and 4 only).

### Inclusion Criteria

Participants must meet all of the following criteria in order to be eligible to participate in the study:
1. Male or female, 18 years of age or older at the time of signing informed consent
2. Have a documented diagnosis of T1DM for ≥1 year before the Screening Visit
3. Body mass index ≥25.0 kg/m²
4. Hemoglobin A1c (HbA1c) level between 7.0% and 9.5%, inclusive, at the Screening Visit
5. Treated with insulin either by means of an insulin pump for continuous subcutaneous insulin infusion (CSII), or by means of multiple daily injections (MDIs) of insulin for >6 months prior to the Screening Visit
6. Must be on stable insulin formulation, dose (i.e., within 10% total daily dose), and devices (i.e., not switching from MDI to pump or vice versa) for ≥2 months prior to the Screening Visit as judged and documented by the Investigator

### Exclusion Criteria

Participants who meet any of the following criteria will be excluded from participation in the study:
1. Diagnosis of Type 2 diabetes mellitus (T2DM) or any other types of diabetes, except T1DM
2. Experienced diabetic ketoacidosis (DKA) within 3 months prior to the Screening Visit
3. Experienced severe hypoglycemia (Level 3 as defined in the ADA Standards of Medical Care in Diabetes (ADA 2022) within 3 months prior to the Screening Visit
4. Impaired awareness of hypoglycemia (i.e., a score ≥4 on the Clarke questionnaire [Clarke et al., Diabetes Care (1995) 18 (4): 517-22] at the Screening Visit)
5. Use of any insulin preparation that is not delivered through CSII or MDI (e.g., inhaled insulin).
6. Significant changes in diet or exercise regimen within 3 months prior to the Screening. Visit or plans to undertake any of these during the study duration (e.g., very low calorie, low carbohydrate, very high protein, ketogenic diets, time restricted eating/intermittent fasting, or other nutritional intervention, or initiating a new exercise program).
7. Prior use (i.e., within 6 months prior to the Screening Visit) or current use of any of the following:
   a) Any medication (except insulin) that could interfere with glycemic control (e.g., monoamine oxidase inhibitors, growth hormone);
   b) Any adjunctive treatments for diabetes (e.g., pramlintide, metformin, glucagon-like peptide 1 [GLP-1] analogs [e.g., exenatide], GLP-1 receptor agonists (GLP-1RAs) [e.g., semaglutide, liraglutide, or dulaglutide], GLP-1/GIP RA (Mounjaro™), sodium-glucose cotransporter-2 inhibitors, sulfonylureas, or dipeptidyl peptidase 4 inhibitors);
   c) Any prescription medication(s), and/or any over-the-counter product(s) including herbal and/or dietary supplements for weight loss (e.g., orlistat, lorcaserin, phentermine topiramate, naltrexone-bupropion, semaglutide, liraglutide, garcinia cambogia extract, Hydroxycut®, or glucomannan), or for weight gain (e.g., testosterone, growth hormone, anabolic steroids, or amino acid supplements); or
   d) Are receiving or have received within 3 months prior to Screening Visit chronic (>14 consecutive days) systemic glucocorticoid therapy (excluding topical, intraocular, intranasal, intraarticular, or inhaled preparations) or have evidence of a significant, active autoimmune abnormality (e.g., lupus or rheumatoid arthritis) that has required (within the last 3 months) or is likely to require, in the opinion of the Investigator, concurrent treatment with systemic glucocorticoids (excluding topical, intraocular, intranasal, intra-articular or inhaled preparations) during the study participation.
   e) Any prescribed or non-prescribed drugs that are known to interfere with gut motility including, but not limited to chronic opioids, anticholinergics, antispasmodics, linaclotide, dopamine antagonists.
NOTE: Use of lipid lowering and/or antihypertensive medications are allowed, provided dosage/regimen of these medications have remained stable within the last 2 months prior to Screening Visit.
8 History of treatment intolerance, allergic reactions, or lack of efficacy (i.e., non-responders) with prior use of any GLP-1 analogs, GLP-1RAs, or GLP-1/GIP RAs.
9. Any self-reported body weight gain or loss of ≥5% within 3 months prior to the Screening Visit.
10. Have had or is planning to have during the study duration any surgical treatment for obesity (of any type) or with a weight loss device or any other weight loss procedure (e.g., LAP-Band®, intragastric balloon, duodenal sleeve, resurfacing).
   NOTE: Liposuction and/or abdominoplasty may be allowed based on the Investigator's discretion, if performed >1 year before the Screening Visit, provided all other eligibility criteria are met.
11. Have obesity induced by other endocrinologic disorders (e.g., Cushing syndrome, acromegaly, or inadequately treated hypothyroidism) or diagnosis of monogenetic or syndromic forms of obesity (e.g., melanocortin 4 receptor deficiency or Prader Willi Syndrome).
12. A diagnosis and/or history of clinically significant gastroparesis, gastric motility, gastric emptying abnormalities, malabsorption disorders, chronic constipation, chronic diarrhea, inflammatory bowel disease, bowel resection, irritable bowel syndrome, or severe gastroesophageal reflux disease.
13. Any of the following within 6 months prior to the Screening Visit: myocardial infarction, unstable angina, coronary artery bypass graft, percutaneous coronary intervention (including participants who may have percutaneous transluminal coronary angioplasty [PTCA] planned and/or anticipated during the study period, such as participants with prior angiographic evidence that may require PTCA); transient ischemic attack, cerebrovascular accident, or hospitalization for congestive heart failure.
14. Current New York Heart Association Class III or IV heart failure.
15. Presence of clinically significant electrocardiogram (ECG) findings at the Screening Visit (e.g., corrected QT interval using Fridericia's formula [QTcF] >450 msec for males, QTcF >470 msec for females, left bundle branch block), or any other ECG findings considered to be indicative of active cardiac disease or with abnormalities, in the opinion of the Investigator, that are expected to interfere with the interpretation of ECG changes over the study duration or may present a safety issue to the participant.
16. Uncontrolled hypertension (i.e., mean seated systolic blood pressure ≥160 mmHg and/or mean seated diastolic blood pressure ≥100 mmHg) at the Screening Visit.
17. Any clinically significant change in ophthalmologic examination within 12 months prior to the Screening Visit, including presence of clinically significant proliferative retinopathy, macular edema, or other diabetes-related eye disease.
18. History of any hematologic conditions that may interfere with HbA1c measurement (e.g., hemolytic anemias, sickle cell disease, other hemoglobinopathies).
19. Uncontrolled thyroid disease, defined as having active symptoms (e.g., palpitations, lethargy, weight gain, or weight loss) and/or having a thyroid-stimulating hormone (TSH) value outside the normal reference range of the central laboratory at the Screening Visit
   (Participants receiving adequate treatment for hypothyroidism may be included, provided they are clinically asymptomatic, TSH is within normal reference ranges, and their thyroid hormone replacement dose has remained stable for ≥2 months prior to the Screening Visit).
20 History or current diagnosis of acute or chronic pancreatitis, or risk factors for pancreatitis, such as history of alcoholism, cholelithiasis (without cholecystectomy), hypercalcemia, or severe hypertriglyceridemia.
24 Presence of any one of the following findings during the Screening Period, as determined by the central laboratory:
   a) Alanine transaminase (ALT), or aspartate transaminase (AST), or gamma glutamyl transpeptidase (GGT) >3.0×the upper limit of normal (ULN) of the reference range
   Note: Participants with nonalcoholic fatty liver disease are eligible to participate in this study if their ALT level is ≤3.0×ULN of the reference range.
   b) Alkaline phosphatase (ALP) >1.5×ULN of the reference range
   c) Total bilirubin >ULN of the reference range
   d) Amylase or lipase level of >2×ULN of the reference range
   e) Fasting triglycerides level of >500 mg/dL
   f) Estimated glomerular filtration rate (eGFR) as calculated by the Modification of Diet in Renal Disease equation of <45 mL/min/1.73 m²
   g) Calcitonin ≥20 ng/L, if eGFR is ≥60 mL/min/1.73 m²; or calcitonin ≥35 mg/L, if eGFR is <60 mL/min/1.73 m²
   h) History of, or positive hepatitis screen (including hepatitis B surface antigen [HBsAg] and anti-hepatitis C virus)
   i) Positive human immunodeficiency virus (HIV) antibody screen
   j) Hemoglobin level of <11 g/dL (male participants) or <10 g/dL (female participants)
26. Unable to abstain from using >1000 mg acetaminophen/paracetamol every 6 hours, per the safety information associated with the study-provided CGM device (e.g., Dexcom G6).

### Study Design

This is a Phase 2, double-blind, randomized, placebo-controlled study to evaluate the efficacy, safety, tolerability, and pharmacokinetics of CT-868 in overweight or obese adult participants with T1DM.

While participants' T1DM may be managed with either insulin pump or by means of MDIs of insulin, all participants must be using a CGM device for >2 months before the Screening Visit and must be willing to wear and maintain a study-provided CGM device (e.g., Dexcom G6) for the duration of the study.

All enrolled participants will be expected to maintain their usual lifestyle pattern during the study (e.g., must not initiate a novel diet or new exercise regimen that is different from their normal routines) and follow the usual standards of care for T1DM, including maintaining all other standard-of-care therapies for their other stable chronic conditions, if applicable, throughout the study.

After the Screening Period of up to 3 weeks (Weeks −7 to −4), either at the start of Visit 2 (or Visit 3 if the discretionary Insulin Adjustment Period is not undertaken), eligible participants will be administered diabetes-related quality of life and treatment satisfaction questionnaires to establish a baseline for how they feel and how satisfied they are with their existing diabetes treatment. Following that assessment, participants may enter a discretionary Insulin Adjustment Period for up to 2 weeks (Weeks −4 to −2) where the Investigator has an opportunity to assess the adequacy of their existing insulin regimens and discern if participants are within treat-to-target goals, i.e., ≥70% time in range 70-180 mg/dl (3.9-10 mmol/L) and <4% time below range <70 mg/dL (<3.9 mmol/L) (Battelino et al., Lancet Diabetes Endocrinol. (2023) 11:42-57). The Investigator has the discretion to adjust the participant's insulin regimens, if needed, during this period prior to entering the mandatory 2 week single-blind Placebo Run-in Period (Week −2 to Day 1).

On Day 1, participants will enter a 16-week double-blind Treatment Period and will be randomized 1:1:1:1 to 1 of 4 treatment arms.

The pooled placebo Arm 1 will consist of 3 sets of ˜8 participants to receive the placebo dose volume-matched to a corresponding dose of CT-868. The total number (n˜24) of placebo participants in Arm 1 will be similar to the number of participants in each of the CT-868 treatment arms (i.e., n˜24 in each of Arms 2, 3, and 4).

At the time of randomization, participants will be stratified by both their HbA1c level at the Screening Visit (<8.2% or ≥8.2%), and their method of insulin administration (pump or MDI). By stratifying on these two factors, the effects of CT-868 treatment on glycemic control in both insulin pump and MDI users across different HbA1c levels can be better understood.

Adjustments of insulin doses to minimize risk of hypoglycemia will be guided by initial HbA1c value and by CGM data (see Table 25).

To mitigate risk for DKA, especially at specified periods of insulin dose reduction, systematic and frequent ketone monitoring with appropriate mitigating measures is recommended (Table 26).

Just as undertreatment of insulin could lead to DKA, overtreatment with insulin could lead to hypoglycemia. Appropriate mitigating measures will be undertaken to further minimize risk of hypoglycemia.

After randomization on Day 1, the first dose of study drug self-administration will be observed at the clinic after all the necessary baseline assessments have been completed. Participants will once again be instructed to administer study drug by SC injection into the abdomen, at approximately the same time in the morning each day.

### Dosage Forms and Route of Administration

CT-868 Injection 15 mg/mL and CT-868 Injection placebo solutions will be supplied in a needle-based injection system with an integrated non-replaceable 3-mL Type 1 glass cartridge, the CT-868 Pen Injector (Pen). The CT-868 Pen is a multi-dose, single participant, disposable pen designed to deliver five fixed doses of 1.1 mg (0.07 mL), 1.8 mg (0.12 mL), 2.6 mg (0.17 mL), 3.3 mg (0.22 mL), and 4.1 mg (0.27 mL) by SC injection in the abdomen. The CT-868 Pen contains a clear, colorless, sterile aqueous solution of 3 mL of 15 mg/mL CT-868 (active only) in 20 mM di-sodium hydrogen phosphate heptahydrate/sodium dihydrogen phosphate monobasic buffer, propylene glycol, and phenol at pH 7.0. The placebo Pen contains the same ingredients except CT-868. Each active Pen contains ˜45 mg/3 mL of CT 868. Each Pen is individually packaged in a carton including the Instructions for Use (IFU). Within each dose group, the CT 868 and placebo Pens will be identical in appearance.

The CT-868 and placebo Pens must be stored refrigerated and brought to room temperature prior to dosing.

CT-868 and placebo are to be titrated per Protocol and in Arm 4, according to the individual participant's tolerability up to a maximum dose of 6.6 mg CT-868. Details of the doses and titration scheme for each treatment arm are provided in Table 25.

Participants randomized to Arm 2, or Arm 3, or Arm 4 would need to tolerate a minimum dose of 1.8 mg, or 3.3 mg, or 4.1 mg, respectively, to continue participation in the study and for their data to be utilized for the primary efficacy analysis.

### Criteria for Evaluation

Efficacy will be assessed by HbA1c, various CGM metrics, body weight/body composition measurements, MMTTs, and PROs.

Safety assessments will include the following:
AEs/ADEs, including injection site reactions
AEs/ADEs of special interest (hypoglycemia, DKA, pancreatitis, diabetic retinopathy, acute kidney injury, pen malfunction, pen use errors, intentional pen misuse, and inadequate adherence to the IFU).
(NOTE: Participants who report specific symptoms of hypoglycemia will also be requested to provide narratives for such events.
Vital signs
Physical examinations
ECGs
Clinical laboratory tests (hematology, chemistry, including beta-hydroxybutyrate, amylase, lipase, calcitonin, and urinalysis)

TABLE 25
Guidelines for Insulin Adjustment

| Timing of Insulin | |
|---|---|
| Adjustment | Insulin Adjustment Recommendations |
| Prior to the first dose of | If screening HbAlc >8.2%, |
| study drug, changes to | no change in insulin dose |
| participant's insulin | If screening HbAlc is between |
| doses are recommended | 7.0% and 8.2% (both inclusive), |
| based on the screening | reduce total daily insulin dose by |
| HbA1c values | 15% split evenly between basal |
| | and bolus compartments for |
| | at least 24 hours |
| Prior to each scheduled | If the mean weekly glucose by |
| study drug dose up- | CGM is <140 mg/dL OR time below |
| titration within each | range is >4%, total daily insulin |
| treatment arm, CGM | dose (split evenly between basal |
| will be uploaded | and bolus compartments) will be |
| | reduced by 10% for at least 24 |
| | hours. |
| | If neither of these metrics are |
| | met, insulin doses would not |
| | prophylactically be reduced. |
| At any other time during | If both TIR is ≥70% and TBR is |
| the study, insulin doses | <4% (i.e., within the stipulated |
| could be adjusted | treat-to-target range), no changes |
| weekly based on | to TDD of insulin will be made. |
| participant's CGM | If both these criteria are not met, the |
| readings to maintain | following adjustments may be |
| their treat-to-target | recommended: |
| ranges (i.e., ≥70% TIR | If TIR is <70% and TBR <4%, |
| 70-180 mg/dl and <4% | the TDD of insulin will be |
| TBR <70 mg/dL) after | increased by 10%; basal rate and/or |
| starting the study drug | the bolus dose (e.g., based |
| | on insulin-to-carbohydrate ratio; |
| | bolus calculators may be used) |
| | may be appropriately adjusted |
| | by the investigator |
| | If TIR is ≥70%, but TBR is >4%, |
| | the TDD of insulin will be |
| | decreased by 10% with basal rate |
| | and/or bolus doses adjusted |
| | per investigator's judgement as above |
| | If TIR <70% and TBR >4%, |
| | changes in TDD of insulin will |
| | need to be made on a |
| | case-by-case basis using the |
| | Investigator's clinical judgement |
| | as both an increase or |
| | decrease in TDD of insulin |
| | could be justifiable in such a |
| | situation given the individual's |
| | unique circumstances. |

CGM = continuous glucose monitoring;
MDI = multiple daily injection;
TIR-time in range;
TBR = time below range;
TDD-total daily dose.
Notes:
(i) Participants on MDIs should remain on MDIs throughout study (i.e., cannot switch to an insulin pump or vice versa during the trial); and
(ii) Discontinuation of insulin is not permitted at any time during this study.

TABLE 26
Recommended Action Plan for Management of DKA

| Blood Ketone | | |
|---|---|---|
| (BHB) | Urine | |
| Level | Ketone ^{a} | Remedial Action |
| <0.6 mmol/L | Negative | No action needed |
| normal) | | |
| 0.6-1.5 mmol/L | Trace or | Treat as follows or per clinician instructions: |
| (ketonemia) | small | Ingest 15-30 g rapidly absorbed carbohydrate |
| | | and maintain fluid consumption (300-500 mL) |
| | | hourly |
| | | Administer rapid-acting insulin based on |
| | | carbohydrate intake (hourly) |
| | | Check blood/urine ketones (every 3-4 hours) |
| | | until resolution |
| | | Check blood glucose frequently to avoid |
| | | hyperglycemia and hypoglycemia |
| | | Seek medical attention if levels persist and |
| | | symptoms present |
| 1.6-3.0 mmol/L | Moderate | Follow treatment recommendations |
| (impending | | listed above. |
| DKA) | | Consider seeking immediate medical attention |
| >3.0 mmol/L | Large | Seek immediate medical attention. |
| (probable | to very | |
| DKA) | large | |

BHB = beta-hydroxybutyrate.

TABLE 27
Dose/Titration Adjustment Schema

| Treatment Arm | Dose and Duration | Dose/Titration Adjustment |
|---|---|---|
| Arm 1 | One-third of the placebo | Not applicable |
| Volume-matched | participants (n~8) will titrate | |
| placebo | placebo based on Arm 2 (the | |
| (n~24) | 1.8 mg arm scheme) | |
| | One-third (n~8) will titrate | |
| | placebo based on Arm 3 (the 4.1 | |
| | mg arm scheme) | |
| | One-third (n~8) will titrate | |
| | placebo based on Arm 4 (the | |
| | dose to tolerability/up to | |
| | maximum of 6.6 mg arm | |
| | scheme) | |
| Arm 2 | Day 1 to Day 7—1.1 mg | No adjustments allowed |
| 1.8 mg CT-868 | Week 1 to Week 2—1.8 mg | |
| (n~24) | | |
| Arm 3 | Day 1 to Day 7—1.1 mg | No adjustments in dose escalations are |
| 4.1 mg CT-868 | Week 1 to Week 2—1.8 mg | allowed up to Week 6. |
| (n~24) | Week 2 to Week 4—2.6 mg | If escalation to the 4.1 mg dose is not |
| | Week 4 to Week 6—3.3 mg | tolerated at Week 6 or at any time during |
| | Week 6 to Week 16—4.1 mg | the 2-week period (i.e., between Weeks 6 |
| | | and 8), and tolerability issues remain |
| | | despite the use of appropriate concomitant |
| | | medications (e.g., anti-emetics or |
| | | laxatives), and the Investigator does not |
| | | believe that the participant will tolerate the |
| | | dose with further exposure, the Investigator |
| | | may keep the dose at 3.3 mg or reduce the |
| | | dose from 4.1 mg to 3.3 mg, as appropriate. |
| | | Up to 3 subsequent attempts to re- |
| | | challenge/up-titrate back to the 4.1 mg dose |
| | | may be offered to participants over the |
| | | subsequent 4 weeks (if they remain |
| | | asymptomatic), but must be completed |
| | | before Week 12, the last up-titration point. ³ |
| Arm 4 | Day 1 to Day 7—1.1 mg | No adjustments in dose escalations are |
| CT-868 dosed to | Week 1 to Week 2—1.8 mg | allowed up to Week 8. |
| tolerability/ | Week 2 to Week 4—2.6 mg | If escalation to the 5.2 mg dose is not |
| up to maximum | Week 4 to Week 6—3.3 mg | tolerated at either Week 8 or at any time |
| of 6.6 mg (n ~24) | Week 6 to Week 8—4.1 mg | during the 2-week period provided for that |
| | Week 8 to Week 10—5.2 mg ¹ | dose (i.e., between Weeks 8 and 10), |
| | Week 10 to Week 16—6.6 mg ² | despite instituting all the above |
| | | symptomatic-relief measures, the |
| | | Investigator may keep the dose at 4.1 mg or |
| | | reduce the dose from 5.2 mg to 4.1 mg, as |
| | | appropriate. Up to 2 subsequent attempts to |
| | | re-challenge/up-titrate back to the 5.2 mg |
| | | dose over the subsequent 2 weeks (if they |
| | | remain asymptomatic) may be offered, but |
| | | must be completed before Week 12, the last |
| | | up-titration point. ³ At Week 12, if 5.2 mg is |
| | | not tolerated, then continue at 4.1 mg for |
| | | the remainder of the treatment period. |
| | | If escalation to the 6.6 mg dose is not |
| | | tolerated at either Week 10 or at any time |
| | | during the 2-week period between Week 10 |
| | | and Week 12, despite instituting all the |
| | | above symptomatic-relief measures, the |
| | | Investigator may keep the dose at 5.2 mg or |
| | | reduce the dose from 6.6 mg to 5.2 mg, as |
| | | appropriate. One subsequent attempt to re- |
| | | challenge/up-titrate back to the 6.6 mg dose |
| | | over the subsequent 2 weeks (if they remain |
| | | asymptomatic) may be offered, but must be |
| | | completed before Week 12, the last up- |
| | | titration point. ³ At Week 12, if 6.6 mg is |
| | | not tolerated, then continue at 5.2 mg for |
| | | the remainder of the treatment period. |

¹ Administered as 2 doses of 2.6 mg each.
² Administered as 2 doses of 3.3 mg each.
³ No up-titrations in dose will be allowed after Week 12 (i.e., the dose that the participant is able to tolerate at the Week 12 visit will be the maximum dose maintained for the remainder of the dosing period without further dose increases). However, doses may continue to be down-titrated after Week 12 to their previously tolerated dose if the final re-challenge/up-titration dose at Week 12 was not tolerated.

### Example 7: A Phase 2 Trial of CT-868, a Novel Dual GLP-1 and GIP Receptor Modulator, in Overweight and Obese Adults with T2DM

### Method

This Example described data from a Phase 2, 26-week, randomized, placebo-controlled, double-blind, multicenter study that assessed the efficacy and safety of CT-868 in adults with T2D and overweight/obesity suboptimally controlled with diet and exercise, with or without metformin.

### Objectives and Endpoints

The primary objective of this study was to assess change in HbA1c and characterize the safety and tolerability profile of CT-868 administered daily by subcutaneous injection.

The primary endpoint of this study was to evaluate the change in HbA1c levels at Week 26.

Secondary endpoints of this study included assessing the change from baseline in body weight, fasting plasma glucose and lipids, and safety/tolerability at Week 26.

### Study Design

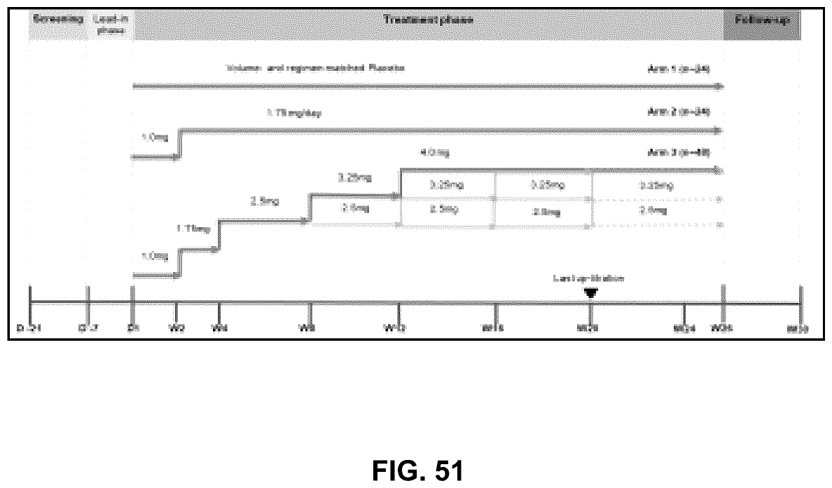

103 participants (men/women: 39/64) aged 18-75 years (median age: 50 years) having a diagnosis of T2D for >6 months, BMI ≥27 kg/m², and HbA1c of 7-10% (mean BW/BMI/HbA1c: 92 kg/35.3 kg/m²/8.1%), and on diet/exercise alone or metformin monotherapy at screening, were randomized 1:1:2 into one of three treatment arms:
Arm 1: Volume-matched placebo (placebo; n˜24);
Arm 2: CT-868 1.75 mg (n˜24); and
Arm 3: CT-868 dosed up to 4 mg based on individual tolerability (n˜48).
   Titration schemes are shown in FIG. 51. In Arm 3, 32 participants received the 4 mg dose and 18 participants received the 3.25 mg dose. Thus, three doses of CT-868 (1.75, 3.25, and 4 mg) vs. placebo were administered. To account for this variability, analyses were conducted on the final dose received by each participant (1.75 mg, 3.25 mg, or 4 mg) instead of their initially randomized group.

### Results

Subject demographics are shown in Table 28 below.

TABLE 28
Demographics and Baseline Characteristics

| | | CT-868 | CT-868 | CT-868 |
|---|---|---|---|---|
| | Placebo | 1.75 mg | 3.25 mg | 4 mg |
| Category | N = 27 | N = 26 | N = 18 | N = 32 |
| Age, years | 50.3 (11.7) | 52.3 (7.8) | 48.2 (9.9) | 49 (12.1) |
| Female, n(%) | 16 (59.3) | 14 (53.8) | 12 (66.7) | 22 (68.8) |
| Ethnicity: | 27 (100%) | 26 (100%) | 18 (100%) | 32 (100%) |
| Hispanic or | | | | |
| Latino, n (%) | | | | |
| Weight, kg | 94.6 (21.3) | 92.3 (20.5) | 90.1 (16.4) | 90.6 (17.4) |
| BMI, kg/m² | 35.7 (6.3) | 36.0 (6.6) | 34.8 (4.5) | 34.8 (5.5) |
| HbA1c, % | 8.1 (1.0) | 8.3 (0.8) | 7.6 (1.0) | 8.4 (0.7) |
| Fasting glucose, | 143.5 (46.6) | 180.3 (42.2) | 146.1 (40.2) | 155.3 (37.8) |
| mg/dl | | | | |
| Metformin use, | 27 (100) | 24 (96.0) | 18 (100) | 32 (100) |
| n (%) | | | | |

Data presented as mean (SD) values unless otherwise specified).

### Glycemic Control

HbA1c and fasting plasma glucose (FPG) levels at Week 26 decreased in each CT-868 arm (1.75, 3.25, 4 mg) with placebo-corrected LS mean difference of −1.92%, −1.67%, −2.30% (p<0.001) and −52.9, −56.7, and −60.1 mg/dL (p<0.001), respectively. Proportion achieving HbA1c≤6.5% at Week 26 was higher (p<0.01) in CT-868 vs. placebo: 52.2% (1.75 mg), 72.2% (3.25 mg), 69.0% (4 mg), 18.2% (placebo).

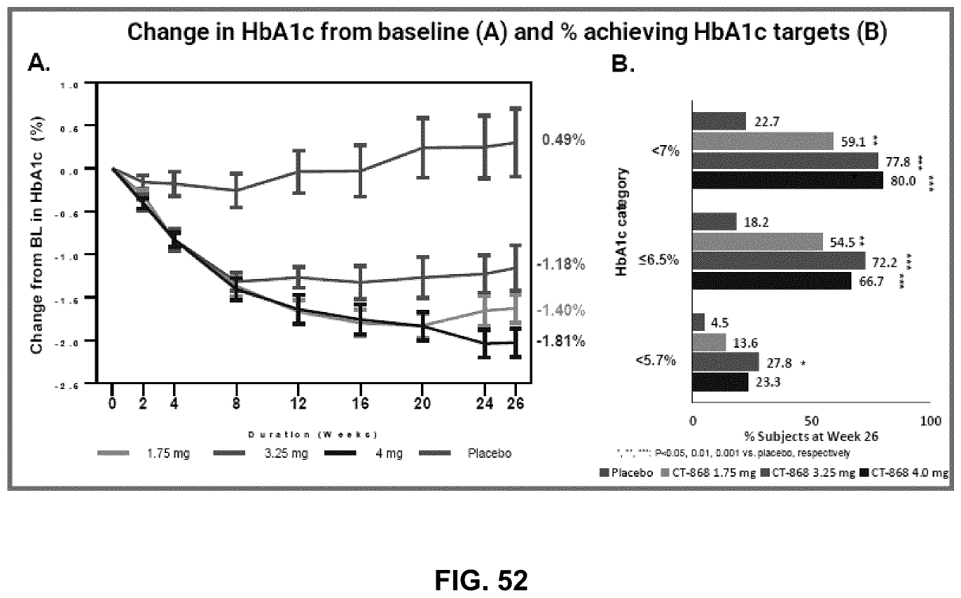

HbA1c improved significantly from baseline in all three CT-868 arms compared with placebo at Week 26. Mean treatment difference in the change from baseline in HbA1c for CT-868 4.0 mg vs. placebo was −2.3% (95% CI −3.03 to −1.58, p<0.001). Significantly more participants achieved HbA1c≤6.5% and HbA1c <7% in all three CT-868 arms compared with placebo. FPG was significantly improved with CT-868 compared to placebo (treatment differences −51.8 to −60.5 mg/dL, p<0.001 for all arms). See FIG. 52.

### Body Weight

Mean % change in BW was −5.1% in CT-868 4 mg arm vs −2.5% placebo (p=0.025). 14% of the CT-868 4 mg-treated group achieved >10% weight loss vs 0% in placebo.

A 4.0 mg dose of CT-868 was associated with a (5.7±4.4) % reduction in body weight from baseline at Week 26. Weight loss in the placebo arm was (2.3±3.9) %. 53.3% of participants on the 4.0 mg CT-868 dose achieved ≥5% weight loss (WL) at Week 26 compared to 22.7% on placebo (p=0.027).

### Lipid Parameters

In CT-868 4 mg arm, mean placebo-adjusted change in T-Chol, TG, LDL-C, and ApoB were −26, −139, −15, and −20 mg/dL, respectively (p<0.01).

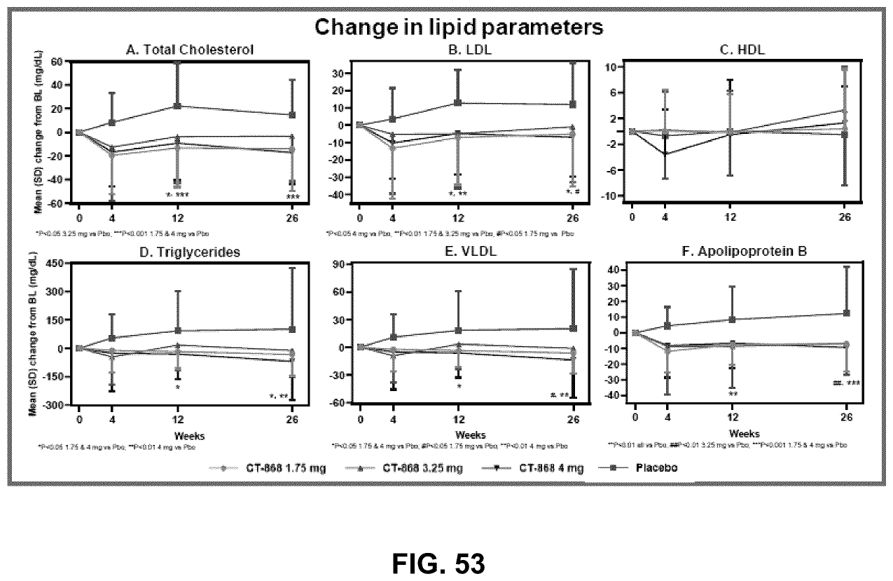

Total cholesterol, triglycerides, LDL-C, VLDL, and apolipoprotein B were all reduced in the CT-868 treated arms compared with placebo at Week 26. See FIG. 53.

### Vital Signs

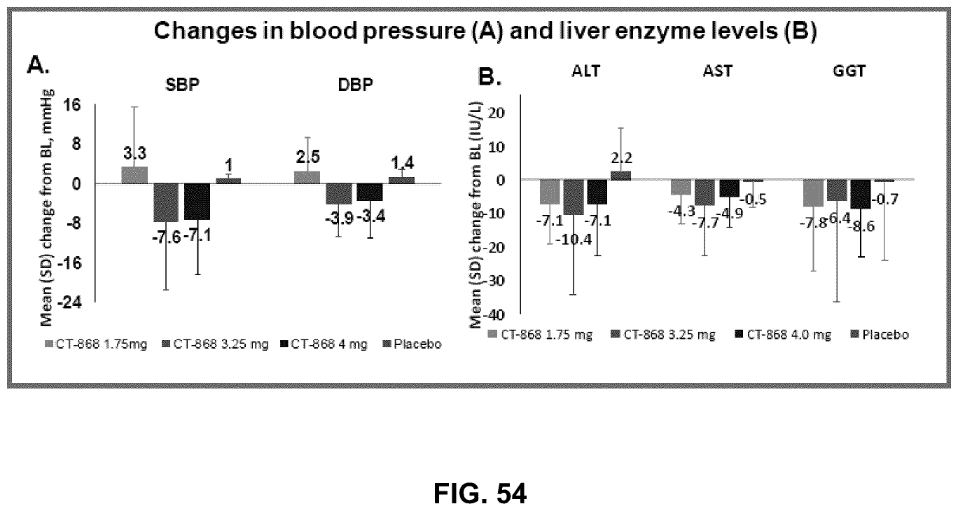

CT-868 dosed at 3.25 mg and 4.0 mg decreased systolic blood pressure (SBP) and diastolic blood pressure (DBP) at Week 26. SBP/DBP was −7.6/−3.6 mm Hg in CT-868 4-mg arm vs placebo (+1/+1.4 mmHg). See FIG. 54.

Mean heart rate increased 1.7±8.8 to 2.3±8.7 beats per minute (bpm) across the CT-868 treatment arms, compared to a decrease of 4.5±8.2 bpm for placebo.

### Liver Tests

Liver enzymes decreased 20-25% in CT-868-treated groups vs. placebo. Serum levels of alanine aminotransferase (ALT), aspartate aminotransferase (AST), and gamma glutamyl transferase (GGT) were reduced by ˜15% to 25% at Week 26 relative to baseline in all CT-868 treatment arms, while no meaningful change in placebo was observed. No clinically significant changes in bilirubin were noted. See FIG. 54.

### Safety & Tolerability

CT-868 was safe and well-tolerated with no treatment-related discontinuations in the CT-868 arms. Most frequent adverse events (AEs) were gastrointestinal (GI)-related, seen across both CT-868 (60%) and placebo (56%) arms [nausea (16-28% vs 26%), constipation (8-20% vs 19%), vomiting (8-10% vs 4%), diarrhea (37-44% vs 22%), with no dose dependence, and mostly mild (Grade 1) in severity. No clinically significant hypoglycemia was reported.

TABLE 29
Overall Summary of Treatment Emergent Adverse Events (TEAEs)

| | | CT-868 | CT-868 | CT-868 |
|---|---|---|---|---|
| | Placebo | 1.75 mg | 3.25 mg | 4 mg |
| Category, n (%) | N = 27 | N = 26 | N = 18 | N = 32 |
| Subjects with ≥1 TEAE | 23 (85.2) | 22 (84.6) | 16 (88.9) | 28 (87.5) |
| Serious TEAEs, n | 0 | 0 | 1 (5.6) | 0 |
| Subjects Discontinued due | 1 (3.7) | 0 | 0 | 0 |
| to TEAE | | | | |
| Fatal TEAEs, n | 0 | 0 | 0 | 0 |
| Drug-Related* TEAEs, n | 16 (59.3) | 15 (57.7) | 9 (50.0) | 20 (62.5) |
| Severity^{†}: | | | | |
| Grade 1 | 13 (48.1) | 10 (38.5) | 9 (50.0) | 14 (43.8) |
| Grade 2 | 3 (11.1) | 5 (19.2) | 0 | 5 (15.6) |
| Grade 3 | 0 | 0 | 0 | 1 (3.1) |

*Relationship to study drug is categorized as ‘Possible’ or ‘Probable’;
^{†}Severity of drug-related TEAEs

TABLE 30
Gastrointestinal TEAEs

| | | CT-868 | CT-868 | CT-868 |
|---|---|---|---|---|
| | Placebo | 1.75 mg | 3.25 mg | 4 mg |
| Preferred Term, n (%) | N = 27 | N = 26 | N = 18 | N = 32 |
| Gastrointestinal Disorders | 15 (55.6) | 16 (61.5) | 7 (38.9) | 22 (68.8) |
| Abdominal distention | 2 (7.4) | 1 (3.8) | 1 (5.6) | 3 (9.4) |
| Constipation | 5 (18.5) | 2 (7.7) | 0 | 10 (31.3) |
| Diarrhea | 6 (22.2) | 12 (46.2) | 4 (22.2) | 14 (43.8) |
| Dyspepsia | 2 (7.4) | 3 (11.5) | 0 | 2 (6.3) |
| Flatulence | 0 | 2 (7.7) | 1 (5.6) | 0 |
| Nausea | 7 (25.9) | 5 (19.2) | 2 (11.1) | 11 (34.4) |
| Vomiting | 1 (3.7) | 3 (11.5) | 2 (11.1) | 2 (6.3) |

*Occurring in at least 5% of participants

### Conclusion

CT-868 demonstrated robust glycemic control (−2.3% HbA1c lowering compared to placebo) with 67-72% of patients, achieving an HbA1c of ≤6.5% (in the non-diabetes range) at Week 26. Improvements in CV risk factors were also demonstrated. CT-868 dosed up to 4.0 mg improved key cardiovascular risk factors (LDL-C, apoB, VLDL, TG, blood pressure), and liver enzymes. CT-868 was well tolerated at 4.0 mg. The favorable tolerability profile supports higher CT-868 dosing in subsequent studies to maximize its weight loss benefits.

### Example 8: A Phase 2 Trial of CT-868, a Novel Dual GLP-1 and GIP Receptor Modulator, in Overweight and Obese Adults with TID

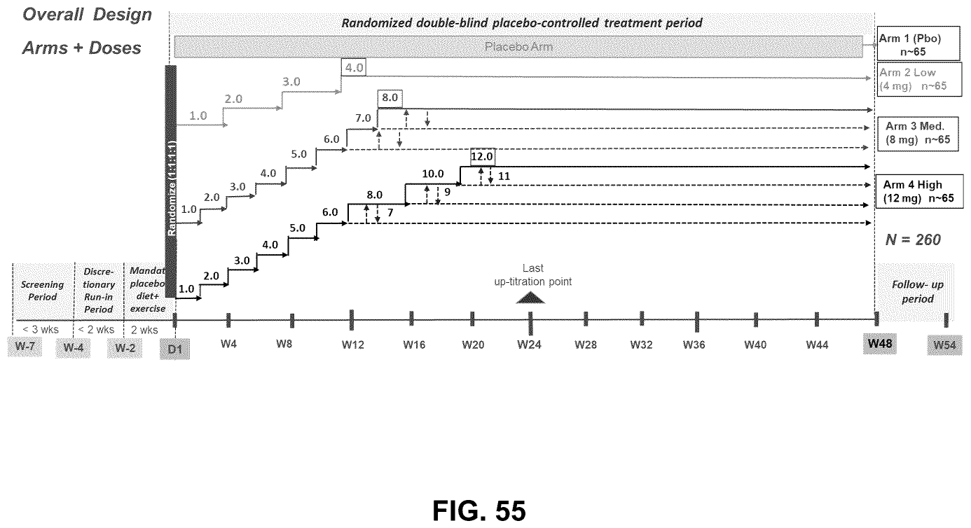

The main objective of this study is to measure glycemic control and weight loss in patients. Treatment lasts for a period of 48 weeks or less. Titration is conducted over two or four weeks. Titration doses are 5, 10, and 15 mg or 3, 6, 9, 12, and 15 mg. FIG. 55 shows overall trial design, including arms and doses. The screening period lasts under three weeks, the discretionary run-in period lasts under two weeks, and the placebo diet and exercise period lasts two weeks. The discretionary Run-in Period includes Instruction in CGM Handling, Insulin Dose Optimization, and Safety of AID Pump Features.

The mandatory Placebo, Diet & Exercise Run-in Period includes Baseline Assessments of all Endpoints and Safety and CGM+Insulin Dose Collection.

### Inclusion Criteria

Inclusion criteria include:
Male or female, age between ≥18 [and ≤70] years;
Has a documented clinical diagnosis of T1DM for at least 1 year prior to Screening with a fasting C-peptide of <0.6 ng/ml;
Body mass index ≥27.0 kg/m²;
HbA1c: between ≥7.0% and ≤10.5%;
Stable use of insulin preparations/delivery method for at least 3 months;
Uses either insulin pumps (manual or AID) or MDI;
Has experience in CGM and willing to use study-provided sensor for several 2-week intervals;
Willing to adhere to all prescribed diabetes self-management tasks including insulin dose adjustment, blood ketone testing, keeping current glucagon products;
Must be a non-smoker;
Willing to comply with contraception rules; and
Willing to inject sq study drug daily.

### Assessments

Assessment criteria include:
AEs and ConMeds;
CGM Download & PI Discussion;
Blood and Urine Collection;
Questionnaires: DTSQ, ADDQOL, and CoEQ, TLFB;
Body Weight & Body Measures
Vitals, ECG, Physical Exam
Patient Education: Diet & Exercise

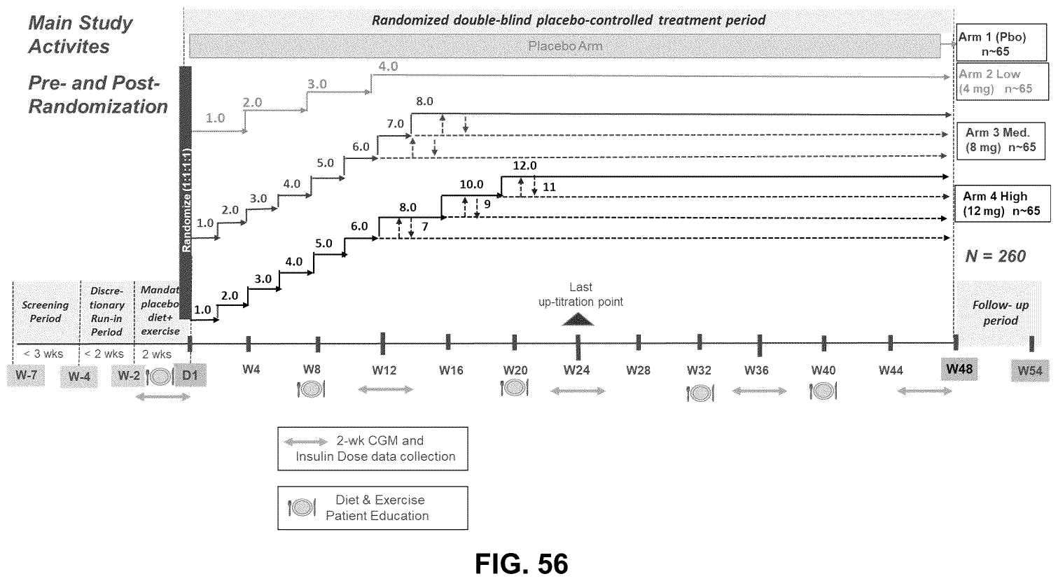

FIG. 56 shows the main study activities in pre- and post-randomization of a Phase 2 trial of CT-868 in Overweight and Obese adults with TID, including 2-week CGM and insulin dose data collection and diet and exercise patient education.

### Main Study Endpoints:

Co-primary endpoints include:
Change in HbA1c (%) FB to 48 Wks; and
% Change in Body Weight FB to 48 Wks

Key secondary endpoints include:
Change in HbA1c (%) FB to Wks 12, 24, 36;
Status of achieving HbA1c of <7.0 or 6.5%;
% Change in Body Weight FB to Wks 12, 24, 36;
Absolute Weight Change (kg) at Wks 12, 24, 36, 48;
% participants achieving >5%, >10%, >15% at Wks 24, 48; and
% Change in insulin doses FB to Wks 12, 24, 36, 48.

Other secondary endpoints include:
Change FB to Wks 24 and 48 in daily insulin requirements;
Change FB to Wks 24 and 48 in body composition assessed by Dexa;
Change FB to Wks 12, 24, 36, 48 in the following CGM parameters:
   Mean Sensor Glucose (mg/dL) and its standard deviation (SD) and % CV;
   Percent and amount of time spent in range in TIR, TBR and TAR;
   Glycemic Risk Index; and
   Composite Endpoints;
Change FB to Wks 12, 24, 36, 48 in LDLC, HDL-C, VLDL, FFA, ApoB;
Change FB to Wks 12, 24, 36, 48 in SBP & DBP; and
Change FB to Wks 12, 24, 36, 48 in glucometabolic parameters: FPG, insulin, C-peptide, glucagon, GLP1, GIP, HOMA-IR & QUICKI.

Exploratory endpoints include:
Change FB to Wks 12, 24, 36, 48 in adiponectin, leptin, hsCRP, PAI-1 activity, CTX-1, MCP-1, IL-6, endothelin-1, Eselectin, VCAM1, ALT;
Change in UACR FB to Wks 12, 24, 36, 48;
Change in eGFR slope FB to Wks 24 and 48; and
Change FB to Wks 12, 24, 36, 48 in PRO instruments: DTSQ, ADDQOL, and CoEQ, TLFB.

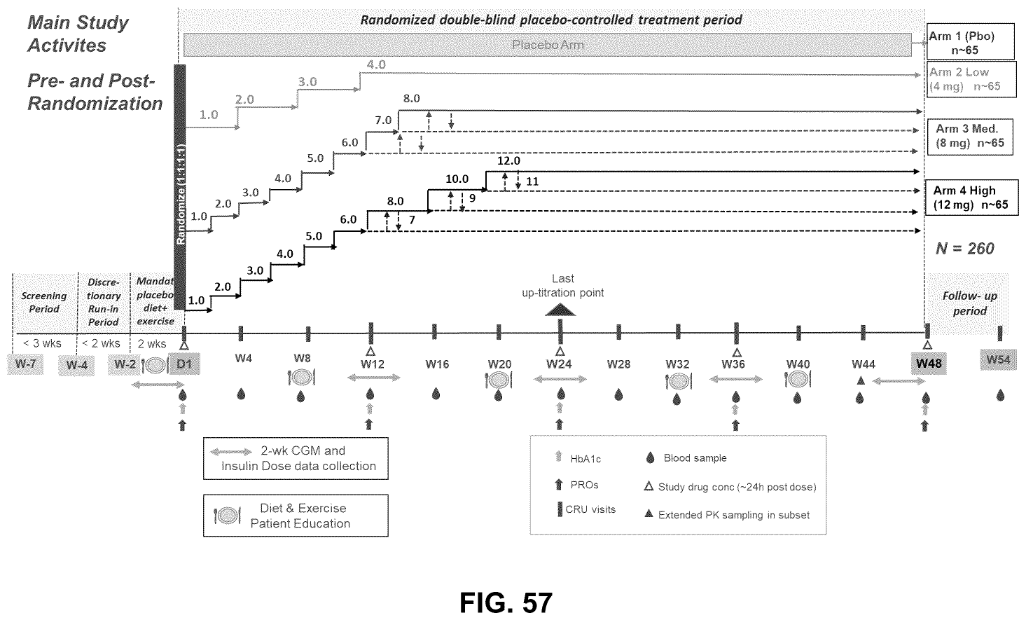

FIG. 57 shows the main study activities in pre- and post-randomization of a Phase 2 trial of CT-868 in Overweight and Obese adults with TID, including HbA1c, PROs, CRU visits, blood sample, study drug concentration (˜24 hours post dose), and extended PK sampling in subset.

Learnings from CT-868-004 to improve subsequent studies include:
Include the full spectrum of insulin pumps, including AID pumps;
Be clear and precise in formulating I/E inclusion criteria to eliminate ambiguities and confusion;
Set up mechanism that Sponsor is informed and can intervene in subject selection prior to randomization;
Educate research sites and patients about the common GI side effects and how to mitigate and treat them;
Integrate patient education sessions about diet & exercise, CGM, insulin pump function;
Limit the CGM data capture to 2-week periods at baseline, middle and end of study;
Reduce the burden of subjects' data collection, use systems where data are entered automatically (like from glucometers, CGM, insulin pumps); and
Use the monitoring digital platform capacities from Roche Diabetes Care in our study's data capturing requirements.

### Example 9: Stability of CT-868 Injections

CT-868 Injection, 15 mg/mL and CT-868 Injection, Placebo Cartridges

The release and stability specifications for the CT-868 Injection, 15 mg/mL drug product (DP) cartridges that ensure the quality, strength, identity, purity, and microbial attributes are provided in Table 31. Except for the Purity test, release, and stability testing for the CT-868 Injection Placebo DP are the same as the CT-868 Injection, 15 mg/mL DP. The CT-868 Placebo DP Identity and Assay acceptance criteria are replaced with No Active Present and 0%, respectively.

TABLE 31
Specifications for CT-868 Injection, 15 mg/mL and Placebo Cartridges

| | | |
|---|---|---|
| Test | Analytical Procedure | Acceptance Criteria |

Appearance

| | | |
|---|---|---|
| Visual | SOP-QCA-023 | Clear liquid, |
| | | essentially free of |
| | | visible particles |
| Clarity | TM-QCA-019 | ≤Reference |
| | Ph. Eur. 2.2.1 | Suspension II |
| | | (Colorless to |
| | | pale yellow |
| | | colored solution) |
| Color | TM-QCA-020 | Not more intense |
| | Ph. Eur. 2.2.2 | color than |
| | | reference solution |
| | | Y5 (Clear) |

General Tests

| | | |
|---|---|---|
| pH | TM-QCA-010, | 6.9-7.1 |
| | USP <791> | |
| Osmolality (mOsm/kg) | TM-QCA-023, | Report Results |
| | USP <785> | |
| Particulate matter | TM-QCA-009, | ≥10 μm: |
| (Sub-visible | USP <788> | ≤6000 per container |
| particles) | | ≥25 μm: |
| | | ≤600 per container |
| Extractable Volume [1] | TM-QCA-014 | NLT 2.88 mL |

Identity [1]

| | | |
|---|---|---|
| CT-868, Injection, | TM-161-001 | Identity Confirmed |
| 15 mg/mL | | |
| CT-868 Injection | TM-161-001 | No Active Present |
| Placebo | | |

Strength

| | | |
|---|---|---|
| Assay: | | |
| CT-868 Injection | TM-161-001 | 90.0%-110.0% |
| 15 mg/mL [2] | | Label Claim |
| CT-868 Injection | | 0% |
| Placebo | | |

Purity [2]

| | | |
|---|---|---|
| Individual Impurities | TM-161-001 | No impurity: |
| | | ≥5.0% Area |
| | | Report all impurities |
| | | ≥0.5% Area |
| Total Impurities | | ≤7.0 % Area |

Microbial

| | | |
|---|---|---|
| Bacterial Endotoxin | TM-QCA-012, | ≤5 EU/mL |
| | USP <85> | |
| Sterility [3] | USP <71> | No Growth |

[1] Release test only;
[2] CT-868 Injection, 15 mg/mL only;
[3] SGS Lincolnshire, IL

Stability Summary and Conclusions [CT-868 Injection, 15 mg/mL]

CT-868 drug substance is formulated as a sterile aqueous solution for use in three drug product (DP) presentations for SC administration: a 2-mL CT-868 Injection, 5 mg/mL in a glass vial, a 1-mL CT-868 Injection, 5 mg/mL/matching placebo in a disposable pre-filled syringe (PFS), or a 3-mL CT-868 Injection, 15 mg/mL/matching placebo intended for assembly as a disposable multi-dose, single-patient use pen injector (integrated non-replaceable cartridge).

A simulated in-use stability study at refrigerated storage (5±3° C.) was completed as part of design verification to support CT-868 Pen Injector use for up to 31-days.

One CT-868 active DP engineering lot and five GMP lots (three CT-868 active DP and two CT-868 placebo DP) are included in the stability program to support shelf-life dating and use during clinical trials. Additional stability data up to 18-months at 5±3° C. long-term storage is submitted for engineering lot ENG-161-001-001 and at 5±3° C. long-term storage for GMP lot 161-002-001, lot 161-002-002 and lot 161-001-001, 18 months, 12 months and 18 months, respectively, as listed in Table 32. One additional lot 161-002-003 of CT-868 active DP has been included in the stability program with 6-months data available at 5±3° C. long-term storage and 25±2° C./60% RH (25° C.). Additional data for up to 12-months are submitted at 5±3° C. long-term storage and 25° C. for CT-868 placebo DP lot 161-001-002. Based on the 18-months stability data at 5±3° C. long-term storage for lot ENG 161-001-001 and 161-002-001 and 12-months stability data at 5±3° C. long-term storage for lot 161-002-002 he CT-868 Injection DP cartridges proposed shelf life is 24-months.

Information on the lots on stability is provided in Table 32. The stability storage conditions, time points and tests conducted are provided in Table 33. Moving forward, Phenol has been added to the scheduled stability tests with the results reported. The acceptance criteria used for the evaluation of the stability data are those provided in the respective stability protocols.

TABLE 32
CT-868 Injection Stability Lot Information

| | | | | Data | |
|---|---|---|---|---|---|
| | | | | Available | |
| | | Date of | Date on | (months) | |
| Lot Number | Manufacturer | Manufacture | Stability | 5° C. | 25° C. |

Engineering Lot

| | | | | | | | |
|---|---|---|---|---|---|---|---|
| ENG-161-001-001 | BSM | 09 Dec. | 2021 | 14 Dec. | 2021 | 18 | 6 Completed |

Clinical Lots—Active

| | | | | | | | |
|---|---|---|---|---|---|---|---|
| 161-002-001 | BSM | 31 Mar. | 2022 | 26 Apr. | 2022 | 18 | 6 Completed |
| 161-002-002 | BSM | 19 Jul. | 2022 | 03 Aug. | 2022 | 12 | 6 Completed |
| 161-002-003 | BSM | 08 Feb. | 2023 | 13 Feb. | 2023 | 9 | 6 Completed |

Clinical Lot—Placebo

| | | | | | | | |
|---|---|---|---|---|---|---|---|
| 161-001-001 | BSM | 25 Mar. | 2022 | 08 Apr. | 2022 | 18 | 6 Completed |
| 161-001-002 | BSM | 07 Jul. | 2022 | 13 Sep. | 2022 | 12 | 6 Completed |

TABLE 33
Stability protocol for CT-868 Injection (Active and Placebo)

| Storage | Time Points (months) | | | | | | | | |
|---|---|---|---|---|---|---|---|---|---|
| Conditions | Initial | 1 | 3 | 6 | 9 | 12 | 18 | 24 | 36 |
| 5 ± 3° C. | T0* | NS | X | X | X | XES | X | XES | XES |
| (Horizontal) | | | | | | | | | |
| 25 ± 2° C./ | | X | X | XES | | | | | |
| 60 ± 5% RH | | | | | | | | | |
| (Horizontal) | | | | | | | | | |

Notes:
NS = Not scheduled
*Release data may be used
X = Visual, Color, Clarity, pH, Osmolality, Particulate Matter, Identity (active only), Strength (active only), Phenol, Purity (active only)
E: Bacterial Endotoxin
S: Sterility

### Summary of Results Lot ENG-161-001-001

Results for all tests conducted on CT-868 Active DP Engineering lot ENG-161-001-001 at 5±3° C. are within the proposed acceptance criteria for up to 18-months. At 25±2° C./60±5% RH, except for pH at 1-month, all tests conducted are within the proposed acceptance criteria for up to 6-months. The pH at 1-month of 7.2 is just above the upper limit of the acceptance criteria. However, at 3- and 6-months the pH is the same result as TO, 7.1. The pH result at 1-month is likely the result of analytical variability. The pH result of 7.1 at 3- and 6-months at 5±3° C. are the same pH results as at 3- and 6-months at 25 ±2° C./60±5% RH. To date, there are no measurable changes or trends observed.

### GMP CT-868 Active and Placebo DP Lots

Results for the GMP CT-868 Active DP lots are within the proposed acceptance criteria for up to 18-months at 5±3° C. and 6-months at 25±2° C./60±5% RH for Lot 161-002-001, up to 12-months at 5±3° C. and 6-months at 25±2° C./60±5% RH for Lot 161-002-002, and up to 9-months at 5±3° C. and 6-months at 25±2° C./60±5% RH for Lot 161-002-003. To date, there are no measurable changes or trends.

Results for the GMP CT-868 Placebo DP lots are within the proposed acceptance criteria for up to 18-months at 5±3° C. and 6-months at 25±2° C./60±5% RH for Lot 161-001-001 and up to 12-months at 5±3° C. and 25±2° C./60±5% RH for Lot 161-001-002.

### Conclusions

Because Lot ENG-161-001-001 is fully representative (same formulation and manufacturing process) of the GMP CT-868 15 mg/mL Injection lots, stability data at 18-months long-term at 5±3° C. and 18-months long-term data at 5±3° C. for GMP Lot-161-002-001 support a 24-month shelf-life for the GMP CT-868 DP cartridges at long-term refrigerated storage in accordance with principles outlined in ICH Q1E. Because the Placebo cartridges are absent the active, the same shelf-life is assigned at refrigerated storage.

### In-Use Stability

In addition to the on-going stability studies for the CT-868 active and CT-868 Placebo DPs, a simulated in-use study at refrigerated storage (5±3° C.) was completed as part of design verification to support use of CT-868 Injection, 15 mg/mL assembled with the CT-868 Pen Injector components for up to 31-days as intended in clinical trials. A GMP CT-868 Injection, 15 mg/mL cartridge Lot 161-002-001 assembled with the CT-868 device components was stored refrigerated, 5±3° C. Liquid expelled from the Pen through the BD 4 mm×32 Gauge Nano 2nd Gen pen needle was tested as described in Table 34.

TABLE 34
Simulated In-Use Study (31 days)—CT-868 Pen Injector

| | Time Points (days) | | | | |
|---|---|---|---|---|---|
| | Initial | | | | |
| Storage Condition | (T0) | 1 | 2-16 | 17 | 31 |
| 5 ± 3° C. | XS | | | X | X |
| (Horizontal) | | | | | |

Notes:
T0 = Control
X = Visual (Appearance, Color, Clarity), pH, Osmolality, Particulate Matter, Strength, Impurities
S = Sterility

To support the CT-868 Pen Injector in-use period of up to 31 days, CT-868 Pen Injectors, 15 mg/mL were stored at 5±3° C., tested (T0) and returned to 5±3° C. storage. On day 1, the pens were removed from 5±3° C. storage and held at room temperature for approximately 30 minutes. The cap was removed from the pen, needle attached, dose dialed to “1.1 mg”, and liquid expelled. The needle was then removed, cap replaced, and pens returned to 5±3° C. storage. On days 2-16, the same procedure as day 1 was repeated except no liquid was expelled from the pens. On day 17 and day 31, the same procedure as day 1 was repeated and the dose dialed to “4.1 mg” to expel any remaining liquid. Testing on the expelled liquid was conducted as shown in Table 34 on day 17 and day 31 samples.

Based on the results, the in-use period is established for up to 31-days. All data meet the proposed acceptance criteria for the parameters tested.

Stability Data [CT-868 Injection, 15 mg/mL]

Stability data to support use of CT-868 Injection, 15 mg/mL and CT-868 Injection, Placebo drug product (DP) cartridges in clinical trials are being generated in accordance with the stability studies described earlier. Data generated to date are provided in Table 35-Table 42 for CT-868 Injection, 15 mg/mL DP and Table 43-Table 46 for CT-868 Injection, Placebo DP. The tests and acceptance criteria in the data tables are those approved for use at the time of testing and defined in the respective stability protocols. In addition, as part of design verification a simulated in-use study is completed using the CT-868 Pen Injector, 15 mg/mL (CT-868 Injection, 15 mg/mL DP cartridge lot 161-002-001 assembled with the CT-868 device components) to support up to 31-days in-use of CT-868 Injection, 15 mg/mL for clinical trials (Table 47).

TABLE 35
Stability Data for CT-868 Injection, 15 mg/mL Cartridge at 5° C. ± 3° C.
[Lot ENG-161-001-001]

| | | Results | | | | | |
|---|---|---|---|---|---|---|---|
| | Acceptance | Storage Time (Months) | | | | | |
| Test | Criteria | 0 [1] | 3 | 6 | 9 | 12 | 18 |

Appearance

| | | | | | | | |
|---|---|---|---|---|---|---|---|
| Visual | Clear liquid, | Essentially | Clear | Clear | Clear | Clear | Liquid, |
| | essentially | free of | liquid, | liquid, | liquid, | liquid, | essentially |
| | free of | visible | essentially | essentially | free of | essentially | free of |
| | visible | particles | free of | free of | visible | free of | visible |
| | particles | | visible | visible | particles | visible | particles |
| | | | particles | particles | | particles | |
| Clarity | ≤Reference | Clear | Clear | Clear | Clear | Clear | Clear |
| | Suspension | | | | | | |
| | II (Clear) | | | | | | |
| Color | Not more | Colorless | Colorless | Colorless | Colorless | Colorless | Colorless |
| | intense | | | | | | |
| | color than | | | | | | |
| | reference | | | | | | |
| | solution Y5 | | | | | | |
| | (Colorless) | | | | | | |

General Tests

| | | | | | | | |
|---|---|---|---|---|---|---|---|
| pH | 6.9-7.1 | 7.1 | 7.1 | 7.1 | 7.1 | 7.1 | 7.1 |
| Osmolality | Report | 307 | 304 | 306 | 304 | 305 | 308 |
| (mOsm/kg) | Results | | | | | | |
| Particulate | ≥10 μm: | 54 | 105 | 136 | 102 | 213 | 153 |
| matter | ≤6000 per | | | | | | |
| (Sub- | container | | | | | | |
| visible | ≥25 μm: | 2 | 9 | 32 | 29 | 22 | 35 |
| particles) | ≤600 per | | | | | | |
| | container | | | | | | |

Strength

| | | | | | | | |
|---|---|---|---|---|---|---|---|
| Assay: | 90.0%- | 104.9 | 103.2 | 105.7 | 106.3 | 101.2 | 99.4 [3] |
| CT-868 | 110.0% | | | | | | |
| Injection | Label Claim | | | | | | |
| 15 mg/mL | | | | | | | |

Purity

| | | | | | | | |
|---|---|---|---|---|---|---|---|
| Individual | No impurity | ND | ND | (0.990) 0.50 | (0.99) 0.46 | (0.99) 0.48 | IND |
| Impurities | ≥5.0% Area | | | | | | |
| | Report all | | | | | | |
| | impurities | | | | | | |
| | ≥0.5% Area | | | | | | |
| Total | ≤7.0 % | 1.5 | 0.0 | 0.5 | 0.5 | 0.5 | 0.0 |
| Impurities | Area | | | | | | |

Microbial [2]

| | | | | | | | |
|---|---|---|---|---|---|---|---|
| Bacterial | ≤5 EU/mL | <2.50 | NS | NS | NS | <3 EU/mL | INS |
| Endotoxin | | EU/mL | | | | | |
| Sterility | No Growth | No Growth | NS | NS | NS | No Growth | NS |

[1] Release data
[2] Annual Testing
[3] The assay calculation was corrected in the method to include peptide content in the calculation. The peptide content was inadvertently omitted in the calculation in the method used in the previous timepoints;
NS = Not scheduled

TABLE 36
Stability Data for CT-868 Injection, 15 mg/mL Cartridge at 25° C. ± 2° C./60% RH
[Lot ENG-161-001-001]

| | | Results | | | |
|---|---|---|---|---|---|
| | | Storage Time (Months) | | | |
| Test | Acceptance Criteria | 0[1] | 1 | 3 | 6 |

Appearance

| | | | | | |
|---|---|---|---|---|---|
| Visual | Clear liquid, essentially free | Essentially | Clear | Clear | Clear |
| | of visible particles | free of | liquid, | liquid, | liquid, |
| | | visible | essentially | essentially | free of |
| | | particles | free of | free of | visible |
| | | | visible | visible | particles |
| | | | particles | particles | |
| Clarity | ≤Reference Suspension II | Clear | Clear | Clear | Clear |
| | (Clear) | | | | |
| Color | Not more intense color | Colorless | Colorless | Colorless | Colorless |
| | than reference solution | | | | |
| | Y5 (Colorless) | | | | |

General Tests

| | | | | | |
|---|---|---|---|---|---|
| pH | 6.9-7.1 | 7.1 | 7.2 | 7.1 | 7.1 |
| Osmolality | Report Results | 304 | 305 | 305 | 305 |
| (mOsm/kg) | | | | | |
| Particulate | ≥10 μm: ≤6000 per | 105 | 87 | 132 | 160 |
| matter (Sub- | container | | | | |
| visible | ≥25 μm: ≤600 per | 9 | 18 | 28 | 30 |
| particles) | container | | | | |

Strength

| | | | | | |
|---|---|---|---|---|---|
| Assay: | 90.0%-110.0% Label | 103.2 | 103.6 | 105.8 | 104.6 |
| CT-868 | Claim | | | | |
| Injection | | | | | |
| 15 mg/mL | | | | | |

Purity

| | | | | | |
|---|---|---|---|---|---|
| Individual | No impurity ≥5.0% Area | ND | ND | (0.99) 0.47 | (0.99) 0.48 |
| Impurities | Report all impurities ≥0.5% | | | (1.05) 0.60 | (1.04) 1.1 |
| | Area | | | | |
| Total Impurities | ≤7.0% Area | 0.0 | 0.0 | 1.1 | 1.6 |

Microbial

| | | | | | |
|---|---|---|---|---|---|
| Bacterial | ≤5 EU/mL | NS | NS | NS | <3 EU/mL |
| Endotoxin | | | | | |
| Sterility | No Growth | NS | NS | NS | No Growth |

[1] Data from 5° C. ± 3° C., T = 3M NS = Not Scheduled

TABLE 37
Stability Data for CT-868 Injection, 15 mg/mL Cartridge at 5° C. ± 3° C.
[Lot 161-002-001]

| | | Results | | | | | |
|---|---|---|---|---|---|---|---|
| | Acceptance | Storage Time (Months) | | | | | |
| Test | Criteria | 0 [1] | 3 | 6 | 9 | 12 | 18 |

Appearance

| | | | | | | | |
|---|---|---|---|---|---|---|---|
| Visual | Clear | Clear | Clear | Clear | Clear | Clear | Liquid, |
| | liquid, | liquid, | liquid, | liquid, | liquid, | liquid, | essentially |
| | essentially | essentially | essentially | essentially | essentially | essentially | free of |
| | free of | free of | free of | free of | free of | free of | visible |
| | visible | visible | visible | visible | visible | visible | particles |
| | particles | particles | particles | particles | particles | particles | |
| Clarity | ≤Reference | Clear | Clear | Clear | Clear | Clear | Clear |
| | Suspension | | | | | | |
| | II (Clear) | | | | | | |
| Color | Not more | Colorless | Colorless | Colorless | Colorless | Colorless | Colorless |
| | intense | | | | | | |
| | color than | | | | | | |
| | reference | | | | | | |
| | solution | | | | | | |
| | Y5 | | | | | | |
| | (Colorless) | | | | | | |

General Tests

| | | | | | | | |
|---|---|---|---|---|---|---|---|
| pH | 6.9-7.1 | 7.0 | 7.0 | 7.0 | 7.0 | 7.0 | 7.0 |
| Osmolality | Report | 296 | 295 | 296 | 296 | 295 | 296 |
| (mOsm/kg) | Results | | | | | | |
| Particulate | ≥10 μm: | 169 | 121 | 162 | 264 | 298 | 425 |
| matter | ≤6000 per | | | | | | |
| (Sub- | container | | | | | | |
| visible | ≥25 μm: | 1 | 9 | 21 | 17 | 33 | 48 |
| particles) | ≤600 per | | | | | | |
| | container | | | | | | |

Strength

| | | | | | | | |
|---|---|---|---|---|---|---|---|
| Assay: | 90.0%- | 107.2 | 107.4 | 105.9 | 103.3 | 102.1 | 101.8 |
| CT-868 | 110.0% | | | | | | |
| Injection | Label | | | | | | |
| 15 mg/mL | Claim | | | | | | |

Purity

| | | | | | | | |
|---|---|---|---|---|---|---|---|
| Individual | No | — | — | — | — | — | — |
| Impurities | impurity | | | | | | |
| | ≥5.0% Area | | | | | | |
| | Report all | — | — | 0.0 | 0.0 | 0.0 | (0.99) 0.47 |
| | impurities | | | | | | |
| | ≥0.5% Area | | | | | | |
| Total | ≤7.0% | 0.0 | 0.0 | 0.0 | 0.0 | 0.0 | 0.5 |
| Impurities | Area | | | | | | |

Microbial [2]

| | | | | | | | |
|---|---|---|---|---|---|---|---|
| Bacterial | ≤5 EU/mL | <3 EU/mL | NS | NS | NS | <3 EU/mL | NS |
| Endotoxin | | | | | | | |
| Sterility | No Growth | No Growth | NS | NS | NS | No Growth | NS |

[1] Release data
[2] Annual Testing;
NS = Not scheduled

TABLE 38
Stability Data for CT-868 Injection, 15 mg/mL Cartridge at 25° C. ± 2° C./60% RH
[Lot 161-002-001] (Completed)

| | | Results | | | |
|---|---|---|---|---|---|
| | | Storage Time (Months) | | | |
| Test | Acceptance Criteria | 0[1] | 1 | 3 | 6 |

Appearance

| | | | | | |
|---|---|---|---|---|---|
| Visual | Clear liquid, essentially free | Clear liquid, | Clear liquid, | Clear liquid, | Clear liquid, |
| | of visible particles | essentially | essentially | essentially | essentially |
| | | free of | free of | free of | free of |
| | | visible | visible | visible | visible |
| | | particles | particles | particles | particles |
| Clarity | ≤Reference Suspension II | Clear | Clear | Clear | Clear |
| | (Clear) | | | | |
| Color | Not more intense color | Colorless | Colorless | Colorless | Colorless |
| | than reference solution Y5 | | | | |
| | (Colorless) | | | | |

General Tests

| | | | | | |
|---|---|---|---|---|---|
| pH | 6.9-7.1 | 7.0 | 7.0 | 7.0 | 7.0 |
| Osmolality | Report Results | 296 | 295 | 293 | 296 |
| (mOsm/kg) | | | | | |
| Particulate | ≥10 μm: ≤6000 per | 169 | 136 | 170 | 266 |
| matter (Sub- | container | | | | |
| visible | ≥25 μm: ≤600 per | 1 | 13 | 17 | 32 |
| particles) | container | | | | |

Strength

| | | | | | |
|---|---|---|---|---|---|
| Assay: | 90.0%-110.0% Label | 107.2 | 106.8 | 106.7 | 103.9 |
| CT-868 | Claim | | | | |
| Injection | | | | | |
| 15 mg/mL | | | | | |

Purity

| | | | | | |
|---|---|---|---|---|---|
| Individual | No impurity ≥5.0% Area | — | — | — | — |
| Impurities | Report all impurities ≥0.5% | ND | ND | ND | (0.98) 0.46 |
| | Area | | | | |
| Total Impurities | ≤7.0% Area | 0.0 | 0.0 | 0.0 | 0.5 |

Microbial

| | | | | | |
|---|---|---|---|---|---|
| Bacterial | ≤5 EU/mL | <3 EU/mL | NS | NS | <3 EU/mL |
| Endotoxin | | | | | |
| Sterility | No Growth | No Growth | NS | NS | No Growth |

[1] Release data; NS = Not Scheduled

TABLE 39
Stability Data for CT-868 Injection, 15 mg/mL Cartridge at 5° C. ± 3° C.
[Lot 161-002-002]

| | | Results | | | | |
|---|---|---|---|---|---|---|
| | Acceptance | Storage Time (Months) | | | | |
| Test | Criteria | 0 [1] | 3 | 6 | 9 | 12 |

Appearance

| | | | | | | |
|---|---|---|---|---|---|---|
| Visual | Clear liquid, | Clear liquid, | Clear liquid, | Clear liquid, | Clear | Liquid, |
| | essentially | essentially | essentially | essentially | liquid, | essentially |
| | free of visible | free of | free of | free of | essentially | free of |
| | particles | visible | visible | visible | free of | visible |
| | | particles | particles | particles | visible | particles |
| | | | | | particles | |
| Clarity | ≤Reference | Clear | Clear | Clear | Clear | Clear |
| | Suspension | | | | | |
| | II (Clear) | | | | | |
| Color | Not more | Colorless | Colorless | Colorless | Colorless | Colorless |
| | intense color | | | | | |
| | than reference | | | | | |
| | solution Y5 | | | | | |
| | (Colorless) | | | | | |

General Tests

| | | | | | | |
|---|---|---|---|---|---|---|
| pH | 6.9-7.1 | 7.0 | 7.0 | 7.1 | 7.0 | 7.0 |
| Osmolality | Report Results | 265 | 267 | 266 | 267 | 266 |
| (mOsm/kg) | | | | | | |
| Particulate | ≥10 μm: | 78 | NS | 66 | NS | 73 |
| matter (Sub- | ≤6000 per | | | | | |
| visible | container | | | | | |
| particles) | ≥25 μm: | 3 | | 1 | | 12 |
| | ≤600 per | | | | | |
| | container | | | | | |

Strength

| | | | | | | |
|---|---|---|---|---|---|---|
| Assay: | 90.0%- | 99.8 | 98.5 | 96.1 | 94.4 | 96.0 |
| CT-868 | 110.0% Label | | | | | |
| Injection, | Claim | | | | | |
| 15 mg/mL | | | | | | |

Purity

| | | | | | | |
|---|---|---|---|---|---|---|
| Individual | No impurity | — | — | — | — | — |
| Impurities | ≥5.0% Area | | | | | |
| | Report all | (0.98) 0.57 | 0.00 | (0.98) 0.74 | (0.98) 0.67 | (0.98) 0.67 |
| | impurities | | | | | |
| | ≥0.5% Area | | | | | |
| Total | ≤7.0 % Area | 0.6 | 0.0 | 0.7 | 0.7 | 0.7 |
| Impurities | | | | | | |

Microbial [2]

| | | | | | | |
|---|---|---|---|---|---|---|
| Bacterial | ≤5 EU/mL | <3 EU/mL | NS | NS | NS | NS |
| Endotoxin | | | | | | |
| Sterility | No Growth | No Growth | NS | NS | NS | No Growth |

[1] Release data;
[2] Annual Testing
NS = Not scheduled

TABLE 40
Stability Data for CT-868 Injection, 15 mg/mL Cartridge at 25° C. ± 2° C./60% RH
[Lot 161-002-002] (Completed)

| | | Results | | | |
|---|---|---|---|---|---|
| | | Storage Time (Months) | | | |
| Test | Acceptance Criteria | 0 [1] | 1 | 3 | 6 |

Appearance

| | | | | | |
|---|---|---|---|---|---|
| Visual | Clear liquid, | Clear | Clear | Clear | Clear |
| | essentially free of | liquid, | liquid, | liquid, | liquid, |
| | visible particles | essentially | essentially | essentially | essentially |
| | | free of | free of | free of | free of |
| | | visible | visible | visible | visible |
| | | particles | particles | particles | particles |
| Clarity | ≤Reference | Clear | Clear | Clear | Clear |
| | Suspension II | | | | |
| | (Clear) | | | | |
| Color | Not more intense | Colorless | Colorless | Colorless | Colorless |
| | color than reference | | | | |
| | solution Y5 | | | | |
| | (Colorless) | | | | |

General Tests

| | | | | | |
|---|---|---|---|---|---|
| pH | 6.9-7.1 | 7.0 | 7.0 | 7.0 | 7.0 |
| Osmolality | Report Results | 265 | 266 | 266 | 267 |
| (mOsm/kg) | | | | | |
| Particulate matter | ≥10 μm: ≤6000 per | 78 | NS | NS | 172 |
| (Sub-visible | container | | | | |
| particles) | ≥25 μm: ≤600 per | 3 | | | 9 |
| | container | | | | |

Strength

| | | | | | |
|---|---|---|---|---|---|
| Assay: | 90.0%-110.0% | 99.8 | 102.0 | 98.3 | 96.6 |
| CT-868 Injection | Label Claim | | | | |
| 15 mg/mL | | | | | |

Purity

| | | | | | |
|---|---|---|---|---|---|
| Individual | No impurity ≥5.0% | — | — | — | — |
| Impurities | Area | | | | |
| | Report all impurities | (0.98) 0.57 | (0.99) 0.65 | (0.98) 0.59 | (0.98) 0.53 |
| | ≥0.5% Area | | | | |
| Total Impurities | ≤7.0% Area | 0.6 | 0.7 | 0.6 | 0.5 |

Microbial

| | | | | | |
|---|---|---|---|---|---|
| Bacterial | ≤5 EU/mL | <3 EU/mL | NS | NS | NS |
| Endotoxin | | | | | |
| Sterility | No Growth | No Growth | NS | NS | No Growth |

[1] Release data; NS = Not Scheduled

TABLE 41
Stability Data for CT-868 Injection, 15 mg/mL Cartridge at 5° C. ± 3° C.
[Lot 161-002-003]

| | | Results | | | |
|---|---|---|---|---|---|
| | | Storage Time (Months) | | | |
| Test | Acceptance Criteria | 0 [1] | 3 | 6 | 9 |

Appearance

| | | | | | |
|---|---|---|---|---|---|
| Visual | Clear liquid, essentially free | Clear | Clear | Clear | Liquid, |
| | of visible particles | liquid, | liquid, | liquid, | essentially |
| | | essentially | essentially | essentially | free of |
| | | free of | free of | free of | visible |
| | | visible | visible | visible | particles |
| | | particles | particles | particles | |
| Clarity | ≤Reference Suspension II | Clear | Clear | Clear | Clear |
| | (Clear) | | | | |
| Color | Not more intense color | Colorless | Colorless | Colorless | Colorless |
| | than reference solution Y5 | | | | |
| | (Colorless) | | | | |

General Tests

| | | | | | |
|---|---|---|---|---|---|
| pH | 6.9-7.1 | 7.1 | 7.1 | 7.1 | 7.1 |
| Osmolality (mOsm/kg) | Report Results | 302 | 302 | 302 | 303 |
| Particulate matter (Sub- | ≥10 μm: ≤6000 per | 72 | NS | 137 | NS |
| visible particles) | container | | | | |
| | ≥25 μm: ≤600 per | 2 | | 25 | |
| | container | | | | |

Strength

| | | | | | |
|---|---|---|---|---|---|
| Assay: | 90.0%-110.0% Label | 99.3 | 99.9 | 101.8 | 101.2 |
| CT-868 Injection | Claim | | | | |
| 15 mg/ml | | | | | |

Purity

| | | | | | |
|---|---|---|---|---|---|
| Individual Impurities | Report all impurities | 0.0 | 0.0 | 0.0 | 0.0 |
| | ≥0.5% Area | | | | |
| Total Impurities | ≤5.0% Area | 0.0 | 0.0 | 0.0 | 0.0 |
| Phenol Content | Report Result % Label | 100.9 | 97.3 | 99.6 | 99.9 |
| | Claim | | | | |

Microbial

| | | | | | |
|---|---|---|---|---|---|
| Bacterial Endotoxin | ≤5 EU/mL | <3 EU/mL | NS | NS | NS |
| Sterility | No Growth | No Growth | NS | NS | NS |

[1] Release data; NS = Not Scheduled

TABLE 42
Stability Data for CT-868 Injection, 15 mg/mL Cartridge at 25° C. ± 2° C./60% RH
[Lot 161-002-003] (Completed)

| | | Results | | | |
|---|---|---|---|---|---|
| | | Storage Time (Months) | | | |
| Test | Acceptance Criteria | 0 [1] | 1 | 3 | 6 |

Appearance

| | | | | | |
|---|---|---|---|---|---|
| Visual | Clear liquid, essentially | Clear | Clear | Clear | Liquid, |
| | free of visible particles | liquid, | liquid, | liquid, | essentially |
| | | essentially | essentially | essentially | free of |
| | | free of | free of | free of | visible |
| | | visible | visible | visible | particles |
| | | particles | particles | particles | |
| Clarity | ≤Reference | Clear | Clear | Clear | Clear |
| | Suspension II | | | | |
| | (Clear) | | | | |
| Color | Not more intense color | Colorless | Colorless | Colorless | Colorless |
| | than reference solution | | | | |
| | Y5 (Colorless) | | | | |

General Tests

| | | | | | |
|---|---|---|---|---|---|
| pH | 6.9-7.1 | 7.1 | 7.1 | 7.1 | 7.1 |
| Osmolality (mOsm/kg) | Report Results | 302 | 304 | 301 | 300 |
| Particulate matter | ≥10 μm: ≤6000 per | 72 | NS | NS | 315 |
| (Sub-visible | container | | | | |
| particles) | ≥25 μm: ≤600 per | 2 | | | 38 |
| | container | | | | |

Strength

| | | | | | |
|---|---|---|---|---|---|
| Assay: | 90.0%-110.0% Label | 99.3 | 98.9 | 99.1 | 99.9 |
| CT-868 Injection 15 | Claim | | | | |
| mg/mL | | | | | |

Purity

| | | | | | |
|---|---|---|---|---|---|
| Individual Impurities | No impurity ≥5.0% | — | — | — | — |
| | Area | | | | (0.98) 0.5 |
| | Report all impurities | 0.0 | 0.0 | (1.05) 0.64 | (0.99) 0.46 |
| | ≥0.5% Area | | | | (1.04) 1.0 |
| Total Impurities | ≤5.0% Area | 0.0 | 0.0 | 0.6 | 2.0 |
| Phenol Content | Report Result % Label | 100.9 | 100.7 | 99.9 | 99.2 |
| | Claim | | | | |

Microbial [2]

| | | | | | |
|---|---|---|---|---|---|
| Bacterial Endotoxin | ≤5 EU/mL | <3 EU/mL | NS | NS | NS |
| Sterility | No Growth | No Growth | NS | NS | No Growth |

[1] Release data; [2] Annual Testing; NS = Not scheduled

TABLE 43
Stability Data for CT-868 Injection, Placebo Cartridge at 5° C. ± 3° C.
[Lot 161-001-001]

| | | Results | | | | | |
|---|---|---|---|---|---|---|---|
| | Acceptance | Storage Time (Months) | | | | | |
| Test | Criteria | 0 [1] | 3 | 6 | 9 | 12 | 18 |

Appearance

| | | | | | | | |
|---|---|---|---|---|---|---|---|
| Visual | Clear | Clear | Clear | Clear | Clear | Clear | Liquid, |
| | liquid, | liquid, | liquid, | liquid, | liquid, | liquid, | essentially |
| | essentially | essentially | essentially | essentially | essentially | essentially | free of |
| | free of | free of | free of | free of | free of | free of | visible |
| | visible | visible | visible | visible | visible | visible | particles |
| | particles | particles | particles | particles | particles | particles | |
| Clarity | ≤Reference | Clear | Clear | Clear | Clear | Clear | Clear |
| | Suspension II | | | | | | |
| | (Clear) | | | | | | |
| Color | Not more | Not more | Colorless | Colorless | Colorless | Colorless | Colorless |
| | intense | intense | | | | | |
| | color than | color than | | | | | |
| | reference | reference | | | | | |
| | solution Y5 | solution | | | | | |
| | (Colorless) | Y7 | | | | | |

General Tests

| | | | | | | | |
|---|---|---|---|---|---|---|---|
| pH | 6.9-7.1 | 7.1 | 7.1 | 7.1 | 7.1 | 7.1 | 7.1 |
| Osmolality | Report | 282 | 279 | 280 | 282 | 281 | 280 |
| (mOsm/kg) | Results | | | | | | |
| Particulate | ≥10 μm: | 1 | 11 | 6 | 9 | 3 | 3 |
| matter (Sub- | ≤6000 per | | | | | | |
| visible | container | | | | | | |
| particles) | ≥25 μm: | 0 | 4 | 1 | 1 | 2 | 0 |
| | ≤600 per | | | | | | |
| | container | | | | | | |

Microbial

| | | | | | | | |
|---|---|---|---|---|---|---|---|
| Bacterial | ≤5 EU/mL | <1 EU/mL | NS | NS | NS | <1 EU/mL | NS |
| Endotoxin | | | | | | | |
| Sterility | No Growth | No Growth | NS | NS | NS | No Growth | NS |

[1] Release data; NS = Not scheduled

TABLE 44
Stability Data for CT-868 Injection, Placebo Cartridge at 25° C. ± 2° C./60% RH
[Lot 161-001-001] (Completed)

| | | Results | | | |
|---|---|---|---|---|---|
| | Acceptance | Storage Time (Months) | | | |
| Test | Criteria | 0[1] | 1 | 3 | 6 |

Appearance

| | | | | | |
|---|---|---|---|---|---|
| Visual | Clear liquid, | Clear | Clear | Clear | Clear |
| | essentially free of | liquid, | liquid, | liquid, | liquid, |
| | visible particles | essentially | essentially | essentially | essentially |
| | | free of | free of | free of | free of |
| | | visible | visible | visible | visible |
| | | particles | particles | particles | particles |
| Clarity | ≤Reference | Clear | Clear | Clear | Clear |
| | Suspension II | | | | |
| | (Clear) | | | | |
| Color | Not more intense | Not more | Colorless | Colorless | Colorless |
| | color than reference | intense | | | |
| | solution Y5 | color than | | | |
| | (Colorless) | reference | | | |
| | | solution | | | |
| | | Y7 | | | |

General Tests

| | | | | | |
|---|---|---|---|---|---|
| pH | 6.9-7.1 | 7.1 | 7.1 | 7.1 | 7.1 |
| Osmolality | Report Results | 282 | 279 | 279 | 279 |
| (mOsm/kg) | | | | | |
| Particulate matter | ≥10 μm: ≤6000 | 1 | 1 | 9 | 5 |
| (Sub-visible | per container | | | | |
| particles) | ≥25 μm: ≤600 per | 0 | 0 | 3 | 1 |
| | container | | | | |

Microbial

| | | | | | |
|---|---|---|---|---|---|
| Bacterial Endotoxin | ≤5 EU/mL | <1 EU/mL | NS | NS | <1 EU/mL |
| Sterility | No Growth | No Growth | NS | NS | No Growth |

[1] Release data; NS = Not Scheduled

TABLE 45
Stability Data for CT-868 Injection, Placebo Cartridge at 5° C. ± 3° C.
[Lot 161-001-002]

| | | Results | | | | |
|---|---|---|---|---|---|---|
| | Acceptance | Storage Time (Months) | | | | |
| Test | Criteria | 0 [1] | 3 | 6 | 9 | 12 |

Appearance

| | | | | | | |
|---|---|---|---|---|---|---|
| Visual | Clear liquid, | Clear liquid, | Clear liquid, | Clear liquid, | Clear liquid, | Liquid, |
| | essentially free | essentially | essentially | essentially | essentially | essentially |
| | of visible | free of | free of | free of | free of | free of |
| | particles | visible | visible | visible | visible | visible |
| | | particles | particles | particles | particles | particles |
| Clarity | ≤Reference | Clear | Clear | Clear | Clear | Clear |
| | Suspension II | | | | | |
| | (Clear) | | | | | |
| Color | Not more | Colorless | Colorless | Colorless | Colorless | Colorless |
| | intense color | | | | | |
| | than reference | | | | | |
| | solution Y5 | | | | | |
| | (Colorless) | | | | | |

General Tests

| | | | | | | |
|---|---|---|---|---|---|---|
| pH | 6.9-7.1 | 6.9 | 7.0 | 7.0 | 6.9 | 6.9 |
| Osmolality | Report Results | 291 | 293 | 293 | 295 | 291 |
| (mOsm/kg) | | | | | | |
| Particulate | ≥10 μm: ≤6000 | 5 | NS | 1 | NS | 5 |
| matter (Sub- | per container | | | | | |
| visible | ≥25 μm: ≤600 | 0 | | 1 | | 1 |
| particles) | per container | | | | | |

Microbial [2]

| | | | | | | |
|---|---|---|---|---|---|---|
| Bacterial | ≤5 EU/mL | <1 EU/mL | NS | NS | NS | NS |
| Endotoxin | | | | | | |
| Sterility | No Growth | No Growth | NS | NS | NS | No Growth |

[1] Release data
[2] Annual Testing
NS = Not scheduled

TABLE 46
Stability Data for CT-868 Injection, Placebo Cartridge at 25° C. ± 2° C./60% RH
[Lot 161-001-002] (Completed)

| | | Results | | | |
|---|---|---|---|---|---|
| | Acceptance | Storage Time (Months) | | | |
| Test | Criteria | 0 [1] | 1 | 3 | 6 |

Appearance

| | | | | | |
|---|---|---|---|---|---|
| Visual | Clear liquid, | Clear liquid, | Clear liquid, | Clear liquid, | Clear liquid, |
| | essentially free of | essentially | essentially | essentially | essentially |
| | visible particles | free of visible | free of visible | free of visible | free of visible |
| | | particles | particles | particles | particles |
| Clarity | ≤Reference | Clear | Clear | Clear | Clear |
| | Suspension II | | | | |
| | (Clear) | | | | |
| Color | Not more intense | Colorless | Colorless | Colorless | Colorless |
| | color than | | | | |
| | reference | | | | |
| | solution Y5 | | | | |
| | (Colorless) | | | | |

General Tests

| | | | | | |
|---|---|---|---|---|---|
| pH | 6.9-7.1 | 6.9 | 7.0 | 7.0 | 7.0 |
| Osmolality | Report Results | 291 | 292 | 292 | 298 |
| (mOsm/kg) | | | | | |
| Particulate | ≥10 μm: ≤6000 | 5 | NS | NS | 2 |
| matter (Sub- | per container | | | | |
| visible | ≥25 μm: ≤600 | 0 | | | 1 |
| particles) | per container | | | | |

Microbial

| | | | | | |
|---|---|---|---|---|---|
| Bacterial | ≤5 EU/mL | <1 EU/mL | NS | NS | NS |
| Endotoxin [2] | | | | | |
| Sterility | No Growth | No Growth | NS | NS | No Growth |

[1] Release data [2] Annual Testing NS = Not Scheduled

TABLE 47
Simulated In-Use Data CT-868 Pen Injector, 15 mg/mL
(CT-868 Injection 15 mg/mL cartridge lot) (Completed)

| | | Results | | |
|---|---|---|---|---|
| | | Storage Time (Days) | | |
| Test | Acceptance Criteria | 0 [1] | 17 | 31 |

Appearance

| | | | | |
|---|---|---|---|---|
| Visual | Clear liquid, essentially | Clear | Clear liquid, | Clear |
| | free of visible particles | liquid, | essentially | liquid, |
| | | essentially | free of visible | essentially |
| | | free of | particles | free of |
| | | visible | | visible |
| | | particles | | particles |
| Clarity | ≤Reference Suspension | Clear | Clear | Clear |
| | II (Clear) | | | |
| Color | Not more intense color | Colorless | Colorless | Colorless |
| | than reference solution | | | |
| | Y5 (Colorless) | | | |

General Tests

| | | | | |
|---|---|---|---|---|
| pH | 6.9-7.1 | 7.0 | 7.0 | 7.0 |
| Osmolality (mOsm/kg) | Report Results | 297 | 293 | 297 |
| Particulate matter (Sub- | ≥10 μm: ≤6000 per | 508 | 1923 | 480 |
| visible particles) | container | | | |
| | ≥25 μm: ≤600 per | 13 | 36 | 8 |
| | container | | | |

Strength

| | | | | |
|---|---|---|---|---|
| Assay: | 90.0%-110.0% Label | 107.7 | 106.2 | 104.2 |
| CT-868 Injection 15 mg/ml | Claim | | | |

Purity

| | | | | |
|---|---|---|---|---|
| Individual Impurities | No impurity ≥5.0% | 0 | 0 | 0 |
| | Area | | | |
| | Report all impurities | 0 | 0 | 0 |
| | ≥0.5% Area | | | |
| Total Impurities | ≤7.0% Area | 0.0 | 0.0 | 0.0 |

Microbial

| | | | | |
|---|---|---|---|---|
| Sterility | No Growth | No Growth | NS | NS |

[1] Initial; NS = Not Scheduled

1. A method of treating type 1 diabetes mellitus (T1DM) in a patient in need thereof, comprising administering by subcutaneous injection to the patient in need thereof a compound comprising the following structure or a pharmaceutically acceptable salt or ester thereof: 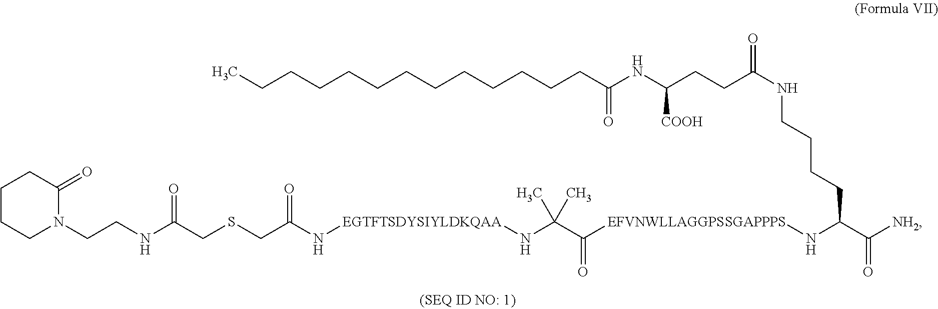
wherein the compound is administered at a body weight-independent dose of 1 mg to 20 mg.

2. A method of treating type 2 diabetes mellitus (T2DM) in a patient in need thereof, comprising administering by subcutaneous injection to the patient in need thereof a compound comprising the following structure or a pharmaceutically acceptable salt or ester thereof: 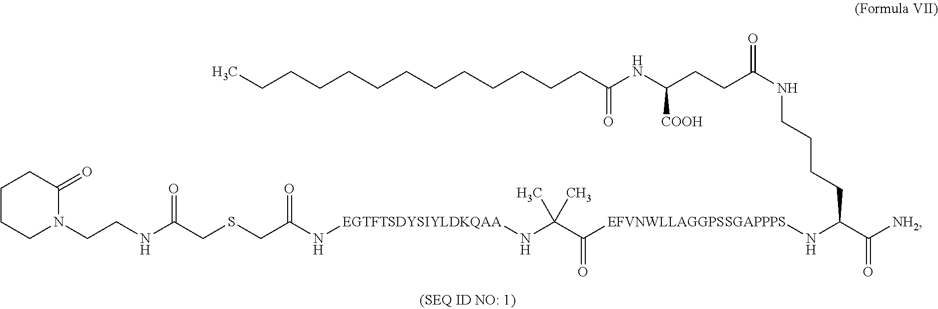
wherein the compound is administered at a body weight-independent dose of 1 mg to 20 mg.

3. The method of claim 1, where the method is used to improve glycemic control in the patient,
increase insulin sensitivity in the patient,
increase insulin-independent glucose disposal in the patient,
reduce the need for therapeutic insulin in the patient,
alleviate hypertension in the patient, and/or
reduce atherogenic lipids in the patient.

4. The method of claim 1, wherein the patient is overweight or obese, and the method reduces body weight of the patient.

5. A method of weight management in a patient in need thereof, comprising administering by subcutaneous injection to the patient in need thereof a compound comprising the following structure or a pharmaceutically acceptable salt or ester thereof: 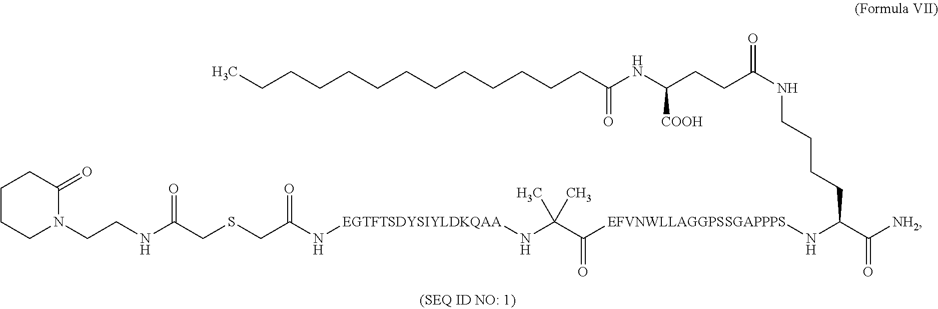
wherein the compound is administered at a body weight-independent dose of 1 mg to 20 mg.

6. The method of claim 5, wherein the patient has type 1 or type 2 diabetes mellitus.

7. The method of claim 1, wherein the administration of the compound is an adjunct to one or more additional therapies.

8. The method of claim 7, wherein the one or more additional therapies comprise insulin therapy, diet therapy, exercise therapy, hypertension therapy, and/or blood lipid-lowering therapy.

9. The method of claim 1, wherein the administering step is repeated at an interval of one to seven days.

10. The method of claim 1, wherein the administering step is repeated once daily.

11. The method of claim 1, wherein the compound is administered at a dose that agonizes both GLP-1 receptor and GIP receptor.

12. The method of claim 1, wherein the dose is about 1.5, 1.8, 2, 3, 4, 5, 6, 7, 8, 9, 10, 11, 12, 13, 14, 15, 16, 17, 18, 19, or 20 mg.

13. The method of claim 1, wherein the dose is 1.77, 3.25 mg, or 4 mg.

14. The method of claim 1, wherein the dose is 1.1, 1.8, 2.6, 3.3, 4.1, 5.2, or 6.6 mg.

15. The method of claim 1, wherein the patient has a BMI of 25 kg/m² or higher.

16. The method of claim 1, wherein the compound is administered to the patient in a pharmaceutical composition comprising:
the compound at a concentration of 15 mg/mL,
di-sodium hydrogen phosphate heptahydrate/sodium dihydrogen phosphate monobasic buffer solution, optionally at a concentration of 20 mM,
propylene glycol, and
phenol, further optionally wherein the pharmaceutical composition is at pH 7.0.

17. A pharmaceutical composition comprising
(i) a compound comprising the following structure or a pharmaceutically acceptable salt or ester thereof, at a concentration of 15 mg/mL: 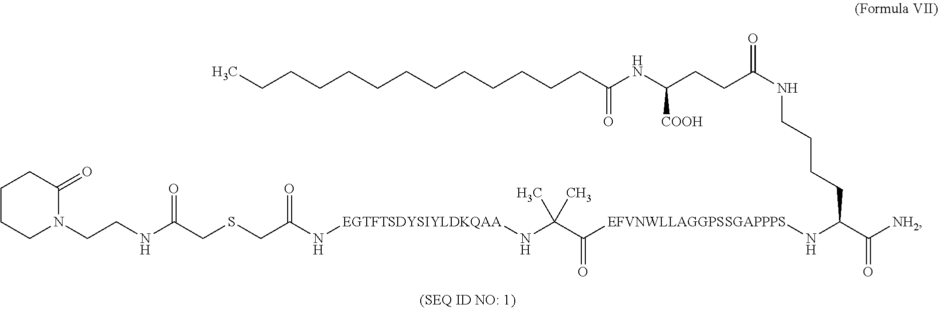
(ii) di-sodium hydrogen phosphate heptahydrate/sodium dihydrogen phosphate monobasic buffer solution, optionally at a concentration of 20 mM,
(iii) propylene glycol, and
(iv) phenol, further optionally wherein the pharmaceutical composition is at pH 7.0.

18-19. (canceled)

20. An article of manufacture comprising one or more units of said dose, optionally a single dose unit, of claim 1.

21-22. (canceled)

23. The article of manufacture of claim 20, wherein the article of manufacture comprises a needle-based injection system with an integrated non-replaceable 3-mL Type 1 glass cartridge and a pharmaceutical composition comprising 3 mL of the agonist at a concentration of 15 mg/mL.


SEQUENCE LISTING (3 sequences)

SEQ ID NO: 1 - protein, 38 aa, synthetic construct
  MOD_RES 18: note = Aib
  MOD_RES 38: note = N6-[N-(1-oxohexadecyl)-L-gamma-glutamyl]-L-lysinami de
EGTFTSDYSIYLDKQAAXEFVNWLLAGGPSSGAPPPSX

SEQ ID NO: 2 - protein, 39 aa, synthetic construct
FGEGTFTSDLSKQMEEEAVRLFIEWLLAGGPSSGAPPPS

SEQ ID NO: 3 - protein, 38 aa, synthetic construct
  MOD_RES 18: note = Aib
EGTFTSDYSIYLDKQAAXEFVNWLLAGGPSSGAPPPSK